SEQUENCE LISTING (5 sequences)

SEQ ID NO: 1 - DNA, 240825 bp, Homo sapiens
  misc_feature 1..2000: 5′regulatory region
  exon 2001..2079: exon A
  exon 4627..4718: exon B
  exon 10115..10233: exon C
  exon 26810..26897: exon D
  exon 31357..31471: exon E
  exon 34261..34404: exon F
  exon 37377..37466: exon S
  exon 39704..40858: exon T
  exon 50436..50545: exon G
  exon 72881..72918: exon H
  exon 75989..76151: exon I
  exon 95111..95188: exon J
  exon 216015..216252: exon K
  exon 237526..238825: exon L
  misc_feature 238826..240825: 3′regulatory region
  allele 1999: 5-390-177  : polymorphic base G or C
  allele 4601: 5-391-43  : polymorphic base A or G
  allele 10228: 5-392-222  : polymorphic base G or T
  allele 10286: 5-392-280  : polymorphic base G or T
  allele 10370: 5-392-364  : insertion of G
  allele 39944: 4-58-318  : polymorphic base G or T
  allele 39973: 4-58-289  : polymorphic base G or C
  allele 41385: 4-54-199  : polymorphic base A or C
  allele 41404: 4-54-180  : polymorphic base A or C
  allele 42232: 4-51-312  : polymorphic base G or C
  allele 67475: 99-86-266  : polymorphic base A or G
  allele 69521: 4-88-107  : polymorphic base A or G
  allele 72838: 5-397-141  : polymorphic base G or T
  allele 76060: 5-398-203  : polymorphic base A or C
  allele 81253: 99-12738-248  : polymorphic base A or C
  allele 83921: 99-109-358  : polymorphic base A or C
  allele 91917: 99-12749-175  : polymorphic base C or T
  allele 95349: 4-21-154  : polymorphic base C or T
  allele 95511: 4-21-317  : polymorphic base G or T
  allele 96190: 4-23-326  : polymorphic base A or G
  allele 97294: 99-12753-34  : polymorphic base A or T
  allele 98024: 5-364-252  : polymorphic base G or T
  allele 98914: 99-12755-280  : polymorphic base A or G
  allele 98963: 99-12755-329  : polymorphic base A or C
  allele 103593: 4-87-212  : polymorphic base A or G
  allele 104398: 99-12757-318  : polymorphic base C or T
  allele 106373: 99-12758-102  : polymorphic base A or G
  allele 106407: 99-12758-136  : polymorphic base C or T
  allele 108315: 4-105-98  : polymorphic base A or G
  allele 108327: 4-105-86  : polymorphic base A or G
  allele 108472: 4-45-49  : polymorphic base C or T
  allele 109196: 4-44-277  : polymorphic base C or T
  allele 114604: 4-86-60  : polymorphic base G or C
  allele 115716: 4-84-334  : polymorphic base A or G
  allele 122083: 99-78-321  : polymorphic base A or T
  allele 123124: 99-12767-36  : polymorphic base G or C
  allele 123231: 99-12767-143  : polymorphic base C or T
  allele 123277: 99-12767-189  : polymorphic base C or T
  allele 123468: 99-12767-380  : polymorphic base A or G
  allele 126738: 4-80-328  : polymorphic base C or T
  allele 128210: 4-36-384  : polymorphic base G or C
  allele 128330: 4-36-264  : polymorphic base A or G
  allele 128333: 4-36-261  : polymorphic base A or C
  allele 128594: 4-35-333  : polymorphic base A or C
  allele 128687: 4-35-240  : polymorphic base G or C
  allele 128754: 4-35-173  : polymorphic base A or T
  allele 128794: 4-35-133  : polymorphic base C or T
  allele 130805: 99-12771-59  : polymorphic base G or T
  allele 133206: 99-12774-334  : polymorphic base A or C
  allele 135386: 99-12776-358  : polymorphic base A or G
  allele 139389: 99-12781-113  : polymorphic base A or G
  allele 157535: 4-104-298  : polymorphic base G or C
  allele 157579: 4-104-254  : polymorphic base A or G
  allele 157583: 4-104-250  : polymorphic base C or T
  allele 157619: 4-104-214  : polymorphic base A or G
  allele 172980: 99-12818-289  : polymorphic base C or T
  allele 180622: 99-24807-271  : polymorphic base C or T
  allele 180809: 99-24807-84  : polymorphic base A or G
  allele 190334: 99-12831-157  : polymorphic base A or G
  allele 190418: 99-12831-241  : polymorphic base C or T
  allele 191397: 99-12832-387  : polymorphic base C or T
  allele 195128: 99-12836-30  : polymorphic base G or C
  allele 203846: 99-12844-262  : polymorphic base G or C
  allele 210151: 4-24-74  : polymorphic base C or T
  allele 210321: 4-24-246  : polymorphic base C or T
  allele 210389: 4-24-314  : polymorphic base G or C
  allele 211168: 4-27-190  : polymorphic base A or G
  allele 215996: 5-400-145  : polymorphic base A or G
  allele 216000: 5-400-149  : polymorphic base G or C
  allele 216026: 5-400-175  : polymorphic base C or T
  allele 216082: 5-400-231  : polymorphic base C or T
  allele 216218: 5-400-367  : polymorphic base A or C
  allele 216322: 99-12852-110  : polymorphic base G or T
  allele 216537: 99-12852-325  : polymorphic base A or G
  allele 221649: 4-37-326  : polymorphic base A or C
  allele 221867: 4-37-107  : polymorphic base A or G
  allele 225645: 5-270-92  : polymorphic base G or C
  allele 229387: 99-12860-47  : polymorphic base A or G
  allele 229397: 99-12860-57  : polymorphic base A or T
  allele 237555: 5-402-144  : polymorphic base C or T
  primer_bind 1823..1840: 5-390.pu
  primer_bind 2108..2125: 5-390.rp  complement
  primer_bind 4559..4577: 5-391.pu
  primer_bind 4891..4908: 5-391.rp  complement
  primer_bind 10007..10025: 5-392.pu
  primer_bind 10411..10430: 5-392.rp  complement
  primer_bind 39556..39574: 4-59.rp
  primer_bind 39877..39896: 4-58.rp
  primer_bind 39953..39970: 4-59.pu complement
  primer_bind 40242..40259: 4-58.pu complement
  primer_bind 41137..41154: 4-54.rp
  primer_bind 41564..41581: 4-54.pu complement
  primer_bind 42122..42141: 4-51.rp
  primer_bind 42526..42543: 4-51.pu complement
  primer_bind 67289..67309: 99-86.rp
  primer_bind 67724..67741: 99-86.pu complement
  primer_bind 69182..69200: 4-88.rp
  primer_bind 69609..69626: 4-88.pu complement
  primer_bind 72698..72715: 5-397.pu
  primer_bind 73099..73117: 5-397.rp  complement
  primer_bind 75858..75877: 5-398.pu
  primer_bind 76289..76306: 5-398.rp  complement
  primer_bind 81006..81025: 99-12738.pu
  primer_bind 81466..81485: 99-12738.rp  complement
  primer_bind 83564..83582: 99-109.pu
  primer_bind 83990..84007: 99-109.rp  complement
  primer_bind 91743..91763: 99-12749.pu
  primer_bind 92123..92142: 99-12749.rp  complement
  primer_bind 95196..95214: 4-21.pu
  primer_bind 95600..95619: 4-21.rp  complement
  primer_bind 95865..95882: 4-23.pu
  primer_bind 96210..96229: 4-23.rp  complement
  primer_bind 97261..97278: 99-12753.pu
  primer_bind 97728..97747: 99-12753.rp  complement
  primer_bind 97831..97849: 5-364.rp
  primer_bind 98256..98275: 5-364.pu complement
  primer_bind 98638..98656: 99-12755.pu
  primer_bind 99111..99131: 99-12755.rp  complement
  primer_bind 103376..103395: 4-87.rp
  primer_bind 103801..103818: 4-87.pu complement
  primer_bind 104081..104100: 99-12757.pu
  primer_bind 104619..104636: 99-12757.rp  complement
  primer_bind 106272..106291: 99-12758.pu
  primer_bind 106780..106799: 99-12758.rp  complement
  primer_bind 108200..108218: 4-105.rp
  primer_bind 108223..108246: 4-45.rp
  primer_bind 108390..108412: 4-105.pu complement
  primer_bind 108499..108520: 4-45.pu complement
  primer_bind 109123..109142: 4-44.rp
  primer_bind 109454..109471: 4-44.pu complement
  primer_bind 114217..114234: 4-86.rp
  primer_bind 114646..114663: 4-86.pu complement
  primer_bind 115630..115647: 4-84.rp
  primer_bind 116031..116049: 4-84.pu complement
  primer_bind 121991..122011: 99-78.rp
  primer_bind 122384..122401: 99-78.pu complement
  primer_bind 123089..123106: 99-12767.pu
  primer_bind 123565..123583: 99-12767.rp  complement
  primer_bind 126711..126729: 4-80.rp
  primer_bind 127048..127065: 4-80.pu complement
  primer_bind 128162..128179: 4-36.rp
  primer_bind 128480..128497: 4-35.rp
  primer_bind 128573..128590: 4-36.pu complement
  primer_bind 128909..128926: 4-35.pu complement
  primer_bind 130747..130764: 99-12771.pu
  primer_bind 131254..131273: 99-12771.rp  complement
  primer_bind 132873..132892: 99-12774.pu
  primer_bind 133305..133325: 99-12774.rp  complement
  primer_bind 135029..135048: 99-12776.pu
  primer_bind 135458..135478: 99-12776.rp  complement
  primer_bind 139277..139296: 99-12781.pu
  primer_bind 139724..139742: 99-12781.rp  complement
  primer_bind 157181..157199: 4-104.rp
  primer_bind 157814..157832: 4-104.pu complement
  primer_bind 172692..172709: 99-12818.pu
  primer_bind 173072..173091: 99-12818.rp  complement
  primer_bind 180248..180268: 99-24807.rp
  primer_bind 180874..180892: 99-24807.pu complement
  primer_bind 184662..184680: 99-12827.pu
  primer_bind 185138..185156: 99-12827.rp  complement
  primer_bind 190178..190196: 99-12831.pu
  primer_bind 190643..190663: 99-12831.rp  complement
  primer_bind 191011..191030: 99-12832.pu
  primer_bind 191441..191460: 99-12832.rp  complement
  primer_bind 195099..195116: 99-12836.pu
  primer_bind 195568..195587: 99-12836.rp  complement
  primer_bind 203585..203602: 99-12844.pu
  primer_bind 204095..204115: 99-12844.rp  complement
  primer_bind 210079..210096: 4-24.pu
  primer_bind 210476..210495: 4-24.rp  complement
  primer_bind 210979..210996: 4-27.pu
  primer_bind 211382..211401: 4-27.rp  complement
  primer_bind 215852..215870: 5-400.pu
  primer_bind 216213..216231: 99-12852.pu
  primer_bind 216253..216271: 5-400.rp  complement
  primer_bind 216708..216728: 99-12852.rp  complement
  primer_bind 221530..221549: 4-37.rp
  primer_bind 221956..221973: 4-37.pu complement
  primer_bind 225554..225572: 5-270.pu
  primer_bind 225827..225845: 5-270.rp  complement
  primer_bind 229341..229359: 99-12860.pu
  primer_bind 229770..229790: 99-12860.rp  complement
  primer_bind 237412..237429: 5-402.pu
  primer_bind 237747..237766: 5-402.rp  complement
  primer_bind 1980..1998: 5-390-177.mis
  primer_bind 2000..2018: 5-390-177.mis complement
  primer_bind 4582..4600: 5-391-43.mis
  primer_bind 4602..4620: 5-391-43.mis complement
  primer_bind 10209..10227: 5-392-222.mis
  primer_bind 10229..10247: 5-392-222.mis complement
  primer_bind 10267..10285: 5-392-280.mis
  primer_bind 10287..10305: 5-392-280.mis complement
  primer_bind 39925..39943: 4-58-318.mis
  primer_bind 39945..39963: 4-58-318.mis complement
  primer_bind 39954..39972: 4-58-289.mis
  primer_bind 39974..39992: 4-58-289.mis complement
  primer_bind 41366..41384: 4-54-199.mis
  primer_bind 41385..41403: 4-54-180.mis
  primer_bind 41386..41404: 4-54-199.mis complement
  primer_bind 41405..41423: 4-54-180.mis complement
  primer_bind 42213..42231: 4-51-312.mis
  primer_bind 42233..42251: 4-51-312.mis complement
  primer_bind 67456..67474: 99-86-266.mis
  primer_bind 67476..67494: 99-86-266.mis complement
  primer_bind 69502..69520: 4-88-107.mis
  primer_bind 69522..69540: 4-88-107.mis complement
  primer_bind 72819..72837: 5-397-141.mis
  primer_bind 72839..72857: 5-397-141.mis complement
  primer_bind 76041..76059: 5-398-203.mis
  primer_bind 76061..76079: 5-398-203.mis complement
  primer_bind 81234..81252: 99-12738-248.mis
  primer_bind 81254..81272: 99-12738-248.mis complement
  primer_bind 83902..83920: 99-109-358.mis
  primer_bind 83922..83940: 99-109-358.mis complement
  primer_bind 91898..91916: 99-12749-175.mis
  primer_bind 91918..91936: 99-12749-175.mis complement
  primer_bind 95330..95348: 4-21-154.mis
  primer_bind 95350..95368: 4-21-154.mis complement
  primer_bind 95492..95510: 4-21-317.mis
  primer_bind 95512..95530: 4-21-317.mis complement
  primer_bind 96171..96189: 4-23-326.mis
  primer_bind 96191..96209: 4-23-326.mis complement
  primer_bind 97275..97293: 99-12753-34.mis
  primer_bind 97295..97313: 99-12753-34.mis complement
  primer_bind 98005..98023: 5-364-252.mis
  primer_bind 98025..98043: 5-364-252.mis complement
  primer_bind 98895..98913: 99-12755-280.mis
  primer_bind 98915..98933: 99-12755-280.mis complement
  primer_bind 98944..98962: 99-12755-329.mis
  primer_bind 98964..98982: 99-12755-329.mis complement
  primer_bind 103574..103592: 4-87-212.mis
  primer_bind 103594..103612: 4-87-212.mis complement
  primer_bind 104379..104397: 99-12757-318.mis
  primer_bind 104399..104417: 99-12757-318.mis complement
  primer_bind 106354..106372: 99-12758-102.mis
  primer_bind 106374..106392: 99-12758-102.mis complement
  primer_bind 106388..106406: 99-12758-136.mis
  primer_bind 106408..106426: 99-12758-136.mis complement
  primer_bind 108296..108314: 4-105-98.mis
  primer_bind 108308..108326: 4-105-86.mis
  primer_bind 108316..108334: 4-105-98.mis complement
  primer_bind 108328..108346: 4-105-86.mis complement
  primer_bind 108453..108471: 4-45-49.mis
  primer_bind 108473..108491: 4-45-49.mis complement
  primer_bind 109177..109195: 4-44-277.mis
  primer_bind 109197..109215: 4-44-277.mis complement
  primer_bind 114585..114603: 4-86-60.mis
  primer_bind 114605..114623: 4-86-60.mis complement
  primer_bind 115697..115715: 4-84-334.mis
  primer_bind 115717..115735: 4-84-334.mis complement
  primer_bind 122064..122082: 99-78-321.mis
  primer_bind 122084..122102: 99-78-321.mis complement
  primer_bind 123105..123123: 99-12767-36.mis
  primer_bind 123125..123143: 99-12767-36.mis complement
  primer_bind 123212..123230: 99-12767-143.mis
  primer_bind 123232..123250: 99-12767-143.mis complement
  primer_bind 123258..123276: 99-12767-189.mis
  primer_bind 123278..123296: 99-12767-189.mis complement
  primer_bind 123449..123467: 99-12767-380.mis
  primer_bind 123469..123487: 99-12767-380.mis complement
  primer_bind 126719..126737: 4-80-328.mis
  primer_bind 126739..126757: 4-80-328.mis complement
  primer_bind 128191..128209: 4-36-384.mis
  primer_bind 128211..128229: 4-36-384.mis complement
  primer_bind 128311..128329: 4-36-264.mis
  primer_bind 128314..128332: 4-36-261.mis
  primer_bind 128331..128349: 4-36-264.mis complement
  primer_bind 128334..128352: 4-36-261.mis complement
  primer_bind 128575..128593: 4-35-333.mis
  primer_bind 128595..128613: 4-35-333.mis complement
  primer_bind 128668..128686: 4-35-240.mis
  primer_bind 128688..128706: 4-35-240.mis complement
  primer_bind 128735..128753: 4-35-173.mis
  primer_bind 128755..128773: 4-35-173.mis complement
  primer_bind 128775..128793: 4-35-133.mis
  primer_bind 128795..128813: 4-35-133.mis complement
  primer_bind 130786..130804: 99-12771-59.mis
  primer_bind 130806..130824: 99-12771-59.mis complement
  primer_bind 133187..133205: 99-12774-334.mis
  primer_bind 133207..133225: 99-12774-334.mis complement
  primer_bind 135367..135385: 99-12776-358.mis
  primer_bind 135387..135405: 99-12776-358.mis complement
  primer_bind 139370..139388: 99-12781-113.mis
  primer_bind 139390..139408: 99-12781-113.mis complement
  primer_bind 157516..157534: 4-104-298.mis
  primer_bind 157536..157554: 4-104-298.mis complement
  primer_bind 157560..157578: 4-104-254.mis
  primer_bind 157564..157582: 4-104-250.mis
  primer_bind 157580..157598: 4-104-254.mis complement
  primer_bind 157584..157602: 4-104-250.mis complement
  primer_bind 157600..157618: 4-104-214.mis
  primer_bind 157620..157638: 4-104-214.mis complement
  primer_bind 172961..172979: 99-12818-289.mis
  primer_bind 172981..172999: 99-12818-289.mis complement
  primer_bind 180603..180621: 99-24807-271.mis
  primer_bind 180623..180641: 99-24807-271.mis complement
  primer_bind 180790..180808: 99-24807-84.mis
  primer_bind 180810..180828: 99-24807-84.mis complement
  primer_bind 190315..190333: 99-12831-157.mis
  primer_bind 190335..190353: 99-12831-157.mis complement
  primer_bind 190399..190417: 99-12831-241.mis
  primer_bind 190419..190437: 99-12831-241.mis complement
  primer_bind 191378..191396: 99-12832-387.mis
  primer_bind 191398..191416: 99-12832-387.mis complement
  primer_bind 195109..195127: 99-12836-30.mis
  primer_bind 195129..195147: 99-12836-30.mis complement
  primer_bind 203827..203845: 99-12844-262.mis
  primer_bind 203847..203865: 99-12844-262.mis complement
  primer_bind 210132..210150: 4-24-74.mis
  primer_bind 210152..210170: 4-24-74.mis complement
  primer_bind 210302..210320: 4-24-246.mis
  primer_bind 210322..210340: 4-24-246.mis complement
  primer_bind 210370..210388: 4-24-314.mis
  primer_bind 210390..210408: 4-24-314.mis complement
  primer_bind 211149..211167: 4-27-190.mis
  primer_bind 211169..211187: 4-27-190.mis complement
  primer_bind 215977..215995: 5-400-145.mis
  primer_bind 215981..215999: 5-400-149.mis
  primer_bind 215997..216015: 5-400-145.mis complement
  primer_bind 216001..216019: 5-400-149.mis complement
  primer_bind 216007..216025: 5-400-175.mis
  primer_bind 216027..216045: 5-400-175.mis complement
  primer_bind 216063..216081: 5-400-231.mis
  primer_bind 216083..216101: 5-400-231.mis complement
  primer_bind 216199..216217: 5-400-367.mis
  primer_bind 216219..216237: 5-400-367.mis complement
  primer_bind 216303..216321: 99-12852-110.mis
  primer_bind 216323..216341: 99-12852-110.mis complement
  primer_bind 216518..216536: 99-12852-325.mis
  primer_bind 216538..216556: 99-12852-325.mis complement
  primer_bind 221630..221648: 4-37-326.mis
  primer_bind 221650..221668: 4-37-326.mis complement
  primer_bind 221848..221866: 4-37-107.mis
  primer_bind 221868..221886: 4-37-107.mis complement
  primer_bind 225626..225644: 5-270-92.mis
  primer_bind 225646..225664: 5-270-92.mis complement
  primer_bind 229368..229386: 99-12860-47.mis
  primer_bind 229378..229396: 99-12860-57.mis
  primer_bind 229388..229406: 99-12860-47.mis complement
  primer_bind 229398..229416: 99-12860-57.mis complement
  primer_bind 237536..237554: 5-402-144.mis
  primer_bind 237556..237574: 5-402-144.mis complement
  misc_binding 1987..2011: 5-390-177.probe
  misc_binding 4589..4613: 5-391-43.probe
  misc_binding 10216..10240: 5-392-222.probe
  misc_binding 10274..10298: 5-392-280.probe
  misc_binding 39932..39956: 4-58-318.probe
  misc_binding 39961..39985: 4-58-289.probe
  misc_binding 41373..41397: 4-54-199.probe
  misc_binding 41392..41416: 4-54-180.probe
  misc_binding 42220..42244: 4-51-312.probe
  misc_binding 67463..67487: 99-86-266.probe
  misc_binding 69509..69533: 4-88-107.probe
  misc_binding 72826..72850: 5-397-141.probe
  misc_binding 76048..76072: 5-398-203.probe
  misc_binding 81241..81265: 99-12738-248.probe
  misc_binding 83909..83933: 99-109-358.probe
  misc_binding 91905..91929: 99-12749-175.probe
  misc_binding 95337..95361: 4-21-154.probe
  misc_binding 95499..95523: 4-21-317.probe
  misc_binding 96178..96202: 4-23-326.probe
  misc_binding 97282..97306: 99-12753-34.probe
  misc_binding 98012..98036: 5-364-252.probe
  misc_binding 98902..98926: 99-12755-280.probe
  misc_binding 98951..98975: 99-12755-329.probe
  misc_binding 103581..103605: 4-87-212.probe
  misc_binding 104386..104410: 99-12757-318.probe
  misc_binding 106361..106385: 99-12758-102.probe
  misc_binding 106395..106419: 99-12758-136.probe
  misc_binding 108303..108327: 4-105-98.probe
  misc_binding 108315..108339: 4-105-86.probe
  misc_binding 108460..108484: 4-45-49.probe
  misc_binding 109184..109208: 4-44-277.probe
  misc_binding 114592..114616: 4-86-60.probe
  misc_binding 115704..115728: 4-84-334.probe
  misc_binding 122071..122095: 99-78-321.probe
  misc_binding 123112..123136: 99-12767-36.probe
  misc_binding 123219..123243: 99-12767-143.probe
  misc_binding 123265..123289: 99-12767-189.probe
  misc_binding 123456..123480: 99-12767-380.probe
  misc_binding 126726..126750: 4-80-328.probe
  misc_binding 128198..128222: 4-36-384.probe
  misc_binding 128318..128342: 4-36-264.probe
  misc_binding 128321..128345: 4-36-261.probe
  misc_binding 128582..128606: 4-35-333.probe
  misc_binding 128675..128699: 4-35-240.probe
  misc_binding 128742..128766: 4-35-173.probe
  misc_binding 128782..128806: 4-35-133.probe
  misc_binding 130793..130817: 99-12771-59.probe
  misc_binding 133194..133218: 99-12774-334.probe
  misc_binding 135374..135398: 99-12776-358.probe
  misc_binding 139377..139401: 99-12781-113.probe
  misc_binding 157523..157547: 4-104-298.probe
  misc_binding 157567..157591: 4-104-254.probe
  misc_binding 157571..157595: 4-104-250.probe
  misc_binding 157607..157631: 4-104-214.probe
  misc_binding 172968..172992: 99-12818-289.probe
  misc_binding 180610..180634: 99-24807-271.probe
  misc_binding 180797..180821: 99-24807-84.probe
  misc_binding 190322..190346: 99-12831-157.probe
  misc_binding 190406..190430: 99-12831-241.probe
  misc_binding 191385..191409: 99-12832-387.probe
  misc_binding 195116..195140: 99-12836-30.probe
  misc_binding 203834..203858: 99-12844-262.probe
  misc_binding 210139..210163: 4-24-74.probe
  misc_binding 210309..210333: 4-24-246.probe
  misc_binding 210377..210401: 4-24-314.probe
  misc_binding 211156..211180: 4-27-190.probe
  misc_binding 215984..216008: 5-400-145.probe
  misc_binding 215988..216012: 5-400-149.probe
  misc_binding 216014..216038: 5-400-175.probe
  misc_binding 216070..216094: 5-400-231.probe
  misc_binding 216206..216230: 5-400-367.probe
  misc_binding 216310..216334: 99-12852-110.probe
  misc_binding 216525..216549: 99-12852-325.probe
  misc_binding 221637..221661: 4-37-326.probe
  misc_binding 221855..221879: 4-37-107.probe
  misc_binding 225633..225657: 5-270-92.probe
  misc_binding 229375..229399: 99-12860-47.probe
  misc_binding 229385..229409: 99-12860-57.probe
  misc_binding 237543..237567: 5-402-144.probe
tctccccaaattcatctgtagagtcaacacaatctcaatcaaaatcccagcagtatttttttgtgcaaaatgagaagtcgactctaagatttaaaatgaaatctgaagaatctagaagatacaaaataaccttgaaaaataaagttgtaggacataaactatctgatttcatcacttatttataagctacaataatcaaaacagcatggtgctggcagcaaaaagacaaatagctcaatggaacacaataggaagcctaaaatgaaacacatacatatgcaacacagattttgatgtaagcacaaaggaaatgcagtagagacaaaaataactttttaataaatgatgctggaacatttggatatgtatacatgcaaaaaaatgaactttggtccctatcccataccgtatacaaaaattaattaaaagcagatcttatcctttgagtccagtaggttgaggctgcagtgagctgtgattacaccactgcattccagcctgggcaacggagtgagaacctgcctggagaaaaaaaaaaaaaagtagaacctagacctgatatacaacctaaagcagtaatatttctagaagaaatcctaggagaaaatatttgtgatcgtggagatgaagaatctatcaaatactaaactttttttaccaccttgaccaaaagtaattggtttatatacttcatcatatcatttaattctaaatctacagagatcaatgtcactttctcagtaaaagtacgtgagtcttcaatgatgccctgaactcacactcccaagtaaaccataacaccatatttccagagtagagtttattagaacaataactggtgataatgataaatattgatcaaagactgagcctaggaagtgggttttttgaggctgcatatactcaaggcaattcttcagaaccacagagggctcattggatcctattaaaagctgagagttaatgaataaacagataaaacagagacctgagtagacggtagtcgatattcttgtacatgtattctacctctagattccatagaaagaactaaaagtacatgaatttcactaccaacatctccatcagttaccagctgtatcaccttggatcagtcaggtaacctcccgcgaatttgcttccggggcaggggatcgcgctgcaggtttgagcctgggagccggcagggtggagcagttggagggccaagcctttgagctccagggggggtggccgggacagtgggtagtgccagccgatcggcgtcctggggattgcctgaatgtgaggtctgggttcaccccgcggtgacctgagtcctgggatgcccctacagtgatttgctgcctcagggatccgaagtctctttcattcccttactggggatttgaggtctggaggtactcctgcgggggtctgagatctcggggtcaccctgtgggggtctgaagcctcgggtccccgctggggtctgaggtatcagagtcccctccgttgggtctgaggtctcggggtcccccatccccgggatcggaggtccggctccccggagcaggcagggcggtgcgtctggccctgacagtaacgtggcgcgccagccccaggtggtgtcgggctaggggggcataacggtgccgaaagtccgcacaaagccgtccgctggggtcccgccgcgcccgcgaggcaatgactgtgccccctccccttcctgatcctcagctcaggtgagcccagatgaggcgccgggtagcttctaagtcactaatggaaatagaaggctaattcaggggttaggggccgtcgtccttcttactcgcaggagaagagaaaaacccacggcccagcagccagaggcgcggcgaggcggaatcgggccccctccccgggggctcagctccctccagcctcccgcctcacctacagagaaatcccggaaacgcggattcagcggagcgcggtgacggcggcgcgctcaccccgcgcatgcccagtgcccgcscgcgccgccaggctcgcaagcaccgcgtaggccagctggccggatcccgccgtctgtcatggcggcccccatcctgaaaggtgaggtacttcctgctgcctgctccagcagcgggagtttgaggaccggcacccctcgtcgcgggcgcactcgggggatcccgtgggaggagccccgctcgcccctccctcgctgcctgtctcccccagaccccctgccgcctccttcctcccccgctgcctgtccccccaaaacccccggctgcctgcttcgtctcccgtgctccctgtccccccaaacccccgactgcctgcttcctcccccgtactgcttgtgccccaacccccgtgctgctagttcccctcaatcccccgctgcctgctccctcccccatgctgcctgtcccccaaatcccgcctttccccctacctgctttcacccctgctgccttagtccctggatctggggctcactggcaggcagagtcctgccctccggaagttggtgtggggccctcctgggtctggtcctgttcgaccccctctgaggcccacctggaggagcggcagttgagtttctatgctaattgttccaataataggagccgccttttactgcggagtctttgtgtgccaggcgctgtgcttaggctagtatggtattgtctgatttttttaaccgctctatcaactctcttatatcattgtacaggcagaaactaaggcattggacgtttaggtgactctccctgtgtgtggctagtcagtgctgacagggccttagaccggagctgctgtcctaaccagtatatgataccgcacgcagtcccaccctctgtgcacctggaagagcccaggagaggggaatagcggacacgtgtcttgtagagtttgaccgtgagaaaaaaggggcctgtattgtggggcctgcagtcataaaacctcatagccaaaagtaaagactagaggctttatacaaagtctgtaatcagatgtggctatttttctaatgttagtattttgttaaattaacctggttttcttttagcgttacccccaatcattgaccaacggcacacctggaaaatgcttttaaacatcaggttttgagaagaggatatccactagaacaggggtccactcactatgccccccaggccatatctagcctgctgcctgtttttgtaagggtctacgagctaagaatgtcttttacatttttaagtgattttaaaaaaaggtcaaatgaaaaattatatcacattcacatttccttctccataaataaagttttattggaacacaggccggcccgttaatatattacctatggttatgtttgtgccacaaccgtgaagttgagtagttgtggcaaatactgtattggccacaaagcctgaaatatttaccatctgtctctttacagaaaataggtttctgcactggaaaaattaagcgtaagaatttggggaaagcaactaattttacaaatgtaaactctcatgtattgtatgggtacagttgttctttgcttaaaattttaataaattccactgaagctattttgaaaaggctttcagtagaaatttatttatgagacagagtcttactctcttgcccaggctggagcgcagtgatgtgatcacataatagctcaagcaattctgcttcagcctcctgagtaacttgggactacaggcactaccatgcccggttatttttatttttattttttagtttattatttttttgtagagccagggtctcactatgttgcctaggctggtcttgaattcctagcctcaagcaatcctcccgcctccaccttgcaaaatgctgggattacaggcatgagctactttgttcagccagtagaagaaacttcatttacttttcttatttttgaggcaaggtctttctctgctgcccaggctggagtgcaatggtgcgatcataactcagcttctacctcctgggctctagggattctcccacctcagcttctccaccctacccacccccatttcccacccagtagctgggactacagccactcgccaccattcctggctaattaaaaacaaaattttttttagagacagggtttcactatgttgcccaggctggtctcaaactcctgtgcccaagtgatcccactgccttggccttccagagtgctgcaattacagcatgagccaccacacctggccagtagagtaaatttttgttttacttttttcttttttttatttttgaaacgggtctcgccctgtcacccaggctggagtgcaatggcgcaatctcggctcactgcaacctctgcctcccgggttcaagtgattctcctgcctcagcctcccagtagctgggattacaggtgcccgccaccatgctcggctaattttttgtatcttttagtagagatggtttttcaccatgttggcccggctggtctcaaacccctgacttcgtggatccacccacttccgcctcccacagtgctgggattacaggcgtgagccactgtgccggcctcggtttactcttaaatgtaaatagaacaaaatctattgggcaggggatgctggaatttcaaatgtatrtttcatgttcatatcttgttttcagatgtagtggcctatgttgaagtgtggtcatccaatggaacagaaaattattcaaagacatttacaacacagcttgtggatatgggggcaaaggtaagacacttattttgctgttgattcatatgacagtcttctgattggtaaaaagttacatttgcattttcttattttgggagtttttacttagaatctggacgaagcaatgggtaagcggtgggagaaaaaagagccaaagtgtgaagaatttagaacagtaggactttcagaactcaatgcctgtgggcattgagtgaggaggaggaacctaggatgaaatgctggattcttacactggttacttgaatgcatagtgctattaagcaaagtgaggaatacaggaaaaggaacaggtttctaagggaaaaattgtaaatttgggcatactgaaaatatctgttagatatttggatatacaagtctggagcttggagtgttcaaggctagagatgatgatctagggggtcaggaccataggggtcatgtgaagtcacaggtgtggacatcgtcccatgtcaggcatggttaggatgaagagtggtgacagaggagcgttgttcagtattcaaggacaggcgatgggagcagggacccagtgacagagggagagaagaatgccaggagaaggagaaaggaagtgtggaagtcaaagtagggagtaattttttttttttgagacggagtttcgctctgtcgctaggctggagtgcagtgacgcgatctcagctcactgcaatctctgccttctgggttcaagcgattgtcctgcctcagccttccaagtatctgggactacaggcacatgccaccatgcctagctaatttttttttttgtatttttagtaaagacggggtttcaccatgttggccaggatggtctcaatctcctgatctcgtgatccgcccacctcggcctcccaaagtgctgggattacaggcatgagccaccgagcccggccaggagtaattttttaattgcctttcagaactagaatggagtaattttaaagatagaatttttaaaaactacagaaagttcaagaaaaataggatgggcaaatgtactttggatttgaacactgtaaggtcattgctgaacttagtgcagttttcagtgaaatgggcaggaatcattgagctatgaggaaatggagatagcaaacaatttgccttattcaaggtttcttagtatagccatctctgttatcagatttactatcacgtactgcttgtgttcaggtagcctctatttgacttaataatgtccttgataccaaataggtatcttttgcccacgcacactaaaccgatcactttgatgacgggttttacaaaagggaaaagattcattcacagggaagcccagctaggaggcagaagagtactcacatcttcattcccaaagataaggcttagggatatttatcagttagggaagtagggtgatctaagctgtggggaaaaatgaagtacatgatctgcacaagcatagttgggattcatggaatgcatgtttagaaaacaggcattattaggaggccaaggcaggcggatcacctgaggtcaggagttcgagaccagcctggccaacatagtgaaaccccatctctactaaaaatacaaaaaaaagccaggtgtggtggcacacacctgtagtctcagtgattcgggaggctgaggcaggagaatcgtttgaacctgggaggcggaggttgcattgagccgagattgcaccactgcactccagcctgggcgacggagtaagattctgtctccaaaaaccaaaaaaataggcactagtaggatccgatggtgaagattttggcctgatgtcaaaaggtcatttcttgggcatttacacaggcctggttgaagagttggtggttgcagcctgtttgaactgtacgggtgctgccccaagttcctgaaaagtaacttaagcaactgttaccgtggtgacatatccaccagaagtttttatcttataaggaagccagtgaaggttatagcatttagtagtatgacttgcagctatatagaaataaataaataaataacaaaaagcaagtgaccaaaagcaagcaaggcaggttaaatttggcagaactaattttcagccgtaaagtgcaagagtgatgatgctggcaattcagatatgccagagaagccttaaggtgctttaagtgaaaaggtgaaagttctccactttaaggaaaggaagaaaattgtgtgttgaagttgctaagatctacagtgagaacaaatcttctaatcttgaaattgtgaagaactatgctactgttgcagtcacaccaaactgcaacagttacagccacagtgcgtgatttttattataatacattgctacaattaccctattttgttatcattattgttaatctgtgcctaatttgtaaataaaacttcattgtatatgtatgtataggaaaaaacagtatataacctgttcagtactagctcaggattcaggcatccactgggaggggttgggggcgggacgcgggcatgtcttagaacttaacccccgtggataagggggaactaatgtgctcttatagggagtttagttatgaacaaatcctgtttatgtccttgtctggcatttgggaggggctgactgataggctgagtgaaagagaaccattaaaaatgggagaaaagataatcgaaggcagggctaggttagggtggagcaagagagctgctgtgggtataaaacttaagaggcgctcaccaccaggcaaagagtgggtgctactgaataccctaagagccttgtttgacctccctaatgcctgtcttgagtaagaggtcagtggagaggaatccgaatataggagcagggcctgcactgcaggaggggagacatgccccctgtaatacactggaatgtaggaacccgagggagtctgcatgttgcacatgcctaacatttacttgggatgaggaggaactactgtgaataagaaaaaagccgttagacaagtgagttgacaaggtggtttgagggtagcattaagatcttagatcttttagaactttttggtttcaccttttatttcaaaaattggcaaacagttcaaagaatagtgaatacagatcaatagtcgttaacatcgtaagatttggatatttgtagtactcgtaatccgggattatcttaagccaatttcaggatttgagatgatttaaaaccagactacaggcctgtgagggtcattataacttctgattcacccttaatctagatgcagctctttgggtctcagcgcaaggtgtaggggttttaccaaacccccttgtctctttgaggcttctcaattttcgtcctgtttagtgtgtactaaatttgataaaagccttgtgggaagatggtctcaaatgctagactcatctctctaggtgtcagtcttaatctagaatctcagcccggtaattcttaattgccttgatagctcccatggacttgatgggggtgtggaaattgagagagagagagagttataaaagtaatacatatttattgtttaaaaagactaacgggcagtgccatgaaattcacaatgaaaagaaagagaaaccagcaacgctttgcagtacatttccttttccatttttcaaagacagctactttcaaatcatctgtttcttttggtatttaccttcatatttccaagcattgtacatatattacttcagtataattgaatgctataaaaatcatgcagatgagttctgcttctggaaaggatacataaaaggtaaaatttttgacaccatgactgtctgagcatgcctttattatactgttacctttgactaatattttagctgagtgtaaaatgctagcacaaatattatttttccttgaaggtatttcccattgttttctcaattccagactgctgttgataagactgattcagttgtcacttatcattgtttgcatgtgatgtgtctctatcctcttcaccttgattacccactcttttaatatttttccctttcgccaaccatgctggaattctgtgataagcttggtgtagtgctgttttcgttcttgtgccgggcctttgtgggggattcttttgatctagaatgcatatcctttagtttgagaaacttttctttgattatttctttaataatattttctccattttgtgtattcctgtattctttaacttctgttgattggctgttggatctcctggtctgagctcctgatgttcttgccttttgtctcctgttgtccgtcttctggttcttctcttctactaccagtgagcttttctcaacttcattgtctgatatttctgtagaaaatttttttacttatatcatcttttcttaatttccaagagctctttaggatcctattaaaaaataatcttctgatcatgttgcatgaatacagtatcttttgtttttttttttttggagatggagtcttgctctgttacccaggctggagtgcaatggcacaatcttggctcactgtaaccgccacctcccgggttgaagtgattctcctgcctcagcctcccgagttgctgagactacaggcacgaacctccacgcttggctaatttttgtatttttagtagagacagggtttttccatgttggccaggctagtcttgaatttctgacctcatgatccacctgcctcggcctcccaaagttctgggattacaggtgtgaactaccacacccagtttcctttggttttaattagctgaatttttccaactttttgaatgattgcacttattttcaaccttcttactttgtatttatgcatttaagattacaggcgtccgccaccttgcacccggataatttttgtatttttagtagagacagggtttcacgaggttggctaggctggtctcaaactgctgacctcaggtgatccacccgcctcggcctcccagagtgctgggattacaggcgtgagccaccatgcccagccatggatacagtatcttaagatatgaggtatttttaattttggttaaatatgtgttctgttttctctgttgcctctgaatttcatttggttttatttttttgatgtagaaagcttttctgaaatgtccattattatctgactctttccatctttaaaaatgtggtgccttctcatggccacattttctcttctgtcctctttatccttgcagggctccaactctattctttcagtaacacttcagagggtttttagagggagtagatgtgaacttgtgtgtatgattcaccgttgtaactggaacagatatgttttaagcagcgttatacattcctttgagtgtttctctgtcagattttgagaaacagaattgctggggtagaggttttttgatcagttgtagttaagttgtgaatgaacagtaatgtacattttgttttctgcattttgtctacaggtttcaaaaacttttaacaaacaagtaactcacgttatcttcaaagatggctaccagagcacttgggacaaagctcagaagagaggcgtaaagctcgtttcggtgctctgggtkgaaaagtaagcagtttctctcttactttttttccttaagtatctagtattgaaaatgkgtggagatatttttcacaggtcggagaaccagataaagtttgattttcatcttttctctgcctcttacctcaccaagtaatttacatcctccagcctcaatttctgtggttcaaaaatggtcatgctataatacctaactctgcctagggggaaaaggagcctgcaggtcctgaagctgggtatgcaaggtggacttaggaagcaagagggaatgtgatgaagcagattgtgttagtcagcaagcgctgctgtaacaaaggaccacagaatgggtcgcttgagcaacagaaaaggactttctcacaactctggaggcaggaagtccagtatcaagttgtccacagggttggtatcttcttttttttttttttgagacaaagtcttgctctgtcatccaagctagagtgcagtagctggatcttgggtcactgcagcctcagcctcctaggctcaagtgattcttatgcctcagcctcccaagtagctgggattcatctcaacctttgcctcctgggctcaagtgattctcctgcttctgcctcccgagtagctgggattacaggcacgcaccaccatgcctggctaatttttgcatttttggtagagacggggtttcatcatgttggccaggctggtctcaaacttctgacctcaggtgatccacctgcctcggcctcccaaagtgctaggattacaggtgtgagccaccgtgcgcggcccacacagttttgattacagtaaatttgtagtaagttttgaaattgggaagtacgagtcctgtaacttgtttttcattttcaagattgtttggctattttgatttgagttccttgctaagattgtttggctgtcttgattgggttccttgcatttctatatgaattttatgatcagtgtgtcaatttattcaaaaaacaaaaaaggcagctgggatttggtaggattgtattgaatctctaattagggaagtgttcataatatttaatcttccagtccatgaaaatgggatgtgtttctttttcaggtctcaaatttccttcagtgatactttctagttttcagtgtacaagttttttaccccctaggttaaatttattcctaacttttttgttcattttcatgtgaatgaaattgttttcttaatttttttaagttgttagctgttagtgtatagaaatgcaggtgattgttgtatgttgatcttataccctgcaaatttgctgaacttgtttattagttctaaatatatttgtgggttccttagcattttctatatgcaatgttgtgtaattttgtaaatagagatagatttacttcttcatttctagtctggccgcatgttatgtcatgtcatgtcatgtcatgttatttgttctggccagaacctccagcacagtgttgaatagaagtggtgagaatggacgtccttgtgttgttgctcatctttggagaaaagctttcagtatttcattatttcgtatgatggtaactgtggtttgtgtaaatgtccttttttaggctgaggacgttcccattcccttctgttgcaggtggtttgtttgtttctgattattaaaggaagttagatattgttgtcagatgtttttctgcatgactgatcatcatgtgatttttgtccttcattatattaatgtggtgtaattgaggggttttgtgtgttgaagcaaccttgcagtcctaggataaatcctacttggtcatgttgtatacgttgtcatcttgcctttttaattgttggaacaggccggatgcggtggctcacacctgtaattccagcactttgggaggccgaggggggtggatcacctgagattaggagtttgagaccagcctgatcaatatggtaaaaccctatctattaaaaatacaaaaattagccagtcatgttggcgtgtgcctatagtcccagctactcgggagattgagacaggagaatcacttgaacctgggagacggaggttgcagtgaaccaagaccacgccattgcacttcagcctgggtgacaagagcggggaaaaaaaaaaaagagtaaggggtccttctctggcttttgtcaagattttctcattatctttcactttcagaagtttaagtgtctcgatatggttttttaaaatttattttgtttagattttacagaacttgttgaatgtgtagttgcctgtgttttctacatatgattcgtttttggccattatttcttcatctatcttttctgccccgtttcctcctcattttttctgattagctgtatatctttttctaattagctgtataccagggattggtaaagttttctgtaaagggacagatagtcaatattttaggctttgcgggccatatggtctctgctcaacagctcagctctcttgtggtgtgaaaggcgtaatagaaaataagtaaacaaatgcttgtgtctgtgtggcagcaaacttataagtctggcaggaagccaggtagtttcccaatcctttctgtgtaatacaccttttaaacgtttgcatttgtccatagtgctctccttcttcttcattattgttcagtctttttttttctcctcagattgatcttttttcaagttcattgacttttttctccatcaaatccattctgctacttagtacctttatttgagatatttttgatttctaacatttctagttggtttttgtatacattctttttatttcggtttcatgtgagatttctcacctttggttttcctgtcttcggttatgagtgtattttctattacctcgatgagtgtagttataaatagttgtcttaatgaccttgtctgataattttgaggttggtatctgttttgtttttgtttttctttgatagtgtgtcacatttttctggctcttcatatggcaaataatttgaggttgtattatgcacgttgtaaatactatgtagactctggattcttttctattgtcgcaaagagcatgaggtttttgttttagcaagcagttaacttgtcgttaaaatgaaacgcacactgtcattatgtgggcagttgcttagatgcgccctttaagcctcaggtgcaggctgatttgtttgcctcaaacacatgttgttcaggggtcagccagagacttgaacttctatactcagaatttggggtttctcctatggttctcttacttcctgagtccttacctcatttctctagtagccctagctgcccagtctccttcccctggtctcttcagcgagaaaggaggccggagcttctgcttgagtgcttgctgcgccacaccagctccctcagagactgtggctgcctttaggggacagacagaaaaagtggtgatgcccagattcttttgcttccttttaaaatttgcctgttctttctttttcttttatttccctccagctttcaaagctctcacatagttggtttattttattttatttttcctgtatttccaggacgtatagcttatagttcacctatatttgtatattggtttgttaggcataaacagaaatggaacttagtatgttatttttgaagcatctgatgccagtctaattcttcttcccttcaaaattatttgatctttttggagactccttagggatattttttattttatcattttttttttgagacggagtctcgctctgtcgccaggctggagtgcagtggcgcgatctgtgctcactgcaacctcctactccctggttcagcgattctcctgcctcagcctcccgagtagctgggatcacaggcacgtgccaccacgcccagctaatttttgtatttttagtggacacggggtttcaccatgttggccaggatgatcccgatcttctgacctcgtgatctgcctgcctcagcctcccaaagtactgggattgtaggcgtgagccacagtgcccggccaggattttttttttaagactcatggctttactgtaatatgttttgaattgatcattccagttctggcttggccttttcaacagattcaggtctatatttctgcaaaagtttctgggaattatagttttaaatattctgcttcgttgttttgcttttcttctgggactccaattatgtttacgttgggcctgcttagctatcttttatttcagtcactttgacttcaacccttttatatttatatacacatacacacacacacacacacacagacacacacacacacacacacacacacacacacaatttttaccccaaatacttatttgacagtatttgtttttgttttttgaagacagggtcttgctctgttgccgaggctggaatgcaatgactcagttgcagcttactgcagccttgacctctaaggctcaatcagtcctctcaccccagccctccctagtggctgggactgtaggcatgtgccaccatgcccagccattaaaaagtttttttttttcttttttctttgagatggagtcttgctctgtggcctagtgcagtggcgcaatctcggctcactgtaagctctgcctcccaggttcatgccattctcttgcctcagcctcccgagtagctgggactacaggcgcccaccaccacacctggctaattttttttttttttgtatttttgtagtagagatgggattttaccgtgttagccaggatggtcttgatctcctgaccttgtgatccacctgccttggcctcccaaagtgcaacccggcattaaagaattttttttatagacatgggatcttactatgtaggccaggctgggctcaagtgatccactcactccagcctctcaaagtgctgggattactggtgtgagccactgcacccagctgataatatttgattcaagttcaagggttttgttatattcttcagttttgtgtttgcttttattttagggagtgtgatgggttttcctcagctgaaatgatttgctttttctttgtttttttaaaatagatttttaaaatggatgtagtctattctatttccattcattgcataggccaggcttgtggccagagcgtcctcttctgtcagttctgctgtcttgcatagtttcttttataggtgacgctggtgagggagggaggagggaggggctcgtgtatctcgtttgcgttttgtttctataggatccttaaatgtttttctcttagtttcttctttttttcactgccattggttcaagggctgccactccccccagaactgatgtttttcagagcctgcctgtcctagtcttgctcccattcagaccccttccctggagtgggtgctgtgagctgtgtgggttctctgttgtggcagttgtgctgggtgtcctctttctgagacttcttttacctgtgcttcatgtaagttctccaggctgtactactttttatggagtcttaagcgtattctccccgactttctgcatccatagacttgcagctgtgttggaatttgattatttttctacttataggtcatctgaatttgcgctgttatctccgtgtcagtgagaatgtaggtcatatgtgtcttttatttaagtttcttttttattttctgcttttttttcggggagggaatggggtaagactcagtatcagccagccatcattgttttctctacctcatcttcttatggagtccattgaaatggcttattgatttttatctcaaaatcgatctctcatagatctttatctctgctgttacagtcgagacaagtatcatgtcttgcttcagttactgtagcagcctcatgcctgtctgtttcattttgtttcttatacataagcaaatgtaaccccttttgttaccagtggaaggtatccaagttaccggcagcaaacacgtatgggtttgcagcaacttcagttcttgcttcctcaaaagaaagaattccacggaggagcataaggcaaaagaagagcctgacgcaagggtcagagcaggagcagaagtttatttaaaaggcgtcagaacagaaagaaaggaaagtacactgggaagagtcccaggcgggcatggaggtctaatttgatgtttaaccttgatcctgggatttgtaggctcgcccttttccgcagttcttcccttagggtgggctgcccgcatgcacagtgcgggaattgagcacaggcagcttgtttaggaagttgtgtgggtgcccatctgaagctttcttcccgtttctccgccattttgtctcttaatgtgcatgcccgggaaatggcctctccctggcgtctgcattcagttaacactttagcacaacaggtgtggactgtcaggaaatggcctctccctggctctggctgccaatttatcacttttagagaggcaatgtgataattgttgagctatcacccaacattcctagtgggtggtagaggcctctcctgccgggcttatgcctaactacctgtgatacttcaacacatggatcagctttatccttctgacaaaatggcttagagttcagtggtctatagcagagaatggccaactatcatcccccagccaaatccaccctgccatactgtttatttttttttaatggcccatgaggtaagaatggttaagagaaaaaaaaaattcaaatgtttactatttcatgatatttacattatatgaaattcaattttagtatccataaataccgttttattggaacacaggcatgttcatctgacgatgtagtcagtggctgcctctgtactacagctgtagatttggatcctgtggcagagaccttacggcccatgaagcctaaggcattcactactttcccctttacagaagtttgctgacccaggtccagtgtgctgcatgatggtccccttcccttccattgtcagctgctcccctctcccttgtttgcgtcttccaaatgctctaggcttccacgtccccaaggctacactctttctgcctttagttcttggcctgtgctgagaactctgccccgtcttcctgattctaaacccagttttgtagtcagctcctttatacatgttgcattgcaaggtcgctttatcagaagagcttcctctgtccccagttcacagttcaagccctatttgttattctctgtctcagctccttttttcctgtgtgtactattaaaacttattttgttcatttgactgctttatctgtctgtgtatctaatcatgcattttgtctttctattgtaatgtggattccaagagcagctacctgtctgtcttatttatggttgtgtttctagtaagtctaacattcatctggctcatagtagatgctcagtaaatatttgttctaacaaattatgaacaaaggaaaatttagttaagtggcgtagagatactagagaaaatatcatgggggaaaatgatttgaaaaaaactacattttaaaagtcgtatagaaatgtggaggggagagtgcagaaacagagacctttactagaagcttgaagtaaatggagatgcatggacaaaattaaaatagtagccatttctgtacctaatagggcctctcagctaaccctacagtggggatggtcactggtagtgtgttctgctgagagttagggattcttactctgctttgctggcccagcccctgactcattctctatcccctttctctctctctctatttctgcccaccactaaccccagcctttctcaaggggctcatgcagaccccataatacttgtaacttcgttatccaaaagcaaagttttctttttcttttctggagactgagtctcactctcttgcccaagctggagtgcagtggtgcgatctcggcttactgcaacctccgcctcctgggttcatgccattctcctgcctcagcctcccgagtagctgggactaccggagcccgccaccacgcccggctaattttttgtggttttagtagagacggggtttcactgtgttagccaggatggtctcgatctcctgaccttgggatccgccctcctcggtctcccaaagtgctaggattacaggcgtgagccactgtgcccggccaatttttatatttttaggagagacagggtttcaccatgttggccaggctggtttaactcctgacctcaggtgatccgcccaccttggcctcccaaagtgctaggattacaggtaagagccaccgtgcctggcaaaagcaaacttttaaggtcctcagaagctcaaaagtgaacttaatcttttggcatttttcttttcttttttttttttttttttttttgaaactgagtctcgctctgtcgcccaggctggagtgcagtggtgcaatcttggctcactgcattctcctgcctcagcctcctgagtagctgggactacaggcgcccgccaccacgcctggctaatttttttgtatttttagtagagacggggtttcaccgtgttagccaggatggtctccatctcctgatcttgtgatccgcccgcctcggcctcccaaagtgctgggattactggcatgagcccctgcgcccggcccatacactttagtcaactttttattacaggtcatttttttgcctgtacatgcagatacatcccactttatatatataagaatattttgtagtagctgtccagtaatttatgtaagcagtgtcctattggtgattgaagtttttcatttcttagttatttttttcaattagaaatattacagcattgagcttctgtatgtattacctttttgcgggtgataaatctttccataggtttaaatccccaaagtgggctgttcatttctgagagtttacacatttaaatatgatagatgctgccaaattatcttctggaaggagtgtactggtttccattctcactggaattatcaaaaaaatgcatgtttcccaatacctttgctaatgttgtgagttatcagttcttttttctaatttgtagaagaaaaataatagtttttatttgcatttctctgacttttagtgagcttgaattttcttcagcagagcatagagataagagccaaactgacctgcattttttatgtcacgtctgtcctttcttggtgaactgcctgcctttccaatgcagtagctcatggtttccactgaaaatgtgaacattaacttcataaggtcactaggtgtcactagaatcccattctgttgggttccttctgggagtgttcattttaagatcagatggcaattgataaaattctgacatttcctttggatgtagaaatttttaccttgaagaaagaatacataaagttgaaataaaggtcagcttggcccccactctaagttctgttgaagacaatttatcatttttaaacaactgcaaactaacagctaggtggggaatacggttcacaggctttgtccttgctaggctgagagttggttgctgaccgaagccatcaccccctgcatttagtgtttgctggaaacagggacatattcctgcataaccacaacacaggccgacattaggggcttaccacggctcctttcctcccggaatcctcagactccattcctatcctaccagcagccagctccacttcccgcctcctcagccttctcaccctgcagccattccttagtctttcactggcttttgtgactttgacactgtttaaggtcactgaccagtgataggaacgtccctcagtttggaacggtctgatgtgtcctcctaatatcacatcaatgtgtaacagtggatgtgtagccatttaggactgggcaaattactcaactgctgggctctaggttcctccagtagctcctgagttaacttcctacggttatttagtgctagaccacagaagttcgctctctgctggcagagcactgttgtgcagacttctctgagtctcctgtgttcttccttgtgtgtcagggacacacgtgaaggatagcgtgcttcgcggctggaatcttcaaggagatgccattcacttttttacctcactaacacagtgccgtttacaaaaaagattaatgtacttttcctgaattgacttactgactgggcctagagaataagatactggtgctgggcagtttggcacaagagtagtataaagaatgcaggattggcccaggtgaaggcatcgtcctaagggtagaatgggagtcggtggttcctggccgacctagcaggtgtactgtgggaagtgctggagtgaatcggctctctggggagaataagctcatcacagcagggcttcccgaggagaacgttgctgctttgatttctgttggctctgaggcagcagcaggtcaaatagttggttctctgtttagagacatctcttgaaacacttttcgttttgaccactagatggtgggataatgttatcattttacatttctgaagaaaaatagaaatctaactggaagcttttttgtctgttcagtagattttggttggacccctggtaaacatgggtttcagtgtagcagctttaatgtgttaccacgtgtgctaaagcatagctgttggcatgcagaacggcattaccagcagtaagtgccacttacttcttcatagtgagtgatgatagttacacccaggtagatgaaattcagggagagcatctctgtgcaccttacatcttatcactctgaaggatatgtggttgggaagcttctcccaaaggaacagaacacatcttccacaactgtataacctatgtcaggcacacgttttcctgggttgaatcaagcccttccttaaactgctaacttaaagaatacttactggttttgtaaagtttggcaaatgatcttctctgctcctcggttttctgtgttgtgcaataggaggcaatggtagtggcttttccagcacggttggtgtgaggcttctcatgagctgggtgacctttgtcctgatgatggtggtgattttaatactgtgtatttgataacacgattatctagggtctcctctacgtctttcgtccagatgcatctcagccaccccccttttgctgttcccttaggcataatagtggtaaatcggtgacattttgcttgagtaagaagaagctgctaaaaacttctcatgcttaaaattggtaattaaggggactttttaaaaagaagcacagttaaaaaacatttccttcctcgttctcttccacccgcctccctttcccatcacttttattagatacagcattctgctcaccccattattgcaggctcagatagttggtttgtttttttaaaatcagctttataaaaacatttacataaaataaaatggacccattttaagtgtacattcacggattttttgtgtatacctgtgtcaccaccacaaccaaaatacagagcattttcatcaccccaaaatctccttcgtgtccatttgctgtcggcctccctgccccctcctcccaccccagggcagccacagatctggtttctgtcattaaagattagtgtcaccaattctggggcttcagatcagtggaatcatccagcgtgtactattttgtgcctgacatcactgaaggtgatgtttttgcgatctgtccgtgttgtttgtagcagtggtttcacttccttttatagctgagtagtattctattgtaggcatgtagcttggtgccaccagttgatggagattgggctagtttgccattttaggttattatgaataaagttacaatggacatttacatttgtgtctttgtatgctttcatttctcttgggtcattacccaaacttttccaaggtggttatggcactgtatattcccaccagcagtgttcctttcactccacgtcttcaccaatagttgaaatttatccatcttttgaattttagccattcaagcagatgtgtagtggtatttcatggttttttttttcccaacattgttttaagatctaattcatatgctacacaatttgtccaattaaagtatacaattcagtggttttaaatatacagtcaggtattgcttgacgacagggatgccttctgagaaacgaataggtgattttgttgttgtggagacatcacagtgtgtattaacacacacctgcatgacatagctactgcacacctaggctctgtggcacaacctgttgctcctaggcataaacctctacagcatgtgcagttgtgaaacagtggtaagtatttgtgtctctgaaatacttaaacatagaaaaggtagagtaaaaatatggtataaaagataaaatatggtacacctacatagggcgtttactatgaattgagctcgctagactggaagttgctgtggttgagtcgttgagtgagtggtgagcgaatgtgaaggcctaggacattactactatacagtactatggactttatacacgtcatacagttaggttacactggatgtatattttttggagcaactgtattaactgatactataacgtttttttaaagacaaggtcttgctttgtctcccaggctggagtgaagtggcacatttatggctcactgtagcctcaacctcctaggctcaagcaatcctcctgcctcagcttcctgaggagctgggactacaggcgtgtgccactatgcctgggtaatttatttttatttttatttttgtagagacggcattcttgctacgttgcccccactagtctccaactcctgacctcaaacagtcctcctacctccgcctcccaaaatgttgggattacacatgggagttattgcacccggctcctcccataagtaaataatctatctctctgttacttgtggtgggaggaaaagaaaaaaaacacctaggttatgtataatacctaatacgagtacttcgtaagtagttattatactgttttttttttttgaaacggtgtcgctctgtcgcccaactggagtgcagtggcgtgatctcggctcactgcaacctctgcctcccaggttcaagcgattctcctgactcagcctcctgagtagctggaattacaggcacgcaccaccacgcccggctaatttttgcatttttagtagagacgggtttccccatgttagcctggatggccttgaaccgctgacctcccgcctcaactcccaaagtgctgagattacaggtgtgagccaccacgcctcgcctatactgtatttttttttaatttggccttactatagcttttttacatgataaactttgtaattttttaaatttttttactcttttgtaatgccttaaaatacattgtacaacagtataaaaataccttatatctttatcagctttttctatgttttaattttaatttttacttttaaacttaaaactaggacacaaagacacacattagcctgggcctacacagggttaggaacatcagtatgtcgctaggcgataggaatttttcagctccattataatcttatgtgatcactgttgtgtatgtggtctgtcattgaccaaaaggttgttatgcggcatataactggattcacagagttgtgcaaccgtcaccacaatttaaaaacattttcgtcacctcaaaatgaaacttgcaccccttagccctatcccctattctcccgccagccaaggcagcctctagtagtctactttctttctctgtggattttccttttctggacatttccaataagcggaatcatatgatatacggccttcatgtctggcttctttctcttagcataatgttttcaaggttcagcatgttgtcatctgtattagaatttcatttctttttatggtggaatcatgttccattgtatggacacgtgcgcacgcacacacacacacacacacacacagaagaactaaatattacaaggcttatcatgaaaaacaatggtctctttcttgacccttttcaccctcaattcctgttccccagaggcagctcctttcacacttgtggctgcttctgcagataagctgttcggtgacctccatattttaaatactgtggccgtattgctgtttcggtttttcagtttcaggtattatctagtgactttctgatagggaagtgagaatttcgtttttaatccgcccctctgagtgcacctcactcccacatacactcatctgctgtttgcatggacacattcatgtgcaggctctttccactcttgattgcagtgtacatgatacattttggttaaatcggtagtttatgtttacatcattatgactgtggaagttgtgtgttaggctgaatctcagagtgaaccatgaatatatttcctttcgtggaaaactttttgttttccctgagcttggcctggtgtcctttgagtccagagcttctcaggctccacttatgtgaacatggacccagtgcccccattggacacagggtggcagtgagtgggcacaggcaaggagagaaggagagtcgctccctcttttcagccttccaccctctgccctctgcactttgccccctgccccaccccagactgctgtggcttcacctgcgcctcctgcccttgaggggttctgagctccaggttctgagctccagatggactcctcccccgccccagctgccaggcttgggtttccctttttttttttatttgtttgatttcatttccccagacagctcttatctactctttatttttgttggtttatgtctttttgttttcctttactatcattttattggggttttgggggtcaagagaaaagcatgtgctaagtccaccagatttaaccagaggtcaaaaaccttccatttttattgtctaaatattattcagttaaggattccccctccccatcttagtccccaactgcctttgctgaatctttagcgtctcctgccacagttattgcagtattccctgactggcttcctcctcctggaccagtgatctgcccacgacccctccctcacacctgtccccatgccccagacccacaggacagggtccaagctcattagcttagaaagtacaacccttggaatcacatgaattctttttttgttgctagtctcctaagttgcattcattcactcagtcatacaaatggtgtatgttttccccacaatgtcaccctgtttgctgcactgtgcttgagtctatgctctgcttccagatggaagatctgtgtcctcccacatctgcctccttgtcagagttgagtctggtgatcatctctgacctgaagctttctctgaaccatactcgttatgcaacctgttgctgcttttctgcctggttgtacttctcttgttacaattactgcactgtgttcttttttaaatttgtacatttttgcagatttctctgatgcctggcttaatagaagacagttgccttctcatatctgcctctgcattcagtgtattggggtggcacatgtcgttttgcttcggaaaattccactgcattgtatactgaggggataatgcgagatgagaaaggaaaatcacacgttagtgttgttataaagatagtattgactttacacaccctcagaagggggtcagggatgccaggatgacattcactaccctagtgtcacttaccacattgcatagaccatactgtgccgtacagaggcacatatttctgaaacttcctttattcctaatatattttgtagaaatttctatatcagtatggatatgtgttttttattgcagtgtactttattttttcaaataactgttcgtgtgttagatgttgaacggtgataggcctgtgagggatagttggagaggtgactagaggccttataaaaacacttaaacagcagatgagtgagaatatgctctaaacatgggagtgacagaaggtttttatctaggttgggaagaaatttaagattaatatttcaggaatgtatgagtgaattagaagaggagaaacaaatagtagggcaggagatcatttagaaaatcataattatttagacttgagtgacagaatgctaagaaggagataagggtcacaggaatccagagatacgaaggtggacaggagaaatggcaggtgtgtccacagggcaggaggaggaggcttggcaatgcggagcattggttgcacacctgggccttggggctgatcgtggtgtctggacagaaacacaaaaaggacaacccaattttggaggaaagagatgtcctctgacttcaatttctttacgtcccttctacctctgaattatctgttttatggcctgtttactattaaatgatccatttaatagcatttacccttagctttatgagtaccatgcactaataattttgaagtatgctacaagtcaaaaattgttgtgtaaaaattgtacttcctttacctgcctcttgcttctgttatacttaaataccagatagagatgattttgggaagtttgatttatactgacttttgtatttgctgttgtatttattttttaaaagtctgttaaaatgacctagctatggatttcttaaattgctaatacatgtgcagatttagtgctgtgtcaatgtataatagaagcaaatactcattagactaccttaatttaattatacagatgcaggacagctggagcacacattgatgaatcattgttccctgcagctaatatgaatgaacacttatcaagcctaattaaaaaaaaagtaagtacatgatttcaatgtagataatggcaattaggaatttattcgtttttattttttatttctagaaaataaaacttctagaaatatattcaagagttgtcttaaatatgctattgatgatattgttcttttcacatagcatttttaagtgaattacagagattattttatcctatgacttcttcgatagcatttgtatgaaatggaaaagcctgtggttggccatgggaagactaaaaggtgccaagagacaagcaaacatttaggtgctttggtaattacttcagaatgaagtttgttatatctgtagtcaaaatacctgcattctgtttagccagataaatctcaaaagtccgatggacctacatccaagtgtgcaaagtcatttattaggaaaatctgctgtacaaatacagttgtccttcattatccacagaggatcagttccgggacccccacagataacaaaatccactgatgctcaagtcccttatataaaatgccatagtatttgcatgtaacctacacaaatcctcccgtatacctaagaaagaattttttgtagagacagggtctttctatgttgcccaagctagtctcaaactcctggccccaagtgattctcctgcctcaacctcccaattgggattacaggcgtgaccactgcacctggctcctcccatgtactttaagtaatctctggattatttaaaatacctaatacaatgtgaatgctttgtaaatagttgttacactgtatttttttttaatttgtgttaaattttttttcttttgaatatttccaatcgcgactggttgaatccacagatctggaacttgcagatacagagggcaaactgtagagttaaagacattgctttcatttgagatagaattcacattttaaccacaaccttttcggctttctatttatgtaaaagttctaattgtgatttctttatctgagggtactttactctgaaacatcacagccagcttgttttcacatgagattctctgttagagggaggatttgatgactttctccaaactgaactacatttcctgtagactagaggagaaataactgtgaacttcacatttcctgaaaatagtcaatgatatttcttcgttacatttcatctcagacaagccatagtttgcccatgcagtgatagatgaacttcttcagtcttacctgattataggtgaacaagtgttcagcagtctctggactccctgtgacatgctaaaatcaagtgtttattgtaaaaacacatcagtagtacatgcatattttctttgtaaaacatttagtaaacacagacttctctttgattgccctccctcaatgtaagcagctttcaatttgatgagtatcctaggtggcatttcttcagtacattacacacatgtacacactcacacatgcatgcttgacgtgaaggggctctgctatcttatgtgtatcatttggtgagttgccttctcttccccaattaacaatatggttttgaccatttcatgtcggtagctttgactctactcagttttctgtattgcattatacattgtgactgtttttccggtattcatgtacttttagtcactgtcagtttttgctaagtatattacttaagccacatatttgagtttatttctccaagtcagatatctagagataaaattactgggtaagaatacatacacattttgattttaccagctccaccaatcatatacaagatgacctatttcttggccagatacagtggctcacacctgtaatcccagcacttcaggaggccaaggcgggcaaatcagttgaggccaggagtttgagagcagcctggccaacatggcgaaaccccatctctactaaaaatacaaaaattagcccaacctggtggtgcacacctgtaatcccagctactcaggaggctgaggcaggagaattgcttgaacccaggagatggaggttgcagtgagcccagatcatgccactgcactccagcctgggcgacagaaggctctgtctcaaaaaaaaaaaaaaaaaaaaaaaaacctatttcgtgatactctgaccaatattggatgttactaatctttttaatttttcctaatctgaagcattaatgattgcttgtacactttaccactttaattttcatgtctaaaaaccttccctttccttctcttttccaaatgtaattgcaaattaaacccgactcaaggccttattcttttgggtccttgagatggttctgtgcctctgtctcccccctcaccctgtctgctgcctgcctgcccagcttgctgttcctcaagcatgccaatcgtatttctgtttcagagccattgcgttatctgtttcctctgtctggaacattcttcccccaaaatccttacacgtgacccgttttccagcctccctatggctttgtgtagatgttactttctctgtgagacctatcctgccacccgtttataccagcagttccttcccacttgtgccaactatgcaagtctcttttcatctgcagtgctgactggctcctcctaacacactgtagtaatgtgggcagtttgatggaatacagttgctagagaagattcacaggaccccaaaataacaatgtgtccaacctgcaccgtacctgagaaccaggaagcgcaagatggagtgtcttcttgtatactgctggccctgagtctatatgaaccagccccactggcagagcctccaggcaaatccttcatctcactactcataaacaatgttgacaggccagcacaatctgtccccaaacttcccggacctgtggctataaagcaccactgtctaattagtacattttgtgtcatgcaggtactttagtgaaagcagtgcaggccggttccaagcctgttgaaatgaacctcccaagacacatacaatttacttatttattatgtttatttgctgtcattccttaccagcatacaagctccatgatgacaaggatctttctaggttgcaagaccagcgcctgacataaagtcatgttttttgtcaataaatgagtgaataactaacagagcaagatccccagtataggcattagccttgagtagctaaaagaagttctttctatgagactggagcaaaagaagttagcgtttacgtgggtagctagctacccatgtaagcaaatttgggctggtagcttcgcgctgaaaaccaaggaacctagacagatgacttaaatttccctggggtcctataagaaagaagtcaggcataaaagtgttataggtaaaatcgatgtgaagttcagtatgtgtatttgtgctgatggctgggctaaagacgggaagtcaatgggcagttccaagaacagaaagtggggtgggtaaggctgggaacgtgaggtgtgtttcaaaggaaacatttcccctgtctgaggatggttaagagtagagttaacccaagaccttcctgtggatatcagcctggggtttcatgtgtttgtgagtgtagttacagtttttgggttttactggctgattggagttactgtgatttaatgacggtagggcaagcataatcatggttcttttctttggtaattataaaatagaaattgttttattactgtgtcgtggtcttgcagggaggatgacgtgagaatagtgctaccaagcaggcagtgggcgtgctgccaacccacatagagtccaagatcatgccacttgttttgagaaaagaaaggctttattgcaagttgcctggcaaggagacaggaggaaactctcaaatccgcctccctgaggtgggggctcaggcagtttcataggcagagaaaacaaagtgtgatctgattggatcttgcaatggggtgatgctgggaggtgtcatctgactgggttgtgtcacaaggtgatgccagggctcaatctgattggatcatggattatgccatcaggtgtttactccttaatttggcccccgttccttggtctaagtgcttaggttctgcccgtggttacatgcttggttcacctgggcatgctcaagtgacgtaacttgcaacttcaggggccgtggcaattaaacagttcaccattttgatacacaaagttgaactagattgggctggtttggtggtaagaacagcaaaaaatcgaaagagactggctaaaaactttcatggaaactaagaatgctaggatcatgaaaatgtctcacaaagcataatacagagccttttatacagtcttttaaattctgtccattttctttataactgcacaaaaaaataaatattgccagttcacatacagtgcaagaaacacctcttttagaattttttattactgatgttataaaaggtatcagaaatgtatgcgaaagggctttttctcctgccttaagcagttgcagtacagcattaatttttgtgttctttttgcacagcgtaaatgtatgcagcccaaagattttaattttaaaacaccagaaaatgataagagatttcagaagaaatttgagaaaatggctaaagagctacaaaggcaaaaaacaaatctaggtaagctaagaaatataatacagttctttgcatttgtgtccatacaccttgtttaatttgcatgatgactagtggggttcagcatgagagagctgatgaagactatgatagctttactctatgaaggagaaaacaaaatgtcaggagcctgcgggagacttggctgggagccataatagagccacgcagcttgagctaatcgaccacagtcttaaccattcatcaaggtggtcgaactttttattttcgggaatgatttcagaagaaaagcaaactttggctaataagcattattgaaataaatacctatttatttcttctttatatataactttgtatttttacctaattggcatttttgttttgttaccctgaataggcaaatcttagatgatacattattttagtgatttgggaaaatactttagaatattatgttctataacaagatgtcttagaaaaaaatatatgtattcttatgtatatatattgttaaataatatttttatatataagaatattatgggctgggcacagtggctcacgcctgtaatcccagcactttgggaggcagaggcgggcggatcacgaggtcaggagatagagaccatcctggctaacatgttgaaaccctgtctctactaaaaatacaaaaaaattagctgggagtggtggcaggcgcctgtagtcccagctacttgggaggctgaggcaggagaatggggtgaacctgggaggcagagcttgcagtgagccgagactgcaccactgcactccagcctgggcaacagagtgagactccaactcaaaaaaaaaagaatattatgaaacattaagatgctttgtacgtttttggtatttctgttatgcctttttcactgtcgtctaaagtcagtatttcctactaattctgacacagcattgctacagataagcaattatggtcactagaaattcctaggaagcattaattcctctagtttttgttttctttgttttaatctatgttactatgtcacagattctctattctgtgttttgaaattattcaaatagaattgtcgagatttattttatttatttttttgagatggagtctttctccatcaccaggctggagtgcagtggtgcgatcttggctcactacaacctccacctcccgggttcaagcaattctcctggctcagcctcccgagaagctgggattataggggcgtaccaccacgcccagctgatttttgtatttttagtagaaacagggtttcaccatgttggccaggatgatctcaaactcttgacctcgtgatctgcccgcttcagcctcccaaagtgctgggattacaggcgtgaccaccgcgcccggccaagatttattttaaatctgtgacgataatgcgacagaactgggtagaacacttagcccacatagtgctgccacataattttccagaaacatggcctgcatcatttgtttcatgctcagccctcccgctgcctcacctggtgcgtgtccatccttccttcacaccagctgtctcgtcttcgtcaaagctcaagccagaaacgtgcaatcgtccttgacatctccttcttcctgacactaacccccatcaagaccatggccctgcttctgaaatagttgtttgacttcttctgttttctccttccctcctctctcccctgatgcctggatcatccctcctgcaccactgcagccactccttacgctgccctccactgtctccttacagttcatctctgtgctgcagtcacaatggtgaaaactttaaaccagaaggacatcccctccctggtttaaaatttcctggtgtcatcccaaggaaaaatattcaggataaaatcctgtatttatcatatcctccaatttactaggtgctttatgatctggcctctctttctagcctcatagcaatattgcacactctcctataattctttatacttttgtcactttggccttctttcctatgtcagtgacagtgtatttgaaaatactttggcaacatggtaatgatagatacaaaattttcttcttagaccaaatatgtatcgtaattaaaaactatatgtataaagtattaatgattcaactaatgtacatttgtatattgtcagaactacagtaagggtgattcaggcttaagagtcccaaaggagaatatattaaatgattcttggtatttttttgttgggggtgagtatcaaagttctgaagggctctttgagcatatgcaaggtagcattccagaaaaaaacacaactctgcacccacacaaaacgagctcataacttcatggttccgggaccatgctgatcccacttcatgcagtcaagttcatgtctgggtctgtgagtgtgtttgagggtaggagtgatggttaatgggggcagtttctgaaacctgagacaagaaacagaaactaaattgcattccagctttacaacttttaacttctgtgtctcagtctttgtcttcaagtggggatactgatttgggtttggatttgaggttggatgcactaatgcatatattgttcttagcacagtgcttggtgagggcagttgctcagcagatgtgagccagcagctgtagcagcaacatcactgcctgtggaggtggtggaggtagaatattagcaggagtaggtaatgatgttgaaagggaagaaggaaaacggggtgtggggggttgttctttaaaaggaatcacattcctgaagtatgaaggcactttttggtcttaaagtggattttttgtttattttcagatgatgatgtacctattctcttatttgaatctaatggttcattaatatatactcccacaattgaaattaatagtagtcaccacagcgcaatggagaagagattacaagagatgaaggagaaaagggaaaatctttcccccacctgtaagtaattagtttgtaaaatgaaaattatgcaaatagccgattcaattatggtggaaagcttcttttttctttgcctagatattttaatgtttcctggtagtaacacattttgacttatttcatggctggctttgttttccagaaaatcttatgcatcattaagatttttgaagcatatgttgggtgtatagtattcttcaagtttaaaatcctatttgttgtagctcctttgtaatttctattatctttggaattttttctttctttttttttaaaaaaaaaatgaatcatgtctttttttttttttctgagatggagttttgcatttgtcacccaggctggagtgcagtggcgcgatctgggctcactgcaacctccctagttcaagtgattctactgcctcagcctcccgagtagctgggattacaggcgcctgtcaccactcctggctaatttttttttgtttttttgtatttttagtagagacggggtttcaccatgttggtcaggctggtcttaaactcttaacctcaggtgatacacccgcctcggcctcccaaacggctgggactgtaatccaggcgtgagccaccgctcctggccgtgaatcatgtcttttgaaggaatttgctttagattaatgtatctaaggaatcagtttgtttttcattatttcttttatctttaaaatttttaattactgaagtgtaattcacattttaataaaacatttatcaaagtagctaatagtaaaagttcatcttgatacccatctaattgtactcttctacctgggggtaacctgtattttaagtttaagtgttttcccagatctgtttcagtgtatcagatatctgtgtatacatgaaaaagatacgggtttggtttctgtgtggaggtgtaatttctgttttacctaaattagataatgacatatgtattattatccgctttatttacttaagagtatcctggagggtttgtttgcagcttagttgttgtagacctatttttgttttaagatgctcaaagtagtctacagttttgatattgaaaatctattggtgggtatttttttcccagttattagaaattgtgttgcagtttttattctttttttaaccatatggtttggttgttcttgtttttttgttaagccattttcctttctctagacataagtctttccagcttcccaccccgactttttactgttataacccctgcatgtgcctacgtgaatccttgtatttctgagtacttcgtgtatttcaataatactaattcatacatgcagaatttgattttttaaagacatagagtctccctgtgttgcgcaggcaggacatgcactcctgggctcaagtacttctgcctcaccctctcaagtagctaggaatacaggtgtgtgccacgatccctggcttattgatagatatagtcaaattatccttcaaaaaatttgagtcatcttattgtcaccagttgtttataagaatgcccctttctccatacttggaaaactgaatggcattagcctgtagcctttttcagtcggaagcttgaaaaactggatctgttcttgaagttacttttgattagaagcaggtttaagtgccttttcatattactgactgacttaccgaatgcagcttttaatgtgatcaactattacctcgcttaattttatgtcctttgtccatctgtatcagttaaggttagtttcggctgcatataacaaagacaaaaaccaatgtgttacaatcgatagaattgcctttctctgtcttgcctagttcagaagtaggcagccagggctgggatgccattccatggtgtctttaagaaactaggttcccatctttctgttgtacctgcctggcttttcttgcaaaatgtgtgtgcctcccagctaagccatctccttttgacagccttaccagacgtctatccaatattcctgtctaattccattggctggaatgtggtcatatggccaccccttttgcaagcaagactgaaatgtagtcttgactgggatgcattgctgtcctgataaaatcaaagttctgttgttaagaagaagtgagaatggacattgaggtagataactagctgtgtcccaggtggacatccaaattgtttcagtgtgcaattatgtgtataaactaatttgccttaaactttactttttctattacttggcagtgttaattctgctactttactgcgtccagtacagtttaaaacttaactgaaaattttatgtgtgcttcccttccttatcttggtttattctcttttttttgctgaagttttctcagaaaagtatccttttgagtctctaaaaaatatctttggatataagatccaaacatttcttttgtttcttgactattgtatgaaccgcctttgaagataatacttacgatcttatttgttaagtcattgacatcctaagtgttttctatgaaacctctaggatttctcaacccagcacagctgacatttgggtctgggtaattctttgttgggggcactgccctgtgtgtggtaggaagctcagcagcatccctgcctctccccactaacactagcagtgtacctactgctctccctcactggcgatatccaaaaatgtgtccagacattaccaaatatctgctgggaccccaacgtcacctctggttgggaagcagtgctctagttttagaggtaactatgatgagcatccttgaagaaaaatccatgattatcaaataagaagactagaacagactggaaatgttcacttaattctgttgagcttctgattagattcaggcaagttgactttaagatcccttctaactttgtgattataggatttaatagaatcacctatgattaataggaggacttcctgctggcttcgtctgctaagaaatactgaaactttatctaatgcagtgtcttggtcctgtttttagcttcccaaatgattcagcagtctcatgataatccaagtaactctctgtgtgaagcacctttgaacatttcacgtgatactttgtgttcaggtaaaatttttattttcctttctgtgatatgtttaagttttgagaataatatgattttctgatttagaatttcatgtagcaacttctgatgagtaaaataattagttaaaactagaacttctaaatttccccctgaaattaggtattataataaaattaaggcatgagttaaacttcctttttggttcctataggtttttttttcctaggcatttgctttcttgctacagaatccattgctctatttaaaaaattattgtgaacgtatatgaactaatctgtatgcagtttaaactacatagaactgaggtcagagctaaggaaatgttgtttcacacaatgtataattaacacaaggaacctgttattgaacggggtcagtgaagtatgtaaagatcgtcaattgaggagataaatagaggatttctaattagaagcagaaagaacactggtaggaattagtgcagttagttccatgttacgcacatacatgtttgtaatgtgggagccctagttccacttaggatggtaatttttcatggtcatatcttcttcgtaccaaatttcttacagtttcttcacctagtccccagtggggctcaagtaagtagcagtgatccctgaaagtactatgttcaaaagtgcttgagatgttatggaaaatttatcatgaaagccacagcaatgacaaagcgcaagatggcatcaagatattagaagtttcaaacaaagcctcctttcagcgcagggttaatccttgtactctcacctctgtgtgctggaattatttacccatttctcttaaacagtctccatctttttattttacacttgttacatttatttcctagaagttggaaacaagtgataataatagctaacattgatttcatttttgttgttgtaggcactcctctaagtgtcttattcactgttatctcatttattctcccattagccttaagaggtaggttccatcaccatcccattttgccagtgaaaaaccaggacacagaggtcaaacagcttgtccaaggtcatgtggtttgtgaatggcaaacccaagcttctaacttaggcagtctgacatcacagattacactcttagtgacatgtcacattgcttatcgggtttttgaaaagtgtgataaaacataaaacaattttagatgctgaataagatatattgagcatctaaaattaaaagtgaccttatttccaattactgccttgaagacacctggggcacagttggaagggaagctttggtggttacctgtgttcttcctttttaaagtagaacttcagtgatttcagacagagagttctaacacttacgtgacctccagattgagtgatttctacaaaacacaggccctccaccagcaagtgctgagcccctattgagggagccagcacgggactagagacttcttcatattcattccagtagcttatagcacagtgacgggcagatgcccacgtaaccatggggcagtatgatgcatgatggtgtgtagcagagggggcaaggccagggagagctggcaagggcagtgggagggtcccagggatgttgacaacccaggtgggtttggaaggatgaattgtatttacccagaataaagtgtggaggaaaggggaaggcccagagggtacagaggagtatagaatatttaggaggtagcagcagcttagcattactctcaggaaatgagtaatccatataagagttgaaacattaaagcctaccaaatggctcacttttgaatatcagtgtaatacgaggactttagtggaagacagggaaggtaagggtgagctgtgttcattgagggaatgtttcatgcaagtctagaactttccctagatcttacaacagtagttcttaggttttagaattattgatctcctggaaaatttagtgacaaactatggatgctcttttggaaaatgtgcacatgcatatggaaatttgcctaaaatttttagaagtttgttacacctcttctctatccccactgctatcccatacacccatcaaagcccaggttctctagttaaaaatactggcctaaaatgtacccttaagtggaaatgagaagaactcaagtgtggttaatagtcttcttaactaatagctgtactttaaaagttgttttattggtcaactgaaagttgaatatagaataatttaaaccacttttaaaagttagctctccgttaatgttttccagatgaatactttgctggtggcttacactcatcttttgatgatctttgtggaaactcaggatgtggaaatcaggaaaggaagttggaaggatccattaatgacattaaaagtgatgtgtgtatttcttcacttgtattgaaagcaaataatattcattcatcaccatctttcactcacctcgataaatcaagtcctcagaaatttctgagtaatctttcaaaggaagaaataaacttgcaaakaaatattgcaggtaaagtagtcacccctsaccaaaagcaggctgcaggtatgtctcaggagacgtttgaagagaagtatcgtttgtctcctaccttatcttcaacaaaaggccaccttttgatacattcaagacccaggagttcctcagtaaagagaaaaagagtatcacatggctcccattcacctccgaaggaaaaatgcaagagaaagaggagcaccaggagatctatcatgccgaggctgcagctgtgcaggtcggaaggcaggctgcagcacgtggcgggacctgccctggaggctcttagctgtggggagtcttcatatgatgactatttttcacctgataatcttaaggaaaggtattcagagaatcttcctcctgaatctcagctgccatcaagccctgctcagttgagctgcagaagtctttctaagaaggagagaacaagcatatttgaaatgtctgatttttcctgcgttggcaaaaaaaccagaacagttgacattaccaatttcacagcaaaaaccatctccagtcctcggaaaactggaaatggtgaaggccgtgcaacttcgagttgcgtgacttctgcccctgaagaagccctaaggtgttgtagacaggctgggaaagaagacgcatgcccagagggaaatggcttttcttacaccattgaggaccctgctcttccaaaaggacatgatgatgatttaactcctttggaaggaagccttgaagaaatgaaagaagcggttggtctgaaaagcacacagaacaaaggtaccacttccaaaatatcaaactcctctgaaggcgaagcccagagtgaacatgagccatgttttatagttgactgtaacatggagacgtctacagaagagaaggaaaacttacccggaggatacagtggaagtatgtgaatctccttttccaagtcaccttcgctaaataaacatgtaacagtgcatccatattttaaatttatcacaactttttcataacttatttccccatttactcctctttttacttaaagaatgtgcatttgatcattccaatgataaactctttaggaatagatgacttgctgtcttgtggaacttctagacttattggttaagtctgttaggaatctatttctccaagacttttccttcttataggtcaaaaggataagtagtccatagtatgaataactgaggggagtgaagtctttttccttattccattggagtcttggcgctgcagcgtgtgtaaagatgtatacgatagagagtattttaaaacctaggttcttaatagtgaggctatttaaagaaagaaattaaggtagattaagccatcgattgtatcaaagagaaagtgtgaaaaactacttttagaaatctgttgtcaatattgatttttgaagaaactttggtcagtgttaactatgaagmaacatttaaacatttttgmtcatttgtaacaagccttgtttaacttgtacttattttgcttgaagcatcacttgaaaaggtttactcctattcataatttaattgtaattataataaaccatatcattttattaaaagtcaaaacaataaaaaattttgcacttcacagttataagcacaaataggttccagcaaccaaaattgaagaaatcttgaactttgaccgtctttacctaaagattaggttaaaatttgagtgagaatgcattctctctgcatgatttctctgctctacaaatgttttaactgcctctttgaaggtggagaagtcatggtagcgtttgaaatcatcacagacatgttacataccttttccttgagtatacgctccccaaaattgtttcacaaaaagaatgaaaataattttatgtttttggcctgctatttatatcttggctttctgaacatatattaaatttgacaagaaactgtattttatgttccattagccttagtatgtgttttcaaaatatttattttaaaatgttgactcaaaagttaatataaaacaatagatgtgtaaaattctttggtagttaagaatatcctgttctgaggtttacattctccatctttccagttttcaccttgtgtattttttaaacttttgaataataatgacatggaaatgtaaattaagtaggaaaaagctggtagcaaacagtgtggcatggcctaaaatccccgtgttgttgggagtgtgctagtcctcggaagcaggtgtgttatgttctagaacactgcccccctgcgtcgacagcctccggggttgggggtaagtagaagsgggtgaggggccagcactagttgactcaaggcaccctggtggggacggagaggttttttcgctcagtggtgcaggccatcaggcagggcccgggtgcaagaaaacattctgtgtgcgctagtgcgagaggatcttctacagtcacctgccttcatgccattacagacagacgggaagtcactgggttctaggacataaaaagacctacatgttggctagcctaaatcgaacccttttgtagtaataaagattcatcaatgttttaaactgtcccctgtcagccccctgggactcaggtgaaccaactctctttgggaatctatcttagaagatgaaaccataaagccttcagtttcagtgtcagggatgcacactctatatctggtgaaattatggaggggtgaaaacttctgtacagcaaactgtacctccaaatctttaatgtcgaaataaagggctttttgccatttctgttttcagttcacttttacttgttgctgttgtcagtatctaagatacagtgtaaaaaaggcttcaaaaacaagttacaaagagcttcaatacgctgatagaacgggaactgagcgagaaacaattttggttttgttttgttttgttttttagttttttttgagacatagtctcgctcttgacgcccaggttggagtgcagtggcacaatctcagctcactgcaacctccgcctcccgagttcaagcaagtctctgcctccgcctcccgagtaactgggattacaggcacccatcaccatgcccagctaattttgttgtatttttagtagagatggggtttcacgtcttggccaggctggtcttgaactcgcgacctcatgatctaccctcctcggcctcccaaagtgttgggattacaggcgtgagtaccgcgcccggccaacaattttgttttctaaaatctttaaaatcattaatttttttcttttttacttttttattctcttaattttataaacagtacacagatacattcccattgtaacaaagattgctaagaagactagaatttccatctcctcacttgcctcttttcactaattcacttcctaactaatgaaagacatgcacccgttgtgtctcaggtgctcttcaagtttgtggggacatagagaatgaagcagcgtgcaccctcatagaggaagacaaatagtaaataagtgtataacaatgtcagctagcaagcagttaatgataaaaagaaaaacaatactgcattggatagataaggtgaccaacgaaggcttccctgagaaggtgacatccgatcacaggcctggggagggagagggagcctgtgactctgtcaaaatccatgtttcagctggaggtaacagcaggaacaaatgtcctgatggaggaaaatgcttgcaggaacaacggggaggccagtacagcaaggacttcctgagctgcaggaaggaggttggagaggggtaagagccagagctttgggaccttcagtctctgacaaggcgggcagctgttttgttttgaagtgtgatgagaagccattggggcttttgaacaggggaacaaccaaatctgatttaggttttaaatgtaaccatggacactgaagaacagactgtgggttggagtgtttgtctgcacgaagcagccactgtccacagtttaatatttccttccacacatttcttgtgtgtgtgtctacaagcatacaactgcaaatagatattttaagagaattttttgcatgcatagaattatattgccttaaaaattgctttttacaaaagcagtatgtcatatatttacatattggtaccagtaaatcttcattttctaatagagcctataggtagggtcagcacactttttctgtaacagatcagatagtaagtttattacgcttcatgggcaaagagaccaaatcgaggtatgtaggtactcatgagatgattacataatgagaaaaagacattttccacaaaatttttattgacactggaatacatttttttttgtaatacaggtctattaatgagaaaaataaaataatttgtggtgggggaataataacatttcatttaattggagttcagactgagtgttcccatcaccaacattgattgcaaatgtttattaaggctgatttgtaataagatagattttacgtatttcacttttgaaaatatcttttcacacagacagatactcctgattcgatgtcagtccacagttagataatttgcattgagcatcttcattgcttagaagacgctgatggaattctcttagattcttctctcgatgcctgcctcttagcgtgtccttatattgcagattcatcacttgcaattgaaaaataggtggaagctcctcaactgtgcagttaaatgggttttgaaataggaaattccggccaggtgtgatggctcacgcctgtaatctcagcactttgggaggccgaggtaggtggatcacttgaggtcaggagttcaagaccaacctgaccaacatagtgaaaccccatttctactaaaattacaaaattagccaggcgtggtggcacatgcctataatctcagctacttgggaggctgaggcaggagaatcacttgaacccaggagacagaggttgcggtgagccgagatcatgccactgcactccatcctggacaacgagagtgaaactccgtctcaaaaagaaaaaaaaaaaaagaaataggaaattccctttgctcttgcactcagtctgaaaagtgctgctgtagtttgggctcaggaagtatgtccacagccagtttgcatgggaatggagatcttcttgttttaacctctgacagcacaagagagaatcgttgcttatttgtggaaatgcgtcccacctgacccttggcactgccaatcacagctcttcaaccaccgaaagtcagtttgaattgccaagtagttaaagccgactggtcatcctgaactagtgcacagcttggcttctagttgcttttcacgaaggagacacagttgtcctataggtgccgtgtgttcactagcaaaagcagaaaagtccttcctataccccacttgtccatgggtttgatacagattttcttctttgctgtgtgtgatggatttttacatgtcagcaccttgtacatacgtgttgtgagcttatctgagcaatttggtcatgtccaactaccagggtcttgttcatcgataatagtcaccagttgttggaggtcaatgatggttaactactcttctaccttctatctaccagatcttgttgaaggcaagtatcagaaagacatttattaaacatttattggcaagcagttaggaagtggtccacaaattgaccaatatgctgaaggcccagttctctgtcctttagtgcagtgtccatactttatctgaaaggtttgctggaggcagacaacattctatgggcaagtttctgcaaacttgcactcagcaccagaccatcgtgtatcctttgaccctgtggtttattatagggtcatttagggattaagccttggataccacctccagggataccagccacaactcatactagatggttatgctctgttctgtgtgggtattgggttcccctgcaatatttaagccaattcagtgttcttgaatccatgaatttaaccaataagaaactgtttctcacatccattatgctgattaacaagctgatgatgtcaccaataaccactcatttttgtcatccattttggcttttaacaaagcatctaatattgggctggaggatttacaggagttggggttttttgttgttgttgttttgagatagcgtctcactctgtcacccaaattggagtgcagtgacatgatcgcagctcaatgcagcctcaacttactgggctcaagtgatcctcccacctcagcatcctgagtagctgggactacagacgcaggccaccacactcggctacgttccccaggctggtctccaacttctgagctcatgcaatctgcccgcctctgcctcccaaagtgctgggattacagttgtgagccactgtgcccagcctatggtatagtacattttgcaaattctgagcattcaagaggaactgtgaattactattgttgcaaataaatagatagacatatattcattaagtatgttaaattgttgcacttttgactcttcaaataattcacaagtgtattaagaaccccctttcccatagcctgccagcctaactcactggggctgcaaaactaagcaatcctagcaacttgatgtgggttagtcagtcttaacagaaggctattgaccacttaactgtttggttgattcattcattcatttacatattcattttttatctgtcagatgtttactccgtatctactatgtccaatgtataaacagtgagagaggtaaggttaatagaaagctctgtcccttgctttaaagaacttagctaagtagggaaggtacagtcaagatagtttacacacaagtatcaggaaattcaaaagtcagagcaattactttcagtgggaattaaaattgatattggaatgacctctacaacgattacaaaggataaaattccgcattatctattgaagagtgtttttgtttttttcagaatgaacaaagtgaacttgatattttaatagatgaatatgaatacagtctcgttagcagagttttacttgtgtagaacccgtataacttgcatatataccaaaggtatctctggaaaggaatttttcctaggtgtcttttaagattctttccagtcttaatattttgcatactacattgtaaaataatttcatattcaaatttttgaagcttagaagacatttctcattggataatgttaagtgtatatttttacatgttaaaattatggattattcagccttcagaagccttttcaacccttgactcttgcatagtgcattgtaagagtaaatactaattgtttaaatgtgttattaatattagcattgttagtcttaattctgtatcttggaagtaggaaagtaggatgtggaggaaaataaatgttaaaaataagagttatttcttcggccttagctctagacaaaatttgacacaagccaagtttctcctacagtcttttcatcgtccacttcttcatctctccctttcctagtatttaagttacatgtgtccttatactgtcttgccctggatctggctccaaagtgatcatattagtcattttcttctcttttccctcagtatcaatacttttccttaatcttgcttatctctgttgagtagctgaaggttgtgatttaactaattcacactgagaggtgagtgagtgatcatttactagctttcattgatgtgtttgcattttgatggtattattaatccaaactaatttccaaatggtgaaatttcagataactgaaagataaaaatgtggggtctgtcagattcatttccgtatttgatcatttcgtgaaaacgaagtcaatgaattgtgtgtgtaatgaggttgggaggaaaatgagaggaagatatatggctttcacagggaaatgctgtggaccaaattgtgtcctttgacccccacatttatttactgaaggtctaaccctcaatgggataacatttggatagggtgatctttggaagataattaggtttagatgaggtcttgaagatgggggcttcatgatgagattaggaccattataaaaagaccagagaactggcttcctctctctctgccatgtgaagacagcaagaaggtagcctccttcaagccaggaagaaagccttcaccggaacccgaccatgggggcaccgtgatctcggccttcaggccaccaaatctgtggtattttgttatggtagccccagccgaagaagacagacattcatccaactggggtgtgttggaggaagagcagctaaagggtgcatgttcgttggaatttcttggagacattcaaaatagatgtccattaggtagttggatatagccagccatacctcagctgggaggtctagacaaggtacagagaattaggtctcttcagtaatggacgactttatgggaagtgatgaaatcaccttggggagtgagaagggagctgatgacaacccatgaaaaaaccacacttaggagcaaacacgaataaagagtcatccaagaagtgggagagtcaggaagaggagggtaggtgtttgtttacagacctcctgccaaaagtggagtccaactaatctttccacagatgttttcagaagtactttgcactctcaactgctttgggtttaccgatgtcaatgttaaaacccactggcaaattagtgtggcagagtttatgaaatgttttaaataaacaaatcatttacttagatcattttttgacttcaggatttgtgaaattgtgaaaacatgttaacaatatcagtctttttttttttttaatatcagtctttcttaagttttaaaagattgtgttgcatttcttagaactttatgtttataaaatgctttacagcctgtttcgttgttcggcaagaactgaggcaagtggctattataaaacttttattgaatacactaggaagctgcaaatttattcatgactcaataacagagcactacgtcccaaattatatctctagtccactgcttttccgattttgacacactcatgcttcaagtaaatatttgttatttaaaaaggaaaataagtgcgtagtagatataattaataattctaattatttttaatcttaaagacgataggagattgcattcatgttctaccccgggggataaagtgggcctgggagaaaagtcagtgcaagtcaaccataaaagatacctgaggaggtacgggatcagtcaggatgtgactggtttgagtctcgagtggattcagtattagggattatggcaaagagtgtaggttggtaggtttgtggtttagaactggaccttaaaatctgtccagggcccaggctgcaaataacaactagcttgaattcaggaaagtattaacatttttattctacatcctttttcactgagataggaccctgtttttgaaaagagtgacagtttttaccttagactctccaaacttagttatagctggctttatagcattttatctgcaaagaagtctttctcatgttatatgatttttaatctctgagggcactgatgttaatttcacgttgcattatatttattcatctgcatctacattgtctattgggttgtgagctccctaagtgtgggactatatcttgtgcattttgcatctccagtgggtagatgattagctatttgttaatcattaggtaatcaacagtgcagtttggctatcacctgcctggcaggttctagtaccccctaggctgctacataacttttgcgtcaaagtttgcattataccattgagaccatgttatggtccatgttagctcctccttcaaaatcccatgtaagtcataaagtaggcaaactgtttgaaggaggaggaagggtgagagtaagaggcaccctctgaggcagtagatgagtcaaatcaaagtacacatttcacatttcatcgtgggttacttaggtctacagaggttagcatctaaggaaaccacatttcacttgaatgagtatccttttggtttgtgtgtcttcatggcaagacgctggtctaaggtggaaacttggggggagtaaaatcatcatccatcatttgtaggttgaagcctgaagctctgtactgaagactattttctagaaaatctcaaactgaccccaaaacttagattaattattgcctctaatatggaactgcctactctgaagagctgttctttgtcattattttaaaatctaagaatttaagtttgacgagtgcgtaaggtatgggtatacattttcttacattatcaaatggacggagttgatgctgtagaacactgtaacctgattgttaccgaccattgaattaagtgaattgcttgggatattggaatgtaataaactgaaagttctagatagatctcaaagagccagatatatacaatttatttaaaaggcctataacttcctgtttccattatgcataaatgtgatttttgttttgcttaagttgtatttggtccatgtaaagttctaactaatttttaatccccttgggttttaggtgttaaaaatagaccaacaaggcatgatgttttagatgactcatgtgacggctttaaggacctcatcaaacctcatgaggaattgaagaaaagtgggagaggcaaaaaggtcagtgtgtaaaaatattattttaaactttcaaatgctgatacatcataatgttcttctctgggtcaatgaaacataaaccagtctatctgacttgtcttttattttaaaaaattgattatgggtaaatgctggaaaactcagaatatgaaactgaaagcgttgtttgcattccagacaaagagttattattgatagagcaagctttctcatatcactttgctaatgcatttcttataaaaatgcctgtagcttctctcaagcagagaatgttggttgtgccagtgtttcttgccattttataatcggaataaatatttactaggtaggaggtgaagaatccaaacattcattcacttttgaactaaccaagtcttgacctcaagccatcagagtgaaaggtttatatactaacactcaggtacacccttcactttgtggttttggctttaaaaccttgctcttcctctgaaagactccgctgatcctcttacatgagtaatagaatgaggattttaaatgtttttatcattcaatatctacttgcattgcttaaatttaaaattagccatatatattataccttgtgcctcatttttatgaggccaaaaaagtataatgtagtgaaacctgaattcagaatggtagggaaaaaccataccgattgaaaagcaacagatgaaaagaatgacagagtagatgggtctgcatggggcttccaggtcctgatacgcaggcttgaacagatgggcggctgcatttgacctgcggaagagaaacctgactcctttgcttcttatcttggcaatggttaaaagacatttaaaattacacagatttcatgaaagttggcagtaacttgtagaaacttagatttctttattgatgctttctggtttgtctcggaaaaaaaagtggagcaagaaaatggaaaggaaccctatttcaggtaaagcaacagatgtggagagagagagactgtcagggtcccataacatgtttgtggcgtgggcaacaccaaggcacctgctctacaatggcgttgcgcactgtgactccactgcagcctgcgggacctgctcagcgcgctgcctcccaggggtggggcccttcctagaacgctcgcaacactgtggctgagtttgtgttttgcgtcccagtttctcagtcttcttcctactgctacatggccgcttgacctagttcatttggaaagaaataaagaaccagtttcctttgcatctactaccgttcccgtgcctctcctgctgatgcgtcgcatggcaccacagctctgttctgtgccctcccgctttactgaccctttaccctctgccagtgtctgcccagggaagccgtggtacctctcatctctattggtactctacgttgtaccatgtctggcttttttttttttaagtgctcagtaaatattgagtgttgagttacttgttactcaccataaaaatactccgtcctgtctgatcaaaaggcatgaggtttgactttctcatttgcccacagtggaagttactgtttcagacgagtggtattgccttcctgtgcctgggatagccctgaatctgatgggctgggtctgtggaagcactgggttagggacaggcatcctgggcgggagtgtggccccttcttccttatgaggcatctcactgtaaatggcatatgaatgggagatgggtacctgtttgactttctggcattcttctgtagatcaaatagtaagtgctccataaatataaggtggtattactgtcttgagtaatgataaaagaatgagtggtcagagagggagacaaaatacacaattacaaatacacacctccatatctgccttcaactgctgtgctcaggaacaaaaatattttcatatattaaactgcctaacttgctcaaatttaagtcttcttttaaaaatattttaagagtattagtaaactttgccctcataatttagaatgtcatttctgaaacgaatccaccacttctggttctgtgtgaagaatcactcaaagcaggttttaaatgcagattttctgggccagtcatggtggctcatgcctataatcccggtactttggggcgggcggatcacttgaggtcaggagttcgagaccagcctggccaacgtggcaaaaccctggccaacatggcaaaatcccgtctctacaaaaaacacaaaaattggccaggcctggtggtgggcacctgtaatcccagctgctcaagagactgaggtgggagaatcacctgaacccaggaaggggaggttgcagtgagtcgagatcatgccactgcactccagcctgggcgacagagtgagactctgtctcaaaaataaataaataaatgctgattttctggccccacctgagaccctcctggccagcagctcccgaccccagtgcggcaccccgtccttaacgtggaggggacgaacacctagtgagggcgaagaatccaccttctgtattgcgtctcgccaatagcagaaggagcaagacctaggtttcccctctttcacaggattttcttcctaatccagtccttattagtgttcaccgcacagcctttgcttgaatgaatcaaaaactcctaatgccctagggtagtgcttcctgactgggctgcgcattggactcacctggggatctgtaaggtttgtggctgcctggccccaagccagacatgctggtgtcattaatatggggtgcaccctggccactaggatttttttaaactcctgaggtgattctaatgcaaagcagagtttggaaactactgccttgggacttttagaatttaaacaagtaatttatcctagaagaagtttcatttctttctaaacatttctcatgtaaagttgtttcatttttagactctaaaattaaagaccaaggcttaaagtcctgatttgcgggctgggtgcggtggctcacacctgtaatcccagcgctttgggaggctgaggtgggcagatcatgaggtcaggagatcaagaccatcctggctaagacggtgaaaccccgtctctactagaaatacaaaaaattagctgggcgtagtggcgggcgcctgtagtcccagctcctcggaaggctgaggcaagagaatggcatgaacccgggaggcggagattgcagtgagctgagatcgtgccactgcattccagcctgggcaacagagtgagactccttctcaaaaaaaaaaaaaagaaaaaaaaaaattcctgatttgtttgcttaaaggttgagtgagtgttttaggagcgcaaatttgatagcaatatagatgaaggacgtgttttattattttacaggttagaaggaagaatgatataaatttcttaaaaggtaacattaaatttattttattttattttatttttctgagatggagtatcactctgatgcccaggctagagtgtactggtgttatctcggctcactgcaacctccgcctcctgaatttaagcgattctcctgccccagcctccttagtagctggaaccacaggcacccgccagcacgcctggctaattttttaagttttttgtagagatgggtttcaccatgttgaccaggctggtctcgaactcctgacctcaagtgatctgccttccttggccctcccaaagtgctggaattacaggcgtgagccacagcacctagccagcaacattaaattttaagtatataacttcccagtagtttgagatcttttgatatgagcatggggagagaagtttatgttgatatgtggtaatgagtccacagaaacactaaaatttagtttcctggttttaaaagtatacagtggaattgtggaaggattgaattggtgaattaaaattagaagcttctgagtagcagcctacaaatataatgttagtatctcaaccattctttttttcccattaaataggttttacctgcttattttgttccttgttagatttcaagataaactgtgttaaactgaaatttggaacttaacacggccttttttgtttgtttgtttgagatggagtctcgctgtgtcacccaggctggagtgcaatggcacagtcttggctcactgcaacctctgcctcccgggttcaagcgattctgctgcctcagcctcccaggtagttgggactacaggtgcacgccacatatttttatgtataaggacatattaaggtattagattctattaagcacaaaattgtttctatttcctaaagaaaacaaaatcttgtaattgaatattaatgttgaaaaagggagagtttacaggaaatatctttcaccagctaatgactgaagcaatgcctctactagaatggagaacagtaaggtctgggcctgacatttttatgttttcacttgagagccagcctacatgctatttctgtagtgaggaaaatgatttgaaactcagatgtgtcccgtggccctaatgactttattttctttttagttttaaatctgaagtagcacttgcaggtaatgtcctatctgggcagccctgcagacaggactgtcagtcgatgagagctgtcagtcgtgagttctgagtaatgtgaaggtgccaggtagaaggtacaaaggcaagaaaggtgggaaggcctggagcctgtgcgaagagcagcacggccttggtgtggcccggggatggatgcagaaccgcgagaagagagaggctgacttcagccacggccacgggctctggggttagactgctctcatctttggttttctgtaggttcattgtgattgttgtaccagagtattgtttttgttgtttatttacttgagagtcacaggccgtcctgtctttgatctgttctggaaacttctccactgtgatttcttttgcctgttttctcacgcctccattgctgggaacgcaatctcgtgtgctatcctttgcttctatagcccatgtctcatgattttcctctatttctttcctcttgtatctccttatttcattctggatgtcttctattggtttcttttccatttcacctttgactctttttaagtctattctgatgctaaatccatatactgagttttaacatgtattatttttcagtccctgctattccatttattttttttaattattttttgtagagatgggggtctctccacgttggccaggctggtctcgaacgcctggtctcaaacaatcctcctacctcgtcctcccagggtactgggattacaggcgggagccttcatgccctctatttgatttataaaaaccatttccagttctctgtcaaaattattaatcctatcttttatttatttgaacatattatgcatatttcttttgaaataactcccttttctggctcccctcaatttctgtttttcttatctgttgttttcaatcatacgttccatatctaatatgcctggttagtttgtctttatcttcctagcagggactgagatgatctggagctggggttctgtctctgtgaggctagctgtcccctgggagtgtgggcttctgactctggttcacctcctcttccatgggtttcttcttccatgactcactgatttagtagctgggcaacgtctgcaaatagctggggcttgtttgtttgtttgcatcttgtccagcttttctgagggctcacagtgaggagcctatttcaaactacttagtccaccattcctggagacgatgggtgaattttaacggccacttaacttttctaaatagagttttggtgtgaatgcttctctgagaagacagcagtaagaggccaagtcaagagaaatgatttttgagatgaacacgtaggtcagtttgcaaaagacacactaaacacctgaattgacattaattcagtttctcttaaagagtgaaaaaaaccatgattccatgaagaattatagaatctcagagctataacttccattagctttttttttggtgtaatgcccatttttaatggcaaaaatcactctataaatcagccagaaaaagagtctgtttttttttagacttattttaaatatacttgtttcaaatttgttgagactttttttttttttttttttgagatggagtctcgctctgttgtccaggccgaagtgcagtggcccagtcttggctcactgcaacctccaccccaccaggttcaagtgattcttgtgtctcaacctcctgagtagctgggattataggtacctgccaccatgcccagctaatttttctattttttttttttttaattagtagagacagggttttgccatgttggccaggctggttttgaactcctgacctcaagtgatgtgcccgcctcagcctcccaaagtgctgggattacaggcgtgagccaccacacccggcctggtgagactttatttggaggatccagttaagcagttttattacctctgtaatcttagttgcagcatgtaggtcattgacattgatagttatacatcttttcagagggagaaatagaaaatattatgacgaattttgacctgttttctttgttacttgttgaatattgtcagacacagaacccaaagaagctatgtatagataccagcactctggtagaaatacacgaatgtaatttttttttctccaagtatttggtttattctactacttctggatttggtttttcaaaatattgattattatcctcaggaacatttttaatgtgagttatcaacaggatagctttttgtaagtggctcagttgtagaatctcattttggagccatctctgccaatccagcttgttgcatgtgaaggcaagctgtgggtcagagcacagaaatgtttacagaggctttcctaagcctggaggcctggagagatgtgaaggaacaaatagagcatacttattttgatagtggtttaaaaaaattaaagaattacacaccacatagaatgcttaaattcctgaaagtttctcaaatagggtgcaaaacaaataatagcttgcatatgctgatagttgcttgttcttacatctttgctagaatatgagcccataaggacatagtctatatcctgttagtctcttaatactcagcaggatatagcatcacaaacaaaataagtgctcagtaaatattttctgagtaaataagagatgcattaatttcccttttactttttcagtgaacatgtttaaaacatttttggtgctcttaaccatcactcagtaatgatggaatcatcatcatgtacttcacttatttttgaatattcttccaaaacttgagagactgtcttctttcagtaaaagatggattctcttctccaaggctgtgcatggcagcgcagtgttgctaaagcattgcccccagagccagatgcctgggttcagtcccatctctgttactcacctgctctgtgggttccatggtgttgaacaaattacttaatatctgtgcctatacttctttgtgtataaaacaggaataataataatagtaccagtctcctcaaagggtttgtgctaattaattgagttgaaacatgcaaagagtttaagatagtacctcatatatagaagtgctcaaaaaatgttagctattttcttcagcaccagcttgggtgagggtcatgtctgcatattgactgtgctttgttctgcagctataacttggagtaggtctctcttacctgcctcctctttgcccactcccagagaccaccatgtgtctttaatgaaaatgaccctcaaaactctgggacagtccacactgtgtttcttgttggacttactgaccacaggcatgccagagccaaaatagagtcttgggcagggggtgagtataggagtatagccttttctaaaagctccttcagtgattctgagctgatggtcatcctcccattgagaacctttgttttgggggtgagatgtaggccattagcatgaaattgtgctctgtcatctcccccaggaggcagaagactgagttctgcggtcagaaatgcccgcttgggggatctgcttcctcagttttcgagagatgctttcctcatctccagtatcattagaaccttcctgaaagaactgagatctttgtgagctgcgatagggtactcacagctgtcatttattgagcattgtgacctctttttagattgagttttctatttctcagtcatatggaaagctgaaaagaaagtatatttcagagagctctaatcatgtctttattgcggaggcagtagattgggaattacagctcatttgggtgtagcatccccggagaaggagccttgcagtggaaagaagataaaagggtcccagtggcgggaataaaaagagtactagatgcccagagggtgggaaaggcctagcccagatgcagtgtggccaggccagctaggggcaggaggaaagagagctgcagggatacagatgccttcctgagcagagaaaatagaatacttgagccaattttcatgtaaaatggattattttcctggcgtttcctgtccttcaagtaaaaggttctggaatgagtacttcactgctgtaatggagacactaatattttatgaatgcagttttacagtttgcagtaatgccaggcctttggctgttttccattagatggtgcacttggctggaagcatatactcttgtagctttgattttaaatttaactttcaagttgaaagagcagtgactcatccaaaggacaggtgatatttatttattttttcttgaaaatgcagcacgggtatgttgttatcacacgtttaggggaattgccacacttcctcgaggatgacaccctttgtaaatatccatgtaaatcatttccattgttcagacccgctgtacgcagaaagataggccctttagtgccgaccagccggccagtgagctctgtaagatcgaaggtgcccttggtttccaacacagctgtttcagtgatctgtaattgctttgataaatcacttttggcagagtgtacccagagctggcagtggcggggatgtgctcgttgtaacaggtgtgcggtccatcagcagatgttgcttgatgaagccatttaaaaaacagctgcctgttgatagcctaacagttgctttcagcccccattagcacgttgtttttttcttgttatgtatgagagaaaatatttctacagaaaacattaaataggatcttcaaagaactccatctttttaaaaatgtgttttatttgttcactaactgattttgcatgcattgtaaatgtgtggttcagaaattgtcaaatgtgttttggactggacgtggtagaaatgaggaccagccagggtggatctcctgtgcctcagtggtcgtctttggccacgtaaaggtagaggccaccgacggaggacatttcccactgggagacccacaggcgctaagagaggagctagccgaagaagtctatttaagatctgctgctttggccaggtgtggtggctcacgcctataatcccagcactttgggaggccaaggcaggtggatcacctgaggtcaggagtttgagaccagcctggccaacatgggaaaaccctgtgtctactaaaaatacaaaaaattacctgggtgtggtggtacacacctgtagtcccagctactcgggagactaaggcaggacaatcacttgaacccaggaggtagaggttgcagtgagccaagatcatgccacggcattctggcctgggcaacagagaagattccatctcagaaaaaaaaaaaaagaaaaattctgctggtaggcattctatgcactgagcaaaggagagatgtggaggcccaatttaaatagttacagctgctagctcctaaggtctatcttactatctgcaccgtttgcggggagtcagcttaatgatagtaaactgtgctaaatgggtctagaaatatccaattaatctgtttgagatattcggaaactcaatagcttgctgaagtagcaaacttgaatccttatttttattttaaaagggagtaaagggactgtagataagtaaaagatgctctgcactgcgcctctctggtaccagtccctctcgtttaggcagcggccacttcccgcggagctgttcacgccaagtgaccctgccactgcgctgctcccaccaccccatgtccaccccgtcctcggacgcctggtctcagcacatcaccggtattctcttcctcttaccagtaattagtttgagactgtgactcacttctgtccaacaagatgtgaagggaagtcttcctgggaggtttctggaaagcgttctctcacttgtgatagccctgggaagaaatgctccccgggtcctcagagctttgttgtggctggacgcatcttctggaactgcgacagcggaggaggaagccaagagagtgaaccaaaacaaggaagggcggagggcgggggaggcctgcaaaccttacggcttatttccactgacatcagagactcatgttaataagtaacaagcggctttgtttgttatgctcctcagacacgcggtaagggagacacacagaaatgcacagctgtacgtatttgtcttgaaggctagaatttactttaaatgtgagtggttttcccaggaaaaatttatgtctgttctcttgaggaataattatttcctactcaattttatctatcgatccatccatccatccatccatccatccatccatccatccatccatccatccgatacagagcctcgctctgtcgcccaggctggagtgcagtggcgctatcttggctcactgcaacctctgcctccccagttcaagtgattcttgtgcctcagcctcccgagtagctgggactacaggcccgtgccactacacctggctaatttttgtatttttttttttttttttttttcctgagacagatcttgctctatcgccaggctggagtgcagttgcgcaatctttgctcattgcaacctccgcttcccaggttcaagtgattctcctgcctcagcctcctgagtagctggtactagaggcacgttccatcacgcctggctaattttttttttttttgagatggagtcttggagtctcgctctgttgctgaggctggagtgcagtggtgccatctcggctcactgcaacctccacctcctgggttcaagtgattctcctgcctcaacctcctgggtagctgggagtacaggcgcgtgccaccacacctggctaagtttttgtattttcggtagcaacgaggtttcgccgtattagccaggatggtctcactctcctgacctcgtgatccgcccgccttggtctcccaaagtgctgggattacaggcatgagccaccacgcgcagcctttttttgtgttttagtagagacagggtttcaccgtgttggccaggatggtccgatctcctgacctcgtgattctctcacctcggcctgtcaaagtgctgggattacaggcggcagccaccgcgcctggcctaatttttgtacttttaagtacagacggggtttcaccatgttgtccaggttggtctcaaactcctgacctcaagtgttccgcccaccttggccttccaaagtgctgggattacagggttgagccaacgcgccctgccctcaattatatttatttctttgcctttccttacgtctttaactcttcacacttttaaaaaagttattgccttccaaataatatttaggaatataaattatttgatattaatccagggtaatttcgatttgtttttaaaaaaggggaataaaaacattattattcagaaggggttaaatacaatgacaaaaactgcaattcagaattaatgaggcgttataatagggtttgttaaaaaaattatgaggtatttaaaatagatttttggcatatccttttgtgacttttggatagacttaagacttagtttatatatcaatagtgagtctgtataggaaaagaatataatattcagtgactgtcaaaccagtgactggagcagcttggtatgaagcgcttcttattctggtctccctaatcagtgattttcaattttgaaaacttttttttgaagttgtgttgttttatttttctgcagaaatatcttctgcttttcattttaaagtatatttgctatttatttgcaatctagttctcatcattaaaagcagtactaaaatcttatcccagaatttataggttgtgtcttttgtcctttttttgtttttagtatttttctgtcactttacttcctcaggtgaagttttaacaaaaacgagggaccatggataggaaagtaggaatgaaacagtttacagggttgaagttgtggtataattctttttttttgttttgtttaaagacagggtcttgctctgttgcccaggctggagtgccgtggcgagatcatagctcactgcagccttgattgcctgggctcaagtgatccctccagccttggcctcatgagtagctgagactccaggcaggtgccaccatgctcagctaattttttttgtttgttttagagatgggatttggctgtgttgaccaggctggtcttgaactcttggcctcaaaccatccactcgcctgggtctcccaaagtgctgggattataggcatgaaccaccatgcctggcccatggagtaattcttgtggagttggaaggtagaggtgtgtacgtgtctgtttctcaaaatagtagcactagccaggaaatccatgaatttgcatatttttccccaagttcagcccatttgctttggtgagtttggggttatacttagagtgggtagtataaggagtttctgccctacaccttagcttaagcaatttgagcacattgctttttgagttcaccaccaaggatccagagctcagaggcagtctttcctgtgcagataagagtgcaccctgcctgcacctcacggtcttgggctctgtggcttctctcctcctgccactgccccttattgtgggtaggctggaattccctatggtcctttgtttggggaagggggatgcttggatgttcccgggtgtcacctgtgcatgccccctatgctgtcctcccacctgccctgtcctacaagcatgacctgcacccttctcccacacacccagaccgcagcttattcttactctccctggccagcccctcttcttggagaggagaaaggatgatgtgaaaataatatctaacattggggctccccagcgacttccacaaggagcaaggagctaggtgcatgtgtagaccccatgggagctttagtgttagataccgagtttgctagatgaaacatctttttaattgaggtggtgcagatgtattgtttgaacactttagacactaatgatgaactacttggatgtacatttttttggtttttttttttttttgctatgaaaattagaaaaaatatttatccaagacagtaagtattgaaaactgatactggtgctgtatggatcactattattgtattatttgaaactgtttggaaaaggtattgtagtttttagaaaaacaaagcaacctgaatattaaaagtctgtgaatttgagtaaaaaacagtccacataagggaaaaaatatataaggaaggacaatgaagttttgaaactgttactataagaaagctaaaggctgagcacagtggctcatgcttgtaatcccagcaatttgggaggctgaggcaggaggatcgcttgaggccaggagttcaagaccagcctgggcaaaggagtgagacctcatctctactaaaaataattttttaaaaatattagttggacatgatggtggccacctgtggtccaagctactagggaggcttgagaccaggaattcgaggctgctctgagccgtgattgtaccactgcactccagcctgggcaagagtgagaccctgtctcaaaaataaacaaaaaagaaacttaaagattttagtctcaattttctacattgaacccatctttagatcatagcatgtataaaattaaaaatgggggaatatcaacattattatatttaatgctatagcttattattgtatttaataagctacttgtttaaagatctggggtctcttgggtccacagactgagtctttctgaaggtgctttacacgatgtagctgccagggatctaggtcatataatatcctcaggatgggatttgaagacatttttccagaatttatcttttgtcatattggattttatttttaaaaatttcctctatagtcaaaatttatataaatatatgattctgatagtaccatatatatttagatgggcttatactgggcgtgaacaaggttaataatctttgtgaatatgtgggttatctccttattttacttattcttaaggaaaattaatttcactgtttaccaaagaactgatagctaaacccaaaagatttcaaagaatgttttgtttttgaaatgtttctatttatcactaataaaacgggtatatctgtttaagttgacctatctttggtcttactaaaacaaaatcagctagaccatttcccaaataatcatgcattcaatactctttttctctctctctccctgctccctcatctctactcctttagaactttcagaacattcttttgtgtagatacagtgtttcatgtctgttattgtttctcactggtcgttggattctttcatgtgaccacctttttcacgtttgctctgattgcctttggatgcgcctaactgtgtgcttttcctgttaaggaaaagaatcctgcatgtttttttctcatcgaataacaatgttaaaaacagaaaagggttgtttttcttctttgcagtaggcattctgtagtagataccttgacatacttaaatttgtgagatgtgtctagacgaatggaagagtaatatctcatattaatatattgctaataataagataaaggtttcagcttcctggagctgtccatataatagaatttgtacttgttttttcatttctgagatcctcatactttggggttttttttatttttttattttttcgagacaaagtctcgctctgtcacccaggctggagtgcagtggcgcgatctccgctcactgcaacttccgtctcccgggttcaagcgattctcctacctcagcctcctgagtagctgggattacaggtttcctgccaccacacccagctaatttttgtatttttaggagagataggtttcaccatgttggccaggctagtctcgaactcctgacctcaagtgattcgcccaccttggtctcccaaagtgctgggattacagatgtgagccaccatgccaggctctgagatcctcgtacttttaaataaaatgttaagatacatgctttatgcttttgctgcctctcatgtttcatgaatacaagtaaacccatgagtaactcatgaatacacataaacttctgggcctccaaacgatgccctgccagtggccatgccacaggaatcagaggctgtacttcactttgtggttgctttattattccaccattataagctttagtagaaaatgtaaagagggttgttaaactgaaggagtgttgtctcaaactgaaggagaaaagtagtgttggtgctgtaagatgtacataaactaaggggtgtcttttctaccatccagttagcaattaggaaagtccttctttgctcataccattccaaagggagtcatcttattctttctctaaatttccttacaatggaggctgctacagtttaagtatcgaaggtccttttttttcagatttcacctgcagtgcctataaatttgggggaatgcctttttttgggggtgaccaacatactcagtggatcttggacctaccaccaagtgaccttccttgctcacctgtaaggctgagaacaccgtaagcaaagtaccaggcttctttccccaagagggctttgtaagcgttggcgccataaaatcaacctgaggacttaggtggctggttatttctgagtaagtgaatatcactctcaaatacgacattccagcaaaggccatggttgcatagccactgtttttagttatgtcctggtaactaggaagatggattgttttttaatctatgcaaataattatattgcgctgaaaaaaatgatactcaattacagtttcacaattctggagggatcaggcagggataataagataccatttccagatgtttcctttctgtttataaaagcatagtcgactgaattgttaggagatacaggcagagggagaagagaaagggttccttatgtatccagaatatagagtgttaaaatagcaacaatactgtaaacaaaagccgcagtcctccttcagtagttcatctgggcctagtcattaatttttgttccacttgatcttgggttagcagtctcatgaatccgtctgcttctcaatgagggttatagaaatcctcttcccctggtggggtctcagcattatttagacaatgccataagaagcctgtacccaaaagtacccagtatagttcttctccacggggctctaacacagccccctcttggtcgaaggtaagtcactctggcctatagctaattgcagatgctgatcagggaagtgtcagagaaacacagaaatctgtaggtgacaaaagattttaaatggctatggttctcgtattactgataattttcaaaactaaatttattgagagttcattacaacagtattggcaactgataagtaaagttagttatggtgtgcaaaacagagtcaacccgaaaaagttctagatacaacatctagaaacaccataattaaccttattttaaaagaacagtggatgttacatctaatttataaaaatggaagaacataatctttacagaaaaaatcttcagatataacaaaatagtcccaagacatartatacaatgaatatgccaagcatataattagaatagaccaagaatatcacatcaagagggttattttagaggggacataaacacctatgtattaataacatatatttaacctagggctggctatcttttttgatgtgacaatttgtcccatataacttatcaatagtaacacatcaaatggatctcctaattatttcaagcatctgttttttattaaagtaaaagcacaaatactttttattttccaggtatgtctggggaatcttagacagttttttgttttgttttgtttttttgagatggagactcactctgtcacccaggctggagtgcattggcccgatcttagctcactgcaacctccgcctcctgggtttcaagccattctcctgccccagcctcccaagtagctgggattacaggtgcctgccaccatgcctggctaatttttgtattttttagtagagatggggtttcgccatggtgtccaggctggtctcgaactcctgacctcaggtaatccacccgcctcggcttcccaaagtgctggaattacagggataagccaccatgtccagcctcagacagttttaagtacaaaatatatcatttaggatttgatttgcggaaggcaaaatatcaaaaattatcaagaaattttgaatacctgattccaataggatcatgtaacttagaaacaatttttgactacctatttaatcaaagtgactgtaaaaggttttaaaagtaaacagagaggtaacatgattgtaaagaaccttagctctttcctaagagacacgaattcttgaatactcaagggtaaaataaagtcaatataaaccatagaaggttattctcataaaacacagaatctttggaatctaagccaattatacagaaaaaagaataagcctttattttttaggtgaatgtggtaaacagtaaaccaaagaaacaggctcatcaatattgggtaaacttttctttgtttttaaatgtttagtctttagttttaagagatcatctgcattttttctgtaataaacttaaaagatatccacttatatttcttcagatttattaattctgtagcattttaagcattgaaatgacagtttttctctcaatcctttttttttttttttttttttttgagacggagtcaggctctgttgcccaggctggagtgcagtggcacgatcttggctcactgcaagctccgttctccccaggttcacgccattctcctgcctcggcctcccaagtagctgggactataggtgcccaccaccatgcccggctaattttttgtatttttagtagagatgaggtttcacagtgttagccaggatggtctcgatctgctgaactcgtgatctgcccacctcagcctcccaaagtgctgggattacaggcgtgagccaccgcgcccagcctgtctcaatccttaacaatgctatatttgttgtatttcatatgtttagctttctcatggagaaaaagaaacataggcataaacctttatactatccgcctgctggtcctgcaacatgagtttaataaagcgttcctgatacttaaacaatttctatgatgtcagcagagagatatcagcaagagtgattgtaaagtagctagccttataagtcaagagttataatctttgatccactgctcaatccatttcaagatctgatctacattattttctagctcttctggtttattgctgggcagccgatgcacaacttcttccttgtaggatgccgtggcttcttcataaagaacttggaaaatctcacactgaatattgtcttttagtttcttctcattataacccctcatttgaagtatttcgtacaatatgttggcatctattctcaacacaaaaactatgtgaaaccagcgttcagggaagaaatcacaaccgtggtaatcaacaataactccacattctctcatttggttatctaactcatcagctactctgtcttcatctaaaatgggacaattatactcttcatcatagtcgtcatacaattrcttttctcaagctaaatcacccacattaatgtatttcaatcctgattttgattctgatatgtggtttttccaacccctggtgtacctggctttctatgacacgtttctatcaccaagtcagaacaaagtgacactttaggactgaactcagggagtctgtggggtcaaaactaatttcataatactactaagactttaacatgcaatgggttcaccttgctgtctccaaaaaaaaaattgcaccactgcactccagctagggcaacagagcaagaccctgtctctcaaaagtaaataaataaataatttaaaaaattattgttaaaaaaagtttgtcaggttaatgattcaatttgattaagcacaaatttacattttttcatagtcttaaactttaggagtaacgttcacttatttgatcagtaaatctgtatagcttttgtaagaacatgtaaaagtagaatagcaatgtatagtgtggctgggcacagtggctcatgcctataatcctagaaatttttggagtccaagatgggaggattgccgagggcaggtatttgagaccagccttggtgacatagcgagagaccccatcttaaaaaataagaataataatacttaatgctgacaactcatagaagacatgactatttttattaaaccccaaatattcaactagtctcatttgccaaatatttacctaaatgtgtgaacttgaattcttaaaacatttacgtttctataggaatactttttttagtgctgttgaaagtattattggaagttcaatttccttaatttctgggaattttaggaagattcaatttataggtgtctctttatttctaagccagtcagaacagaacatccttaagagctatcacattctcacttggtaagaccatctcatgatggttatcccaggatgagagacaatagctgctttgaaagttcccctgccacactgggcttccagtaccagtgcagctaatgaccctgccctaacagcaaatgctggggagcagggtgcaagtgtttacttgggtgcccttcacgggcactccttttacgtggtggacagcctgatgctttgttctctaaaccagtatcaggcattcctctcatgggagatgtgcttatcctggcagacgcccttgtggctcttttctgacccctctccagtttatgactgcctgaccatcgctctggtgctcagagcctgcccttgtgttcctccccagcatcccggggaaaacccaggtagcctgggagagcccctggttcttcagatggaatgtgcaaattcagcacaccaacacgataggaaataagttccaagatttattacttccagatcctagagagggagggcgccatgagtcgggagggcaatgctctatccccaggtcaccagaagaatgaatgaagtgtcaggcatagagcaagagagagtgggacccatgggccaccacctttactgggggccagggcattgtccaagcaggtttcctgcagggagttttagttggtgagtttaaaacaggcagccatgagtttcaggatcacacagcaactgagaggtggtccctgtggcatactccacagtccatgtggggtgtggggttggcagggcagccaggtagactgtctcttagagaggccgtcaccagaaagaggaggtgtataaggcagatccctggatcaaccccattgaggactgggggtggcaggtggaagctgtcgagggaaactaagccctgtttctggtatgagaaggttaaacttatcatcaaaatagatgccaaggctatatgaaactgtcagtattcactacagtggcatttccacagtacaatacagacatacaaacagacatagataatttgtaagctgtaattctaaaatttcaggccaggcgcggtggctcacctctgtaatcccagcactttgggaggccgaggtgggtggatcacctgaggtcaggagttggagaccagcctggccaacatggtggaaccctgtctctactagaaatacaaaaattagctcggtatggtagtgggcgcctgtgatcccagctagttgggaggctgaggcatgagaattgcttgaacccgggagatggaggttgcagtgagccgagattgcaccattgcactccagtctgggcaacaagagcaaaactccatctcaaaaaaaaaaaaaaaaaagaagaagaaaaaaattcagtcatagaccaaacttaaaagcagaaatataaaattttactcagatgtctacttcctgatggcatgaaattcttaattgttttgaaaccaaagtagaaaagcagacaaacgaaaaatactagcaaatcagattctgttatctttcacccaacagagacaagatctctataaaccagcagtccttccccaaatacgtagtatacaaaccgcttcatgtctgtcattttcgtcaaccctggggtccttcaaatgccttttgttccttctcatttacttcaccttgacttttcaagacatattggttatactacacagttggttacatttgaagtatttcatgtaaattacaaaagtatatgaataatgtgaattcatttttgtttatatatgtatatgcatgcatacacatacacacacactcctatagagtgaacatttggctgaatatactgccaaattgttaaacaatagtcatttctagctggtggaattacaggaaaatttgtgtttctgattatatatttctatagcatttaaattttttgcaagtcagcgtgcatttcttagataagcaaaaaaaaaattaaacattttatttaaattttttttcaattccagttaatagcagatgtcaatagaacaaataagttcccttatccatgcttctgtatgtgggggattcacttgacaggtgcaacagaagcacaagcattattgtgcacctgtgtctgaaatgagaatgaggctgcctagaagtcttgagaaaagtggctgacgagtctacaaaaacacccttcttaccctttctcactttgaagtgcatgaagacgttgacacacttggaggtctgctggctaactggtggaacagattcctgggggaaatttttttgttttgctcttgtacctcatgtctggattattttggattgctttggggacagtatctgagtttctatctcttggcctgttttttccaggaatataaaggttttttttctttgacatatgcttaaatgtttatttttaagtgatgtaacttttcaaaaaacttattacagtttatttctgtgggaaaaatattttttakgtttttgactgttttttgttccttcttgtttgaaatctctagccaacaagaacattagtcatgacaagcatgccatctgagtaagtacttgttttgatttctgttcaatgtaaaatgttaaccttttctctcttatactctaattctgggtgcctttaggcaacttgtcaatctgtcctgtatcacttttactttataaaattaatatctgagttagaagatcactgaaaattaaacatgtaccaaatgtgagcgacttagccttgaaaactctggggttgtttaggcagcattaagaggtgtgtgctcgttttggtgttcttttgcttgcttgataccaaatagcttcatgaatgttcaagaagtggaacatcattgaccaaaacatttcccttaaaggtcttaaagcaatactgcagcagaaagctttccacagcagtgttaaagttgctatgtatgcattttgtggaagggtcaatagcttgttggcatgctcttatcatctcccttaaacatttaacacaacaaagaacatccaacaaaaatacagtgctatattctttgcaacagatttttgaattcctgtttaaaggggaaaaccatgtttttgatatcaatcataggttttaaggttttaagacatccatcaaaacattggaacatttcagtgaaaaatatgctgcagagagggcacctttagaacattttcagtagtgggatccttttcctgcctggggcttagaaataaaagcactgatcatcaaacaccatacattatatagtgaaaaagggggtcactcaaaatttttgtaaatatattatgaaatatattgaacattctaaatagtctaatacagaagcgaatattgaatatatgtgtaatattttttaaagtctttgtatttttccaaaataaaagaaaaattactagttaactgcttattttctcattcaagatttaaaaataaaacttttcatttaggccatcttcttgtcttactctttttttctccacatggacttcttgtgatacttaagaataagacctggacattctgattttatgtggattagctgagccttgcagagacacttgttacttactggcacatccagcaagcagctgccagcctcaggatggagttctagggagtgtgtagtttagagctttttactttttgtttttgtttttgttttcttttatcatttttgcctttatttctttccaagtttaattatttttcttgactcaagcacacattctcgggttgaagtagtgatgaggcccagatcttgactcacacatcttttctaccctaaggatctcttaagaatttaaaagcatgatataattcagccctttcattttacagataaagaaacaggttttgagatggacatacctaagatcactagagataaaactaagaaggctgggtgtgttggttcacggctataatcccagcactttgagggtcccaggtggacatattgtttgagcctaggagttcaagaccagcctgggcaacatagcaaaaccttgtgtctacaaaaaaatgcaaaagttagccagacttggtggtgaattgcctatagtcccaactacttgggaggataaggcaggaggatcacttgagccctggagatcaaggatgcagtgagccatgattgtaccactgcactccagcctgggcaacagagtgagaccctgtctcaaaacaataaaataaaactaaggaacaccatcatttggaaggaagagtgttagaggcagtctgtataagcatagacaataacctcttcccctttgtaatataatttttggagaggagagatgtttatttctttttctatttatttatttatttatttatttatttatttattttgagacagagtctccctctgtcacccaggctggagtgcagtggcgcaatctcctcccactgcaagctccacctcccgggttcacgccattctcctgcgtcagcctcctgagtatctgggactacaggcacccgcgaccacgcccggctaatttttttgtttttttagtagagacagcgtttcaccatgttgttgtatatatcacagtgtggcttagaaagccctccattggggattttttaaattttctgggagagagggaaaactaatgtcagaactaatggcatagaaaggttattataaaagggaagaaagaactgagggttgtttggtaaggaagttggacggaaagaatatatttttttaaaggatattttaagtattaagggaatgacagagcaggagataagccataatggtcatgagctttgtgacaaataggtcccagatttgatttgatgatttaataaaaagggtcttttttcccctcttagtagaaaaactatgtgttgatactcaataaatattacattttcaaaataaaataagtgaggttcttggttctgagcatgcacagataggttcaaataggcctgaaaaacaaatcattgccccagtgggaagagtgttggtctgatgtcaggggcctggttcctttttttcttttttctttttttctttttttttttttgagacggagtctctccctgtcgcccaggctggagtgcagtgacacgatcgcggctcactgcaacctccacctcccggattcaagctattctgtctgcctcagcctcctgagtagctggaacaacaggcgcgtgccaccacgcctggctaatttttgtagtttttagtagagacggggtttcaccatgttggctaggctgatcttgaactcctggtgatccaccggcctcggcctcccaaagtgccgggattacaggtgtgagccaccgcgcccagccaggggcctggtttctgatgctggctctgtccctacccagcccagccactgtgggaagccattgacagcctgtgggcttgtcttctcagccattaaaatagaattgagatctgaagtttatttccccaggtttcaaagcattgattataagtcagttaagatatacgtaccataaccaaaatcagtttcaaattttggctttctagttttattagtactaatattgagtgtaactgctttgatgggcatgtgcaacaaagtcattcattttgttaatttttcccccgatttgacagaaagcagaatgtcgtcatccaggttgtggataaattgaaaggcttttcaattgcaccagacgtctgtgagamcacgactcacgtgctttccgggaagccacttcgcaccctgaatgtgctgctgggaattgcgcgtggctgctgggttctctcttatgattgggtaagccctgtgtgtgaatgcgtattttaaaacaaggcattttgatagagtgggtcaccctgaggtgccgacatcagcactcaggccggcgtgcacccttgtggatctgcacactttcctgtgagctgggaacacccgtctttcctcctgttggtctcccgtgggctgctacccttcaaccagggccaagttctggggcaacaggaggacggggagggtagagagcaggaagtgagtagcctctaagataaagcagaagcaagattacaaagatgctgaaagaaacgcaaaatgcatgttctcacagtcaaagagctttcctctatgtgtgaccaagaaacattgtgagctgtggtggtggtggtttgcagagccaaaataattcagtgattgtttgtacagatggatttacttaggatgaaggatgttcttttaatcccatttggataggttttatcctatgtatatctatctgtaacattatttgcccttgtttctgtagattaaagatagcttttaaaaatacataattattttccttattcataaaaactgaaatgaactgttattggttctattattactttcatcctcaacctaaggttgctccaaagcattcctttctggtgacagtagcatcacttgttacgtatgttaccattctgcatctgtgggatccgtcttccctcctcctctcccaagaatgtattctattcatactcatactgtgttcatttaaaccagtagaattataacatgcaaaagctacacatgtattttcaagaatggccgtcgtcttttttccgtgttgtgacagaggttaaagagattagtgcttctagttgtgaagtggaaaacgttgaaattccaaaagtaagcactgttcatttgcattggtggcaatgggggatcaccttacctgattatatattagtactgctttatgtttatttggatgaaagacagtagtgcccctctcatccagggttttgttttgtgtagtttcaggtaccatggtctgaaaatattaaatgggaaatcccagaaaataacaatttataagtctttaaatgcattcttttctgactagcatgaagaaatctcaggttatctggctccattctccctgggatgtgaatcgtccttcagtccagcctgtgcatggagtaggtgctgcttgccctcacttagtagccatcttggttatcagatagaatctcgtgattttgcagtgtttgtcttcaaggaacccttatttggcctaataatgttccccaagcacaagagtattgatgctgacaactttgatatgccaaagaggagctccaaggtgctttctttaagtgaaaaggtgaacgttgtccacttaatatggaaagaaaaatggtatgctgacgtagctaaaatctatggaaaaaatgactctttgacctgtgaaattgtgaagaaggagaaaaaactgtgcatactatatatatagggttcagaactatccacagttttaggcatcccccagggggccacggactgtgccccctttggatagggtggactactgtctctttaataactctagcatcagtgaatgagttctgtgttttatttctctccaattcaaatcgtctctgtgtcttcatctgactactctcccttccctcaggttttggaggaaaaaatgttatttctaaggatatgcatctgtacaggattccttacccaacttattcttctgggacttggagcagtccatagaggtcagacgtgagaacgtactgcctttgctgtcgacatggatagagacctgctccctggttgtctgcatgtctctgctcagtgttctgctagtactccacagctaatcatacatagaaacagaactgggtgaaattttaggttattgtatctcttctgggattacctgatatgataaaggtgggcattaaaacacattatttaataaacttctcacctttagtctagactccttgcctggagggaagaacctggggcactcagacacataagtgaatgaatgaggtacaaggcaatcagacaagaaaagataataaaaggcatgtaggttagaaaggaagaaatagagttatctctatttataaaccacacaattttctatgtagacaagtcacaagcaatctacaaaacagcaattagaggtgacagctgagttgagcaagtcatccagatgcaagaattccattgaaacttcagtataaagctaataaaataagtgcaggatctgtgtgctgaaaactacaaaatactgattttaaagctcaaagaactaaatatattaaaagacatacaatgttcatggattagaagacatagtacagtgaacatgtcacttcttcccaaaatgatgtatagatttaacacattctcattcaaaatctcagtggactctttcaagatacagacaaactggttctaaaatttctatggagatattaaggagccagaatagccaaaacaatttagaaaggaaagaacaaggaggaggactggcactacctgcttttggggcatcctttcaagctgtggtcctcaaggcagtgtggtattggtggacacacagaacagacagagaatccagaaatagacccccaaaatacatcccatgggttttcacaaaggcatgaaggcaattcagtggagaaattcagtcttttgaacaagtggtgctggagcagttggacatacacaatcaagaaaaggaaccttcccaacactttgggtggatcacctgaggtcaggaattggagatcagcctggccaacatggtgaaaccccgtctctaccaaaaataaaaaaactagctcggcatggtggcacctgcctgtaatcccagctactcaggaggctgaggcacaagaatcacttaaaccggtgagatggaggttgcaaagagccaataccatgccactgcactgcagcctgggtgacagagagacaccctgtcaaagaaaagaaaagaaaaggagaggagaggaggaaggaagggagaacctcattctataccttacacgagccacaaaaattacctccaaatggatcatagacaaaatttaaaggtataaaacttctataagtaaacatacaagaaaaatgatcttggtgtaggcaaagagttcttagatacaccaaaagcatgatgaataacagaaaacatagataagttagatttcatcaaaattgaaagcttttactctgtgaaagatattatgaagagatcagaagaaaacgtttgcaaatcttatatctgacaaaagatttatgtctggaatatataaagaactcttaatactgaacaataagaaaacagaacagctcaaacaaaaaatggcaaagaaaagatttgaatagacagtttactgaggacacacagatggcaaataagcatctaaaaagatgctcatcattattgctcacttcagaaatatagtgagatccactacatatccattagaatggctaaaagaaaaaataacagtcgcactctagcaaggagccagggcagctggaacggctgctggtgcgtgtgggaagtggtccagccgctttgagaaacagtttgacagtttcacagaaagctaaatgtccactcagcagtcccactcccagatatttgcctcggagaaatgaaagcttgtgttcacacagagtctgtacgcgaatatttgtagcagccttacttatcatcagctggacctggaaacagcacagctgtccctccagtgggtgaatggatcaaccagctggaccaaccatactgtggagtgtcactcaggagtcgaaaggaatggtgataggtacagcagcttgcatgactctcaggggcatcatgccaagttgaatagctggtctcagaaggtcacatgctgtataaggccatttctttgtcattctagacaaggccaaactatagggaaggagaacagatgagtggttgccgcgcattaaggtgggagtagcatctgcctctgcagaacaatagcagctgtcacatctttggggcattggaattgtgctgtgttgttagtggcaatggttacagaatccatgtattaaaacacagagaactgtacacacatatgcacacacgagtaaatcttattgtttctaaatttaaattaaaaagaatatctaggcggggtgcagtggctcatgcctgtaatcccagcacttttggaggccgaggcgtgtggatcacgaggtcagcagttcaagaccagcctggccaagatggtgaaactccgtctctactaaaaatagaaaaattagctgggcacggtggcaggtgcctataatcccagctactcaggaggctgaggcaggagaatcgcttgaacttggagggaggaggttgcagtgagccgagatcacgccactgcactccagcctgggtgacagagtgagactctgtctcaaaaaaaaaaaagtatatcttacatatctaacgtgctttccaaatggagatgtttgagcactggtaggaccgggctagtgtcttggtttcagaactaggtttccttctgtgtgctgaagtttacaggctcctgtaccttcaactgctgcctctgtacctatacttcctgttagcactgaagcttcatcccagcttttctatcttaaaaaaaaaaatgaaaagaatttaaaaacataactttctctaaattgctctttgccctctgtgctacctttttttcccctcattcatggcaaaacgtcacaaatgtatgtctgtattgcccttgccttactgatgatgtcgctatttgttaatagtatcaactcttgggagattgcgaaggctcaggtggcctatggcttcaggtgaaatatctgtttgtgtgattacaaggtaaccatgatggcagtcaggtatatcacacatatataaatgacacaaacagatataaatatatgtttgtgtgattacaaggtaaacgcaatggtaaccgcaatggtaaccacgatgactctcgctggcacaacaggagtattgatgttcacaggttgctcctgacttgcaccctcaaaaagtttagaaacaagccgagtcactttctctgttcatctcmgtcttcaagaagacaaagacgactgctgcttcttgcatggcccccctcctttaacttttaaataaattgaatagtacaaacataagaaatttgagagaggatagttgccaccaccatttacaaagccattctacataatttttaaagcttagcacccactttaatatttatctatgtcttgcatataacttcagatataaacttcacagttccaatttcttttagggtcaagatttaaagtatccatatcatatattatatacattgactttgtgtacaaggaatctctctctctctctctctctctctctctctctctggcactctcgctctctcgctctctcgtcctcctccttctaaccctgtctccaatgtagttgggggattcttaaaatattctctttggctagcagtataaactggcctccaagaaaaacactgctgagcatgtttttatttcagggtttgtgtggtattctctggaaatttcttgtaaaggagatttgtagcagttcttcagaattagatggttgtatgtggcccagctagtcttatcagaaactgtggcgattttataacaaagttcagtttgaattttgacttaatatttttgagaagtttattggcaatttttccatgtttacagcagttcacacctccagtgttagcgctactgttttcaggaaagagaataatttatgtttttcctccttcatgactgaattgtctggcagatacatggaaatagaaaaccatgccaggagttgccgagcttcctatttatgggagacaggaagtaacacaacagaaaaataaagaaattaatttgaccaaagtgtccctttagactcacattgttttgttatgtgttgttcaagcatagcacaatttgaacctttaaatactctttatcccactctcacttaatttgatgtttcctgcactttcctgtgacttgtctaaaattctactttccctcgaaacccttttgtggatgctaacatacaagcagagtgtcctgtgattcagtcttccctttttccagctaccactccgtgtcactctgtccagcacagtgaggaataactcagcctgtattcagattttaatattttgattctgaacagcttatgaaaaggatctgataatagagatttaaagctaattcacttataaatacaagtgtagggcttaaaagctaaatcagctttacaacaaaatgtcaaggccgctaactatcaacagataatctagtgttttcttaatcaaaaatgatgtcatgatgactattttcttgagataatgtgatccacattgaacttagtaagcagtgagtcagatgagatatgtttttatcagtggtgagcatagaatcaatgaactgttagaataacacactcagttcattccgttcacgcgtctcattttacattaaagaaatgctgagccgctctcctaaaattataactcatggcagaaccagaactggaatctcagcttttcactggtgttagttcatcaccctgcattcctaagtctgttcaaaagggatcatcttgaaaaaccattctctttttaaccttcagttggcagattaacttcataactcatgttaggaagaatcttcaggcacattgtacttggtgtgtcacactgacactgagtttctgagggtgcccttcaggtctctctggcagacatttattgctcgcacttgcaagctgactaggatctcaggcctgggtctctgaactttcacggcttgatttcaaagtcctttttatcctgctacagattataccttggtaaaggactttatacttcacagagtgttttcacatgcactgtctcactggatcctgacagaacatttttgcagccgagaaggacgctgcaaataattagtgagtttagtgatggagactctgggcaaaaatagcttgtctgacttgaatgtggatcttagaaacacatctctgtcaaggcattgttttaaggcagtgactatggtcttacatttatctccaggacacctaatttatactttttcctgattaaaataatggattctggttttgcccagacatagaacccacagagtttgtctgcttctttcacttgaggtggttcctgagcagtgccagagctcattctctgcggaggctcctgcaggctgcggcagcgtggcctctggccgctgggagcatgggaagcaggcgctgcggtctaggtcctccatccccctgtctgctgctcctggcaagaccccaaggtgcgcatttcccaggttggagccgctgtgcttcccaggaccataatctgctgattgaggacagataccaaaaagtgattcatctgtaaaattgagggctgtggtgctgccctctaggaggacatttggaaagatgtggagaaacctgtgagtgctaagaatgactgatgttaaagtttgaaagagtcaaagtgatttttttagtgggagaagactgtggagtcaccctgagatgcaaccacaggcttgattagaaataaagtttgatcaccattttcaaatttttacattaatattttttaattttcgaaaggtgctaaacagaatctacttaatgcacctggcacagaaaaggcagtgcccgggtcctaaggctgcacctttgcaagaaagagmaatacctgaggcaccgggagtgaggaggacaggtgttggagaaggctgtagggccccagtatggctgtgtagttcaagacgagggatgcagaagccatcggactattttaattacagagtggcagcttttgtctctgtggcctctcagcaaagaatggattgcagggaggtaagaacagggtgagaagcaggaggcagctagggtcatcgaggtgaaaaatgactgcggctgtgtctagagggggggttgataggtggagaggagagagcaggtcggcgcccttcctaggaagatctagtggaatctgtaacgtcaggtgtgtgggaatggagaagtcaagaagactcccacccaaattttttcctggggcgactaactatagataatggtgccatttgcagagttagggaattctggggcagaagattgtgtgcaaggtttggggtacaataaaaaattgatgtaggcatattaggtctgagattcctactggacattcaaatagagatactacatatcagattatatatatgtacatatattcagaggaaaggttaactattcactccagccatggtacctggaagggagtgtgaatgaagaaatgaagaaaacagtgagtttaggtttgatctctgggctgtgccctatgcagaagtcagggggaagggggaggcagggggacccgggaacggctagctagcaacctgggggagacaccaggggaacatggcatcagtcagaagggggactgtctcaggaaggaaggatgctcagctgtgctgagtgctgctggaaggtgaataagaggagacagaagccactgtttgatttcttcaggtggatgttgtcagagaccttgaaaaaagcaggatgaatccaatgactaagacagttgaagagtcaatggtacataaagcagtggaagcactagggttatgtgtaatggtgcgatttgctgagttagggattattatcagacatattgctgatatgttattcctagacataatgctgctgctacatcagagagattggttggcagcgaatggggcactgtgaagtgtgactcgagccttctcgtgttgccaactgcaacacagatcatcgtcctagtgcttggcgatgtggttgcattatggtgagttgagtgtggccttgggaagcatctgaatctgttggctgagttatcagggaaaaaaaatttaaaaagtaaactaagattatgtatattaatgaaaaagttgctgtatttggcaaatactttaaatggataaggctaaaaaccaacaagtcgagagggtacttgttgccacccatccttttccaaatcatggccttcaaggatcacactgttggtctttccttttcttttaacttggatcaactgtgaagtaacacaggtcttcagtgtagatctcagttccccaacatttgccttatgactgagacctccaggacgtcaacttggtccatgctgaactgcagcacaaattccaagctttgaccatacctcaaggtgcactttaacctttgcagtgttctgccagacatctgaactttcacttttgtttctgacatctcaatcacacagttctcactgtaaatattaaataatagcacagaatattttaacttcaggtattcattggaaaattcaaccatggtttggttttatctgtcacttcaaaaactgtcttcagctgtccatcatttagatgtcatttagatgttcctcagggactttggggacattgttaacaatctgttatttcaaggcttctaaactctatccccaagttaaaatgatttccaaggaacatcatacttctcttacagtctgtgtgtaagcaccctctgtgaattcggttttagggacaatgttagcttttgaagagagctgatgtaagaaatactagattttaggaaactgttgtacttttttcaaagctatatttgacgacattgtacattttgctacctgatacttttgatgtatgatccacctaatgcctttctcctaaaattaatttccagtgaattgaataggaattccaaatgaaatgaatttcataggaaaatctcatacagaaaatttgttaggctgtccttaaccagagaatgagaattatgtaatgcggttttgtcagctagagtaacagcttgccataggttcataatagagctgttttttagttctttttcttgggttcttgtttctgaaagaaagtttctctgccagaatattgaagtcgtgcctaagttaataatttaacaagcattgtatatattaataatataatatcaataattaatgctattaatcattaataacaattatttaatattaatattaaatacttaatattaaatttttagaatattaaaatttaaaatttaaaaaataaaatttatcaaaaaaaattttttttttacttttgaagcattggttttattaaactttcaaagtagtatggcaaaaaggtggccacataccaaatagtgtcatacatttcttaaaatctctcctagcaaataaacttaaattgagatcatgagtcagttgaaaagacaatttaatttttttgccatacaattaaagtatttctgagaagtcagagtgctttgcaatgtttggtgaataatttacacaattccagaataatgtctcacttatggagaatacacctaccacttacttcgataaacagaagtagagtctatggtttctttcttttttttttttttttttagctgctaaagattattattaggacagaaggacaattagctttaaaagcattcctcagaacatgtatttttttttctagtattcttttttttttattatactttaagttctagggtacatgtgcacaacatgcaggtttgttacatatgtatgcatgtgccatgctggtgtgctgcactcattaactcgtcatttagcattaggtgtatctcctaatgctatccctccccactcccccgaccccacaacaggccctggtgtgtgatgttccccttcctgtgtccatgtcttctcattgttcaattcccacctatgagtgagaacatgcggtgtttggtttttttgtccttgtgatagtttgctgagaatgatggtttccagcttcatccatgtccctacaaaggacatgaactcatcatattttatggctgcatagtagtccatggtgtatatgtgccacattttcttaatccagtctatcattgttggacatttgggttggttccaagtctttgctattgtaaatagtgccacagtaaacacacgtgtgcatgtgtctttatagcagcatgatttatagtccttttgggtatatacccagtaatgggatggctggatcaaatggtatttctagttctagatcctgaggaatcgccacactgacttccacaatggttgaactagtttacagtcccactagcaatgtaaaagtgttcctatttctccacatcctctccagcacctgttgtttcctgactttttaatgatcgccattctaactggtgtgtgatggtatctcattgtggttttaatttgcatttctctgatggccagtgatgatgtgcatgttttcatgtgtctgttggctgcataaatgtcttcttttgagaagtgtctgttcatatccttcgcccacttgttgatggggttgtttttttcttgtaaatttgttagagttctttgtagattctggatattagccctttgtcagatgagtagattgcaaaaattttctcccattctgtaggttgcctgttcactctgatggtagtttcttttgctgtgcagaagctctttagtttaattagatcccatttatcaattttggcttttgttgccattgcatttggtgttttagacatgaagtccttgtccatgcctgtgtcctgaatattattgccaaggttttctatgctatagaaatagcatatttctatgctattcatcattaataacaattatttaataatattaatattaaatagttaatattaaatttttagaatattaaaatttaaaatttttttaaaaataaatattttatattaaattatcaaataaatattaataataattatttaatattataaaattaataatctttcattattgaattattgattgagttaagtaattaattgattaactgataaggattattgttaaattattgtactcttgggtagtacagagactgcatactgcgctttgccatgtaaatactattgtctacttcctggtacgtggctctagggaggctatggcagagtcaagtgcttttgcccttaatgtgaacaaaaaatagtgattgctcttagtagccataatatttggtttattgtctgtgttggtaataatttctgctgtgttttcatacagtgaagtgatgtttctgctgtttattttagttgcattggaatttgttatatttatttctttgttttccttttgataagagaagtacgcacttagttatttataaagatgtttggacttcacatgtgagtacagtggtgacatgctgggttttcctggtcattgcttagctgtatttataaagtgaatattactgagcagttaagccttaacatcgagaatcacccattttcatttttgaaaactggaaaggattaggtagaatgcaaggagaataaattgaacttaaatgtttgtgttcaattgaggtgagctttttcataagaatattcaagcctaggtcaacatgcagcttgttttccctctcaccacctggaattcagtctctatcggtcaatgtcttctaaaagggaaatgggttcttaactatatacttttagtactttattgcttatcttccctttcttggttgaataggctgtgttggatatttagcttcctgcccctttctttatgagacagctagggcagtgcttttcaaaaccttactaatgtgtggatcacctgggggatcttactgaagtgcagatcctggttcagtgggtctgggtctgctcaggcttgaggtgaggtccacgctgctagtcctgtgacccagcattaggtccccaggatacaaaatatgaccggggatctctgtcgtattcgggggtggagatgagacagcgtcccaatgatgttagtcacatggaacatttagagatgcggagtactttgtcagtgttttacacatcgtcaagctgttagtcaagacagtaatcctctgtggaaactgtgggttgaacactttcagtaaattgctcatggtcatagtgcttggaaatagtaaatttttttttttttctttgagacagagtttcgctctgttgcccaggctggagtgcagtggcacgatcttggctcactgcaacatctgtctcccaggctcaagcaattcttgtgcctcagcctcttgagtagctgggattacaggtgcatgccaccacacctggctaatttttattttttgtagagacagagtttcaccgtgttgtccaggctggtctcaaactcctgacctcaagtgatccgccgaccttggcctcccgaggaactgggattacagatgtgagccactgcatcctgccagaaatggtgaattttgaatttgaattcagctcttcctcaattcatagcccacattctttctagcatctacttccaaagatagcctagagagtattttttatcttctatagctgtaaaccttgatatgggcattctctgatggcctgtgtgttttgaaaagattaatggataaggcagtggatttcactgctaaccttgctacaccgtagctgtgtaaccttgggtaaggcagtttctttatctgtaaaagaatggaaagatcacctaaataaagtactcagtaaacactcaataaatattaaatatcgttattattcaacaagcatttttgacgctgatcactagccttcattaaaagtataacttggatgaacgttgaacacaccgagtgaaaggagccagacacaaaaagcacatgttgtataattcctttcagacagtatatccagaataggtaaatccatagaatagaaaactaattagaagttaccagggatggaggggagagagggatggggagtgattacttaacaggtacaggatgtttttctggggtgatgaaagcattttgaaactagaaagaggagctggttgcaccgcatcatgatataaaatgccattgaattgcacactttaaaatggttaattgtatattatgccaatttcacctcacttaaaaaaagtcatatatggaaaatagctttaaggcaccactacaactactaaataggtttgtatttttaaaagaactttatggaattataggaagcatttcttgatgttatgagatgtgttggaaatacagaagaatagcttattttggaacagatattattggcttgaaattttgccagttcaagctggtctctttggaagactagacctttattttctggcttgaaaatgctttggacataagtaccctattattttgttgttaaaaattatactattgacatccccaattttttctcctgaagttcagtataacctagaaataacttcattgctacactatttcattaactacatgggtgcttttttagttaataatgatgcataatgtcttcatgtggcagaaacactaacctgccccttgtcataaatctgtaaaaagatggacattggtttaaacccagttgttgaattctgtgcctttaaccagtatgttacactgtctagttggggaagaatcccaaatcttcttctttctttagaaaaatccaaaacagcatacaaactagcaaactctcataaatgttgtttgagaaaatcaattgccctaactactaagacaaaggatctataaaatctgatgagaacaatctttgtaatttgatttttataattttgtcagcttaaattagtaaaaagttaataattattacttttgttacgcttataataaataatgtgtttctacaccttccataaacacctacaaccacactttttaccacagttggtggagtgaagggtggatggaggagatagtggcaaaaacaccccaatcactttcagtgattaaagtaaagatgtgtctaactttactcctaaagtatcatccagtaaagtggaatgtaaaacatacttttgaactgtttgaaatcaactacattcctatggcttacgactgtgggacaagtttctaactatcagatttgatttttaattaatcagtgatattttataccagcagtctccaacctttttggcaccaaggaccagttttgtgaaagacaatttttccagggacttgggggttgggagggtgagggaggaatggttttgggatgattcaagcacattacacttattgtacactttatttctattattattacattgtaatatataatgaagtaattatacaactcactgtaatgtaaaatcagtgggagccctgagcttgttttctgcaactagacagtcccatcggggggtgacgagagacagtgacagatcatcaggcgttagattctcataaggagcatgcaacctagatccctcatctgcacagttcacaatagggttcgcacttctatgagaatgtaatgccactgctgatctgacaggaggtggagctcaggtggtaattaaagcaatgggaagtggctgtaaatacagatgaagcttccttcactggttcacccaccactcacctcctgctgtgtggccccgttcctaataggccacagactggtaccaggacccctgttttacacgatgtggagtcttttgtatgcaaagaatattgttgactttcgccacacggaagcccccccgccccgcttcccccgcctttttcctttccagttacattcccacaggtattcttagtaccacaactgcagttgaatttcacagtatggtgggtggtaagctatggtgggcggtaygcttggataagcctggctatttagaaatttggaataaatgtagtgttatgactaacagtaatgttgcctatcaaaaattgtgaatgttaataaatgttttcaacacaatcattaatgctttccagtgagttaaaccagcttcatgttacagttgtattttccatcccagtagggagtcattattaaatggggtcatgttttcaagcccaacttaaaatccctcttacagattgccttccccaccccacccccagttttctctcatcacttatacattgaaataattgcttattgttttccctctttaaattttttttgagaagtcaaaaattgagtaccttgttcagtgtttttgcttatgaaatactttgtgaataaattttgttcttagctgaagaaaatttcttaggcagttaagaaaatactaataagctaattaatgaataaaaactaatttcattggtcctgattggaagtgcaacatttaccgatatttagctataatccttttgatcagtcagaaatttgtaattattctttgagaaataaaaagttgagagggctgggtgcggtggctcacacctataatcccaacacgttgagaggccgaagcaggtggatcacttgaggtcatgagttcgtgaccagcctgaccaacactgtgaaaccccattctctaccaaaaaaaaacaaaaaaaaagaaaaaagaaaaaaattaaccaggcattgtgatgtgcgcctgtagtctcagctacacaggaggctgagtcaggagaatcacttgaacctgggagacgatgctgcagtgagccaagattacaccactgtactccagcctgggcgacagaggaagactgtctaaaaaaaatagaaaaggaagttgaaaacagcttagggaagagctgcaaccactgaccagcaccagtactccatcataatatatgcttttcacttataaggaactgtaatgtaaactgtggactttgggtgataatgatgtgtgaacacgggatgactgggtacaacacatgtagcactccagtgggagacatcaaaatgcatatgtggcggcaggaggtgtatgggagctctctgtaccttcctcttaattttgctatgaagctaaagtggctttaaaaatacaaatacagaaaaaaacttgtgctttctatagattaatttgaacatagacacattaatataatagatacattgatttgaacataggtacattaagttgaacacttaaggtttttatgatgtcctataccacaataaactgaagaagtctgccttacaaatttgttcaaagaactctcaatgctctcactgctccttccctgccttgaacaggaagtgtcatccagtgcaataagggggaaaataaaatgtgcatagcaatcagaaaggaagaaataaagcagtttctattcacagatgcagttcctatttaaattcatcagcaaggttttggttttatgaatgataatattaaaatgtaaaaaacactattttcattatgtaatgtgtcacctacaagatgctgaattcctgttgcagcggatgctgaattcactctgcccttcttataagaaatatgttgggccaaccttttgtttttaagtttgcttacagccttacctgtgctctttcaaagtagattttcactattttgaacactctattaaggtaaagatgtgttcggccaatgaaactactagagcaaaatgtttacactgtatttctgatttgattgttttaatacaactgaattagtgttttctcctatctctatgcaatattaattcctgggatgtctgtgtaaattaattaatttactgaccagaactctactttagcttcttatggttttgttttcttaacatttagaaacggctaaatttagaggacataaattttctccatgagattgtttaaattcagttgactttttaatgtggattatatttgaacttgaatgccgcacgcatttttaatgctggttcatggcttctgtcactggtacgttgtatttctcactgtactattcttttacgttgcctcttgtctgaaatgaacttgattttaaccttttattttctggtctaattatatgagcttgtggggagcctcacatattgttagtatatctccttaaataacatgcattgaggctgaggtcagcagatcacttcaggccagaagttcgagaccagcctggccaacacggtgaaacccgatctctactacaaatacaaaaaaaattagccaggtgtggtggtgggcgcctgtggtcccagctactcaagaggctgaggcaggagaattgcttgaacctgggaggtggaggttgcagtgagctgagattgcaccactgcactccagcctggatgacagagtgagagtttgtctcaaaaaataaataaattaaataaataaataaaataaacatgaattgtataatccagctttgttattttagctctaaacttctggtgtatggagacagattttcagggagtttggtcctggaggagagacggctgcagaacctcaaatattactgaattaaaaaggaaaagattgtattgatcattttaccgtgtggggattcaaatactaagaggataatgatgatgataatgatgacgatgaaagcttgtttatgggacattttactcttccaaagtctgggaaggaatttcaagtgtattctggggacttctgaaaatattagccaatgttagaaacaaagtcgcaagccaaagggattgcttttgaatttaggcttgtgatccatcttcttttaattcactgttttaattaataaaagtctggaatatttacagaggattgtttataaaacttcacaaattagaaacttggaattaaaaatatatatataaaatatttcatatgtgtaaaaacaggataatatttaaatatctgacctcatgagaataatgactcagatttcttgttatcgtgagactttttctcaatcaactttttattaatattcataacgtttatgcaacatgaagattctgaagggactttgttgtctgagaacacatctatttcagatctgcggagtgtatcactttttgctgtgtcttcaaagtgattcttggtttattgcctgctaaggctaataaatgtataataaatctgcttgttgtgtcacttgcaggtgctatggtctttagaattgggtcactggatttctgaggagccgttcgaactgtctcaccacttccctgcagctcccgtaagtcagatgttgttttacgatggtaaatgcagtttgctgttctcaagaaattattataaacataagggtggacttaagtttttatccagtcaagcacaattatgcccataattaaaaagacattcacagaacttaacaccttttatcaatttattcgygagaacaaatgtgagaacgtgagaccactgtgcaaaaagtagtgaggaatgcagtccaaagaaaatttgacgattaacatcctcagaactgagaaaaacaaaaatgaaaaaagactgaattcttgggcaggtagtcttatatcttgcttaatgtttttactkttaatagaaatagaactgataggtataaagattatggcttgctggtgctgtgataacagtatttatatttttatggctttcctaaattccacttcaactttcaaatgcttcattgaaaagttctgggttctaattttttttaagattaagtaataattaagtggataatttaaagtttgcttggatacaggattgtgcagaagttgcctttcctgttcaaaaatgttaatttgtttgtcacagtttattcattcaaaagattaatagctgaaagataaatggtgatttttatctgccactggtgttgttatttagctgtttgagtaggccatatgactaaaacataacaaggagttgaactgtgctccctgatcactgtagttatctaggttgttgggttgttttgttttcatttttaagattactgtttgatttcctttcagctttataaacattttcttaaggagagacaaaagctcctctcagcaaaactgtttgtttgaaataccgtgtaaggaactgaagtgtaaagtaaaaacacaaattccccccattctcgctcataagagattatatatgatgcacaatgacataatgagatttgtccttgaattttttatcacctgcctacaaagagaattgatataaattgtgttgttgccagtttttcctgcattarcgtttccctacctaagtatccatcactcttgtcattgagatatcctagaaacttgttgttgtctttcgaggctgtgaaattttcttattttcagttgtttttcaacttgatacaaggccatgataccgttgttgaattcataaaaccttcttaaatataaagtagatacagttctaagatagggaggttcttaactagttaaatagttgttggaaaagtgcaccttggtggaaataaaacagagccttgactttgccagagtccatcattgactccaaatatgtagcaacacctgtgtgttctaaaactacgtcaagtggtggggagaagttggggtaaaataaattagattttgaaatggaataaagaaaaaataatggtagaacactgtaaggtgaagacagacatatagtagatgctagttacagactggactctgaacttccttgcaaatgattcagaaaagaatatatgagaaattgcctttaaattataaagctttacacaaatgttcattagtattaattgtactatgaaaatttcaaaaggagttaaaactccaggagtttatggttttgtagtcccgagtataaagctgtgttctcaaattttcttttctttctttttttttttttttttttccgagatggagtttgcttgttgccccggctggggtgcagtggtgcgatttggctcactgcaaccttacctccctggtgcaagcagttctccctgcctcagcctcccgagtagctgggattacaggtgcccgccagcacgcctggctaatttttgtattatttagtagagacagggtttcaccatgttgtccaggctggtttgaactcctgacctcaggtgatctgcccaccttgcctaccaatgtactgggattataggtgtgagccactgcgcccagccctgtgttctcaaatttttggtaaatatttaaatatattatgaacatcagattttgtttttgcactttgaaacccttttttttttttcagtttgctgattgacataaaaaaacttactagtgtcaattatttttttccttaagtaaatttaagggtgaatcttgagacatatagctttgtaaawttcttaaatagaaggcttttctcaaccagaaattaaattgtagtctagttctataaaaatatatcttactaggaaagaaaacagacctctgttttagaatagtgagaagatagtaaagtttctttgtcatagaatgaaatgtataattttcctcatcattaaaagtaagaagtttccttatcacaaggcacaattaggtcttttggaaacaaattataaaattgtaaatattatcataaaagttaaacataggcatatcccctaataagttatatttaattactaaaaataccttcatatttaacaatcaggcagaaaaaaatagtacggtctgcatataaactaaaatggcacgtttctgttgataatttcagagattctggaagtttctaccatataaatttgaaatacgtatttgagcattaacttataactaagctgtcaacataaatgtaaatacgctgtttttgaaataaaaatttaaagcacctaagagatggagtaaaaatgcactaactgtttttccaaatattaaacttctagtaaccccttctcagaatatccctgaatatgtctttttatggcttagagagtttttttcttccttttaattgtgatagtgatggtgaattcaggacatatgggtatttacacagtgtataaacagtgctcagaagaatgcagttccaagatgatctgtattgtataacataagtgttctgttttccakttatttactgataaacttgcacataacattcttggttgtgacagcagcgtctgtaaactgtcagtctgattctcagcctcgggttcatctttgcataggtgttctgtctaatcacaattatggatgtttagggtcttgctttggtccgttaagtgatgcaagtttaagtgataaagtttacaggctctaatctggagcatgtgggtcccgtcagcaccgagcacacgccctctgtggtggaagaggacacagtgcgcaccgtgactttcagtgcactgggcttaagtctttgaaaatagttcgagacagttcctcaggtggactgggatgtttagaaatctgctggtcggatcatcatggttgtggccttgagcgaatagcctgagcctttccagtagtaccatttaatgccgttgaacttatttgtgttctgcctctgtggatagtacattccgttcaagttggaaggaccacatgcatcaaaccaccagcctgtgaaagtaaaacacagaaggaattaggaactaggtgatgccagctcccaccacgaagacagcaatactcagctaaggcaggaggcacactgcaggcgtgtggagtaggcacatgcagatgatggtgagtataggatgtgcactggcagagggattgttttccagccatacacccatgacatcacagttccattacggcaaatgcttttacaagccttcttccaccttttcccttgtgctgtgtggagaggcctgaattctccacagtcctatttggtaagcccacagtgtgtacacacttacagcaggagtaagcaaacatctgaggcacagttggaaaactctccttcaaccaggattactttgcagtcccagcaacatggtgggctggactcrctcaggctccccttgctctattaaatgattttttcggttgaagtttaamctaaaatattaagtactcagtggagctacataaaaaggaagtctctatgtttcagagacaaaaaggaaatttaaagtgagagtgtgtgctcgctcagctaaagccagggcaggagaggtgtccagcacaggggctgtgggagtgaagccccatctgcaccttaatttctgggcttggccaaaaacaggagcatgctggggtttgtgagagaaagaaacacagtagtccccccttatctgctatttttgctttctgcactttcagatacctgaagtcagctgggccaaaaatattaaatggaaaaatctagaaatattctataagcaggggccgggcgcagtggctcacgcctgtaatcccagcactttgggaggccgaggtgggtggatcacgaggtcaggagaccgagaccatcctggctaacacggtgaaaccccgtctctactaaaaatacaaaaaattagctgggcgtggtggcggacgcctgtagtcccagctactcgggaggctgaggcaggagaatggcgtgaacccgggaggcggagcttgcagtgagccgagatcgcgccactgcactccagcctgggcgacagagtgagactccagctcaaaaaaaaaaaaaaaaaaaaagaatgtataaaccttaaattgcatgccgttctgagtaacgggataaaatctcctgatgcccacttcatccctcccagaacatgaataatctcctctatccagtggatccacgctgtctacatccggtctcctgatcacttagtagctgtcttggttattagatcgattgtcacagtatcgcagtgcttatgttcaagtaacgcttatttgacttaataatggccccaaaagtgcaagagtgatgatgctggcaattcagatatgtcaaagagaagctgtaaagtgcttcctttaagtgaaaaggtgaaagttcctgacttaataaggagagaaaaaaatcgtacactaaggatgctaagatctatagaagaacaaatcttatatccatgaaattggaagcaatatgttgtatattattcagttttattattgttaagtctcttgtgactagtttacaaactaaactttgtaagtatgtgtgaacaggaaaaaatatacacatagggtttgatactgtgtgtgatttcaggcatttgctggacatcttggaatgtttcccctaaggataagggaggactgctgtaaccttgattttacatatgttaaactgaataaatctcaaaaacactgtgttggaggaacacatacagtatgatactccttatattaatttttaaaatagagaaaataattatgattgatatctccatatgtaggaaaattaataaataaagtgaattaacctcgacccaagcgtcaggtagggaatggcactggcagctcctctttagccttacccgtaatgcattatttcttaataaaaactctatgccaaagaatatatatatatatattcttatgtatatatagaatatatacatattctttatatatgtatatataaaaacatacacatattctttatatatgtatatatataaaaacatatacatattctttatatatgtatatatataaaaatatatatatattctttatatatatatatgttgtgtttatacattggtttgttatttcatccaggttcctacattctttcttggtggtaacagctcagtgacttcatttgattcaggtgaatgcagattggacggaagtttgcgtgttctattcagaatccttcacatattcaggactttgacagattcataggtcagtgccttctggagcttgtccaactagagaagttgctgtccatgcaaaatggagctgctcattaggctggttcattcatggtccagaccactggctggaatttgacctcttcacaggcaagaccactccactttctctcttgggctgtttttcctctccccagtctcttttccaattacattctcagtccctaaatcttgatttgcgtaagtaaatatattgtttccttggttattaatgcaattctcctactctcctgagaagctcagcacatacgggtggtctaataagcacacccttctcaaggagagagctgggtccagcatgtggggaaatggtagacaggaaacaaagtcctaggtgtctgtggctcctccacctgaccctttccctgctgttcagctttaaaaaggatgattgtgccaggatgaaggaaacaggaagcttttgcaaaatcaataggagggctttgctcattggtgtaataatggtgtaacatagggaggacctgtggtaccaaatagtagtcatattatctcaggaaccagaggattgcttttttttttttttatgaggcctgattctttcagcataaaaggcatgaaatttaaagacatgaaaattactgaatttcatattattttcattactaaatcctccttttgactgttaatgatgctttttttttttgagacggagtctcactcttgtcgtccaggctggattgtactggtgcgatctcggctcattgcaaactctgtctccccggttcaagagattttcctgcctcagcctcctgagtagctgggattacaggcgtatgccaccatgcctggctaatttttgtatttttagtagagatggggttttgccatgttggccaggctggtctcaaactcctgacctcaggtgatccatccaccttggcctcccaaagtgctgggattacaagcgggggccaccatgcccagccctattaatgattcctatagtgtaaatgcatcataacttgggtcatccatttgtttaatgtagtaactttcatttataaaacatgttgaccatagctgttacctttggttttcctgggtgggtaacatattaatttttgcagatatgatttatgttctctagaaattaaaccctgccaattttcctgttattctttacattcatcttgcactattggcagagtttttgttgctactttaaatctttcagtgtttttcaagaactaacttgacagcatttgtcacacttttttcttgtctcagtcactaagtagcgtttgttcctgtcagtgaatttctaaacttttaacaaatcagaaaaataacactttcttttcttttttttttattttttttgagtgaattcttgctctgtcccccaggctggagtgcagtggcacgatcttgggttactgcaacctctgcctcccaggttcaagccattctcctgcctgagcctcctgagtaggagtagctgggattacaggtgccagccaccatgcccaggtaatttttgtatttttagtagagatggggtttcaacatgttggccaggctggtcttgaactcctaacttcaggtgatccaccctccttggcctctcaaagtgctggcattacaggcgtgagcaccgggcccggccagaaaaataacattttctaaaactttattcctatgtttgaactctcaaatgtttctgaataccaacccatctgttttaagtgactactacaatggtttttggcttatgagtgtggttttcattgtctgttttatggcagtgtaataccaaacctacaatacaagaaaggtctcaaagtagaagatgactcattttaatttgatttactaaaaaaggcggattaactcatttgtgtttataggtgttgctatatattaatggaatcttttttaaaaagacagctggggccgggtgtggtggctcacacctgtaatctcaacacttctggaggctgaggcgggcagatcacttgaggtcagcagttcgagaccagcctggcaaacatggtgaaaccctccctctactaaaaatacaaaattagccgggtgcagtggtgggcgcctataatcccagcactggggaggctgaggcaggagaatcgcttgagcctgggaggcaggggttgcagtgagctgcgatcacactcccttctggacaacaaagtaagactctgtctcaaaataaataaataaacaactggagactgtgtctctaaataaataaataataaatgacagctggaaattccttctttgaacattaaattattagttggaaatatttctataatctatattactgttgtggttgctacttggaatttttaactttttacataaagcaaaatgtaattaaaccatctctctagtatccagcaagcacaaacgcaggagagcttgctaagaatcaaatatcccctctccttgccagggctaggtcctgaggagacacagttggcttgctgacaagtctagctccatatcatattctcacttaaaacttagtctaaaaaaagtgaaaaacacatttacctatatcaagctagtgtgtctacatatgaaattgtggacatcgttacaaatcacaatttgtagtccaaattgccagcctttccctctatgaaatcattccttgccaatacaaataggaagacagaaagtcatccctacctcctgttagcatttgtgaacatttgcaaatacatttgtcgttgtctccatcctttgtgctaaaatcatttcctggttggctgatgctgcttattttgccggctgtccctgtaagtcctttraggtgaatcctgtaagcgtgcaaagaaaaaaaacacattggctagggtcattgatttaccgtagtggcaaattttttgtgatgaagaattccattctacagaagcgtgttctgtactcgttaatggactaatgcatactctggacaaaatattttgcactggtataaacaggaaccaacttatcatcaaatccttcagcaaagagggatgttttcatgaaaccttcaacacatatcacttgcacaactatcagaagcgactgtagagccctgtaatttattttcctgctgctttcagataaacagaagagaaagaaatgcagcaccaggctcctcctcccaggtctccagtcatcttccatagagacggagtcctgagacaactgggcaacctcaaacattattttccgcaggggccccgggggggatggagaatgcagcagacaaggaatggccactgagtttggggaagaaatctacagaacggtgctgaaaataaatccttgtggctacatttcctcatgtctgtatagtagggtaatgtaattaaacttttagacattgagaaaggaacaaatgtcggagtaagttagacactatttacaatacagacgatccctgacttcccatggggctatgttctgataagcccattttctgttgaaaatgttgtatattgaaaatgcatggaatacacctgacctttggagcatcatagcttagctctggccttccttaaatgtgctcggaacactcacattagcccacagtcagacagagccatttggcaacacggtgcacgcagygtctgttgttcaccctggggatcacaggactgactgggacctgtggctcgctgccgctgcctggcatcatgagggagcatcgtgccacatatcactagccagggaaagatccaaatttaaatcccaaagtgtagtttctgctgaatgcgtatcaccttcacaccatcgtaaagtcgaaaaatcttaagttgaaccattgtaagctgaattgcaaaaatacggcttacatcggtcatctgtgtaccagcaaggagcatataagggaagggagaagacaatatttttgaggttgttttttcttttttttttttttttttattttccataactatgctcaagagtttctgctgcaaagaagcttcttggcagatggttcaggacagatcagagcaggcattcacgtaatggggtatgccatgttggcacgttgggtcctcacgtcctgatggagaaacaggcacacgaagacccaggcgaggagcctacaaagcaaatcctgcaatggtggcaggagaagtgtacttgaagcaccaagatgatgcccttctttgtaaaacctgctaatgtttgcaagctgccacattggaataatataatttctaacagtttgtattggaagaatacaaagaagagagaaaatgttcttttagttttacctgctggtcgggccaggccaggtgcttacacctgcatgcacactggatgcttataaccacgtgcagtggtggccgccatctttgttttggcactgaaagtcactgaggttcagagatataaacttgtccgaggtcagactcttaagtcatggaggtaggatttgcaccagatgcagcaaatgcctctgccatgtttcaacactggtgcacacctaaacagagatgtttgtttgttgaagaagttgtgaaaagatgagggtagggccatgtgatgtggagttccgtaagtgttgctcctaagtgacttcagtattaaggcagccctagaaacttcatcctaaggcatgaactggacatgtgagtctcagtattttcccacacgtttcaaaagtgagactggccgtagctcagtctctaaatgcctgctgcaaaatgctaatgtcataaatactcatctctgttgggattttgaaacactgtactttctttccattgtcttcccattaatcatagacaggattgagatgaaccacttcccttgcttatcttttaactctctcttgtctcctttgaacatgtttagttctcatggaacttgttaaattatccccagaggcaagaaaaataagggagaatactattttttatgagtctctgttagaaaggttttgtgtaattttaggtccttttgtggcccactggtttaaagtgctttctttaaaatttggttattaagaatggccatgttcttgaagttgctttacattggtatgggttgatttttttttttcaatctctgcagctttgccagggatgattttatataacagtggagtaaagaggtaacatcaacattaacaattaaacctcagtgttatataaaactgccagaatgtgtgtgaaaagtgatgaatttttaagatttaatgtacgcatagcttttagtttcactagaaagaatgaaattctattgatgcatttatgcatttcttataacatgtattttccagttttccaacacttggggaacatttcttctgggaaaaaaaaatcccttacatgctgcatacaactggcgtctcaaagcatttgcagttatgagaagtttcagtcccttcacagttctcttatcatgctagcatcatgttttattaggataataattttcgatgtaatatctattttatcttgccaagcaaattaagctttaaaccaatgtgtgtgtttttctaaatggcctatccaaaaattgattgcatttctaaaggaaatatctgttaagaaccatctcagtttaaaatatttttataatgtcagcrtacaagggtaatgacccattttgtaaaaatcttyttatacaaacagcctaatccttaatttttgtgcttcttttttttttttttttttaattcttctgttgtagattcctaactgttgccagttgaaaaaatatttaacttggaggtaaaacactgaccaaccacttgtgtctcaaaattcattgaagttttgatctctttggagtcaagttggaactgctgtgaggcccaaacacctatcttctcattcatctcggttgttgcctctccaggagagcctgatctttccataatgagaatagtgaatatgcttcactgatgtttaaagagtcacatccatgtatatctgtttctcaaacatgcttctgaattttcatccactgtttgtacagcaggacatactgggcattgtagagttttcagttggttgttcaggcaacttgacatttagccgcttctccgtgctgcccaccacaatcctcccctgggcagcctgctcaaggactttaacattgtgtctcctttcagactgttcaggtcgtggagcggagtgtctgacttgggcattaatgagatgaagacgagactgtaggtcagatgatgactgtttttgtgatgttcgtgttgaccttcatttgctaatttctgacctcaaagtgggtatttcataatgtgtgctccatgatcacgaggcgccaccagtctgtgctctttagactcctttaggctggcgttggtgccagtgggcacacagtctcacttctctgcccctcccgttgcacacacatttcggagtgcctctatgtgccttgtgtaccagcattactgtgcatgtggcttcaccgtacttatcttgcacactaggttgtcaagtcccattgctgttctctctctctactctcatggcattttagaggcagaaagtaaattcccagtcaaggttgcccatgctattacttatgattattgctgccaaatgggtgaggacaaggtaaacacccagggaatgctgtgaatctgatgtatttcctgtagaggagagcagagttgactaaccatcccacctaactctgccatctctaaacttgacaactaatcttgactttgagattgaagacaattgaatgtgtttaaacttcataaagacagactaacttttgaaaccttttggaataaaacagcacagtcacaagtatccatcatttatgctattcatgtgacatattatcatgggaacacttactattcactgattttacaaatacctatgaaagccaattatctaccaggcagagttcttctaggctttgaagatacacagtaaacacaatggacaaaatactgtttgtatgaagtttcttttatattgttataaccaaagttagaattttaaacccagagaaacttaaagaagtaatagtttagatcttggttaaatcattgtgtttctcattttctggaatagtcacccagcaaaccttttaattttttttttctttttctttttcttttcttttttttttgagacggagtcttgctctgtcgcccaggttgcagtgccgtggcacgatctcggctcactgcaagtccgcctcccgggttcacgccattcttctgcctcagcctcccgagtagctgggactacaggcgcccaccaccacacctggctaatttttttgtacttctagtagagacagggtttcactttgtttgccaggatggtctcgatctcctgacctcgtgatctgcccacctcggcctcccaaagtgctgggattacaggcgtgagccaccgtgcccggccacaaatcttttaattttttctttcaattaccatgaactcacttacctataattgagttcttcacttgagagatagaaatgttcatacaatgagtaagcctcattcccttcccagtctttaaggtgtattttaagcacrtagcgttgctgrttagtcagttgcgaaacaaactcatttcccagccaatattctcctgaagggttaccaaatccctgtaatgcaagttgttaaattcaattatttcatgtaattttttctttgtatatttgaagtggatagtccgtcaacttaacayagaataactatcaaatagcagaaattccttctggtgctgtgacaatttagggtccttcccaaaggaaaatggattttaaataggtcagttattagatactaagctgctgctggaagaaaacttgtattaggataatgagaactacttggggagccaccagcagaagccttggcataaacagctcagttcatgggaatgtgaagcaccattaaacagtcggcttaccaaaaaaatgctgagtccacctttaaaaataagctaagtagtggcagccttgtttatttgagagtcttactctgttgcccaggctggagcgcagtggtgtgatcttggctgactgtaccctctgcctcccaggttcaagcgattctcctgcctcagcctcctgagtagctgggattacaggtgtgcaccaccacacctggctaatatttgtatttttagtagatacagagttttgttatgttggccaactggtctcaaactcctgacctcaggtaatccatctgcctcggcctcccaaagtgctgggattacaggcgtgagccagcacgtctggctgcagcttttgttttgatacagtttaccttatattggccattctttaaaggggagactgaagcaccaattttaaaaaccatgtcaaaagtcattggttagtttgggattggtggttaaggttccgcagatcttgaaagctatttttcacaagggaaattctttyctgatgccttaaagaatgtccttaccactttatattctttccaagtcctctgaaaatcaacgctgccatcctcacgtcgctgaataattgtccacccgcctcctccagcttccatgtcacagtaggcctgcaaacaggaatgcagggtataagtgacagagcccccccactccccccttacgtagcagaagcaggaggaatgtagaccctgagtgcaggactcagccgagagggttctctgggatataaggcacggagtagaccatcggggttgtctaagaaacagatggtttcaaataaattgaaagtgatggattaaaatgatggtaaaacataaacagtaatataatataaagtgttgatgagaatatgactcacttgtcatcatctgttccaggctaaagaccccaccctgatggctggtccaggagattgctgtttttttagagattcattatgaatggcacattttggcagattggccccgaaccccacatctccatcctgtagaaaaccattgacttgtgttcacagccttgaaacctttcactaaatgccagcccctgcccctccacagagagctatgtgaaggggatactctttgatcatagggtttggaggactcctgttacattctcgtactgtggggctgtttggcttcctttatgtgcaatactaatcagattttttggttcacatctgagcacaagggccttgaggctccagtgtcctggtgcaaggtgtgtgacctgggcttggcttagcaccttggcctcaagggcatgacttcacgtttctctgtacatagctcactgcccacagtttttctctaagcaactctttttatttctgctcttaaactggctctgtgggtttaaactttgtccagaacacagaattctttcttaagctaagcgcatgcatgctcaatttcaactcagctggagttttattatgaaattaaaaacccctgaaaataagattacataaatagttttaaataataaagattacagtagaacgaaacatgttttccagaaagtaagataaattttctgccacatacaaggtaggaaatattgaagttaaggttcaaaaccaattgtaaatattcttcttgtgttgtgtcccatggtctttttgagaataatggaggcgatttggcagggtaagcttcaacgcacactgttctctgtttacgagttagaatcctaaagaggagagcgaaaagagaactaagagagtgttattcctgttctttcatttcctacatgaggaaagaggggtgcagaggaagtgcagtggctgcctgatgcctcattgccagtgcaaccagagaactgtccctggaacagcctggtctgcaggggacttctccccagcatggttctggtctctctccaggaaggtcacccggctcgtattgccttccccagtggcactcatgtttggaaagataggtgccattaatgaaagttacatttattaagaagaaaattatttcctcattttaaattaatctgatgtagaaaatcagtctgcacaagctaaccccttgttaccgctctgtattgctatgatttttattatcattgctacacaccactctcatccaagtactctgccagatactttgtgtatattatctataatctcacagcaactctgctgtagtatcataattctgcatttttgaaaggagaaaagatttagcaaagttcgattttgatcagttactcaccttctaagtgatatataggatttgaataaggtctctttgattcctgttatgttttttttccactgacatcatgctgccaaaaatagagaaacctgaccctttggtaaataggccaaaagtccctcaacacagttccaagtttatatcagttcatgaataatactgcctcttatttgcctgcagtcaacaaaatggtcagtgctgctcacttctatcaatatttctttttaaaaatctatttcataaaccagctgaataagctactttggtttgaggattcatgtacatatattgaagttaattctgtatcctaaatggtatgctcttgggttgaaagcattgacgtggctcttggtgagcctatccttgttccaagaatgtttgattcttcagcgtcaaaatcactgctatgaagttaccagtacttaaatacatgttctgtcttgttcagagaacaagtttatcttgttatggaagtcagaggcaaaactcttaaatgtctgagagtcactgccagcacataaatgatttgagccatatgagtattcgctttgattccattagtgatgatgataaggttattagaacattttcttagtacttcatccaggtttttagaaaaaagaacagaggatttgtaaaaactggagtattatggttaattggactataaaacttgcagagaaagatagtgttcaaatagagttatctacccagccagaagatactgagtaaaagtgctgaaattgattatatcaggatcagcaaagcagaagtcctcagatacttcccaagaccttaccactccaattacaacaaacctaagggcagttaatatctttaatctgtccactggtgcacggtgcaggaacctgatatctttctgtaaagcttgatgtttttcagcaaataatacttgacttgcttcaactctgaggcaatgattaagtgacgggttaaatagcaaaccatagagacaaacgttaggagtcaggtgtcctgtgaaatttagggaaggaaatgaccatacatgcttttgataaatgccatcttgcagtctctctctcgtagaagaaccaaatgaatttttcaaaactaaagctgcagtattttggcctttcaggaaaagatctgctcaaagaccaattgaacattcttttcttgaattagataaatgagtgcagaatcgggtctcctgccaggcagagaagttgtctggtagtctttgaaggcagcgaaaatggtgaccactaccatttactgtcccaagttctaccccaggggctgtccctgtgttgtcacaacccccaaggctgagggtatcattgttctgttacagatggggaaactgagctctcagaaaggttaaatgactcgcccagagccacaaatggcagagctagaatttacatccaagtctgtctatctctccctggatcagattgtgacttacagagtcttaagtctgcaagggaatttagaagtaacaattgacaccacttatttttcaagatgagaaaattgaggtttgtgaaagcttattgttccctgctgtgtaaatgagtttcttttactgcaaatgtttaaagggaatataaatcctaatgtttccaaccatgacctgaggctcatataatcccagagtactatatattttcatctttctgcagaatatttcagttattctgggggccatggggtgggacagtgcactggggtgggaagcttgcacttagactgagaaacatagatgaaacaatgtgatggggctgcatggttagcggctgtccctctgcatcggtgtccatggcatccccatttcacatgcatcctctgtcccccctcttgacactcctgtccccaggccaagaacacgccaacatctggtgacagacaatggcatgcacagaattgtcatgaaaaccagatagcaaaagagattgaaaggcttagcctagagtctgttcgttgccttttcatctgcagcagaccgtgttgtttgtgggtttgttcccttccttccctgttgtatggcttctagggcgttagggacattaactaattttcagggttgatttaacacagcattaatgaattagaaaggtttctttggaaagcattatgttgaatagcacaatatttatcttttccgttcataatcaagatacatgttactgtttcaagttccaggtctttaaaaccctaatgcttgtatttttaaagtgtttttctttgatactgttttaaatacttaggataatactcaatttaaagagataatacttccaagagcctcgaaaatttccactttggtacagtatccactgtattctctgtagttatttgtgttggttcaatatgtagctgcttttacatttatatgcaaacatatttatagacatttaatatatacagttatccagtaattacataacacttcaccacactgattctcctgtaatatcattcttccctatcaaatgtttaagagaagccacattgaaatattctccggaagggtttttttttttccttatctaaagttcagtgtctcccaaagcaccttcaggagtcaggctctctgagtgaggctgcagaactaggaatgactgaggtaagctgtgttgtggctttgcctgctgtgagtactgactgtccactcacgttacatgcagtaattggacatatgccttgaagtgaactccgctgctggagaagaaaatgaacactgtctttatggtgtgtttcgagtcttccagactgcgttagatgaggttagaatcgccttccccagcggttctcatggtgtggacctcagaatcctgcgtgtccctcagaccatgtcaccaagtccacaggttgaaactgttttcacgatgctaagacaccaccggtctgtggcactgtgttagcgtttgctcagatagagcaaaaaccatggtgggtaaaactgccaccatccccgtgtgaggcagggcagcggtagctaactgtacaagtcactatgcagtcacacacaaaaaagaaaggaacaataatatcactgaaaaatgactttgacacagcagtagaaattattaatttcatgacacctctatggctgatgcactatggttgttcaaccttcagtttttggaagatactttctttaaggaaatgagtctgacacctcaggaaaaacaactgacagtatgcattattcaagcaaaatgcaaggagaatgtatttgttgccgaagataagatagacatttctacatcattcatatgcagcttttgtattttctgcacagcagcggcacttagagcccttggcaagcactctaggcgaggtagctgcccagtaactatggctttgtactgtgtgaggctggggaagatctttggggagagacttctgctctctggttcctcactctctaaagtggcctgcctagagccagggagttagtaaggggagacgaatacctcaccttgatctcttctgtagaattagggaatgttaacgtgtagatgccattcgtggtgtgtcctgatttgaatacttcagcacagtctctgaagctgatttgttcttctttagcaacagtggggtccttagctgcttttaaaaaatagtaaggcatttaaacggagttcatgaaaagacaaagacttgttatttcaasagccaatcatttggtgagttttattactttggaattcttaagtaagcaaaaggctgtaccacttttttaacctttctagaaagtttcctttcagcctgttttcttcttaattctcaaaagattaatacttactttttggtcattaattccatgtaattaaaatacttcaaataatccaaacttcctttgctgatgatcaattacatgtaatgaaagtacttcacaatcacataaattaaattattacttttgaagatctttcatcttgagtagaatagggtaaacttagtatggaagaatttaaaaagaatgtccctaaacactgttatctgtatcatgaccccattgcctgccccttcaggccattatcccactgaggacatagtggggtgcagtgacacatctcagcttaccgcatcctcctcctcccaggtttaagtgattctcctacctcagcctcccaagcagctgggattgcaggtgcccaccagcaagcccagctaatttttgtatttttagtagagacaggatttcaccatgttggccacgctggtctccaactcctgtcctcaagtgatctgcctgcctcagcctcccaaagtgctggaattagaggcgtgatccaccatatctggcccttccctccaatatataagagacgctgcaaagtgaaacaataataaggaaggcaaaatgtgcttaagaacctggcaagataagggaactagcatctcttaagtgccagtgtattatctcatttaatcttaatggccatcccatgggtctgatattatttttcccatgtaaaacctaaataaatgaatatcggctgtggtttagtaatttgcccagtctcatccttctaattaatgatggaactaactaaaagtaggctctttactgccatgaatcaaaagtatgcttggggtgtttgcttcataataattagtataacatatatttccccttctcttcttccttcattttaattggtagatatttcatgtgaaatatatgagaaatagcgccttttctgaaaggtgagaattttttagtcttttgagtgttttactgactaaaggttattaacgctgaagaaagcatgatatgtraacttacagtttgatgtggacatcatagtcagtaagttattaactgtctccatgagatcatgttgctgcttctgaagaactgaattattcaccgtggcagtcactatttttttttctagttcttcaatgatggaattttgcttggatactaacacctgtagctgatctttctcttcttttattgactgtagttggatgatgtgcttgtcttccatagctagcaccttcttttctaggaaacttgtaaggaaaagaattgttagttagtgaaggctattctaatgaaatattttatatttattgaatttctacttctccaaggtactctgttaagatattgtagtggttataaagtaatatgatcttaccagagccctaaggaatctctgaaacttgctgagaagattagatatataaatgtgtgtatatatgtaaacgtataagcatatatgtatgtacatgcagacttatgcatacacacaagaaaaggtaccccatctggtccaggataggtgggatatgggtgttttttgtattagatgctacagcgctcagaagaaaggtgctgctctttcaagcttagtgctcatgaagtgcttttttgagaagggagagtttcaactgggctggacccttgggtaggatattagctttctcctaaactatttatattttaatattaatcctaatgataataatagcacttaatgctatgtgagaaatactccttcatggggaggtgaatacttctcccagactcaagtcctggcttaccagccctgcgacttggaacagtttacttagtcaccctatgcgttaatgtcctcacctgttaattaggatactatcacctacgtcatggggttggtgtgaggaacaaatgggttttaaaatgtaaatgctggccgggcgcagtggctcacgcttgtaatcccagcactttgggaggccgaggcgggcggatcacgaggtcaggagattgagagtatcctggctaacacggtgaaaccccgtctccactaaaaatacaaaaaattctccaggcgtggtggcgggcgcctgtagtcccagctactctggaggctgaggcaggagaatggcgtgatctcgggaggcggagcttgcagtgagctgagatcacgccactgcactccagcctgggcgacagagcgagactccgtctcaagaaaaaagaaaaaaaaatgtaaacgcttagactagcgcctgtcatacattaacactcaatgaatgtttgttaacgttaatatagacattattattcccatttccaatgaggaaattgaaacttagggacattgagggccaggctcagtggctcacacctataatcccagcactttggaaggctgaggcaggtgtatcactagagtccaggagcttgagagcaggctggccaacacggtgaaaccctctctctactaaaaatacaaaaattagccaagcgtgggggtacatgaatgtaatcccagctactcaggaggctgaggcaggagaactgcttgaacccgggaggtggaggctgaagtgagctgagattatgccattgcactccagcctgggcaaaagagcgagacatcgtctcaaaaaaaaaaaaaagaaaagaaaagaaatataggaagaatgaatcacatacctaaagtcacacacagcaggtggcaggggcagaatacaatcccagcactttctgactctgaaatctgcttctctccttttaatgtggccccattccttctctaaaaaatctaaccagcctatcgcatgtacttaatacataacagttaatatgtgagccaagcccttgaaaagcttttttttctctttttttgagatggagtctcgctctgtcacccaggctggagtgcaagggtgccatcttggctcactgcaaccttcacctcccaggttcaagctattctcctgcctcagtctcccgagtagctgggactacaggcgcatgtcaccatgtcaggctaactttttgtatttttagtagagatgggcttttaccgtgttagccagaatggtctcgatctcctgacctcgtgatccgcccgcccctgcctcccaaagtgctgggattacaggcatgagccaccacgcctggcagaaaagctttttaaaaattatttagagagctggtaaaattatgccatgtaagtcctaagacactttattaatggttatatagtttgccttcctaatttcaacttataaacatacgttgctataaatatgttcaatgaagagcataccacttttaaactaaaaatagttcctgtccattaagccagaggaaacaaatccaagagagtagagactatgtatttgagaatgttaactgtttcccaggaacaaactcaaagacatgcacagtcaaggtatttggcagggttttttgttttttgttttttgttttgagatggagtctcggagtctcgcgctgtggcctgggctgttgtgcggtggcgcgatctcagctcgctgcaacctccgcctcccggattcaagcagttctcctgcctcagcctcctgagtagttaggattacaggtgtgccaccacgcccagctaattttttgtattttaagtagagacgtggtttcattatgttgtccaggctggtctcgaactcatgacttcctgattcgtccacctcggccttccaaagtgctgggattacaggcatgagcaccgtgctggctggcattttttttttttttaataagatacaagaggaaaattggatagcctgacactacattattcagcacctaaagaggctttctgtgataattgcaggaaaagcagcaactaaagatgtttcaatatcttcattttgtttgtacaaggccagtaaataaagctttcaaaatatagacacttttaaaaatagaaaaacagtgaccagatgtcagattcctctctctgacattttccttccaatataaagtttagtacacatgaatttgcacattgcagagttttgttttaaaggaaggggacctcatattcccttttttgagtcccgtataagtcagctatcttatttaataatgaaatatgtcaatgatggcatctttatgtttcagaattattttctgtctactaacaagttaccacagcttctgttaatgtcacattagaagctggtgaaatattctatacatttcactagcttttctgcgaaggcatatgaagagcagagaaacattattttcccacctgcttgataaagaaaccttgaaccggccatttaacactgctgtgagttatctgaagcctcctgagtcactttgcacttactttcctaggaaccgaaagcatgtgaaattgacatacacgtttcactgagtgatagttgggttcagatcacgtcttaccttccgtttaacagagatgtattgaacacctaccatgtacgaggtgttttttagggttttggagaaaaatcaagaaatgaaagcatcatgaaccatagtcttaagcctgcggaaatttagatgttttgatggtcttcacatcatcaagctaaaaagacaaggctatgaatgtctcccttgaggaaaaactacccttgtggccatgtaaggtctgtaaatagaagttatcacagggaatacatatgaagatcatggtttcactgaagagaaaatggagaccctgagaagtcacctttggtgtcacgagcaccttcaggtgaaaggaaggagccttaggctgggaatcccacctctgcacatggcttcctgtgtcacatgggcagccaccctgctgtggacctcaggtgcatgtctgtctaggtgaatatctatctaaataaagctctatgtaaaatgaaggcattcgatttcatggcctctcgggccccttttagttcgaatgatctggtaaatccacctttttttgacagtaacattttctgactctttaaccctgcaaacaatattaaccagccaaggaactggctacccattacatgtctcgcccataagcaataacaatcagtattaataataattattagatattcaattgtagctcttaaatgtattccagccccctgatcgttgtaaattagtatataattttggagagatgggggtctgtctttgttgcccaggttggtatcaaactcctaggctcaagcgatccacctgcctcagccatccaaataggagagattacaggtgtgtgccaccacatctggctaatttttgtattttttgtaaaaatgagctcattatgttgccctggctagtctcaaactcctggcctcaagaaatcctatcactctggcctcccaaagtgctgggattataggcatgagccactgtgcctggcttgaatctattctataaagaaagcaattgcacttttggggaattataaaagattatttaaaatgtggtttgtccaatgtgaaacaccatttgcatatttttgtaatgatatacttgcaaataaaatcataggccagtcagaatttaaggtagaaaacacagcatgcagaactcatacacctgtaaaatcatcaacactattttctttttttattatttatagctgttgatgaaaaaaacctttttatttcctttcatatctgtgacaaaaaaatacgatttctacatctgatgagaaaaagcttattcttcctacaggcatagttgaaagccaatatgattggaaaactatttgcaaagatgatatttgggggacataattgacccaaattggtagttttagcattgtagcatgctaaatttgaaacccaagtggggaaacagtattcagtattagggtatgttctacaaactggacatatcctaggtttgtcacggacatcattgtataacaggcaagagaaaagtaatctccagctcccatgtgttccgggaatcactgcagcattttgaagagaacattactaagtaagactattaagaaaacgacgccaggacggtggctcatgcctgtaatcccagcactttaggatgctgcggtgggcagaaggcttaaattcaggagtttgagaccagcctgggcaatatggcaaaaccttgcctctactaaaaaaaaaaaaaaaaaaaaaaaaaaaaatcagctgggtgtgtgacacatgcctgtagtcccagctactcaggaggctgacatgggagaatcacctgagcccaggaggttgaggatgcagtaagctgagatggcaccactgcactccagccagggcaaccagagtgagaccctgtctcaaaaaaaaaacacagaaaagaaaatgaaattagcaggattgttatatctcaatgattggtctcaaatgttcatttactgtttgtagaggagaaatctgaaacatgaaagaaaaatatttgaattttaaaaatctatttgcttttcaaaaccctaaatcaataatgacttaaacttggtatcctaaggacagaaagaattatttcagcttagttcttgattaacagtaaagaacaattattgaacaagaagtttatcatttttggttaagaataaagaattatttaaattgtcaaataggatatattgttatagccatgttccatgttgtatatacatgtcttcattaaaaacaaggaaataggcacaccaggtatgtgcataaaattatcctcttttgtcccaagtggaacagacatatgaaaacagtccccacctatcccctacaattttttttctattgttgatcttgagatttttctatattttatttaaatattaatataatcatgtttaatatttttggttttactttatcgtgtgtttgaagaggaaacattggatcataaaatgtgcattggcttacagtataagtgtagctttcatactatagaccattctgcgttgagtgaagctaagtccccaagggcaaaggatcttggtcaagttaatactgaaataaaatgcctgggccagtggttctttcactccacagcactagctgtatttttataatagattagcatgtagaatactgaggcagggtttggaggattactctaagaggatcttttgggccagtggttctttcactccacagcactagctgtatttttataatagattagcatgcagaatactgaggcagggtttggaggattactctaagaggatctttaaggggccagggaatgaaaggtaaaatccaggactgtgttaggagagctgtgcctgtgcaggaattttctccaagccctctcccttctcctccctcatgaggtttctgacccttacactagacatgaagaaactcaccattctgataattcatcatttgagaccgactttcatatctggaaagtgtgcagtcctgaattataaawgttttagtactgttattacctgttcttatcttgcaatttgtttatttcactggtctggtccaaaatctgtttttccaatttgtttgtcgagagggagtgttccaagagctgaagttcaagtctcgtggtctgatttaatacctaaatgtaacaaaatgaagttcctattaattattttttaattagtttaactttctaacttccttttcattaaagtacccaagctacaggaaaacataacaaaaacattatttattaacccaagtatcttattttggcatatttttcattttcagaaaaggctcaatgtcttagatcacatctgagtgtgttaaacctttttactcttttccccacgtctctattttttttttttttgagatggaatctcgctccattgcccggggtggagtgcagtggcatgatctcggctcactgcaacctccgcctcccgggttcaagcaattcttctgcctcattctccccagtagctgggattacaggtgcgtaccaccatacccagctaattttttatatttttggtacagatggggtttcaccatgttggccaggctggtctggaactcctgacctcaagggattcacctgcctcggcctcccaaagtgctgggatttacaggcattagccactgcacccggccgttatgtctctatcttggaaagtggttagtagttctggacaatggggtctgtgccaaatactaaatgttatttttctagtctgccatattttatttcatacaatgagacaagtaggagtagaaaatggtcatatttcataggtcgaaagtattttccctttgccgaaaacaaaatgctattctcatatttatttgtcactagacagagagattggaagtcacatgcttccattatataaaaatatagataatttttagcctgggatttcctcatttgtcaccacttgtttagacttttatttcttcttgccatttctccttcctgttttaaaacttgtttgaaccaatcgaagccgtatagcgtgagtgtgaagcggascctcagccttgccgtgcgggcctttgtgagctactgcgtggcatgagcagtgcggctctcccgcggattctctagcgcctggttgcccttcagcaggaagaatcgaytactcacttcctccatgtcatgcttattcaggatgtgatatcacaygcaaatgtcagtcagcattgttgccaaggaaccggggaccttgaaagaatcattgtttgctggtgtctttatgtcatttgcaggagccttggctggtccacagcgtgagtttcagggatggtcttatccttagagctggtttagttcttatcacaaaaagtcttctgtgagaataaagtccttggccaacrtaaggttttgtttgggttttaatattaacacctggaatatagatttggcctacgtcttctttgagtccaaacattctatgttggttatttctaaaaggaactggaaaattgtgtcctgtttaattcataagggttataacatgagtaaaatcccgtggggaggcagggaaggatggcacataagtcatgattggcccagtagtaattgtaaccattttcacatcacttttctggagagcatcaaaccgctggaccagcctgaaggcgtccatctgcaggggactgtaaattacccaggccaggtaatgatctctcattccctttaagatatgagacctccagccacccattgttgctcaatttgatcgtctctcattctgaccggcttggagaatcttgcttctaatcagaaattttcagatttgaatttaagtctgtttcacaaaatcagtaactgctcagcaagtaccttcaaacagagtgggtacataattcagtttctttgcggccttccttaagctcagccatttttcttttttttttttttttgagacagagtctcactctgttgcccaggctgcagtgcggtggcaccatatctgttcactacagactctgcctcccaggcttaagcacttcttgtaacctcactaagcctcccaagtaactgggtctacaagtgcacacaagcacgcctggtaatttttttttttttttttttttggtagagatggggtttcaccatgttgctcaagctggtctcggactcctgatctcaagcgatccacccacctcggcctctcaaagtgctaggattatagatctgagcaagcgtgctcagctggctcagccattttcatgtgttcaattgggcttcacatggaaaaactgcttactttccatctgttttcttattttcctgttatcctggataacatgatatctagtttcacaataggcgttttttttttaaatcatatgacgcaacacaagtacatcaaatgctatgaagtctctgaccgctataggatgtagcaaggtttgcattgctgctctgtcctaacactttttcattactattattattttttatttttttaaatttttgccaagctcccatgcttggatctaactattattttaaaatataagaaatgttatagtttaaaaatgcttatgagacattttttggatgagctattcaattacccatcagtgttagtatcaaaaggtggggcatgtgacttaatcattactaatttattttaataggttggtgcaattttgccattgaaagtaatggtggccaggtacggtggctcacgcctgtgatcccagcagtttgggaggccaaggcaggtggatcacctgaggtcaggagttcgagaccagcctggccaacatggtgaaaccctgtctctactaaaaatacagaaatttagccaggtgtggtagcctgcacctgtaatcccagctacgcaggaggctaaggcacgagaatcgcttgaactcgggaggtggaggttgcagcaagtcgagatcacaccattgcactccaacctgggcaatgcagtgagactctgtctcaaaaaaaaaaaaaaaaagtaatggcaaaatctgcagttacttttggtccaacctaataataattcgctttagatatatattgatatattgacttttaaatctttagtttttatgacttcctaggatttaaatttttagtaccttatgatccattatgtaaaatatttatgtatgtttttcctgaactgttgtgatattgtggaaagacctggtaatcaagtaatttgttattctattctcttatctgtaagtcttttgttaatctatcatttcgctactgttttctctgacctcatccaaccatttttaggaagacaatgaaagaacagctgtgtccttctagaatgagtcttacgagagtggcagggcttatggcatctcccctctcatgtcctctcctggctgatgtctagcatttcttgatccttttagctgaagtagcatttaggaataatatggagtggggattgtttcacttaaatctgctcttttttttaaaagcattccttgtagcccagagtaggaagccactgacttcagaagcatgtaaagaagccaggatgaggagtcagaaagcgggcttggccgccgagagtcacgaccacggctttgagcttggagcgtctgcatttgtactgctaatagcagcttttccctttcccacccaggccgttcgctgggtcacatgttgtgcatcatttagcatgtctctcggtgaattttcttcttttgaaattttcctattttgctgttattttactagtttctttctttctttctttcttttttttttttttgagttgaagtctcactctgttgcccaggctggagtgcagtggcacgatctcaactcactgcagcctctgcctcctgggttgaagcaattctcctgcctcagcctcccaagtagctgggattgcagatgcccgccaccacacctggctaatttttttgtattttttagtagagacgggttttcgccatgttggccaggctggtctcgaactcctgacctcaggtgatccacccatctcggcctcccaaagtgctgggattacaggcgtgagctactgcgcctggccactagtttactatttcagtcttctttctgttattattaatcactagctcatagaatctcacagtggaaagagaacttagcaatcacttgtctggcccaaccctttatattatttgaggcccagaaaaggtgagtgcctcattgtgatgcatttatttggttagtggcagacctggagccatggcagcgctcagggctcttgctcgggcgtgcaccatcttttctgtggctagacgcttctcactgtcccacttgtctccttctccataatctcattccacaggctgtgttagctgttgagattcaggtttcatcttaactcaagagttagatttaaggccagagtttctagctctttgcctcagtgcttttcatttctcaaatgttcaaagactttaggacttagaaatggaaaatgattcccggagtccagaaagcaccagggagacagagggggtattcatcttgcagtggttgggatgcgtggcatgaaaatgactcacatgtcttcagtagatagaacacatgaaatttaacctcagtattaaaaacaaaaacagatttactgatttttaattcataagcagccatacatccttaayttcttatcaattcattccttttctcctgtggtggtgctttctttagtttctcatgccttcattgaggaagctcctgacgcgactgagtgctagtctctagctgcagggacaccgtgtgctttatgtggcattacttacttgggcttccacatcagttaacttccgcgtttgctccgctgtttggttcaacaggtttgtccctatttctatcatcacagccgtctggttctgtactgcattctgctgtatctctaccatttctttcttcatgttgtcctggatataattctcaagctagaaaagaacagtgttggaaggcagtcattagtcaaatgaccggaaacctgattcctaaatgtttgtcatctcctccctatctttaaaaaaaaaaaaaaaaaaaaatctatcaaaagacttgtaccttgccttcccttttggaatcttactatttttttttatcattaggaaaatacagtgtgattttatttttatgcaaaatctggcaacttagtcacatcatgtaaaggagggagacaagctactggttgcttctgtgttcttctagaagtccatgtcatggcaggccacagagggtggtgagggcagccacagggactgctgggtgctgccactgtggggttgtgtctgtcctacccagctgcaactctgaccatgcagtcaggaaatgataatttgacacaaagaagcatcactatttctctcacattctagacttttggtttctccacatagacttgagaagacactctaagacagcatataaggagaggagcacccttttgattttccttttaacctacggaatcaccactcagttccacattctgtggggtcttccccaccttcctccgtattgagttaattcgacctattaaatttttcctaacatgtatgcatttttcacaattttgtcatttcatgtatcaagcaaacttttaatcgcaccttggtccatttatcacctaacgtgccatgggctggttcttctctccctcagttactaaagatgatgatcatgccgactaattttagcattaactgaaacacaagagaaggaagaagctcatttcactgccattggtatagctatccctgtctatggcagtaaaattacatgattatgtataactgcaagacaactgagtacgtgggaagagcctttgggcttggagccagggaagcctgccctctgctttatagtcttggttctaggaaagttgcttaaccttttgggaccctagtttcctcatatgtaaaatagggtttctggttggtcagaggagtgtcttaaagaggggttaagctgtgcttttaaagtcattgtgtatgcgtaactccagatacttagcgtttagtttctttttttttttttttttttttttaaataatctaatgatgggaaccattcttccattccctggtccaaagtataagctcgtgagtgcacaaascatgttttcttccttttcacatagtgtaacaaacattgtttattacattgaataattgaaagatgattataaaactggttctggtgccctcctttaaaaacttagaattctttatagagraamcattcgtggagtcagtcatcagacatgatttcccccaaaatgttaaccactaaataattctgtgctttctgtctttaagagtaggaaaataggatgggaagggtagagtttctctcttagagcttctttgttgatgcatttcatagattgtgtcttgtgactggtatcagatggttttaggattaggctggaactataagtttcctgtttccgatgccccctcgccatcgactctgccccacttctctaagctcccagctmcctgcatgcccctcagcctggtcactaaggctgcctccctggcagtcgttctcccgtggatattggatgggtcagatgagcaggatgcatgasaggcacagtcagccctccatctgtggcctccacatctacgggttcaaccacaccatcaatatatttwaaaaaaaaataacaatacaacaataaaaaacaaaaattgyaaaacaatacagtatagcaactatttacatggcattgacattgtgttaggtattctaagcaacctcgagatgatttagagtatacgagaggatgtgtataggttatatgcagatctaccctgttttacggaagaggcttgagcaccgtggattttggtattctcgggaatcttggaatcagtcccccacagacatcaagggacagttgtactagagctccaagcatgtgtaaaatcattttgttgaaatgttactcaagccatccacccgcctcagccccccaaagtgctgggactacgagcgtgagccagcacatctggctgaagccttagttttccatatgaaccaaaacagagtagaccactactttaaaaaattaaagtattaaaaaatttttaaaaatttaaaaaaataaaaaatcagtcactgatacccggcaggccagcaaccatctctattataggcttcataaaatatgaagagtctgaaatcttactaaccctttctcagagttagctcaggctttttagtgtgtgtgatctttcttaattcattctttctccttcctcccctgcctttataaaactgtaacttttgtgattgaaataaactatttaaaagaagccataaatagcagttcgtaattctcccctccgctcatcgccatgggagtaatggaatttttgaggttgcagttaaagctgtgtgtcacccagaggcactgtcttagttactcctcacagcaccccagccaagataatatttaaaaagtttcattccgggaggcttggaactatagagatagactccagctggagtttagtttaagcccatactcagaaataataatttacaaagtggtataaataaaaagtcttaacctccttcttgatttcagtacttaagagctaaataaaaattattgtattttgtcactctaaatcatacaaccagagagggaaaatgaatcctctaatactgccttcccccatttctagagctactgagtcagatgtgtttgcaactctccagagatatgagaggattgtttataattgaaaacttaaagtcaaattccaatttgaaattaaacttaggaactttgaaagacatacaggcccaattttaaaaaataaaatttcttaacctgccatattgttttctaaacataaaaacaatagaatgcaagatcctttttaaattgctactttttagctattcaggatgactaagtataggttcacagtgggtgagctaatgtgtgtccatttatgttaatcttacataaaagcagattacaaatacacatgatgtgtgtatatacagataggtatatagcatatatgtatatagtgtctataaatatatacagctcttgaagcatgtatcatttaaataaaagaaaattctgtgtgatactgactgcattgctaattaattgaagtctttgggagaagaatggaacagaaccaaaaatgtgcagtagtagatattttgtgttgatttaaaaagatatttgagccagtcgtgatggctcatgcctgtaatcccagcactttgggaggccgaggcaggaggattgcttgctctcaggagtttgagaccaggctgagtaacatggtgaaacccatctctacaaaaaatacaaaaaaaaaaaaattagcttggcctggtagtgcgagcctgtagtctcaggtactggggaggctgaggtgggaggactgcttgagcccaggagagcaaggctgcagtgagccatgatcgtgccactgcactgcagcttgggcgacagagtgagaccttgtctcaaaaaaagaaaaaaattaaaattaaaagtaaaaatacttatgttcttactcttgaagtcattaaattaaggttttaagagaaatatatgatgtgacagtcaggtactctttaaaaacaaggaagaatactgtatatttagccccagaaacactagcgacaggaacagccacagtaatggtaggtactgtttcttggttgccgkcactgcctgtgctgtatgggaatcgctgtgtcgggatcccaggcgcctcacatcagcacaggtggatgcagggctgagcactggaatgaccctcagcaaaatgttagctcaacccagaggccgcttcatacttttccagccttttaagagccaaaagtgatatatctcaaaattggcttgagtataccttccaattccaggcttcacaatgccttaagaaaacagacagaccacccacccctcagtggagggccatttttaccaccagaaaagcccagaattaaagatgaccaatgccaattctatcttctgggagcatcctgacaaaagaatctgtgttttcttccaaagattagtagtaatttttagagatacagaaagactatggatgtccatcatatagtataaaaatgaacatttccaaataaagatgtcccatttaatgtagcctttccataaatcaccacgtatcaaggataatgagaacaaacctagaaacaaagccatctggctcatccacttggatagacagaccttgaaatttccctgtctcttgaccttgatgaattagttattttctagtttattgtcctagaatgtctttctgtttagtgtctctcttatttttactggctgtgactgaaacccagaaatatagaaacctgcccagaaatatgaaattccattctaagtataaggaagtcttagtacaaggaaaaaaaaaaaaaaaccaacccagtaaataagccatcctccactggcagcaccaaactccacttgcctttggagaatgtttcccatccctgtcatctgcaccgaactgctctcatcaaaacagttccaagatacttgaacctcccgtgggaggggacccggctctttccaatttcacatgcatagcatgtgaaacatattcatgtttcgcaggaatgtttgccatcgccttcatatctgaagaggattattccatgagcgtgatctgtaggcacacgtgtctgaataggtcctgctgtatatgtgtgcgaggacagtgtgtgttcattttgtcctcttcttgatggttgacacagtcggcaaagtgtggggccttgggctgttcttcctttctcagaactcaagtgagttatgcaagtttaacattgagggccacagtgatccttctagctgcatggtttgctgcttagtgttatttgatttgctaaaagagttgcgccccagacatagtctttaaaacttggcagcgcatcgaaactcaagcaaccaggatgaaatattttaatgcaacatatatatatatatatgtttacattaatatatatatattttagtgcaaaatatgttctgaagttttttattactcccacaacgttttgaatgatcaaatttgacaggaaaaataggtccatttgtgaggcaactatggcagattgattacacatttaaaagtttatctggctatcttccttctcaccaagattgtcatcattattttttataccaaaagaaaagtaatcttgaaactggctcagtaaaggaaaacatagataatatatgaaaactatccccaacttggagattctgatgttgatttctcaccaactgtagatgctggttgagagatcctttctatttaaataaaattcaaggtccttagacctttttactaattagtttttgtccatctgagtgacctaaggtggacaaaaactaattaatcagagggtctaaagaccttgaattttctggtaaattaacaaataatttgagatttccttggaaactttttactgttgcccatttcaatttcgaaataggattttgcaaccatctctcacacacacatacacgtttttctatcccaatgatccatccatcttcccacccagcccttccatttttctagtaaacccttgaatttttctagtaaattcacaaataatttgagatttccttggaaacattttactgttgcccacttctatttagaaataagatattgcaaccatctgtcttacacacacacatacacgttttctaccccccatgatcccatcttcccgcccagggtccactgtcctctctgtctcttgctggccacgcatcccccagctgctctcctttcatcccgctgtcagagtcaggaatccacatgcaaagctggtgaccgcagctcactttcttcccttgcaggtttgcctgaggataaggtccagattccttctttttaagacacacaccgcctcctgactggcgcccctgatcttgtgggcctcagacctgggcgcgcactgtccttggctgtcccagctgcccagcggcctcataccacggccgcctttgtatctcctccttgagtcttctcttcctcctcaggtcccaccatcccccattgcatgccctmagcaaagatactcgttttgtgtttccttttgatatcaaaaccattttgtatttgtgtcatttcattttaatctccacagacaataggttaatgttcttgcttgcttggtgaagagtgaacagaatcctcaaactctgcaaccattctacatatacaccctagtaacaacaagcaaaacatccactcttagaattagtttgaaaacttgagtgtaagattattaaatccagggatattctatttgggaggcttttgacctaatgttcttggttccctgtcatgaggaaactctgaaacatcatttgaggtctccagacagaaaagtggcaaaactgggctctcctccccctccttttagagttgggcttgtgtgtgtgtgtgtgtgtgtttattctggagattttgctgcctaagcagctgtgtactcagcagtacttcatggcagaggctgagcctaaagagggaagggctgggagatgcggattttgggcagcactttgtcctcctaaacccctcgccagagcctggggggtaggcacagtacccacagtgagaggtgatgttcacatgccctgtgacgtgggaagcaagttttctccatatattgatgccagatttgaatttctagaacctagaaaagcccatgccaaagctacttgccatctgttgactgtttttatagtcttggccttttcttcacgttcagtgtaaggccctagaagttgaggcaaaagctaaaggccgagggagggaagcctggcctctggtgccaatttcctagtgggtattgtgacttctcttagggagcacacttgccttcacctgccctgaccacatggacgcctgcccacatagggtcttttaagcacttcctgaaatggatctgttctgatctagcctttttgcttttttctagtcatacttttttattgtcttttttttgagatggagtctcgctctgtctctcaggctggagtgcagcggcgtgatcttggctcactgcaacctctgcctccctggttcaaacgattctccccgcctcagcttcccaagtagctggggttacaggcgcacaccaccatgcctgggaaatttttgtatttttaatagagatgggttcgccatgttggtcaggctggtctgaaactcctgacatcaggccatctgcctgccttggcctcccaaagtgctaggattacaggtgtgagccactgtgcctggccatttaataatttatgagtgactatctgatactgtatctagataaccaacccctttcctactttcgctagtataagagactgaaagttcacttttggccactatataactccaagatgtattaggaaataagtttgtgggcctcagctggtggcattctaacattaatagtccatgcctctcctcctgtggataggtacaccctacagtaatttgagtgtaccagaatgtctgtgctctggcaaatcctatccgctttgctcttctttgagtgcagctgcatattctttgcattaatttttttcacatatatttgaatatatgtttttccacatatattcatatcattttacctctttgtgtgtttcccttaccactactccaaaatttgataaggaaatgtgcttttcccttcaaaatgttccatttattttctactgataaagtggctatttctcatcaatagcaggcattttaaatatatgtaagtttaaggagactgctgtagtaacctcatgtaaatttctttgggcatttcatatgcaaaaggtgtcacattttacacgagtgtcttttagaggtcttgtagggcacatgtatatttaccagatgtctgtgagcgtgcagcctcatggcacgttatgcatacctgacacttgcacagattcctggaagatgaggagcaaatacagtgcaacagacgttgtcaggccacgtctgcatatatagatatatacacagcaagaatagttacagcagcttacaatgacaaaatgcttctcagtgtgtatgtgtgtgtacctctgtctcaccagattctcacactgccttagcttgggtttccccaaaagcagagcctgagacaaaggcaggcatgcaggaagtttatttaggcagtggtcccagagcgcagccatgccgaacaggcgcgggaggcaggggcgccgcagggtggttcrcacacgtggactcaggcggccaccgccgcgctggctggtaagaagccccacaggatctcccaaggagccctgggacagtgtctcagaacatccacctggggcaagaatggggactgctgtccccagggggcaggtggagcctagtgggcattcatgacccaggttttggagctgtgcttgcgagagtgccgaggaggctctcatgggtgtcccgaggcagcttggagccaacgtccctaggcatggcctgggggtttgtgggaaggcctgaggcaaggcctgtctctgagatgtcctgaagagcaagttgggcccagagggttaattccgagcagcacaagagggtgaattctgagcagcaccagagggtttccctgacacagcaggggatgctttgaggcccctttaatgaaggagaaaaatgaggcttagagaaagtcagtgcccaccccaagtctcatgggccccaggctgtgggcagtggctaaagacaggctagtgggtaactcggggccacgtggaaggggagcttgtatttatagcccccagtcagcagcgctggagaggagaggagaggaaaagcagtgctctgagaaagacaatatttctagtagattggggcagggcaggcctggagacaggaaaccaaagccagggttgtcatgcaggagtgagatgaggttgcagcagcagagcgaatgcggagacgctcggcaggtccttggtggcctctgagttattctgcagacttctgccattcgtctattttttgggatactttgttaaattctcagcttagaagatagtagtgatgttttccctacagagatagaagaaaatataaaactattttctttttaaaactgtactgaatgtagggccgggcatagtggctcactcctgtaatcccaatactttagggggccaaggtaggaggatcacttgaggtcagcagttcaagaccagcctaggcaacatggcgagactccatctctacagaaaatttttttaaaaattagctggacatggtggctcatgcctgtggtcctagctactcaggaggctaaggtgggaggattgcttgagcccaggaggttgaggctgcagtgagccgtgatcgtgccactgcactccagcctgggtgacagaatgagactcaatctcaaaaaaaaaaaaaaaaaagtactgaatgataaatgattacaaatagaagcagttaaaatttagctctaggaatgagattgatgcatttaggctacaatataccaggaacttcctttttaaatgaaactagagcgttcttgcctttctgaatttaaggcacactgaaagaaaaaataataataatgtaacaaaatgtctcagtgtttttctatgccaaatagaatcttatgtatatctgtctagagacatatatgcatacatttgtctacacatgtgaggtaggggtgtgtgtgtgtgtgtgtgtatctgtgtgtgtgtgtatgtatgtgtgtgtgtttcagttctctaagaaacagacattccaaaacttgtgtgtgtgtgtttcagttctctaagaaatagacattccaaagcttggtgggcaatggcgggaggctttagaccctgtaatattgtcggagtgtcactgtaagagggacgctagcgcctgggtcacagtgctagctggtagcagagtactaacttaaaccctggtctcccaacaccccatccagcactctcatcgctgtactgatatgcctattcttatcttaaaaaaaaaaaagtgctgtctgaggagcattaacattcttactttttcatttttgaaatgaagtataaagatactgatggccttttacgtctcctctctgccctgtttttgctgtctctttctgtgttacatgggtttgccaaaatactgggtggagctctgtggaggaggtagcatgatcatctctgaagtgggcagtttttttctttttccaataaactgaatttacttggtcacaatgactatcctaaatggctagaaaagggaaaaggctagcgaaacttagatgattttctaaatttagataattttctagaagacattttcaaggcaaactagtttttgctgtcctttataaggccggcaggaagcgtgtgttgtttctgttttaaaaagggagaggagcggacttgggaatgctgatgggaatgcttgagaaatctcacagcagggctgtgcgtgccctgccgggtcccactgcctctggacagaaacccccgcaactccacccccagccaagactttctgcttctttatctcctctttctgctagcacccaaaaagttgaaagaattccaatggatagaatttttgagataatattggaagatgctcaaaatacacaggattaatttacacgaagactcagcgggaacacaagccatcttctgtacatgaagatgcactactgacccgccgtccgcaaatgtgtttgtacagttactttctcagtatgggtgatggctctccaacgaactgctcctcgtctcctgcctggacacccttctctctgtgctttcctgggttagagtaaatggatgcaaacacacatttccgtgctctgcagcaacttgagactcctgtgagcaaaacgcactgacgggcaatgtgcgtgggtcgtggggagcatccagctcccatctgcggaataaacccgcccaaaccatagggaaaagcgctgtcgtataaggccaggggattttcagaaaagaaatgtgttctttcctctttgatttttgtgttcataaagctgtaggtgcagctttttttaatgtaatgatttcataaccgctgaagttcgtgcttttctgaactatttaggaagataatctaccccttgtattggatgagatgatctgtcccttcgacctctggttcagttcccattctcccaagtatttaaagctgcgagttttttcatattttcatatttatttacataatttaaaccccctgtgtgcatggactttaaggagctgtacatctgcctgggctttgcagaagctgaaagggcgcaatctttttataactcacattagaaacacagattatttaacggggctatgttttgcaccttaatctttaaagttgcaatatattttaagcattttaaccttgttttagatctgatcagcagtagaatgttttcagataagaaacaatggagcaaaagcaaaacaatattcaatacctagatgatgtggcaagacagagaatagtataactttttgttttccaaatataacttttatcttcatctcttgatctgaaatttggtaggaagtgtaacaagtacgaatcaacatatttaccatttgccatttcaaatgttgatagtgaagctgggacctctgtttattatggaagaccatagaaaaccccataaacacgttctacttctgtctgtggccagcagtccagcaaaaatgttctaaaagcacatgcactgtgttccgtgatgattatagtttgactgtgctggaaagagagactgtgaactgcacatggtgattatgactttgggcaaatcactgaacttgtaattatgtcttccaagacctcctaacccaaaataagagagtattttactacaaaatatatgttacgtcaaactgttttttacaaaataccagctctagggatgtttccaagtcattttcggagagagtttgtcgaagtttttttcagggtgtgtcattcatgtattggagggggagagggttgagtaagaaccgacatgcacaacttggccatgaaatgaagcgcaagcacatattttatttctataggattcctcattctaaagtaatttttacagaaaatggcactctaagaaggaattcattaagataaagacacagatacagcatttagagttacactttgccataaaagagcctcccttacctcctgacttgaatctataacatctgctgaactgtcgacatcaggaagactcgaaatatrttttgaggccaattatgtcatttcagattgaacctgctaacatcagattctttggtcagtagtctcactagttttgttctcacaatggaattattattttgatttttaaatgttgctccatggagactggtatgatgagctcatgctctgcagttccattttaacaaataacataatagatcgctgtcaaatgaatgccatcacagacatcatgttgggtcacagaagccacaccctaggagtgcttactagatgatccatttccatgaagctcaagaatggaccgaacttactaaaggtgatagaagtcagaataatgtcttccgggtgggaggggtttgaggctgtgaactgagcagtggcacgagggagccttctggagtgctgaaaatgtttcctagatcttgacctcggtgctggttacatgagtgaatccatatttttaaaagttatcaagctgtaaatttcaggttagtatactttatccatttcctgtgttttatatctcaaaaattcttttaaaaactaaaagacatttagaaatgaaatgtttgacaagttttgttgtgacactatgaccctagttactatgggtggttttatttgtctctgcagttttcatctgggagcacctaaatcatgcctaatgaaatgaactttggaaaagtaattttaaagtaactatcttagagaactgtggattaaaccattccagccatctgatgagggttaaaatgtatattcgtaatctgacattccaaaacacgattctttccggatcaagcataaaaggcatttgctcttggaagaccaagaaagaattcatgtggttcccattagtctaaaaataaataataaataaataaatgtctgagtcatgtattggattttgttggatttcagtggcttcaagtataggaagaaaatgatttgtgctattaataatagttctacccattgcccattggaataaatacaagattactctgagaaaagtgaaatcgattgaatttagttctgctttgagctactgcaatgcaagtgtttctgacttttgagacatagtataaaaaactgaataaaataactttgttcatattgaattgagttggggaagtagcgatatttgtgacattggaagccattgtcattacagattcatcattacagtacagatttgagaatcaaacacacccggtgtgagtcccagttcagttccttaggaactactggctcaatactttctaacatcacatttctggttggtaaaacaggtatataaatacctactgtgctatgctagtgtgaaaattaagtggaatgagttagcacaattcccaaactgtgtgccaaggcaccctgggtccttgcagcaaacaaattggagtaagggagacagtctgaatattcaagggcaacccagcagtgttcaatgactgtcagccactggaagaattcatagctcaaagcagctcaccgtttcaacagtatcagcttgtacctctgtaaagctaggtttttggtggttgctgtttgtgaaaagcaagtgctacaggaaaattagcatagagcgtaaaatgcaggtggcagtgtccaatttgattccaaagtttgagaagccaggtgtgcctaactggcaaatatattccattcatacgtcactggggttacttaagaaagaaaataaaggatcttttttttaaaactcaatttatatgtatattggtattttcaaatagctactaaattgtcagaacataaatacttaaattatttggagctaaccgcttaatacaagcaactgttggcctagagataaatagagaaaaaatagtgaatcactaagggtcccatgagctgagaaagtttgagaacaactagcctagaaccttgcacttggtaggatataaactcaacgtaccctcttcctccccttcccccaatccaggtattgcctttaattgtaatctctatgatttgatatgtttatcggaaagcgagtaagtcaaaaagaactaataaattgtgtaagaccttcattaagatgtacccttccgtgttttcctaacttctgaaatcactaggaaaaacagccatgttccttgcaagctggctggttagtcctgtcttctttcaggtgaacagcatttataaccacggtgtacctcggaagaagcgttctcagagcaacatgcacgtgttccgtgtgtaccgtggttgccttcgggctaatgcgtttttggaagtgtagattggtgccagttttacaaaactcatgtggcctatttctgcttgtaatttatagtttgcctcctcaccatcctcacttgctctaaggtgaactagttttataccattagattatacagaaaagccaaatttacttgcatgccacagcaattcgggaagtaaactttcagtgtgattctccaaatgcttgtggtaaaagtacagagacttgaatcattttcccataattagtttcagttctggaggcccgccccctctctttaaccctcttgccttgcatatgtgtctcttgcaatggaagctagtggaaatttcctctcccctttcactccactgccataagttataaaaagcatgccattcagaactgacgttttcttctgcatgcttgaatttttactcaacaactggaaggggaagaagttatttccagagatgtttctctgtttatcaaaggggcgcagagtcacagtagcactttggaccaccgtagaatggctgactcacttgcctcatcagtagaggggcacatgttcctatcaaacagtgagtgcctttgagtgacggtgtgtgacacacagcacagcagcaccatttctcagagctgcagcaacactggttcacacaagtcactagagattcccatctccactgactcactcggtgggaacaaaagctcccatgccggtgggaatgcggggccaggggagcaccggaagaagggagccgtggcagaggttttcctattgttaggtttgtttgtttgtttcagtgagtcatcttacccccatttttttttttttttttaaccaaaactcactgtggttacttctctagtttgggttatgactcctacgagccagtttaattttatcagtggcagtgaattcttgaacgcttccctcagttgtagaaatttagtttcacatttaagtggtccaagtgccagcttaaactttgtggtttagtgtctttactgaatcccccctagtggaagaacctacattggagttttggctgctctttgggattcaaattatgagtagttggttccactggaatcttggccttctcctggagtggcttgaggcggcacactttgactttagaaggccaaagttagaacctacgtgaaggctttgtccaaaatgcctttcctgcaccctggcattttcaggtggtgtgtgtagacctgacaggactctactcgtgcgtcacttcccagctgttggtcccctgccacttagatgcctttcatgggcaagtccatgctagcctaggaaatttcccgaaaatggcagtgataactcagaattaggattttgatcctcatctcaaccattatccccagtggtcgctagctctcctctgcccagctcgaggaaaatgctgggtttttcattgcagcttacttgcatttcagtggtcccagcaagcactcaagaggaaagatcagaccagccaaacttgcaaggcaggctgatgagccaaggtacagaaagtacacctgatggatttttgtaatgatggccgtaagtcatagaaggtgaagcttaatgcaatttagaaagatttgaaaaaaggaagaaaagtcctcgtgctcacagaagcaagctcccattggcaaaattattgtgtataacaagcatctctctctgatgatgctggaaaaaaagaaggtgctatcagggagggagggaagatttgaaagaccagagtgagcagcagataggcccttgggtccttccttttattcccaccctcttttccattggctgttgataaagtttcatcactttttagctgcctgttgtatttcactacctccttgttaacctctggttataacctggggggaattatgtttaaccagtacttaatgaatcaatctattcctcaaaagttggttctgggcatcacggaataaacaccaagaccactcagcgtattttctgaagagtggatttcattagaaggcaggttagtcttggaactcagtcaaaacagctttcagtcagtccaactccacagatttcagagatagcagtacaagaaaaatgatacatgggtttgtacagttgagtgttgaagtgttgacttccataaaagaacacaaatgtcaatctacagcagtaccatgggtacgtaaattgtgttatacagcgaaacactgcacggtgatggaaaagaagaaactttaacatgcacaacaacatggatccatcacagaaataataaaagagaccaaaaaggagtatgtactgattgttccaattacatgacattcaaaacctagcaaaattaacccatggtgggtgacagagcagagtcaggattggcgttgagaagagggggatattgacgaggaggggcctgaggaagccacgtgcagggtgggaagccttctgtatcttgagctgggcagtcattacacaggtgcgcacatatatggatgaggaggggcgtgagggagccgcgtgcagggcgggaagccttctgcatcttgagctgggcggtcgttacacaggtgcgcacagatacagacaaagaggggcgtgagggagctgcctgcaggggaagaagccttccgtatcgagctgggcggtcattacacaggtgcgcacagatatggatgaggaggggcgtgagggagccacgtgcagggcagcaagtcctctatatcttgagctgggtggtcattgcacaggtgcgcacagatacaaaaatttaccaagttgtacactcaagatttgtgaattttattctgtgtaagttatatctaaaaaaagaaaagaaaagaaaaagctagattccctaaaacagagacagcagggctcgagtctgagctagctatagccatgccagcagctagatccatgaaaaggttggggttggctttgcccaggtgatcattcggggacgggggacgtgctgtgaatggaagatgtgcctgctgtcagcactgatgttgcccaccctttatttctacaacgctgtcttcaaaagaattacatttcaattttataccaactatcgtgcctcctcatgaatcccttccccgcacaacctggaaaccctcgcctggcgtcggctccatctccagatgttactcactggctaccgctaggtggctgcgaagggtggcggcgtcactgatgcgcactcaggcagcagccatggggaggttgaatccccggggcatctgcctctccctatgtgtgtgggtcctgggagtgaggcagtgtggcgtggggctgttgcacacacccccgactgtagggctgcacccagacacgtgcggtgaccccgtctctacagccgcttgttgccctggcaccaagccaaccactcagcatccagcgcgtcctcaccctccctccggggtgaagcggaaacaagggtatgtgccaaaactggcctgctcaccatttcccagattttccacatttgttcccactcggggtgaggggtgtgcttctggtgtgacagctgtgggctgtgtagggtggcgggcgttggtggtgaagtctgtcggccctcctgacccacacacgagggggtgtggattttatattgaaatctttttaaaatctgtttttttgtaagaggctctgaaaggaagaaattttatcagagttttgcggcctgtgtacgttctgatacctctcagagctggagtttcttacccatataggacaagctgttgtgaaattgagtgagacgatgtaagcacatggcgtgcacctgataaatgccagctgccaccacagtgatggtcagcagcgtggtcaccactgtcgtttcacaattacagcccaaggagcccaaggggaaggagtgcctctctctgttttgaccttctctgactgctgtcctaataaacagtgtcctttctacaagaaccctgtagacttttgaaaccaacaagtgaaggcactccaaggcccttgttttgagaaggggtaagtgtgctaggtaagggatttccttgggtgcttaccttccacggctcctgggcccctgactcgaagctgaccatctgtgctgatgctgacttaggattttaaatcacttaaatttgagctggatagagaaagggtcctagttaagctgagagggctgcttattcgtgattttttttttcttctttctcatgcagagactgtttattttagtggtagcggtatttaggggtgaagaaggggaaaggaagaatagtgtgccatcaattaattctatgcatgtcagctgcaacgccttcatggcacgggacaggccaattatgtaactgtaaacaaattatatgtattaaaagttgtccaattaaaggaaaaaacatgcatggatttatgtgtttgttattacccagaagggagccatgctgtacttgaaaatatgcaaaatttcacatcacaaaatcaccagttgttgtttgaggggctggtgttctgattagtcttaattttttttaactcataacatttttgtcccagtcatcaacactgttaagaacatgtcactggtgcagttaagttaaaaatgattcaggtcaggaattcctgtcattaacaattttttatattaaagttggaaaagtttaaggaaatttaagaacctattccttaatagttaaaaatagtaaggaatttcatatacccccaaatattaagcataggtaattagcttgtggttgggatttgatggttttctgtttttcagcaaaatacaataacgtactttctcgagcagaatttttacaccaacatttcccattaagaccagtttgtttagggaatttttaagctacatctgtatgtaataattttttgagattccaaagactacgcagtctaataaaactctaatacttcaactatcttcagactaatgtttataattacccggtagatgaccaagaattgatatcatctgttgattccagaaattatggcagagaaaatgctgtcaggaacccaaagaaaatcagaggaaatggtacctctaagaaattctgaatcttttctactaagatatgtggcttgactgcttaaccccaaaatgcctgcttagaaggtagtttggggctatcttgtaatactcatttagttcctgccttcttctgccatagaaacaacatgcagaagcagcattgcttacgactcacactgaacctgaagggatgaaattacatatgacgatggaatgtggccatattcacgcagtcacagcagtgtgttgcccaatgacagtactggagcagtttccacagaggcactcatgcaatatgcagaatacagacattttacacacacacttacgatggtccttttcattgtcgaaaaggaattcattatctttcgagtaaacatgtgctttgaggtatataactctgaggtatagaagttagaacatttaacccgattagggtgactggaattataacctttaactaatgtgagatatagtatagatcttgataagtgtctttctggtgttcctattaaaattcattataattaccgttcctgcaattgtgtagcatcttacagtttccaacaccctgtgctagccatcatcttatttgaaacacataataaccctacaagttcactgatgtaggtaagaaaactggtaccgtttctgaagatacacagtgattgtttcggccagttaattaaggcaagagatcactcaacaattgttctacagttattcctgcttttttttttttaactcactcattaagtgaaagaagccagtctgaaaaggctgtatattttatgattccaactgtatgacattctggaaaagggaaaactgaagatagtaaaaggatcagggtttgccaggggttaaggggaagaagggctgtgcaggtagagcacagaggattcttagggcagtgaagctgctctgtgtgatgctacaatggtggatccatggcttcatacattggtccaaacccacagaatgtacagcaccagatgtcaactgcgggctctgggtgataatgatgggtcaatgtagattcatcagttgtaaccagtgcaccactctggtgcaggatgttgatcgtaggggaggtggctgtgtgtgtgccacggaggggggatatgggaactctctgcactttactctcgatgtggctgtgaacctaaaactgctctaaaaaacatagtcttttaaaaaatcatttactacatatgaaaaggaacaagtaaagcaacaacaacaaaatgttattgtgtactttcagattgcaccagtaaacctagccagccctcactagggtcttctgatggttacatagttaaaagtacactagcacaccgggagaataacttcagaggcttgctggtctaatggtaattgcgtcggcttcacacgtcaacatttttttaaaaattagattttcttgaatctgatcatgtccaagatacctcttattttggtatagaacgcctttattcaaacaacgggagaacatgaacatatccctttgccatagtttggctaaattcctgaggctggctggggccagaaacaaaatccctgaaatggtctcaaaatttttttttttttttttacctctccccttttccttctggttggtggtctttggggcctacgaggccctcaggcagaggggaaatggcagtttccccatccccttttgggacttcttgagcagaaaagcgaatgtcagacggtccttataaagtcccacgtgattcagccactgaggatggcactggctgtggatttacatgtaagacaacttcatggcgtattttcgccttttgctgttgaatataactaccaagatatggtttgggcagacaaaatagaaatcttctgtgtgtagcatgtccagttggatactgttagtgacatagagagacgagcgcacaactcaggtttaaccttcatccctgaaatttgccggaacagtcataatgaaggtgctaatgtatttcctgaaatactgagtacttcagacagggagatatgggtggtatctagtagccttgtgataagacccatattagactaatagtagtcttatcaccagattaaaccacctggatagcccacctcaagtcatcaagagtgttaacatgggagtaagtgtgacaaatgcccaggtggtctggactaaatgtgacaaaattgagaaatagaccctacaagatctggattttaaaaagagagaaaaaaaaaaatggaaaggctggctgcttgcttccttttaagactttgttcacgttctcgcccccaaaagccaattatgattataatttatcagcccacaggaaatgattgcttctctatgagacatcgtcaacatgataaaataatccatttcccaagatttctatatcttagtatctcatctctttaaaaagctccattgtccataaaaaattataaaattacatatttttacatgacaggtaatttttaatgtatatttttaatttggttgttggtttttaaaatagtaaaatattaaatatcaactatgaatattttgtggtggtaagttgtcaggttaatgtaaagattccaaaaataattcacagacatgtggaaagttgctcagagggagaaccagtctgattttggagaaagtaattaccatcagagcagccctcggagggagcgggagagtccacaggtttcaatcaggttctagatgaattgcaaagagaaaggttttagctggttgcaggaggggctctggtaaaaggattaagtccagttctcaggagttttttaataggtttcacatcttttgtcaactggtgcaaggaaggattaggacagaaaagaaaggtgatttcatggagaaatatctaattaaaatattaaagatagtcggatggcacacctgacctagagtccaggcagtggtaggcagagttccttccccttttttttaaaccacacataaaacagtcattttaattccaacaaatggttcatactggtattctaaaccactactcatgattttttttactctttttatttacatcaaatcattcaacttcacatcattttctttttaagcattaacataatccaagtgccaggccatttttggtgatccaatctgtagaatgtgagatggacaataacaatcaaaccgttttcaaactctaatagtgggaagagaaggccacatggaacttccctgaggctgaatttcgtcgtcctgcctttcaagtggtgtcctgtgaaatccagcgtttccccctgtcaacttccagaacagggctgtaactagatgtatggtttgtaagaatatcccatgtatacttcctcttggttataacataatttgttttgcggggggtggtttgcccttttttttttttggagacaggatctcactgcgtagcccaggctggagtaccatggtgccatcctggctcactgcagcctgtgcctcctgggttcaaatgatccacccacctcatcctcctgagtagctgagactacaggcatgtaccaccacgcctgggtgatttttatattttttggtagagacggggtttcatcgtgttggccaggctggttttgaactcctgagctcaagcgacccacccgcttcggcctccgaaagtgctgcgattacaggcatgagccactgcacccagccacataaatttgtttttagtcttctgaacgattaaatagttgtaccaattataccaattgcaccaattctattacaaggtggaatttcttatcgttcctttacaaacaggatattcccagttgcttgtttttgcttgttttcctagcagcttcagcaccatcctcacatagaagggctggcatctcacctatctagaggtgagaacaaagctgtgctctcagcaatcggaatctgtcaagtctgctgtggggacttggtatctcaggcctgatgctggcctaggagtgccctgcactcgtctcaagatcgatgtcccagtgggcgagaattgctgccaagactaaccaagggtgtcaaccagtgacttaacttctcaggctcacttttttttttatttttaataaaaacaaattgttaaagaggtaatttaaaatatgtactatataataagtactacagcatatacagtgtttacatacatataggcattaaatattaagaatgtttatttcagaatcatataattatacctgatatttactttttgtcattctttgtatattctccattttttgcagtatctatatattacttgggtaataggaatagcaaccattgagaaatagttctaattgattttccttttataaaagggtttccgtgtagtgaccaaggacttaacatcatccccaccccacagtccctcacacgcctgactccctttgtgctgtgtttaatttctcatttcattcatttacccttctgtgcagcacatactagctgctgctacactacacgttctgaccaaagcatagtgtccccctggggcaagactcttgggaattgtctttttttatttttttttcattttttagggtctcactttgttgcccaggctggagcgcaatggcaccatcatagttcactgcagccttgacctcttgggctcaggcaatcctcccacctcagcctcccaagtagctgggaccacagctgcgtgccattgtagagatgggggtctcactctgttgaccaggctggtcttgaactcctggcctcaaagtgtcttcccatcttggcctctcaaagctctaggattacaggtgtgaggcactgtgcctggctttaggcattttcttccccctctgacttcttctaggcacctagaaccaacactgcctggacatgtgaaggcactcgataaatattttttgaacaaataaatcaacttgcatggctcctgccccaaactggaaaccccacctaggaggggtgggcggggtcatatggtgttcactcacttacgctaatgaactgagaaataacgcacttctgcccaaattcatgttcattcacactcctctcagcagttttctgcagtcttcccagccccacggaaaattctgcttttgtcagaggaggggatatgcgtgctttcccgtgtttgctttaccgctgggcaatccatacaaggctactaaactgcagagggtactggtgttagcatgccccgtgtttataagggacttaaaaaaatatacaggcttgcatccaccatacctaccatacttgtgtactagagatattctcggggcaaaatgaggtgaggtgtggaaagtgctttaaggtgactcagagccaccctgttgcgattgctgccttcgtgatgactggtgtggctgcaaagttcagtggctgtctttatatcagaataattctagaataatttaggagaaaattctcattgttaggttccttcaagccaaaggaggatgtagtgaaaagagaataggtgttggctgtctagatgggccctgtttaattagagtcgactgtatcagttgccaaatgaagccaatcttacagggccatcctatagaacaaatatatattttttatatttaatatgatatatatgtgtgtgtacacacacacacacacacacacacacacacatacatatatacagggagagatagaatggtttgcctgctgacttgccattaagtaccgtaaacatcctggaaattgtgaacagctaattggaaaacagtctgtccgtgttcatgattcattgtatgcatcctctagatctcaactcaggaaatccacaaagctgaccaggccctgctgtcattttgtggccagatatggaaagatataaaccacctcctttcttccctgtcaaaacagttgtgccacgtcctccccctcttcctcatcttgactgactccctcacaggtggtgtctctgtctctcctgcccctgcccccacacgcacctttagttacctcagtctttcaaattttgctctttgttcctaagtacagtcttccttccaacctctcgtcatgtcatttttgggccaggaaagatcctgattatgctataatgccactgtacgtgttttaaaaagaaggaacgctgtacatttgatattaaatttggcattttaaataaagggctggtaaaaaaatctctgagtgctaatctccaagaaagggatggaagactggggaaagagaatctacttcctatttccaccattttaatagcctgacatatttttttaccttgcccatatcttactttcataacatttttgttttattttttaaattactcccatggcggtagagttgatttgaactcttgtttttcaattttaaatgtacaaaatttcaattattttatggattaaaataagcaccccagaccatcctgagcatctgatcaccaatggtaagaccattatccttctcaagtttcatctacggtaactggcttacagataaacttgtggattacaacctgtttgacaactgtaaagagccacattgattaaaatcagaagattttcagagttcagtatttagactatatggattatctagtgtctcaatagaaggtaaggttatggaaatccatttcctagttctaaactctgcaagcaaacaatcatctccccatagtgtgatatctaaatagttaatccagtatgtcagacaaccccatttagtaaacaaagactacttgaccatagaaaacatatgatatatgtataatatataatccatatagagtaaacatgtattatattttatatactgtataggcacatatcatactatacatatacatatcgcataagagatacagtaaactatatttgtattttccaaaattaaatatgttgcagttcccctaccatagtgaaactgtctcttctacattccttactgcattccttactatatagtaatactaacactgagcacaatcatatttcaccactcaggatgtagccagcggatacagtaatggttcttgtcctccgcaggaggaccacgggagaccagtggctgtgaatgggatgggatttttttctttcctctaatgaaccaagccctgggttttattgttgttgttttaatatacagctattgagtgttttgtagccacacacgacaacacacacacacacacacacacacacacacacacacacagagtccctagcaagggcagggtggggctagcgggctgggttcccctgggagcccctcaccatccgtttctcccagtgacggcagctatgtttgaagagcataactgcatggtttcctatgcattcattcgtgagtagtagctctcatatattattaaaaagatacactattattacttttaaagaaagaaaaggattgcaattcacatttacactttccagcctgttcttgtgttgtttaaaaaacaaacaaacaaaaaacgatggcagaggaaatgtttgcctccgtagtaggcatcaactttatttttcaaatcattctgttttaacgtgttcatagactgcagttgtttataggtatgaggcactcatcagtgtgaaatagttctttcctttccatatttcctcttatcagaaaaaaaaattcctgtggtctcctagcaaaatacaatccattttgctaaattatttgtgagtttttataaagtgtgtttaatatcaccagggcagaggttcacactagttgcaggattagcaagagagacgtagcatgagtagtgtttggtccactgcagtgtgttttgtgtgctagcgatcatgagtttatctgatccttgtttaactactacacagtgagtaagctgtcctgtattgttccattcatattcctctgagttcattcagaagcctgacacttcctttgccggacagattaaaggggcagcgtgggaccttttgatgatgtgaaacctgctttcttagtctaagctccctaggctatgctgaccactcagaggttgaactactatttatttgccctaaaatgaaccagaaacttggtcttagtttccttcctgacacatgttttaatttcctaaaagtgtacggattttgtagtgggttgtttttgaatctttcatttttagtgctgatccaggagagaaaggagatatggaaacatttttttcaaaaaatagctcaaaagaaaatatgtaaaaccatgaaaaacccagaattgtgctgctgctttctgtgctaattaaatcagtgggtgttaggttgtaatgataaccctttaactgtgtggcttatctctcattccattttatattattttcttcctcatgagaaaatcagtgtttattatcacaggtgacaaaacacaggagaaaaacaaacagtgaggttacatttaatcactttaagtgggtttcatctttgcttttttgttttcattcccaagccagaagccgtaaaccgagcgagagtgcaaattgcctttctcaggtgcacgttgctgagataggctgggagaacaggtgtggagcccgtgaaaagataaacattaagtcattcttggggaaacggtatttagctagacagctgaagacggacttttgaaataccattgtgctactgctgttcaaatattgactaagtgaacctggaaaggaagaaattttggtcgcctaacatagaactcgttgtctttttctgtctttaaatgttatctcaaagacccaagagaaggggtagtttacctaagaaagaaatatgagctttgcttatggagtttcaggtatacctaatgtaagttaattaagcaaatacaatgtagcagccttgcatttggcctagcattcttttatgtttcctggctgtttcttcgaggagatgacctgcctgtcgggcagattagaatatttactgcagtgcatctttcatgcctcgctgtgactctgtaaccacggtggatgtgggaaagccattaaccatcagcttgacggtttacaaagaaataggaagttcaagttaagcagatatttaggatatagtttgccttccacatatttcaacctgtgttgctgcatactttttaagcttagcgtaattattcacacagctatgaattttagaagatgtttaaaagcaaaccacagtgacctgggaaaggagggaaacttactggagcgcttagccaggagcttaaaaagacattgctagtgagttttatgtcacatgaaatctacatttgataggtcattttggtaagtttttgttgttttaaatgactcctcttgacacagtaaccagtggtgctgggaacattcattcacattcattcatttaagctcatgactcaaataataacttagtcgtttcctctctgaaggtaggggaggtaatgaggagcaccgattaggctccaagatccgttctgagattcagataaggtgtcctaacaaaaggtttatggtgaaatgaaagagtgagaaaataattgtgctttttctagggtcatgcgtcaaatgaggctcaccaacttttaaaagactttacatagctttagataatcacattccctgccatgtaagcattgtgatgtaatggcatcatcatgctacttaacaattaatttatgcattttgtttaaacttcctttagaatatatatagtccatataaagaaaattccagggtcgttttggattttgtataaatagctcccatgtttacatgtgaaaaaaaattatttatgaaagaaaaacagagctttcaatatcctattttggttacgtctccataaaaactctaggaaacagtgggatcatctgtgaaacagtggaatcaccccaagaacaaactgtcagacagaccgtcctgtcgtggcatgacttgaacataaccgtcccacgtggggacgcattccgcaccggttgctggaactgacgggggctgcagtgctgaatacctctgggacgcttgggaactgtgcccctgtttacagacggcaagcccttagtggtagggccctgagattctgagaaacataaggtctgctttatttaatttcctctcgtttaccaagagtcacaacctattttagtaaataaattcaggaaattggtaaagcactttactccatccgttatgcctcggtcatcagcatggttgtcacggtctctctggctcacgggctgctgcggctcacagccttccctcacttgcctgcaaccagctgagagcctccctggtgatgggtgttactgagcttaaacgatgtaaacaaacagaacggcacacaagttgtgcagggaagtatatttcctctaccttgttaataaagatttctaactttagagattttctgtattgactctggcattctttccaaataattattttcaccccggggactacccacacaccctgggatgaataaaagaaattatctttcatttgagggtaccagcaacccgctctccagctctaatcctcttcatcctccttctttttttatttttttttttttttttttttttttggttgttaaaacctgagctgctgccaagctgatcttaatagcatgttcacaaagacagatggatttttttcctaccttcattagccactgagtgttgttttccatgatgttctccagcacttgcagcctctgcaccgagtcatcgtattcgagcggcgcgtccctctgcacagcattggacacgtaggggctggaggaagagcggcagttgtccatctctggcaggaggaaagtgtagctgcaggacccatgctggacctgatattgcttctttcctatgctgtccatgctcttccgaaagttgttataggctgcggccaagacaagatcacagctcagagtaaagaaaacaatctgccacattctttcttcagtaataaaccagcagcttagcaaasttgagggcaaacacacgtccagagtcccgagctgctgccgtctraaaygcagggctgctacgctgccatggctgggtccgtcartgaaagtcttctctttcctctttttccagtagcaaacctggtttttactgctgtgttctctccaggcatgcagtaaactgtcagattgcagtgggaagaacagtcctgctcacttgggagggctgtgtcagcttttacagagcagctttcacggtcctttgttcctctctccccagatcctacagtgtcagtatccgaatcaatcactttcctttccttatatgataagttgataagagcagccagacatgtgtagtgggctgctccgccctcctggcttagccgttatcttcctgtagggggtcactagccaggcaacaggaaaaatcagagcagaatgcctgccctccaaccaggacccatgctgcagaaaccctctgggaaaaaccgatctgttacaggacccctgggcatttcctaggcaccaccccaattaagtatttcctagagagagcagttgatctcttttgtctgaaactgatttttgccgtgctaagctggcaaaatatctgaggtaataactttaatgttgaagtacaatgaaagttcctgttttttcctttaggaataaaaatactacaaataggtcaggacttcggtttatttttgttattacaaataaagaggaagaagtttggctcctgtaaacgtgtgccttttcagagggaaaaatagattcattgattttagttgattcttgaaccactagccaagttacaaaagattttcatttccgaacagttggatagaaagatctgttattaagtcacgttagaaacatcagtttctgagctctgacctttattctttaaaaaaactccacttggatattcactctaaaaatacactgtactgattaagttcattacattacaatagagaaattagaatttaagtgtctgtgtagaaagaggaatacaaactttttttttttttttttttttttttttgagacggagtctcgctctgtcgaccaggctagagtgcagtggggcaatgttggctcactgcaacctccgccttccgggttcaagtgattctcttgcctcaggctcctgagcagctgggattacaggcacacgccaccacacccggctaatttttgtatttttagtagagacagggtttcaccatgttaggctggtctcaaactcatgaccttgtgatcgactcgcctttggcctcccaaagtgctgagattacaggtgtgagtcaccatgcctggcccacaaacttctttattgtgtcagaatttgttgacatctcagcattttgtaacacattatcaattacattagtcccccttggtattagactcgggcaagtcacttccctgttttaattaagctctaatgttctcatctgtgcaattcaaggggtgcactcacaagatttttcaccttcaatcctatggctctgtaagttctacaagtcacttcctttaacaactaaaacttaatacttcagagattaataatatgttaactcagcagcccaagtgtacatagggaaaaagccccctgcctttgctgcggtttgtttatctctcaaggtacaaggtttattattcccagcgagcgctgaatagctggtacactgacttaacagaccacatctacccataaaagatctttattttttactaagctctaaccgaaagacagcctttcccttatcaatgaatagttaacgaacaacagtgtgaatatctgtgactttctcatcctcagaaatcagctctttttatttgctgccacaatactcagaactacatttttattaaacccagccctagatcttgctactgaacattggaataaagtagcatgtgtcttcttttgagaaggtgtttataggcttcaccagacaaccaaagggttctgtcacacagaaaagctggaagacatgctctggaaggatctcattagtagaagaggtagtatgattccaccaaggttctggacatggtttccactaagggaaccaattaaagatgctatacccatccggacagtgcaccgtcgaagaaagcatataggtcttaaagatgagacctgtgttagaaccctgcttctgtgtgacctccagcaaatgcttccattcttggagcctcagtctccctagtcataagatggagatcattttttctctgtagggtttttaggattaagatataattgtatgtttagaaatatttgttccttttctttacaggcatgctccattaaatggggatcagttcttccaccatcaaatagtataactctgctattctctgaatgcaaagcagtggcagtggcatagggtacaatttttttatttcctgtttgaaaaagcatattgtaaggtattaatatcacatatgtggttttaccttttttcaagattattttttgtagagacggggtctcactcttgttgcccaggctggtcttgaactcctggcatccaatgatcctcttgcttggcctcccaaagtgctgggatcacaggtgtgagccactgtacctggcctgcatatatggttttaaaagtcattcagttgtcttccaggcaaatagagtagtttaaaaggaacaaatagaaagagggcacaccacagtactttttgtctaccagccttgtgtcagacaccatgctatgcactggggatagagattaaggcaactgggtgtctgacctaaagaagctaatagtgtatggagagggagagacccataaacaaattggatgtggtaagagagagcagtcggagcacaaagaaagacagtgatgcttccgggtgcacaatatgccatgtgcagctggcatggactccatccttttcaaattccctcaagttccgaacatggaaggaacagctctttgtagattcctaaatggaaattatcggaaccagagagtcaggggaagctctagtagagccggagttggcaaactctgtccagtgtcagatggtaattattttcggctctgcaggctatacggccaccatcgccaccactcaaaggtaatgcggtgcaaagcagctgtgggacagcattaactgagtgagcgtgctgggctccaataaagctttattcgtgatactgaaatttgaatttcttgtaattttcacatcatgaaagattctattttctgcaatcatttaaaagtttaaaaaccattcttagctcataggctgttagaaaccgggcctggactgtagcttgctggcctctgagagcctaggtggtctgtgggggtaggaggtgctgggcaaggccgtccacagtgcacgcggtgtggtgaccgtgtgtggttggcaaggctgtcagcagtacacacagtgcagcaaccaccgtgtgtggtcagcaaggccatccacagtccacacagtgcaataaccgtgtgtggcgagcaaggctattgacactgtacgccgtacagcgaccgtgtgtggtcagcaaggccatggagagtgcacacagtacagtgaccacctgtggtcggcatggccatcaacagggcacacagcacggtgaccatgtgtgatcagcaaggccgtggatagtgcccgtggtgtggtatccatgtgtgatcggcaaggccatggatagtgcatgtggtgcggtgaccctgtgtgattggcaaggccatggatagtgaacgtggtgcggtgaccgtgtgtgatcggcaaggccatggatagtgcacgtgttgcggtgaccatgtgtgatcggcatgaccatcgacagtgcttatggtgtggtgaccgtgtgtgatccgcaaggccatggatagtgagtgcacacggtgcggtgaccatgtgtgatcagcaaggccatggatagtacacgcggtgcggtgaccatgtgtgatcggcaaggccatggatagtgcacgcggtgcggtgaccgtgtgtgatcggcaaggccatggatagtacacgcggtgcggtgacgatgtgtgatcggcaaggccatggatagtgcacgcggtgcggtgaccatgtgtgatcggcaaggccatggatagtgcacgcggtgtggtgaccgtgtgtgatcggcaaggccatagatagtgcacgcggtgcggtgaccgtgtgtgatcggcaaggccatggatagtgcccgtggcgtggtgtccatgtatgatcagcaaggccatggatagtgcacgtggtgcggtgaccgtgtgtgatcggtaaggccatgatagagcatgcagtgcggtgaccgtgtgtgatcggtaaggccatgatagagcatgcagtgtggtgaccgtgtgtgatccgcaaggtcatggatagtgtacacggtgcgatgaccatgtgtgatccgcaaggtcatggatagtgcacacggtgcggtgaccatgtttgatccgcaaggccatggatagtgcacacggtgcagtgaccaccgtgtgaacaggggaggactggtgcctcggctcagccttctgtgtggctgcttacaggggcttactaacgggatagaataggtgcttagagaaagtgccacactgaagtgaattaaggatgccaggtggggagaggggccaggaagtggcctgggatgcaagtgtgcatggatgggcgctcagctgtggcccctagggaagtggagacatggtctgccaggccactgaccaggaagcctcggggagccaatgggaggcgcttgaaggcattaggtgcaaagcctggagttgtgggtgtccagtaacaccaccatgcagcctgggggtctgaggccacccatctgggaccccttcactctaaatgaggcttgactagggggatctcagaagtccacagaaaatcttgggtgttcctccctgctgtactgacgggaccacaagaggcaagtgagactgtcagatgagaaacattattacaggttcccaaaatccacctgcctacccacccaatttttgtctgtaatagttctgctgaacagctgtgcatagtgcaatttatttccttaatactgtttgttttctcccccatattctgtgtcggcaactgacatttcagaggttcccatgtgttctctgtggaactgtctcaagttcttattaccctggttgacgacaccagaaaaaccatagctacctactcccagaaagaggccagtgttacaaagaatctcgtggccagcccttttggctcagtttgcccagttggaggccctaaggcgcaaaccagaaaagccaaagggcctcctgaggaccgtggaagtgggtggcgcgtggacccatcgctagctgaatgtggaatgtggacccatcgctagctgaatgtggaatgtggacccatcgctagctgaatgtggaaaaggacttatgacagtcagaccatcccaggttcccccagagcaatccgtgcagctctcataagcaaccagaaaccaaaaaaggatgctaagtcagcacaaagtggagcagccccccagctatgggttgccaaacagaatttgcttgtgggccccgtgacccctgctgttgtccagtttaatgctcagcatttatccagatcaagggatggaaatggggccaccagcctgacccaggcccggggtcgttttgcttttccaacctgtaccatcccagcaatgcattgccagcgtgcaatttgaaaaagccctgccgagctgaaaaacacatgggaagggctcagacacacttaaaggcacattgctgccctgcatttatacggcattttgtgctgacatcgttttccatcaggcctgggcagcccctcctgagactgtctcccgcctgccgtcctcagcacggcctgcccggctacagtctgctttcctcccactgcccctgcctgcaggccttggaggcggtgactgctgcagacttatttgggcagcctggccttaatttttggaaagtgccttgttgatgtatgaggaacttccacggctgaaacagtctaaaaaaatgaagctgggacactatgttttgattttagccatttgcagacagaggggcacactcgggactcttgggcgcctggcacactaagctgggagggacttttgagacatcttggccatctaaatcagtcaacatgtttatatatacaatttaatgttcagtatacagggaaaaccattagaaggttagctgcacataaaactgttgttaaagttatttttattacttccccccacaaatcgtatgcaataattaataagaactagagaaatagccacaactggcacaacacctgcccctctgccaaaagaaaaaaatcttctttctgaaggcaggctccctatatagtgattcctttatatgcctcctggaagatctgtttcgactccattttgatatatgttgaaccagatttgaagacccacaaatgcagtctagagccattttgcaaaagtgttgctgcatcaaccatttccattccccagtgctgctcatcatgttacactagtgttaaatcctgactttggaatgcgaggaaggacagttccagccatgggatttcaaaaaagtaccaaaggaaagccccttcaagttaccgttaagacagaagaaaaggaagaaaaatataaacacacacgtataaacatgtaaggtagctttggtccctataacagacaaggaaatcaaggctccgtgaagagagagacaagaattcccttagccaagtgcctgtgtgtgtctgtcttttatgttaatggttatgaatttaaggagaattgaaagcaataattttgcccctctttaacatggcaaatacagcctgctttagagatgatcagcaatcaccatttagtactggccgtcacctctgtgcagcacaaacacacatcccgagtgacagaagccatttcactgccagagactcttagcggccttcagttctcttgagctggagccactgggtcttgtatgaaagctcaccagacatctcatgtggacctcgggcatctgagccgggaccatcctattacaagtgcggaaaccagatcattaatgcagagctgaattcaaattgttacttgctagcttaggaaagaatccttggaaatccaacatattgtctaaatggatcagttaatcttactatgtgcattctacataccctttcattgtttgggcttaaataacttttctgctttgtctggtttaatttcatccaatgtggatcgctggaagaatatgatgtatgttttagaatagaaacagttctgagatgaagttgagcacaatttcctgttctagttgcaattaaatataaatatagcatttgacataaaatagctggcccgatatatttagagtacaagttaagtgtcatccccttagaattgggcattgactccgtagaattcccctttgtacaaggtgagcaaatgtatattttgttaaaaataagtatctgactgccaaaacggacagaaagctctttgccatatgtgttttcaggccatttcctttcctgggaaacagccatttcccccgcattatagttgtgttttcatttgcgggtagatagagtaagcgcaggagttaaaggacgcgggcctccacagccaaggccttatctgggacaattatctttctccttgcagctgtgtaacttctgtttgacacagaaccacagaaaccctgttagtgggaaggatcacagttaataggagaaaaatcttcattgttcatgagacttctcaggtgcttggcattcttatttaggtggcttaaaaaagttccaagtactcattcattctaacttatctgtgttcattgtgaaatcgtgtgtgaatgacatttggagcagatggattgttgtttttttttttttttttttttaacaaacttaagagattcccgaatctttcacagtttgtactaccgcaaaccagcataacatctgctaaagaatttcatattttaaagctgcactgtacatcatatggaaccttaaggactttgaagggaagagctttttatttactggtagcttgggaaatatccaagtaactattttttaagaaaaaaaaattccttgagtttttagaaatagtttatataactgttatgctgtttgatttttaaatattttcattctctagtattattatggaatattttatcttcccatcaaaaaaatgccagaaggtcaagatagaagtcacaacattaaaagggagtggatacaattgtaaaacaatagatgagtacatttgcctgataatatttttgccagtaattctgtgtcctgttttctccctgtagaatgaaatgctaaacatttttttcaatggattgatgtcagtgtttactaacatgacctgtgttaagtcaaataaagtatttcctttgacaaacaccatatttcattagtggctttgaggtgggcttatttgttataagtcacattaaatgttcccaaatccatttcataaatgttgtcgagatctcaaactccgttgcttctaaaaaaatatgtccagtctctttgtcataaccatcctaataaagatctaaatttcttagagtgaattttcatttgaaagtggcttaatgccagctagattaattcttgtttaatctaaatttataaaatttttatcttaattattgagaaacctttttaaaaagagataaaaatgtcatatgtgctatttacattaagatatattatctctcttggttataggttaagataaataaaattgcttatgtcaaagaagtaaaaaaaagtccatgacctccttttggtatccccatccatctggcggacttaatatgaaaaaatcttcctgtgggaaattaggcttgattatagagttacaagtacaaaaagtagtttttgaagaattataataaatagttacacataaaaggaagtgatgtttgcttgaagtatataaaaatattccttgtcactcttgtcccctcatgaatcttagttgtctgatgatggttcaagtctttcctaataatccagaatgtatccctccactttttctcttaaaaacgctatttcaagcattttctttggtaccccattaataataaagcatacttccccaaaatgttccatttcaagtaaggggtctaaaagtcaaagaccgactgatacaaaagagaaaagtaaattgtacaaagactgaagagaggatgcagtattaaacgtaccaagttcttgacatcggtttccctcaagaaaaaaaaaaatgagtaacgttttttgaaagcctgaaactattctagtaaaatatttacggaaaaaataatatgcgctctcctcccaaatcctgatgcgcatttaaatcaccttttttatttatagatcaaaaatcttgcttgactacaataaaaattaaaaaatggtacctatttaagaatgcaagtatcaaatccacttgtaatactcactagctccctctgctgatctcctatcaagcgacaggcaaatctatccatgattgttattacaattgttaatggaaatgataggtaatttaggacctacatcaattgcaactaaaatacaagctacaatgctttcattttaattttaatgcaaaagcacatcacaccatatacagatgttaaagaccgacgtgcacacacacagtgaaaaaatatttttaggcattcatttagcatacatagacctaggagctgtctctgtatcctcaggtgataaggttactactattacaacagcagaaaaagaggtctgtactgtctgtctccataaggagccaatttagagacccaatcctgttcaccccaagcttacagtctaacgaggtgaacagatgtcccatctggatgcacaagcactgctggctaaggccctgggtagtgcaggagggagcccccacacgggaagcctcccaaaccacgtaagggctacgtgaacagcaagaatagtttcactgtttatttagatccacactgttacttatttaagaagaacatactctgccctttctccctccctgaagaaagaccaaaactgagggaaattatattccaggctgagaaaattgcctgtgcacttaaaaaataaataaataaaaggcgagaccacggaagttaaaataaattaacaataattgagccaagagggaggagatgggtgagtcggagatgcggtctggaactagctgctgaagagtctgcttaggaattggggttgtaccctggacataaagcatttggggcgggggagtgtcctgatgtgactgagaaaggactgtggagtgctgtgtgcagtagggcttagaggaggtgtgagtagaggcagagagaccagagcagaagctgctacagtaattcaggttagatattatagtggcctgtgctagaatattaacatcaggctcatgatgttaaagaggggtgatcaataggccttctagatggatggaatacagggagtccaggaatgggattggtctggtactgggactgactcttacacttaaaaatgctaaaataaaataacttggcaatagtttttaaagcatctgtaaaatgacaagataataaacacatattctgctcaaacttatgtgaaggcaggaatcctgtgaactattaaagagctttgccatcaagtcatttgccaaacctggccaacttaagcctacttcaaggcctgagtggttcagacagaagacaaaggccaggacctaaagaaatgggagcatctgatgagatacctccttccaggaaggctctaccccagtgtcagggaagcagaagtaaacctgcccaccccactctccagagcagacaagaaaacatgcctggatgttaaacagaactaaaagaggggagcccatccctgagaatttaactacaagctcacccttttgggttttacagtacacataaggtggccagaaaaaaccacaatgaattgttctaaggtggtcccaggctgatcatcttattcccctaggtttgtggaagaagcaaatgaaaatcctttctgggagaatgcactttcatcatgggtctcaaaacattcttacaatttcccaagaataatgggcaactcacagacaaaaataaacacacaagaaaacatagtgctattagcaaaaatcagcagaaagaagaaatagtaaaaacagaccagccaaaaacatctatccctgctgtattggtttgctaaggctgcggtacaatgtgccacaaaccgggtgcctaaaacaatgggaatttattctcacagctctggaggctagaagtctgtaattgaggcgtcacagggccatgctccctctgaaacctgtagggggtccttccctgtcccatcctagcttctggtgttgctggcctcatcgtctcatggtattctccctgtctacacggccgtctttttataaagatgcagtcacattggattaagagcccaccccactccaggaggacctcctctcaactagttaaacctgcagtgacgctacttccaaataagcccacatgctgagttgctgtggcttaggacttacatctttctatcaggaatgtaattctatccataacacttacgatgtttaaagatgaaagtaaaattttgaaaacacctaaaggggacagaaaactaaagaagaaaatcttgcagatttaaaatgaaaacttatagacatgaaaaatacgatggttggaatttataattcagagtcatgtttaacaagattagacacacctgaagctgaaagataagtgaaaacgagtcatcccaggtgtaggacagagagacgagataaggaccaggagagaaagtgaaggaagacctgcaggatggaggaagagtgtctgacaaagtccatccaaattccagaagaagggagagagaacagggcaggcaatatccagggagatagtcactgaagcttttcccaaagtgatgaaagatatcaagttacagattcgaaaaacggcaaaaaatgacaaacaggataaataaaatgatgagataaagcagtaagagggaagtcaagaattactggttctgcagtttctggcctggcagctgcacagataggattctcaagggctcggaaggggagaggtagcagagaggcaatgcatgcagcttgcacacattcagtttaaattggctatgagacttccggttagagttttcctattgcatatttgggtctgagctcaagagagccacctgggttggaagcagcaggaagacctattttcctgattaatctcaatgccagcctcattacacaatcttaactaatattaaacagtatatgaaacaggtgaagaagaacagctgtataaattgcataaagcttagcaatgtgggtttttctagacaaagttaagcagcaaagcagctccattatgagggacccttggccacggtttcacaggtgcaggttctgcagatcatggcatgttgtcctgttctctggattatggctctagaagagataatgataaagaagacccagggtggtcagtaaaaaggtcctacgtggtgtctatacaatgttgcaagtgactaaaaatgagtaaaacttacaagatataattagtagcatgcaactcttcataaatttgtcacttctttgaaggtccttgttatgagttgaattttgttctccgaaaattcatgttgaagtcctaaatgccctcagcacgtgaccgtcttcggaagtagggccattgcagttgtaatcagttaagatagggtcatactggagtggagtgggcccctaatctaatgtgacagatgtctttataagaggacggtcatgtgaagacagatacaggaggaacgcctcgtgacaacgcaggtagggacagggtgaagcttctacaaaacagggaacaccaaagatgagcagccactgccagcagttagcagagaggcgtgggacagatcctgcctcgtggctttggcttccagaaggaaccaaccctgccccacaccttcacctcagatttctgctctccagaactgcgagagagtggatttctgtttaagcaagtttgtggtactttgttacaacaaccctagcaaactaatacagcctaaaaaaaaaaaaaaaaaaaagtaataggaaaggaattaaaatataacgctaccttgcagcctccaccaaacactgttgccatttggttcttctccttcttgttcaacctcaggagggggtgaaaaaagtccaggcagctcctggtgatagctatgcaaagcttcattctgcagcagtaaaagtgtttcctagaagtactaaggctcgttaattgcagccaccctataaaagaaggtcctctttcatgaagagcctgtttctctgcaggaagatggggctgacctcagggcctccagcacttaggcacttatccatatgtctgtaaccattgttgtgaggttagttgataatggctcattatcctcgctaaaatgaactcgttgaagtatgaggccaggccttattggaatccttccctttccctttcccttcccgtttccttttccctttcccttccccttccccttccccttccccttccccgtccctttagatgtagtctccctctgtcccccaggctggagtgcaatggtgcgatctcagttcactgcaatctccacctcccgggtcaagcgattcttctgcctcagccttctgagtagctgggattacaggtgcccgccaccatgctctgctaatttttgtatttttttttttttttttttttttttttttttttttttttttttttagtagagatgggttttcaccatattggtcagggtggtctcgaactcctgacctcaggtgatccgtccgcaggtgagccacccgcctcggcctcctaaagtgctgggagaggcacaggcgtcagccacagtgcctggcctactgtcttctctaaaatggcatctgtgcattcatctcagccgcccctgctcagataaaagcaatggcgcctcctttgaaatctgagagacgcagggccctgcccattctgcggaattccttctccctgctgcctgctgtgaggaggccccctttgccacggaacctgaaattcctgccactggaattacgctctggacaagcggcaagatactcctttcagtcccagccactgggttcctgctgcacaggaggccagggtgctgtgaacctgctctcagccccgggcaaagggaatctcgttaatccaggtggccagcgcctcttcctcagagcatctgcagtgctgcagacagggcctccctgcgtggggcttctgtcctccacactgtggtgctgctgggatgttttcatggggcctttcccttcccgtcaccacgtgtgctccagaacccggtgcatttggatgaagccactagatgtataggtcagcagctccacatagaatcgaattatcaaatgcacactacctgatccagaatagatcgtcctggggtaaacacattcacatattctgaatgtacaaatggctgtctagtaaacacactggaacttccataattattgtccttccagataatttttcaagattatatgcacgtattctgccattccttttcaagacaactttagaacttcctttggacagctactgtaagccaaagggcttgcatttgaatatcttgcatgaagctaaatctttgttcatgaaaggcagaataattttatatgccacaaagctgcagtagtgtgttaggtttagtagatggctaagcactacactgtattattctaatcctattttcacaatttaacaaatgtgagacaccgtgctacttgtacaagagatacaaattaaggaatcttcaatgaccttgtagcctagaaagacctttagtaattcttcttaatctccctacagagctaagtgatccagagctgaattaatccagaatctatgtcttcctccgcctccggagtagctctagaaaggtcaaacccttccgagatggagtgtctgtgggggtaggtcctctttgctgtgtgcgatcctgtgagacagcgggatgtcctgcatctctgaatttgaagcgaggagtttttctgctatgtttggggagagcctcactcccctgctcagtagatcagacgtgttctcttctttcaccacagctacaaacaacacactggcattgtttcccagacactcgactgtcccgatgggcatttggacatggtctatgagaggaataagctccagccactgtagtggctcatgggagagggaaatgggtagaaattctttcccaaactggtatttctagtaaagcactcagccagagcctgcagctgttcactattccatatcaattctaaacagcattttcgttggcaaaagaaaagtgagaaaacaacaaagcttgaagccyaaaactttgggaaacccctttcctgaatgtgtttacttagggcttaaaaatatgcctgttttcagaacagaagaactaatatccatgttttctatgccgatttttcagagtacattttaaatgtaagtacatttagtgattaaaagggaaaaatacttgatcgttttctaaacataaccaaaatctcactatgtaattgttttttcctctatttaagagcagaatatttcattgctaccaaaatgctagtattttggagaaaatagaagaactagaataagtagtcagcaatacaaaaccctgcgtggaagatgtgtattttggataggtgtcaacatgtccaagctctcagtgacaaacacaggctcattacaggtctgagcaaatgtgccacttctcaggaagacaaggcagatcaatgtaaaggcaggtggcacctggtatggctcagactcgcacgtggttctccacagagctgctctcggctcctttggaagaggttcaacgttgggagcacaggttgcttctctggcccatgttattcctggagctactacttcccagggcagagttcgtgtttttcgttcataaatggcctggaaatcctagcattgggccagccatccagaacagtggagctgcatgatctggtctggggatatttcaaagggaatagaatactgaggccctgtgggatggaggctgcttcccgatattgagaactgcaccagactgagctgtgtccagaggaagggagaacgtctttcattcacttaaaactcacccaacacctgacacctccatcttggcatcatccacctgtagcctctagccctcttcatctgttaagtgagagtaactggcaggttatttggagagtgaagtgacatcggcagagttccaggtatggtgtctgatgcgtgagttcgccccctttcccggtccccttctcctccatttgactaattatcaaagaaagattgctttagtgaatgagacagtttagatccattcccttgggaaattatggtggtcagccctccgctcggtctcacttttagataccagaaactatatgtccttgtgttggcagagctggattgtctgtcgccctctggtgcaatcctgcattagtaagggaagtgtttttctggggcgttctaatgaaaagtgcttaagcatttgttttggtgcccagataatgtgactgtagttagtatgtagtgtttggactttttgctcatgcttttgttgttgttgttgtcattgcagaaataaaattaaccccttaatcttatgcttaatgtacacaccaagtggtttgcatattatactgagaaaataaaaagattgttttagaaaaaccaaaggacaccaacagctctttacagccccaaagcaggtgtcgccagaggtcacaggaggggttcttagttatcagcaagggaaactgaggctttctcgtttatgcagaagtggaatttattgaataatattaagggggctatgtcgccaatgccacagtcacactgcccacacagaactggcctggcgaggtgttactttgaccaccattgctgggccaggacgctgccaccaaggccgtgcccctgccagaaactaaatgtggctgccccatccctggccctttctgtcagtagggtcaggttcaaactcctgggtagtcagcccagctctcattgactcagtctgaacagctgcctgttccctagaatccacatgcgctgggacaatgggaagtatcggtagacgctatggtgggaagatgactctgtgtccaccaaggttcttgggctggggaatggtctgagcatatgacggcctcagaccccagccaaccaaagggaaaggtctcccctgtactcacgaagcctccacgatgtccatcagcactttcttcctccgttgcagtgtaggtcagcccttcgcagatgctcacaattccctgatacagccggttgccctttgttgtgttaaactgaaagaatttcagagttggggccaggcatggtggttcatgcctgtaatcccagcactttgggaggccgaggcgggcagatcacgaggccaggagttcaagaccagcctggccaacatagtgaaaccccgtctctactaaaaatacaaaaattagctgggcatagtggcgtgttcctgtaattccagctactcgggaggctgaggcagaattgcttaaaccgggaggcagacgttgcagtgagctgtgatcatgccactgcactccagcctgggctacagagcaagactctatctcaaacacaaaaacaaaaacaaaacaaaacaaaaaaaaactcagagttggagaaggactcggacaaatgtcatattatagaggaggaaaaagatccaggaggcagaaagacttccctgagggccatgatggtagttagtgcatccattaaatacaagtcttctgcttcttattcctgtaaataagtttgcatttaacatttttgtacattaaacgttactgattcatagtcaatgattatggtcagccctccacatccgcaggttctgcatctgtaggttcaaccaatcgtggaccaaatatattcaagaaaatgaaataaaaatacaacaataaaaaagtacaaaaaatcgagtacaacaactatttacatagcatttacattgtattaactatcataagtaatctagggatgatttaaactatgtgggaagatgtgcataggttatatgcaaatactccattttatataaagacttgagcatccatggattttgatatccaaggtgggggtcttggaaccccacaaataccaagggacaactgtgtattattttcataacccatttctgcctagtgttccattagtggaatgctaaccatgtgggaattatttatatcctactgttcaaggtcatcaccaaggtctgatttttcacacacacacagaattgcaacctccagcataaatggggatgaatttactactaacatgtagtttccatccacaaatccaatgtccctatgctatttgtaactgtggagccaagagaagctgttgaatcatgtggtgaatatgatcaagaactcaagattagggataaaagcaatcattctgttattcctttttaaaaattattagcctgtaatttaaacatcaggatctcatgtaatacagaacaatatcttctgacatttttacaatactagtattcttacaaaacacagttaggaagttacatgaagaaaacacccagactgtgtgtggctaaatctttagtacctcatttccatagtcttagagaaagtttaaattatattgaaacttttctcaactgctatcttaatgtgttcaggctgctgtaacaacatatcattcaaactgggtgtcttataaacgatagaaatttatttctcacagttctggaggctgagaagtccaatatccaggcagattccatgtctggtgagggcctgtttcctggttcatagatggcgccttctctgcgtcctcacatggcagaaggggtgagggagctctctggggtcccttttataaggacactaatcccatttgtgaggattttcactctcatgacctgctcactttctaaaggcaccacctcccagtactcttgcattggggattaggcttcaacatgaatttgagggaggcgcaaacattcagaccatagccactggtcaacattaggtaacctgcagtgcttggctgtgggatgggaagcctgtgttgtaaaggacgtctgagtgggaacaggggtctcaagctgccttcacatctaacgtcagcacactagagatggacattgcagctgcaacctactgtgcctgtaaagcatttagaattacgccttgcatacacaaagtgctcaataaatgttaactgttattatggttgggcatcagccactttaattatctctttcaatcctcatagtaactcttcaacataggtagccttattttgcagttgaggaaactggagcttagcaaagtttagtgacgttgcagagctagagttcaaacccaagtctgactccaaagtgcatctatctgtgtatttgcttatttaacctcagacacacagaatcggattaattagagtccttgattcagcacacgttctcttcattgatccttactcctttattttattttttaatgctattttttgtttgtttgtatttaatagtaagataaacactgtgaactcaccacttacctctcatcatgagagcgctggtgcccacctccacctccgagttccacatatcccattaccctgccttccccgtccaaggaaaccactgtctggaatccttcgtcattcaagccttttcacagtatggctctttccagccttttatttctctactgtttcgcttggaaactctacatttctaagacagtgtggtgcctctgagctctgtggcttttgctcctgctagcctttcttcataaagtctttcagccccacaagtgtcgcagcttttcaaagcctttcccatcctttaaggtcctacttttcttttccatgaagtcttctctgggccacgatgactggggaatcctcactgtcttctgaagttctgcacgtacttactctgcacatagtcggcggtgaggtattcatcacattgaaatcgagttacatgtggtcctgttctatagtcaaccaaaactcctggggtaaaaatgctgcttttcatcttggcaatctctatcctaaccagcacagtgcctcgctgaatattagaggcctgagaattttctttgtttttttttcagagtgatttttttttctctgctttatttgatactttgaagcagcacacatttcagtttgctttatgcttgatttttttttatttcttctaaacaaacgagatacatgtgcagaacgtgcaggattgacacaaataatagctggcagagtgtcctaggaaagactcctcagatgttataaataatacacaaacaaaaacacacacaaatatttactgaagacttttcttgctctgcaaggcactggctgtgtgatgcagaataaaaccgacaaaattcttgccatctgggatgtgcatgttatgtcagcacagggaagagatcaagtgtgtgtgcataggacatcaagaatacaataaaacaaagtggacaaaaggaagcgagggtggtgaacacaggacacctgaatgaaggacagagttgttggaaaaggacccctgagtgcccaagggaggagctggcctggagtagtgagggcaaggtgattgcaaatgaggccttggtgattggaaatgagctcatccccacatcttataaatagttctccaagttatccgaggcaggttattctgtggcaaagacgcctcagctaactggatgcagaagagacaactgaatagagcctcatggtctcggagtctttttttttttttttttaagacatatctttggcattttgtacctaccttctgttctaaattttgcatttttactactttcaagtgggtggactttgttgtggtgggtagttcaagattcatcatacaaatgtgattgtgcttcgaaactcccaccagtctgacgcacgcatgggttttctggcaacatttgccatctacagcactctctttgatcaccttcatcatcttccaacattcctgccacagtcacttcccagaaacttgctaatctgtaatagaaaccctcagattcctatggtgaatttgtaatcaaaagtcacatattgatttcaaaatcaatacacactttaaaaataacactacagatttagcagctcagggaggaaggaaaccgtaagttcatctggtgcagctacccgtctgggatgtgaattcctcctcttcatgaaatgtttacattcatatcacagtctagggtttagtgaaccataaaaagctgaaagttaatgcaaacagaagtcgcccccaaaacatataccaactgatttaaaaggagacacagcagatggagattattgtgaaaagaactcttactggacaatttttttgttattttaatctctgcttatcccaattcttttagctgcatatactgagacacttcacatctataataaacttggtaccagaacacaattcattccagacctaactcttttagatcattataaccgggggaggaaaaaagttaaaaaggcttatctatcttaagaagtatttctcagtgttcgctacacgtcacttaatcttttccaaaatttgacaatatacaaagcagtttgtagtgacttttcatagtgactctacaataaaatgggcctgtcctccttgcttttccaaatgcagtcatcatctgacaaggtttagctatttggggaagtccttgcttgcaaacgtagttcttttgccaaacaggtttggtcaaactgtgtcccctagttgcacagttaccccatatttgattaacaaatagcaaaacagagataatctcagaaatattcaagagtctcaaaccccaaataaaatataggcatcctcctgttgagtcgaattggcaattttgattagcaaggctcatgaagcagtagatatcccctctgatccccatcccagtgcgagggcacagtgagttgtattttctaagtataaactattctctagcagttcggctggagtattgggagcaaaactgtatttttctaatattttcagactaagacagtgtctctgttttctggacttttccgtggcaaatgaaggatttatcagcaatacaaagaaagttctcccagtgggtactccacggggagaggagctggggtctcactagtgcacagccataaaagacaccacaagcatattacacgtgaagcaggatccgtgcccaccacagcagttgtcccaggagtttcctgtttgaatgagacactttgggtggatactgcagggagggagaagctgtgtgtggccaccacagctggaagcgtggcctggtgccctcacagctgtctgggagccccttcccgggaacgccggcttttcccgggtgcaccattgcagctggagccgttgtcggccgcctcgaaaacatgcagttgggctgctctggcaggcttctccagccctcctcccaaggtttacctctctaaatgtcaaaagggagagaatactgtatttgtttttccctctactgaaatttatttgtgacatcaggcatcactttcaccttagtcattttggctggattcccatactcaattaaatatccttccttccatatggcccataggaagagagagaaattacatgtaactggtctttcctcctctttataaagtctggtggctgagcaacttggcctgtacttccttcatgacccaccatcccatgactgcagggcagttttaaacacagcagcttggtttctattgcacggaagctggccaacagtcacagtgtgcatttttctattgcacctccttgtgttaacccaagttcactcacagctgtaactacagaagtttttctgaaagcaagtgaagccatccttcttttattgagtttttgagctagggtctcactctgtcacccaggctggagtgcaatgatgtgaacatggctyactgcagccttgacctcctgggttcaagtgatccttgtacttcaacctcctgagtagctaggactgcaagcatgtgccaccatgcccacgctttctgatttttttttgtagagacagagtctctctatgttgcccaggctggtcttgaaccactgggctcaagtgatcctcctgcctcagtctcccrgagtcctgggattacaggtgtgagccaccatgcccagctcatccttcctttaaaaccggcagctgggcaataatacagatgggaccaactaagtttctcagaccactcagggaagctagtcttgcatagacaaaatatacaccctcttacctgccccacctttaaggctggtccccagggtccgcgctctgtcctccagcctccacgcttccctgtgactagcctctgtggtcaaaggtgcttgctgatgcagcctctgtacagcctccatgcagtgcgtgtctttatgtggaggagaccgcccttctttcagcagttattgagcatctacccactctgtgccggtcatagggcttagaactgcatgtctggggggaattctgcaaagagagcctgaaataaaggcaaacagtgagagacggccaggagaaaccatgagcactgcagtgagtatcaagggacaaagctgaaaaaggaagactgaacgctgagcttcaagccattcatttctatgggccgcgggagcccttgaaagtctgtgggcaagttttggtgagattaagctggtagttctgttcaggacaggttgaagggatgagagattaggacacttaccacctgaatcctgtcgctggctttagtttaaaccacccgtaatgtagacatcctgacttagaattccctgtgctgcttcctttctgatggaaacagctctgctaacagagtgcaggctgtgggagccgagccccgttgcaggcagcctgcaggccgcagtttcctcggcttaccacccagcgcttttcattcggctcagcgctagggacctctgcttccacttctcggtgttggaaattgccatttatttttgctgtcgatgatctgtattgacttggcctgagtatgcgtgcacgtctctggtggtctgaattatatagaccagaagggtgtctgatgccgcttttataaaaaataataataatttgaaaggaaaaatgactcactgaagtctggcaaatacagagccctctctctgaatcgacttctcacttggccatgttgaattccaactgggtgtcctcagacatttctatcccaagatctactcctggcttagaatctgttttgttttgtcttatttcagctcatggttcttgttcccccagctttatggggtataatttccatacaatagaattcaacactttcaatgtgtggttggatggcttttggcaattgtatacagttttgcgatcacccctacactcaagatatagaacactgtttctcgtctggtgattgctggacattgaattctttccagttttcactgttatgaatctgactatgatttttggcatggatttgtatgtagctataaatcacttggtaatttttcagaagaatagcagtcttggggcctggatggcttattgtggtctcaaaaagttcctgatgataaggttgcagcctcatgcttctttataagaatgcagtattacttgcaagggagcttgggtagataagaaagcaagaaagtccatgtggagaccctgtccagagagcacagacatggactaagttaaaggatggtaaattagcaatgcccaaaagcacatggaggagatacttcccctcctgactctattggtgatgcagtttatttgtctgagctatctgagcaagtttcctctcacttacgtgctggggacagcagattccaatgcagagtccttagagctcaggctcccctcaacctgacgcatctctcaaccatttgtcttaagctgtctgaagtcagcttcccatcttggggaggtagaagtgaaagggtttccactttgccaagtgagcgtatatggggagactgagggtgtggagttgatgatggttgtggggtggctgacagtgtccacagggctagtcttgaggcaggctgacactggggccagatgggaccactgtgcctcctgtcccctccaccttctcctagtccaggaagggaatagcagcagctgctctcagtggggcattctttttccagagacaggccagcccagcagtgatcccttgataaagcaagtcaccgttatcagagcaagaactatacattcacttaaaacttttttttttttttaagtgtaaaatgggactgcaacaaaaagaaaattgtgcttaggagaatgtccctcagaaaatgtactttatgattgcgaggaatatttgccaaggtctttggggtaggctgagccccttcacctccctggggacatgctaggatggcaagagaggatcagacatctcccagggaggctgtgtccagccgggctcctggagtggcgtaagtctggttgaaccagcactgaactgcctgagtccatgtgaacgcattgaactgttaaaccgtgtctctggcggccacatctccgggcttcacccgctgctctcccctgtcctgcaggtacaaagtcaatagtcaacctcagttttgaatgttacaaaattattagcctctccatagttcttcccatggcttctcacccaagccttctgctcctctctcctctctgcccaggtctcaccagctgcccttgggccaggtcactgcagtgtctgccagcaccacgacaggcaggctggaggcccagttctcacagaaaagactcgaaagggggctttccatcctttatagtctacctgctacttataggccaccaggacaaaggatcaaggtggcaaggcagaaattgcagcacagagcgaatggaaaggcagtcactgaagggattcttttgcttttacaagtagatttttcttaaacaatcactgtatgaaaacaaaagtacaaaattattaaaacacctggatgatgaattgacaacaagagtttttctggaacatcctcctgtgggctcggggaagacagtttttttctgtggtgatagatggtcaggaaatgtagtgacatagaagtgaaggcattttacagagctcaccttaatcaatggctttttcacttattaagttttcttttattttttccttcttcaaaaacgactgataccttaatttatgggaattgtttccagtaaaaattgggacaatgatagtgagtggagaatatttatatgctatacttcctgtcttccttcatcttttattactgaggatattgacatgaaaacaggatctttgtatccaatgagttcatcgacggccgatttcccaccagaaattccaggctttctgacatcagcgtgcattgctctgcatgtcacttggagcaccggcatctggaaatgatgaaatcctgaacaacaaagtttgttttcaggaagacaaggcagtggggaagggaagggtgctaagcttcagtgactgcctactgtgtgccaggcattttcatcttccatctcgatagaatgtctaactgtgctctgagatgagaactataaatagctggtcagccaaaagttttctgctttttcttagtgatctcaagtgtttccatgacacgtgctgcaaccaaatacattatgtgtaaattgccaaagacctgttgatttccaaaccattatatagtcatgggaatgcttgtatacctgaattgtcataaaattgatgagatgcgaagatacagcagaatatatcagataattctgcagaactcttattatggaaatgaaaataattcaatagagaagtctcgattcataaaagactagttttactctaaagtatctaaaagacatgcattaaaaagacatggcactgtccccgaaatgatcttgctgtgttgcatttcaaatggtaccttcattttgaaactttgcacattagcacgttctttataatagcaaaaagtgggggagtgaggaacatttggtgccggaagaatgattaggtaaagcacaccaagctgaaaaaagtatttttgcagagcgttttcaagagcatggaagagtgttataatgttaagtgaacaaaaaaaaaaaaaaatacagatccaactatgtaatcattacacatagaaataaaaatgagcaataaagccaggatgtcagtgaggatggagtggagggaatgtcctaaatgtgcgttggcccatcatcacctcatgcatgaagtgaatggaaacatttggtttatgttttctggaatgtctcataagccattgtaaccaaaaactacaccatgaacaaaaagcaaagcaggccctgcaggccctgggtgggaagctgaggaggttggcagtttctcaaactcatgtcagatgcccctcggccactagacagaatctgctgctatttgggttctggttgaccagaggcctaatctggaatctggttctaaaaaccaatttttgttatagggcttgctggatacaaatctgcaatgagacattgtcacaagcaatagcttaagaaaaacataaaggaaaaaataataataagtttttggaaataagcctggaaaagcagtttattgccatctgctaactcatttgattcttgcagtaaccctagggtaggtatgatggtgatctctgcttcaaagatgagaaaatcgaggctgcctcaggtcacttgacctcctcacaggccagtggagactggcttcagactcgggcctttggacctcaaggccctggtcttcttttgttgtttgtttgtttttgtttttgtttgtttgttttcctgagatggaattttgctcttgttgcccaggctggagtccaatggcgcaatctcggctcactgcaacctccgcttcctgggctcaagtaattctgcctcagcctcccgagtagctgggattgcaagcatgtgccaccacacccggttaattgtgtatttttagtagagacgggtttctccatgttggtcaggctgttctcaaactcctgacctcaggtgatcagcccgccttggcctcccaacgtgctgggattacaggcgtgagccaccacgagcggccaaggccttggtcttcctatcgcattttgacaccttgctcagtacgatgagtagttaaaatcactgtcattggctacatgcctactttttatagtcactctacttattgtggttttggctacatcctagttgaactctagggctagtgtttattaaggtcttgatctcatatggcatttgtagacaatcgaatgttgagtgataagccctggtaacgtgatttctcactgctggcccgtgaagccatggaaatgttcccatggaaatcaccccatgtgtggaatgaatggtcagtggaacataggcatctttctctcctgtcctctaggttttaaaatacctgaatgtcctgaaatgcaagagtatcctaagagcactttagaaatatctttgcggtttctttctggtgtgctttgtgggttgggtgaggtaccgtattccaggacacgtggcccttagagaaacaaataatttcctttcctcctgcttcagtgttattggtaaagtgggaaggtagccccaagacactcagctcctgcactgcatttggatagaagggcgttcaaattccaccagggacaacttcgtctaaccccctagaattcctcattttgacccttggcatactctatatttgttgaaatacaaaaaaaggagttgaaagtgagtctatctatatgtagtaggtatatcgtgttcactgtaaaattccttactgtatgtttaaaatttttcagaatacaatgctgggggaaacctatggaacagaagtagggaaaaaattcgacaacgcaaagtgagagtgggaaaccatgtgaagctctgttagagtatcatcactaatctcttttttccttatacctatattcatgaaagcaaatagagaacaatacaatatagtgtaacaccgtgtacccatcactcagcattgctcaatcttagttatcattatggttattattattattattatttgagacaggaccttgttctgtcacccaaactggagtgcaatggggtgatcctggctcactgcagctcaacctctcgggctcaagtgatcctcccacctcagcctcccaagtagctgggactacacgtgcgtgccaccacacccggctaattatttttggtagagacagggttttgccatgttgctcagggaggtctcaaactcctggactcaagcaatcctcccaccttggccaattttaatattttattatagttgtttccattttttgtgtttttcataaattaaatcttgtaactattatatatttcacagaatattataaagttaaagctccctttgcatctttccctctccaattccattcttcctctctctctaaaagtaactgctgtcctgaatttaacgatgatttttaaagtcatctaggctctcgtttttctttcttttttttttttttttttttttttttttttgttgctgttgttgtttgtttgttttaattgaaaaggggtctcactctgtcacccaggctgaagtgcagtggcgctctgtgggctcactgcaacctctgcctcccaggctgaagtgatcctccaacctcagcctcctgggtagcagggaccacaagcacgtgccaccacacctggcaattttttttttttttttttgtatttttggtgaagacgaggtcttgccatgttgctcaggctggtctcaaactcctgagctcaagtgatttgcctgccttgtcctcccaaagtgctgggattacaggcgtgagccaccgtgccaggccggctcttgtttttctcttccccctacaccccaaataaacacagagctttattcctgcctcagtcaaattgctgcttcaaggccgcagtttggacactatgttttttagggtgtggttttttttttttttttttttagacagagtttcgctcttgttgcccaggctgcagtgcaatggcacaatcttggctcactgcaacctctacctcccgggttcaagtgattctcctgcctcagccttccaagtagctgggattacaggcatgtgctaccatgcccggctaattttgtctttttaatagagatgggatttctccatgttagtcaggctggtctcaaactcctgacctcagatgatccgcccacctcggcctcccaacctgctgggaatataggcataagccaccaaactcaacttataatttatgattaaggctgcagtgcaatggcgcgatcttggctccctgcaacctctgcctcccaggttcaagtgattctcctgcctcagccttccaagtagctggaaatataggcacacgccaccacgcctggctaattttgtatttttaataaagatagcatttaattatgttgtccaggctagtcccagactcctgacctcaggtgatccacccacctcggcctcccaaagtgttgggattataggtgtgaacccctacagctgacccagacaccatgtttttatggctggattttgtctttgctctggttgcggtcttaggcacccttataaatagagctttgaagagaacattaccaatgtatttttaatgaggtcatgttataaaattgtcgcataggacttctcaagaaaagacagcctcttccttgcaagatactttcttttgcaaagattgagatcattccacaacaatagacctctgttcattgcttccttcttatgcaaaagtggccgtccctcccatcagaaggacccccgctggcactctgtcaggtagacagaagcatggatagaaggctggtggtgagctccaggtgccttccctattgtctcttctctcctataacctcgtataaccttcctgggttttcctgggtgcatgtttttttgttgtcattggtgttttgacagctggctgtccaggcaaggctgctgtgtttgagcagaggtttgctgagttgagcaggggtgtggctgcagggcctagcctggcctcccaggagcccccgctccccgtgtgcccaggtcatacccaaacaggagcattccttatgctggtcctggacagcgtttctattaaagggttctttgtgttaggaatgttcagcagagcgccatgagccgggtgagagtggaatgagtggtttacccagggcacctctggaccctgggagtcacagctgtggaattttactggagttttcactgcagtgcagcccagggtaggacacagagggcttccactcccttggagcatgctaatctttccaaaacactcattcgtgggccctcatagaagctcctagggcattaccaagaaatagcagtccttgatcatatccagtgaattctgaaacagtgaaggaatttagatctcatgtgtccatgttgctgagggcgtcctgggcacagagcctgctcgcatcaggccagattgtttggagtattgccaactggccttttttctggagaagaaagtactgacgctacgaagacttcagtgttctcctgcaggggactgcaggggactgcaggggaagggaggattggcctgtcacttgccatctctcatttctgcgatgctacagagagggaaggggaggcatacatatgtcagaatctaaattacagcatgtggaaagacctgccctcggggtcagagcacaccaggctggggaggacctagtttaaagggatagaagagacattactttagctccttctcttcaggggctccataatggttttaaactgttctttaaaatcgaagtttttctaatctacttttgacttatgtattaaccaagaacctcttgtaaatcttaagactatatagttgtcaaagacaggcaacttgaggttgagtctgttgctaactaacttactgacttcgcacaaatcactttgtctttgggaacctcgctcccctatcagtaaaacagagatgattgattgattcaaaagcattcactgagcacccactcagctgcgaggcactgcctgcatacttgggatatgtcagggagtgagagaggcaaggatccctgtcaacatggagacttcattccagcagaggagacacacaggaatgagtgaatggaataagcaaatagggtatgtactgtaggagcaaacaagggatagaaacatgggagatgggtcaggagtgtggactcgagtccacagcgctaaactgtcccattgagaaggtgaatgagttaaaagagatcaggaagttggccaagtagatgcaggggaaaagtgttccctggagagggagctggccagctgcatgcacaaggcctgatggacggctgagccagtggggaagaggaggcagcagcacagtcgggatgaactggcaggtgggcttctgctccagaagagatgcgggagccctgcaggtttgagtgaagagtgcatggtccaacacggtcttcagagcatcgttccagctgctgcaggtttgagtgaagagtgcactgtccaacaaggtcttcagagcatcattccatctgctgcaggtttgagtgaagagtgcactgtccaacaaggtcttcagagcatcattccggctrctgcacagggaagagcatcaggggcaagagttgatgcagacagtaatagatggtaatagagtcagatacaattgggcaggcaayggccctttacagatgacgaagcatcagaaaagttagggtgcaaccatttgttttcagtttacaaaaagggaagacgattaatccccaaaaaggagcctgtgagagtcagatgaagaaattaagaaatgaataatatgggtcacatgagacagtctctttctttttattcatttatttatttttacaaaaaagtatgtttctgtgtccttcagcacagtttgcaggagcatttagagcacacccgtggagtggcccttttatgcttgccaagcatgctgaacaccgtaagccacgtgtgacacatcttccatggacatgaaagatatgttgatcattttattgggctccagtctcagctctgccacgaactggcactgtgccttggaccaagtcacttcatccctttgggtttgcgtttgctcccctggaaggtaggggaggggtgcagtgagctctggcgttcttcttagcctctgctgcagctgcatgagtgggtctatggcacagccccctgcctgcatcatggcaggttatacacagtaaagagatgaaaggaatttttctgctaagggaagtagccccatctgtcaggatagttggctccattgtgtctaacgtaggtatcttataagcctgtacacatggcagccaaggggacctggccgccagagccgtaggagatgacccagcacaatgggctgggcagtaaggaagccagactctggagccagcgtggaggtgcaggagctcgtgagtatgagggcatgatgaggggtgcacagaggaacccctgggctaacaggggcccaggagacagtattacggcattgggctttgtattgccggagaccagcacagatcccacaatgcaacgatgccaaaaaacggtagaactgaaaaccccagccagatcaacgcgagaataaatctcttttctgctgaaattgatagcctcctaaaatgctaagacacatgcagyggagaataatcattattgaccatgaaatagctaagaaccagctgagaaaatacagaaggacacacagtaagaatgaatgagaaaactcttgcatagaggatacggtcagagttagcaaccagttgcttcttcatgtaaattaaatcagcggagaatctaaaaccatcccgtagaccacatttagagggtaggaaggatgcaatggggcaaggtgggcaggagatgggcttagcatccaagcaggctggactcacagccctctgcctggtgtgtgatctcagcacttcttgtaccttatctgagcctcaatgaaggtaataaaatcacctgcctataagcctgcagtgagaattagaggagcaaatggatgagcctcagtcctgtgtggggtctggctgctcacaaggcaccatggacgccgtctttaccatcatcactgtcgacccggagccaatggtgaaagcaggacacaggcaagccccagcgtttcccaccattgtcttattttttcggcttcaggaagacattagacttctaggaagagattccttaaagccaggactagaaggtagactccagattttggctacaagtggcaaatatgtcttgtaagatgaattttatgtacttgtgccaagtgccattggaaataccgaagactgtgcaaaaataaaagacaacaaacagccccaggaacccggagccctctcccagcccagaacattcaccagctcggccaagagttctgctgggttttctctgggggctggtgctgctgtggacacgacaacccggaacacggagggagggctcagcgctaggaagggagagggaatgaagaggagtttccctctctttgctaatttcttcgtctctgggaacatttccttcaacagagtcctgcttttctcatcctcacacctcactgcgcccctcctgaacccactcctttctgaatatggtctactgtccttccgtgacccacatcaccttggtcctctccctcataagcacatcctaggtgggcctgcccttcacttacccatctccttagaagaaacgtgagctctccaaagggaagggcagaaccctgcttgttggtctttctgcccccagcacttgacctagagccttgcactgaggacgtgccactcatgtctgctgaataaacagccacatttccagatgacgatgtccttttccagccaacatcagctcagcgggccttcacgtatttagttatacttgtgcccccgctcaacagggtgaggatgctcctggacacagaaattagctctgaggcaggaaggaggaaaggggatgcttctgggaggcaaaggcggtcaatcagagtgagcaccagagactccgtgtacctgggaaatacgtgggttcccacaccagccttggggagccagggtggggaagagggtctgcagagcaagtttaggatgcagcacatgccaagcttttcagagtctcacagtcaggaacagaactcatgcagggaggggagggattggaaagtaggaggcaaagcagaagccccgaacccaaagacagagccggcgaccggccagagtgcagctctgagcctcagacatgaggggagaagaaggggatggggtggggggcggtcgtgaggaatgtcgttgtccaggctccacccggcccaccagctccgcagaggaaggagtgggctgggagaggcacacaccagaacagctctcctcggggcaaagcaggctttcttcccgaacacccaaggctttccaaaaggtaaacaccatttcccccaagcgaccccaatgtttgctgaagcaaaacctctcgtgtgagccggcgggcggcttcacgacaggcgtgagaaggccatggccctgtgtgggtgaggaagcgcagtgcggctcccccctgcgtggtgggactaagaagagccccctgccacccgaaaggcgccctaacacttcagagagcggatggctgccgagggtggccaggctggagctgcggcttcccacccgatgcattgcagaatgtaactttccaaaatgcattgctctcatctcagctcagcgttaaaacacatgtgtgcacacacgcacatgcagccccgctgagctgggtggtgaaaagaccctaattagttctgattccttaaggcatgtattttaaaaagcgtgaaacctattgagatgctacttcctagcgcgaatacggggctcttaaaagtcctgataaaagtgaaaatccgaggcgcgcctgggaagtgggaatgttccctccaactcaggcttccacggtcatgagtaggaagtcctcttcctaatctcagtatcttaaaaagaagccttgatgttgttacgtgattacctaaaaggaatgccttcctccgcggaccggaaggatatttttaaaggaatgtgaagcttgtgacaggaattatcgatacctttggaatttttttttccaagtgactcaggcttacttgaagccattacctcggagttagtcagggactgcatgacgccaggccccaactgtttaaagcagagcgcggcttagtgaaagaatgaaaaaaccgaggatgttctttgtccattattctcaccgtgatgaatgatgcttgttttcctctccactttaattagaatgtttctacatttgccaaagaaaatgttggaatggagacaaaaacctgaaattataggaacagggcttgatgtaatagcttatttgtaaaggaaacacaacttgtttggcattttattgaaacaggaagttcagaagcttagtacacacaagtacaacaaattctcaggtgcttgttgagtcatctgttgttggaaatagtctcctggtagttttccccttgatttactttttatcttcattttgtttttttgaaagtagtgagggtaggaagttacagagagattcaattagagattatgtgtatttttaaaaatcagctatcaagattaaataaagcaagcgggaattctctccttgctcccatgtaccaatttttgtaattatgtacaagatgagggaaaccaaagaaaaacaataacttgcttcaatgcaattactaattcaaaagtaaccattactctggggaattgtattagagattaacaaagaggaaaagtactgtggttttctttctctatgttctatttgctaggaagcggtcaataaagtaaccttttccccacaggagctggttaatagttcgcttcatgctaaataaaagttacagaaatatctggagctgagttgctggagacacagaaatcttcaggttggaatttcttgcccttttccaaaggattaggccaggacattgctgtcaaatctgcaaaacctactcatcctggcaagagtgcggtatttttaggactcactagtgtgctacttctaatagtgcttagtcagggacccccaggggagtgcaagggagagagggtccccagcagggacgccagaccttctctagctggccgtgggtgctggcctggccacctgtagccctcagcgcacaggtggaggtgtaactggtattcctgtgggagtgacagtgtccatctttgacatttaagagcctgctccttcagatacatttaccattgccaccattggggattggggcagtactggccacccttggcggcacatctccagcttacagcagagtctgagtgtctctagcatacctctgactgaggcasgttaggcttgtgacatcacatcttcctaggtggggcagagactttacaatacatgtgacaagagaaaaaccttacagctttgtattgaaagatttcttaagtttttagtttattgactaaataacactgaacaaaatgattctactatgaaacgaaaggattggacctctgtgagggttgtggcaatgtttcaatagctgagcaacgcaggaggcacacaggccatcgttgggggcaggttggaggccttcagttcctttacagctatgggctcccatcaagggtgagtgcattgaggagacattgcctagaactactggacagacatctcacccaggagacgggagcatggtactcaacacacttccatgcaccgttcagaatcgctaaacacagcagtgcagaggcagatgacaagggccattacggggtcaccaagggaggaaatagggactggagcccccaggaaggagagctgagtctccctgtgggctgggggctggctttgtggccctgcagccaccacctggagatgagagacctgtccctaggcctccctgcagccaccacctggagacagagtccctagacctcccagctgtgcccacctgggcagctgcactttccagaggattattcctgcagcttccaccctcacatctctcagctgtctttgcaggtgcatctctggaaaacagttctcatcagggcaccctgtgcttcccagtttctagtcatttcccttctctgaaggttctagttcagactcttgagcaaagccttcaagacctctccttaaactgccctcctcctcttccgtccagccacctggttgcctcctggctcttcctgccctaataccggctgcccgtacgggactgctcacctcctgcagggagccggacgtctgtggcgatctccctcccgccatgacaccccctacctgtcctccatcatatgggacacacacacacacacacacacacacacacccctacgcacacccacaccccacatgcacatcatacatacatgcccaccagaaatacacacaccatacacaccacccacccacatgcacaccatacatacacatacacacaacacagacattaaatacacatgccactacacacagtgcataccacacacaacacacaccacacacacacacccaatcacatcacatatacccacaccacacacacacacacccaatcacataccacatatacccacaccacacacacacaagcctttcctaattatctaaaggagaagcttttctggaaagcattccccagagcttctagagaaattagtgtcaccctcttttatggtttcatagtaatgtttttatatcaccagtataaatactatcatataaaaagggtaatcagtgtaccatagtaattaattctttaagtatgtctcttctgctagatgatgagcttcctgaatgcaggctctgaagaattttttcatagttttaaatccactgcatggaatagagaaggctctccataaacttcctgagtttaaatggaatcggattggaaggcagtagcaaggcacaaagtgcagtgagagccaagctcaggaaaaccagtgtccttgagcagaaagacttaggaagggtgctcgctagcgaggagggaggcaacaaggggccagcccgtggggagccttaagcaccaagagcagggcggtgcacactttgtctggcacgggctggagcaggagagggaccgtccttgcattctgtgcggatttctatggcaatgacatggagggaaatgaaggtaggatcaagagtcccactgggaagtggcctggcaaccagaggtgtccgcaggacacctgagcctcagcagtgtctgtgaggataggagggaaagccagaccccagcctctctggggagaatctggatgcatgcgggaggaatggatggaagggagggtgtggggctgagtggcggcggctgggctgtgctctcccactcacagagccttccccaaagcggggaaggctgcttgccttttggttcatttcctttctttaatacacagcaaattcctggtcaccctttgttgttggctggttgggtttgtcgctttccttgttgtttacaagctccaggtatttgtgacagatcttatcatctccttccctcttagtcacctcttggcccaactctgcatattttacctttttaactctgctcctgttctgacctccccactctcggaagcatatttgcttggtgttttcagattttatttcattttggctatttaaagagatgcaataaactaaatatggcctggcaagtctggtcttaaaatagaaaatatatatatatgtatatttgtgtgtgcatgtgtgtctgtgtacataggcgcatgtgtgtgcccttgagtgtgcatccgtgtgtgtgtgtgtggccggtgtactataaacccagggcatcagtctcctgacgtcattgcttgcactttttgccattctcccccaaacactagttttcagcctgtattttctcagtttccccaaaaatgattttttaagaaaagtcaaatcagaaagtgatcagcctctaccgccggactctgcttcagtatccatccatgtctctgaggtcttggggctcataggaatgtgcttattttcatagtcccattaacatgaatagtttcagaagggccagctcagttttgtcttcagttttctcactggtgattgtgcaggggtggaatggcaatggaatgcataggggcatgagtgaacttttcgggatgacggaagtactctatattttgattgaggtgttattaattcaatgtgtcaaaatcatcaaaatttttacatttcatcattcttaaattatacctcagtaaagttgattttaaaagttaaacacataccctttgctcgaaaatgatcctgtagagcgtttatgcctttatatgaatttagctaatgcattctctccccagggccatttgcattttaggatataactgatgatgtggaaggtactagcaaggaagtatgggatgggaatctggggatggaagtaccttcctgctttcagtaagttacataggcactccttattcataaggctgagcttggtttcagataaataatcagaaagtaggttgtgcaaggttttaagaagaggatccaaactgggacttagtaacgaactctgaaactgccacttgcattctctgaacttcacatcaagtcaatactctgtatgctacaattccatcttacattaaaaagcaggtctactaagggacccgattcccaagaaataaatgtgctttttacaatgcttgatttgcaagtcagtttcaaagataatttggtgaagatatcagagttatttttacaagattaaaaatcagtattcaacaaattattttattcactttgacttttttttttttttaacctgtctgtgacatatgtctcctttgatccgcacacacaccctggccagtaggaaacaggcacactctgctggtggcagagggatggggactggagcctgatcttggaccttccctgtctcatctagctcagcccccatgctgtcataggccgcagccaagtggccttccacagcccctccatggagccatcgcagacacagcttctccacggagccctgttctcagccctggaggccggcaatgtgcttcacccactgcctgccacattccagccaacagaagaacttttgaccgagaagtagaaactaggtgattcagatcagatctctgttgtagactccactaccctaatgatgaatttttaaaattaaacattccctaacaaacctccaagactctttgcttgggtcggtcaaaatacagtggaatgtgagagcacatgtcagaattctccagcctacgtttgctgttgttgttgttttgagacggagtctcactctatcgcccaggctggagtgcagtggcgcaaactcggctcactgcaatctctgcctttcaggctcgagtgattctcctgcctcagcctcctaagtagctaggactatacgtgcgtgccaccacgcctggctagtttttgtatttgtagtagagacagggtttcaccatgttggccagactggtctcgaattcctgacctcaggtgatctgcctgcctcggcctcccaaagtgctgggattacaggtgtgagacaccacatccagcccagcctacttttatactatgaacaaaacttcttagaattaccaacttaagtacaatagaagcttttgaaattagctggggggaaattgagtctctaagtaaggaggagtaagagcaagaagatcagaaggaaccacagaatcaaacactttcaaaaggaaagaaaattaggaaattgttcggtgccatcccttcatttcagaggggaagaactaaggactagagaagtcaggtcaccccgacaggaccctatgtccctccttgtcgcctgacctctccctgtgagtctcagtggtcctggtcccacagcaggtgcttggggacccagaaagaggccaggtctcctgacacccagccccgctcttgttgggtccctgaatctggaatggttactcatgttgggggaattttatattcttttttccaaaagttgatatccagctagaatctgtccttcctgagagcttgtcactgccctttctctcctccctgcctgtactcctgttcgcttgggactcacactccttgcaaaaaagcttgtttcacccaggggtgagttttgtaactagagcagggagtccttgcctttcattccaatgcattccccaaaagcagaaaagtgttatgcgatgggagtttgcattttggaccaaagactccgcagcaaataaatcatggaaacgaacaatatgtccttaaaccaagatgtaactgtaaacctctactgtcttatgaaataacaatactgtgctttgagtagccagaccacatagtagctggactctagactctaagcagggatgaagtcagtggctgctgatctgggccttccccagaaggatgccaagagatcaagttttgtttttaagttctgtgaatcacagacattatttttgtaatctttttttttatgacacagagtctcactctgtcacccaggctggagtgcagtggcacgatctcagctcactgcaacctccacctcccaggttcaagcaattctcgtgcctcagactcccaagtagctgggattacaggtgtttgccaccatgcccaactaatttttgtatttttagtaaagatgggtttcaccatgttggccaggctggtctcgaatgcctgacctcaagtgatctaccccccttggccccccagaatgctgggattacaggcatgagccaccatgcctggctttgtaaaaaatttttaaagccaatttgcttgtttaaaaaactgaatccacactggtaagttttgttttaataaaaaaattgtgagtaagttgtaaagcttttgataagttcagtggctcctgtaggcagacaataaattgctaagtcccaaagtgttgcaagattctggagagtactttgttcatactttgaagaatatgcctgattataaggcaacacaaattactgaagccttgaaatgatgaggttgtttccatttactcgcacataaaataatatatctaaaacatctagcaactctcaaaagaagagagtaaaaagcttttgagaaatcaaatacaattcattccaattcaacttgaaaattcccaacagtccgtgttgcattttatacatcttgaaccaaaccatggctttgagtaaaggcttcatttaaaaacctaacctatatatggtgggtgttcatgttctattaaagcaaggtccctgtcctagttggagggaacttccctaggttcggcagcataaaccagtgcctgtcgaccagggagtgtcaggaggatgtgctgcttcctgccccctcccgcacagggagcaaggctgtgctgaatggagatattctagtaaggaggagagtgtatgtgagaaggtgtatgtgagaaggtgtggcatccacaacaaaactaataaagcatcagcaaccttaggtgatgcggtttggctatgtccccacccaaatctcatcttgagttcccacatgttgtgggaggtaattgaatcacagggacaggtctttctcatgctgttctcgtgatagtgaataagtgtcataagagctgatggtttcataagggggagtttccctgcacaagctctcttctcttgtttgccaccatgtgagatgtgcctttcaccttccactatgagtgtgaggcctccccagccacatggaactgtaagtccattaaacctctttcttttgtaaattgcccagtcttgggtatgtctttatcagcagtatgaaaacagactaatgcatttggaaaccaagaggctgatggtgttcaggacacactgtccccatttatagcaccttggcatttcagaaaatcgcaaaagcaggaaggcccctctcactttcccctccttgcccttctcccctggggcaggttataagatcctcatttgggagagtctttcccaatacttggaggaaaggaacatccttgtctctgaagacacagagcacagagaagaatcagaacaaacaggcctttctcagtgaccccagtttatcaccattagctcactcccagtttgtctaatcacctcctccaccactatccactcttcatcaaacctaagtacaaaatacccaagtttgcctgtttctgtgggtcttcctttccttgtgataactcctgagtcacatgaaacacatactaaatatgtgtgcctgttttcctcttgttactctttagttacagggaagggccccagccatgaacctagcaatgggtgaggaaagaaatctttccttccctactgatatggtttggctgtgtccctactcaaatctcatcttgaattgtagctccctcaattcccatgtgttatgggagggaaccagtgggagataatcgaatcatgggggcagtttccccccatacagttctcatggtagtgaataagtctcatgagatctgatggtgaataaggggaaatgcctttcacttgcttcccatttttctctcttgtctgctgccatgtaagacatgctgtccaccttctgccgtgattgtgaggcctccccaggcaggtggaactgtgagaccattaaacttctttctctttataaagtatccagtcttgggtatgtctatatcagcagcatgaaaacggactaatacacctaccaggcccggatttgtttggcaataaagtgatccattcacgcccaagaagtgggtggagctgggaaaggccagaccaaccatttggaatagtgttttttgatccacccccaggaggtgaggattggcaggggctgaggggagtgctcacctccagcaaggtgagctggagcccacagcaggactccagcctcagcagaggaactggagagcaaaccaggaaaggcagacagagctgactcacgtgcgagggtgggagaggtcgcacggcctgcccggaccctgatgagctgagcacagtgaaaacaatgccaggcctcacctgcccgtgcttaccggctggtggcaggggggctgagcaggtgttgaggtgttcacaggtgagtaggagaggaaaggcagacgtcggcctaaaggcaatcgcaaggagaaatgcgttgagaattgtagcactgtatccatcaaaaaggaagctcatctttcactgggtgtctttctaattgttagacttgacactgcatttgctgccctgatttcttgtcctaaccttcaagcttgttagaacagggactcagggactctgttttcttctcctgtgctcagtgcagggcagcaggactcacttgctaagtgctcactgacagatgtaagattattgttagagatatggacccgcttgctcttctgagcttccgtgattctcattcggtcctttgctgtcattagaatcgtctggggagaattttgtcactcctgctactctgaccaaacctcgtatacttcaatcagaatgctcggagttggggctgcagcaactggaattgtttcaaactccccgggtgactgccctagcagtcaagtttgagaaccacgggcatggtaaaatcttttctcagcctgagcagcccattagcttcacctagggagctttaacaatcactaatgcctaggcctcaccaccctccatcccgtgttctgacttaattagcgtggggtggggcccctaaaacaacattctaacagcttcccaggcgatgagaatgcacagctaggatgagcttctcctctgaagcatgaagacccacagaatactgcagagttgctgggggtggccctgcccaaattctcgcctaaaaccccaactttcaatgacattgtggacctgctttcgtgttattataaggtttacaaatttctatgccacctatcagaccattttttaaggatgaaatcaaagtttctataagttgtatagttctttccctgtgcattttatcgtaatattgaaaaacgacagtgaaaagcaaccaaggcatctcggcagcatgctgctgactagttcacgcagttaccaccaaagcgcatggacgggacccagagcatsagcgtgtgcccactatcggggacagaaacctaccgcgttcgagttttgacatatttctcgcagttgttgaaaactatgaggcatgaaatccagatttatgactttttaaaaagttatttgtggattcccaagacgattatgttcccatcacttatgtagccttaaaagaaaaaaacctcaaatgatgctttaaaaaaatccaagtttggcgctcattgagttccagtgtcagttgtctgaatcgccttcagcgaaagtcagggggaaaaaatacattccgccttcctttaactgctagttcgtcatggagaacagaaagtcccatttgcatgtggcttttggaaaagctaagccgggagcgattatcctgatgcgcttttactttttgcataaaataagaatttgaggaggatgtcccgggagagtgagccacttctcatttcccaggcctcgcctgccatgctctttgacaacatcatagattttatttttgccgggaatctcattatcaaagcaatgccccccgccccccccccccacacacagactgccaggtaaaccacagagggtgaggggggtgcaggtcatggttgccttattacacaccctcctctgccatcacctccttttttgtctggataagttctttggcagttctctcaacttttatttctgaaacatcctgaaacatctcagtattaaaagcaaggccgattatataaacgatactcccaggcctgacaacacatggttttgcctgaggcctttactgccaagagccgtaaggaccctctaagtcatgttcgctatttttactggccttgagagtctccttgctttgacatcctcttgtctccattgtcagactgttaaatgctcatgcttctggttctcttaaatagatgcagatgtgtggggctgggttgccactgagccctcttctcttttgcaagagctgggatgcagacagaaggcggtttggaaaacacgagccaccttgattttagacaaactctaagttacaatcaggtgtcttcatttatgacatttaacttttacttaacctaatcaagccatgttgttggctactgattagaatatccttttataacttaccttaaatctcactacttgttccaaccatcccaaagtctggcgtcaactgtcattgcatgctgctcttttcagcctttctagttcgactcttagcaaaagccataatcttcctccagtctgtttcctttctgcagtgacaaaattgcccagggaaaggaaaaagaacagcatctatcttctttctttttagctccctggtttaaggctttcttttcccccatgatgaaaaactataatcattctgcttagaaagtacagacccctaagcccacttccaaaagaaggatgcattttcaagtctgttatctttactttcccagagcctgggggtctcccaggccagaagttgacagaactgtcttcatacactcgagacaacttcatgcccatttccttaaaactaagaacataagacgctgatttttcttccagaaaaaaaaaaacctttcttgttctttcaagaactgtttcacggacagtgtttcatattacaaaattgaaacttgggacttttgaactgcaaatttagcagaaaatgaatccatgcgcttgtggctttgcttgtcacctctactcagatgtctcccagacccctctccagctgcaagctgcaggcagaactgttcctctaaaagaaaacaaactcctgtttttcctactactgctactgcttctactgttgctacacacacacatacacacacactctctcacacacacactcacacacacacacacacactcagaaaacacttctgacaccaaatgtatgggtttttttcatgccaaacaattctgcagttcactgcagacaccagctgagtgtcctacaatccaattgtggcaccgcctgcctggagttagcaggtgaaggactcagccccgcaagcctgcccccctacccatgccaattgcttgtcccagatccccgttctaactgaccagcggtaaatcaggggttgccacaaccccctcctgggatttgtaacttgctacagcagctcacaaaactcagagaaacacttaacattgaccaattcatcacaaacgatattttgaaaggatgtgaatgaacagccagagaagagatgcacagggcccggggccggggagcagggcatacggagctgccatgccctctcagggggcatcacctcctgcaccagggtgtgttcaaccccaaagctcctgaaccctttaacgtcaggatttttttttatttttttttaaagacatagtctcactctgtctcccaggctggagtgcagtggcgccatctcagctccctgcaagctccgcctcccgggttctcgccattctcctgcctcagcctccccagtagctgggactacaggcgtccgccaccacgcccggctaattttttgtatttttagcggagacggggtttcaccgtgttagccaggatggtctcggtctcctgacctcgtgatccacccacctcggcctcccaaagtgctgggattacaggcgtgagccaccgcgcccggcctaacgtcgggatttttaaggagcttcattacataggcaggactgatgaaatcattggccattgagtgaaccccagaccttgcgggggtggggctgaaagtttcaaccctccaaagattgggcacgttcctctggcactcggcccccagcctccaggagccacctcattagcatacacgcaggtagggttggaaagggcttgtgataaatgatgaaggacgttcttctgcatcgctcggggaattccaagggtttaggggctcactgccaggaacccggggcagaaaccaaatacatatttctcgttatagcacagtgtcaccccctcactctgcctaatttggtgactagctgccccatcacattctgcctatttaagccaagccccccttccccaaggccaacctcctctcctccacagccagcccacttcccgggcgtgataactcttctgcctcagctggagagttgttctgaggctttcatccttctccacgtgccgcctggcagtgctgctgcctgtcttttgagggctacccctttctccattacctctgcgacctggctagtccacatcctccccgacccgtgctcttcagcaccggtgcctgccccgctcagtgcatgtcctcatccctgcagcctccaccctgggcttcctgacccccactgcgtccggcaccgctggttgcgggcctgctccggctctctctgcccagctggctggcctgcctctgttccgacctcccctgcctggcctggtgttctgggcgcctcctccgctcacatcgccgcttcacctgcttttgctatctgcactttccatgtcctgctccttctcccagctggtggtgcctctgagaagaggactgagaaccgcctgtgaaccccgcaatttcgtgggtgtggtggaagcaaaggcagagcgtgtgagtttagtgggcgtgcgccactctttcaagaagttttgttacaaaaagatgcaaaggaagtgaagagggaaggggtttgcaggttgggagaaataacagcatttgtgttgtttgttgttgtgacggttttgagccaaaacatgacaaacgggacagaaggaagacctgatggagcgtgtccttgagaaggcgagaggcatggggttggcctgctgggggatcggccttccatatgggggttcctctccagcagcctggggttctgaggaaggcaggcctgaagcaggtgccgggtgccgggaagcaggagacatctctgttactccactgtcctcagtggggagccacggctgagcgtgagaaagggcttataggctgaaggccaggcagacgggaatggccaggcagaggaggggaggacgagccgggtagaaacagtggatagaaacacggagggccacacggccaacggtcaggggactggcacaccagccagattcacccgcggcgatgccggtgcagagaagctcggcatctgaatttaacccgggttgtggtttgactcagtctgacgtggagagaagggccagggagtcacgggggggtggtgggctgtgtgctggtttaggggctgggacatggaggggtgaaggcgggagtcagtcgcatccgctgggcaggggcctggggctgcagacaaggtgggaggtggcagctacggaggaagctacaagggattctgcagttccccggggaaacaggagcccaagggaccggggggtgagggggttggaaggggcacctgtggatgttctgagacttccaggaagtgggacaggatcagtgatggagatagagacagagtcatcagggccgagaggaatgacagtaacagcgaggttgaagtgggcacccccgtctagcagcacggggtgtggagctggcttgtggacggccagggaacaggacgctttgaggtggcagccaggggcagggatgcttttgatcgccaagggagaagacttgatgcagagtttcaggagcctccatgacttccccatctgaagaccttttttactttaatgggattgaagtgatcaccagaatagttaatggtgtgctccgttcctatttctctggtttttctaaggtccacaggctgcagacatcgtttgtacttctccccggtgccaaagaccagttaatgccgactttgatgggctcagtgcaggccacattgtcacgtgtaactctacactgagaattattttagaaggttagactcctaaaaatgttttgtttttccaaatggtggcctctgggtctgacttcacctcttttgcaatgatcagcactaggatatggttttggagacggttgtgcagagccagggctttcaccaaagcttggccgctcggacaggactcacgatggaagacggtcaggtgccccaggtttcagatgcctgcctcctcccatgcgtggtgaggggcctgcctcctttatagctttccgctgccaggctggcgcctcctcccctcacccccatctcctccagaggaagaccaacttaatcaaatcttaccacaactacgtactgcctcctggaaaaagcctgatttctcgccccctcttgtccctccctgcgtggaggcaggccctttgtccagtgcccatgtggcttggtgggtggtctttctaagttatcagaggacattagcaaacacacacgtccattggcctaacgcccaatctgcagccagccttatgaataatcaacgtgacttgtctctgtagttcaatgcctatatctgcctctcagttgttattgaagctgggggcaaaaaagatggattattcattggaaacctcaaaacctcgacagctgagctttcttacacatgcctgtgtggcccccgtggtatcttagtgttcacctccccatttgcacacaggaagccagtcacattactggattcctggtgagtttgacttttcattctgtcttgaatctccctcccttccccaaccccataccccaccctactccatccctttttcttgggtcttcctgatctcaacccctccatctgtcctccacgttgtctgcatagtgagcctcctaacacacggatccccccatggccttgtctgctcaggtttctaaggtccccagtaaccacgctcacactgcgtaacacgaacggtctggtccacacctcatcacttggcgtgcatgtgaatgttttagcaagttagctcttgcaattattgcctgccgatcccctgggctgcattcacacatgccgtgagtcttcagacacccaggtctcaggacctgaggggctcctgtgtgctttccgtgaggaactgtctttctgctcacgactccatgtcacatgccaccatcaggaagtcctccctcaatgccccaagcctactcaggctcccactttcctgcccatgaaatgtgtgtaacttctagggtgtcctgagaagcaaagaccatgtccctgcatttttgcatcctcagaacttagcctgatactcacaatgaaatgagttcacttaacgacacaacgaacgaatgtgcaggtacttctgcagggggtgatgtggggatgcgtgcattgattctgtggctcagccctgagttgggggcaggaggcaggtgctgggaggaggattttatgtcttaggaagcacaggaaggccttgccaggatccaagaaaaaatggaaagtagaycaatgtaagcgttaaaagaacacattttatcttttaaatgtgtgtacacagtacagttgacttttttgtatacaattctatgagtttaaacacacatatagattagcgtaaccactaattataagattgtagggaactggggaaaaaatgcatgcattaaggaatgatayggcatatttgggggacagagaacaggcttgatgaggacagagtctatttaaaagagacagtgggcacsgcaattggaggggaaggcggggcagggttttagagaacccctgagtgctgggctacaggattcagtaaagttattgatgagattggctgcattgtggattctgaaatatttatttaatacctcgaggagggtgtgagtagattgtgctgatgatcgcataactctgactatactaagaaccactgagttgcacccagagcttgcattactgagcgctttaccagttaggaaggtttcgcgtattccgtactttaaatctaaggtgacttgactgtaaggcctgcgagtatttcctggaccactcagaggaagaatgctgtgaatgagaactacagccctgtaagacacgtcctgtatcgttgttgagatgggaaagtgcatcttaagacggttagcaggccgaggagcgactttaaagggtgagctctgcctagagggaaaagcgaatgcactaattgaaatccaacaccctgggctggagtaaatgaaccgtcagccacccatggggcttcatttcttggtgatggataaatagctgggattccttgaagctagaagccatggggaaattctgttctgcttagctttgtcaacagtacagtctgccttaactgacttggaggtaaatagattcggagagtgtgagctaaaacccattaaatcaggtgaagacacaaaggcaagcacagccaatgtggtttaaggcaaagctaatgtccttcggccttaactgacggactttcctagcagtcctcaccctctgcaacccagggctcctrggaggagctcatggcagagaaagccttctggcttctgccactgcctcctcaactacatgtatacatcagtgtatatgcatgggtatgaaatgaacattttatgtcaccattagcagaggaaagctggaactctttcaaaccccacccaaaattcactctgactactgagcagtcctgttgtttattttggaggccacttaaccctggagcagtccataagctccacttaatcccctcttctttcatgatttcttttaaagagacatcttgggttctgtaggggaacatttgtgcttcactgtaaaactccatttgaggcctgctcacggcctgccaccttatctgcttgcagccttcattgcttgggagctgttttacagcttcataagttgtaaatagctgctggcaatgcaaacgcgcttgtctgtgggcaggaaatgaattctgtctggtagagggaatgcttcctaccttgtaggaaagccaatattttttgtccattagcaagtttatatcagtattcctaatcattaaatgtgttcttcggattgtcctttgaaccagttatagcatttgagttaagtaaaatgaatacactgttgtttattttatacctgtatgaaagttatgggttttttggtggggggggggtgttttttttgttttttttttttgttttttttgaggtggaatctcgttctgtcgcccaggctggagtacagtggcgcaatctcggctcactgcaagctcctcctcccaggttcacaccattcttctgcctcagcttcccaaaagttatgatttttaaaaaattatcttttaacattttttagctagaaacttctgggtcaatatataaatagatgagcctggttatatctgaggttttcactgaggtaacaacaaaaataaaacaacacgatgccaccgagccatcgttccccaacttacgtctgtcccctccacatgtcctgcacacactcctgtttctggggtgtgtgcatgtgtgtgtgtgtgtaaaggtttgcaatgaaattagaatcattggtttttgttgggggtggggagttgtattgttttgagacagggtctcgctctgtcacccacgctggagtgaagggtcacaatcacagttcactgcagcctcaacttcctgggctcaagtgatcctcccacctcagcctcccaagtagcggaaactataggcatgtgacaccatgccgggcttgcttatctatgtctgtctgtctgtctgtctatcatccatctatctatctatctatctaatctatctatctatctatctatctatctatctatctatctatctatctatctttctatctatctagatggggtctccctatgttgcccaggctggtctcaaactcctgggcttaagcaatccaactacctcagcctcccaaagtgctgggattacaggtgttagccactttgcccagctgaagttagagtttagagcacattgctgtaaattgcgattaccaagggtattgaaaaatccatgaaaataataaacagcaagttgacttcagaatttgtgcgtttgaggcttttcgccttgatctccaggtaacacacaggctccttggcgagagccagtggtgatacaatgagaacaccgcctgctgcatctaatatttgcagcttagaattcacagctaactttttaaaatgtaccagtgtgggggaaatggtgctttatttgctggataggaaaattggccaagatcagaattctgaaggcagtgtcacagcacaaagaaactagctactgaagtcacatcctaaacattcgagaggttgatttccttttctactgcattacaaaaaggtttatttactgcttatccatatagtgagatagagattagatctcagtttttggttaagaacaagcattatcataaatgtgtgtgtgtgttgtgtgtgcattttacaggatttttaaaaatacacagagaatttttcacagttgttaactctggtaaatggtggggaaggcaggggtgagaactgatctattattcataatctcaatgatgaacaagctatttccaaaaataggtggattatttaaaattattattattaggatattttgggcttctagaaacaaaaacttaacaaaaaagtcacttaaagaatttaggggtctttttttctgacatgaaaagaacaaaataaaggatgatttcagtttggtccgtcagtgacttagaagtgtttttcaggacccaaggctttccgccttcccactgggccattttcagcgtgtcccgtggcctctgggggcttcagtgatccaggcgtcacattagacatgacagtgtccagcaaagagaagtatttctgctttgcatctgtttataacagtgagaaaaactcccccagaatcccaccagcaattgattctcacgttgcattggccaggattgaggccagctgtgccatgcttagcgcagtcatttgtattgcgatcaccgtgattagctcagacccatcctgggacttctccttgggcttgaagacatggccaggtggagatcggtgccccccagaagaagtctttgttctgccaataaagaagacacagacaacagtgtctaacaggaaaagcccctttttactttatacccttccgtattgcttcaacaatcaaatactttattttattgtttgagacagagtcctgctgtgtcgcccaggctggagcgcagtggcgccatctctgctcactgccacctccacctcccagattcaagcgattctcctgcctcagcctcccgagtagctgggattacaggcgcctaccaaaatgctcggctagtttttgtatttttagtagagatggggtttctccatgttggtcagaccggtctcgaactcctgacctcaagtgatccactcacctcagcctccccaagtgctgggattacaggcgtgagccactgcgcccagccttttttttctttagatagagtgttgctcttattgcccaggctggagtgcagtggcacaatctcagctcactgcaagctccacctcccgggttcacaccattctcctgcctcagcctcccgagtagctgggactacaggtgcccaccaccacgcctggctaatcttttgtatttttagtagagacaggtttcaccatgttagccaggatgatcttgatctcctgaccttgtgacctgcccgcctcagcctcgcaaagtgctgggattacaggtgtgagccaccgtgcccggccagatactttcataattaactttttgaatgtatgtgtgtcctactttaaaatgaaagatactctttcttgattccatttccatgcagcttggccccgtgatgctagggaccatggctttttcttgcagtgtgactcaccatttgccaaagcaaatctcttgccttgcatcagctcagtctctttgtctgcaaattaaatcaatagccctttccactgcctatctcgcaggatatagtgccaaaaatactcacaaagtcaccatccaggaagaatcatttgcccctgctgccactgtctcctgcaaggcacatgaaagctgctgaggctcggtatttattatgctataaaattcaacacaaggggagagaacaagcaaattccatgagcatatataagtgtatcggatctactccattgatgctggagctatattttcacagtaggatcctcttttgttaaatattacagtagtaggaaaacctagcagaagaatagttcactgtttctctgattttgtgagtgatgtgggctgtggaatttactctttgctgctcttcccccaacctgcaccctacccctgcctccgaggtcagccttgcctgctgcccctgactgagaggaccccgacgtcaccccaccccaggttatactcctctgagaaggtcccttcatcccttccccgaaatacatcccctcaaatctctaatttgtgtgaaccattaatttcagatattgtaggaaaaataagcagggaaaatacgcaaaacaaaacgtggatggcacataacccatagcatctcgcagggtgtgtacactgaagaagtctttaccaacccgtagttaggaaaatgcgtgttcagaataactgggccttcccgcggtcctctgagtcaaacagatgaccacacattgccagaatgagaagcagagcagcttcacatccctgcttctgaaatgtttcccaacagctcattgaaacaatctcgagacacctctctcccccaaacccagcgtgtttcgggaatggctctaggaattctacttttgcattgcctcactctccctttccccgtccaaaccatggtattggatttacagcatttcttacatcctataaaagtccttttctgccaagagcctggagcgcgctggattgaatgacgctctcccagcacagccggcatttgcagtgcattagaatcttgccgtcacttgcacacgtcaccaagttactttagtgagagttcagcctagctatggctctgctgtgctaacagttgcttttcaatattttgtttgaggctttggaataattcaaaggcctacacttttttttttctaatttgtttccttggagttttacgcatggctacttcagaaaacgtcagttttatgtcattaatgtcatcatcttctctggattctcagaattcaaaattcacaggagcatggcagccttacattcagtctattcttttcataaaaaaggaagtaaactgcaacagttcgcctacgctatggagactggagtggtcccacctctgtaattctrtctstgtctgccccacagctgtgccgaagygagtgccacttgtctgcagggccgtaccgcggaaccctctttgccgaccagccagygatgtttgtctcgcctgccagcagccccccagtggccaagctctgtgaactagtccacctgtgcggaggccgggtcagccaagtcccccgccaggccagcatcgtcatcgggccctacagcggaaagaagaaagcmacagtcaagtatctgtctgagaaatgggtcttaggtaagaatccaggcacacagacgctgtggtgtggtccagatctgtggacaggtttccagggagggcggcktcaggctcacacccccttccacgcagctggggcacctgggttgatgtctcagcctccagcatctgccctggcagcgtcgtgtggtcaccctcggcattcccgctccttgctgttagcagacgtacagttcacgaggaaatgggaactctgactggacttccccacttgacttccctggctcgtgtgaaaaatccaggctacccaaagccaccccrggccacccctgtgggcacagactctccgggcacccctcttagaccctccctccccagtgcctccttgtcctgcttcaggagtccctggcagcgcccggcactggggcccaagcccccgtccctgtcatctcctctcccaggtacatctcatgatcactccgtctgctcatgtgctcaaagggtgttaaaagacgtcaaacgactccatcttttatttgacaaagtgagcacagtgtgaccgtaatgtcccactctggcgttcatggagctgcgccaggcgccgtgtgcgattctggggaggaagaggtggtaggagctgagctgagatcggaggaggctggaaccccacgccgtgctaacacacgggctccaggagacttgcaggtgatccccggagaagagggttaaggaagagtgtgaagcaaggacggcctggggaatgcggaggaagcaggggcagcgtctgtgctagaaattacctgccctgtggtggagtcatatgtggcgggacaagcctagggctccactgtggggaaatcccacaccctcctccatggggttgtgataaacatgttagtttgcttgggctgccatcgcaaaatactacaggctgggtggcttcaaacaacacgcattgtctctcagttctggaggctggaagtctaagatggggtatcggcagcgttggtttcccctgaggcctctctcctgggcttgcagacagctgccttcttcctgtgacctcacgtggcctttcctccatgcacacacatccctggtatctctgtgtgtgtccaaatgttctcttctctaaggataccagtcagattggattagggctcacccaatggcatacttttatttgcttttatttatttttttgaaacagtgtctcgctctgtcacccaggatggagtgcagtagcatgatcacagcttactgcagcctcagcctctctggctgaagtgattctcctgcctcagcctcccaagtagctggaactacaggtgcacaccacgatgcccagcttttctttctttttttttttttttttttgtagagatggggtctccctatgttgcccaggatagtctcaaactccggggctcaagcgatcctcctgctttggcttcccaaagtgctgggattacaggtgtgagccactgcacccagccccagtggcatcattttaacttgtctttttcaaggccccatctccaaatacagtctcatcctgagttactgagggttaagacatcgacatacgaattttgggcagacacaattcagcccataacaatgaatcactctagtttcagcccctggggccaagatccttacccgactttagaggtacatcccctctctctctctcaatctctctctctctctcccgttctctcattctttttctctctctttgcttccatctccttccatgtttcctattcagtctcctttcttagtacttttgcatgtctctaaatcctaaacttctggcttttctcatccgctgctcaacattatcccttaatagacaagtagatactgtgtttgttcaagttacattcgtatctaactacggacattttacaagtatcttttacatgactgatggtcatcctttcatatattttagaagtgtggcaatcaaaagtaattttttactctggtgcagagtaattcatcttttgcctggaaaccaacttccaaaaaaaaaaaaactatgattttagtcacagtccaaaagctaagaggctgtttactcttttctaaatgccaagaatataaccttcaaaacatcctatgttctgaaacagaggttgttgttttgtttttctggagaagtgtattatcaaaatgccacggactgcagaacagaactgggcctgaaagcatgtctgggccagctgacggaactgtgcacacgattgatatccacagtgcatatcaacaggcagtctttttggagtttgcaaagcgtgtgccgtgcagtgcccgagcctgcctctgcactcgtgtttccaggttgggtggctctgacagccccttcctgtgggtcctgcgtccttgtgtggagtcacgcttgctcggcagctgctcacttcctccggttgttttgccgctcggctctcccgcccgtgggttttcaggaggcgaatgtctacctgcttaatcctgaggcttcgatcccgcaaagcccttcagagttctctgacttccaggccctggccacaggccccagcctctttttctttcctcctgtaacttgtgtcctgtttctgatttctcaccaattatgccatctgcctgtgcccttggtaacatctgggtattgtgtgtgctgcagacctcacccatgtgagacaggtcccctcactcgccggccaccagaccccagtgtagtgggcgtctccagcgtagtgggcgtctccagtgtagtgggcatctccagtgtagtagacctctccagtgtaccaggcctctccagcccacactctctgagatgtaagatcacgtagttctcaagtatttattggcttgtatttttctctttgtgaagtgaattccaatctagtagctgcagctatgtacgaataaagaagggtttatttttctgtccgtacatacttctggcttttctcaccctctgctaaacattatcctttaatagacaagtagatttttttgtatttttctctttgtgaattgaattccaatctggtagctgccgctatgtacaaataaaggaaggtttatttttctgtccatacatacacacgtaaacctacagaacacacagtccagggcattgcgtttcctgcctcatccaggtccaggctatttgcttattctctaaccagaaacaaatcatatacttttttttttttttttctgagatggagtctcgctgtgtcaccaggctggagtgtgcagtgatgagatctcagctcactgcaaccttcacctcctgggttcaagtgattcttctgcctcagccttcccagtagctggaattacaggccccgccaccatgcccaggtaatttttgtatttttagtagagatgaggtttcaccatgttggccaggctggtctcaaacccccaacctcaagtgatcctcctgcctcggcctcccaaagtgctgggattacaggcgtgagccaccgtgcctggccgaaatcacctattttctgtggaatgcatttacttcatgtataaaacagagtcatagcctccaccttgcttaccccacatgctggttaaaggaggaaacacagagagcgcaaatgccctgtggcaggcgtaggcttcttaagtgtggcagattgacggtatccatggatgtgtcctcatcatccctgccccttcgacaaagcacattgtgtcttttggagactttttttcctcccgttcatttccattataacaaatgcttctctggacaatgtttcattctcaaaatatcgcaatattgaaaaactaggaatatatcaaaccattttaaagcaccaaatcgaaaaagaagttattttgtttaaataaattatgaaaagacaatactcaaaaaaaaatcaattaaatttattcaaactggaatatcaactgctttgtaaggtagggtccctgagcgtcttagagtaatttgagccgggcgtggtggcccatgcctgttgtcttagctacgtgggagcttggcttgagcccataagttcaaggctgcggtgagcaacgatcccaccactgtactccagcctaggcaacagagcaagaccccatctctaaaaagaaaaaaaaaagaatcatttttcagtgcctttatattgtttctgtatcttaacagtcttgttttgcagatgtcgtaaactcacagggggtggagaaccaggagttttttagccactaggaacctctctgagaagtttcttttcttttcctttctttattattattagtattttgtggccagaggagggaaaggaaggtgggtactgaaacgacagctcttcccctgggactgcagcatccgagcaccacagtccacccgccagcctttgttcctgcacagtctgcctctcaagaccaacaactccatatctatgacgataaaaattgttagtgattattttacttgtaagaatttctttcgacctcagctctgaggtgaccctcagctcgcccgccaccccagctgccccaccttgctggcatagaacagggagtggaggtgtgaagtcactcaacagggctcagtatacaaaatgtaagccacgcctcactcacttgctccctggagaatttcatctgcgccgcgttgcctaataacggggttatcggaaagggcatgattacgttccctcttcattccctggagtcttttttccctgaaactgtattgtacttgggccaagattcttgatgaatcattcaaccagaaggagaaatggggttgttgtttggtttttttgttttgttttttttttttttttgcgttttgagagagcacacttgtgggtggttgaacatggataaaaataaacgggaaaacaaaaatcaaattcccggccctaggaaataaaatgttacctttacctgatattgataatacatattatatttgaaagcatttgctaatggttgcattttccccccaacactcccatgacatataattcccattttataagtcacgaaacgaagaccctggggtctgaaggaacttggctggggtgaggatcacaagcccttgggtggagctctgagccctggcgcggtcctcaagggtctgcgacatttgtgctgtggtcagctctgtgcactcttccctccctgctgctgttatcacgaaaggctggcttggcctttctcataggcgtatttccactctcaggcgcccttttattgtctgggctccattcaagtgataagacatacatttatgctattgtgggaacataatgtaatattctcaacagcattgccaaacaaaaaaaaagtttagcctctgcctgattttcttataacttataaagaaaatttggtttgaacatgtcccatgtcgatgttttcaggaaaaagatccgatagcatgcaggccttctcatgctggcmtggctcattcatcgtttcccctaatgactgactgaccagaaaaatgcacgacgctcccatggggccactcgggaggcctcaggcttcgggcttcctgattcagtagatatgtgaggcttgatcagtcaccgcagtccacatctccattgcctcgataaggaaccagtcgcagagaggggaggccatctgcagaagctgtggagagtggcagagaggaragtgaggacggggactgcccccttccagcccctctcctccaaggacggcctcattttatccccacccaggtttccacacccaggagctcagcaaccgctcagaaaatgtttgtagaattcaaagacataattcagacaatatgaagaattatttttcctttgagttgttcttaaaacagacgaaatctaccagcatataaatgaatgagaactaaaactggtgggatttggtaatgtcgacatctgagatgtttaggcttttaaatatatatctcagccaggtgcggtggcccatgcctataatcccagcactttaggaggccgaggcgggtgggtcgtttgagcccagcagctcgagtccagcctgggcaacatggtagaatctcgtctgtacaaaaaagtacaataattagcgggcatggtggtgcaagcctatagttgcagctacatgagaggctaaggtgggaggatcacctgagctcagggaggtcagtgctgcagtgagctgtgatcatgccattgcactccagcctgtgcgacagagtgagaacctgtctaaaaatatatatgtgtttatatatatatatttatataaacattagtgggttttaaaaaaaattaactaactgctagctcctaaaacagtattttgccattagctttggaaaggtttgctcagaaaatgaatttctaagcactcccttcattgcatttattggtcaaactaatggtcctggatggttatctttgaaacttcctaacctgttgggtccccgtcgttaaacttatgccaacagaactaaactcactggatgtgaattgcatcagagatgtaaacatttaaaagcgtattaaggctgggcgcagtggctcactcccgtcatcccagcactttgggaggccgaagcgggcggatcatgaggtcaggagatcgagaccatcctggctaacacagtgaaaccccgtctctattaaaaatacagaaaaattagccggtcgtggtggcaggtgcctgtagtcccagctactcaggaggctgaggcaggagaatgcatgaacccgggaggcagagcttgcagtgagccgagatcacgccactgcactccagcctgggcaacagagtaagactctgtctcaaaaaaaaaaaaaaaaaaaaaaaacattaaaagcagaccaagaaaatcctagaatacaggagtcagctgtctattcaattcagaataagaaatattgtagacaaggcaacattttatgtgtattagaaatgtggtggttggtttgagaagtgaaaccagccatgtatatgctgctccaagcattttggttgtggcaggaaactttgaagactattttgctgtacaaattcacaaagccccctgcaaacactcccgtgcttggggtgaatgcccaagtgtgtcacagctgccttgcagctctgaggatcagaaaggttaatggacataaaagaaacttcaaagctcaacctcctaatgggaagctgcccttggttttaggctgtctttgcttactgaccgacttaattcatgctttgggttatgactgtaggagagattttcctgtgtctttggagtatgctgaacttgtgtttctttttgttgttgcatattagacagtcagtgttgaaactaaagtgacctaaagtgacagagctcatgttatgggctgaattttgtctccccagaattcataggttgaagccttcccagtccttagaacatgattgtatctggagctagggcctttaaagacataaataaggtaacatgaggtcataagggcaaggccctaatccaatatgactggtgtccttatacgaagaggaagaggccaggcgtggtggcttacgcctataatcccagcactttgggaggccagggccggcagatcacttgaggtcaggagtttgggaccagtgtgtccaacatggtgaaaccccgtctctactaaaaatgcaaaattagctgggcatggttgtgggcacctgcaatcccagctacttgggaggctgaggcaggagaatcccttgaacacaagaggcggaggctgcagttagtcgtgatcccaccactgcactccaacctgtgcaacagagcaaaaccccatctcaaaaaaataaaaataaaataaaggaagacaaagaaacaccaaagatatttttgcacagagaagagtccaagtgaggactcagggagaaggtggccatctgcaacccgagcagtctcccaggaagcctcaggagaaactaacccctgtgacaccttggtcttggacttcctgccctccagaactgtgaaaaaatacatgtctgctgtttaagccacccaccctgtggcattttgttatggtagcctgagcaaactagttcagcccaaaatgaattctgatatcacctgcagaaatctgcttttagacagcaggaaactgagggcctctgagtttctaggccagagtcatgcagtgaattactgaaagacccagaaccccagtcctggcccctgattttcagtttagaatcttccttggtaagaagcaggatcttaggctgggcccagcaagtggaaaactcttttttatttacacagccactgactgttgtggtctcagactgtaccacagaacctggtgttccacaaacttccccagtttggagcaagagaaaaaagtagttggatgaaatgatctcattttattttttagtcaatttttcttaaatgttggtgcttgaaaacaaatggatggcagtaaagtaatcctgaagaacacaggaggaaagaaataaaagaggcaataccaaatgttagcaaaatggcagcaaggcaaataagaggctcagcaatagcaaaaaactgagttctttggctgggaaaaacttataaatattaaaaatcctgacaatgttgaaaaagaaaggcagagatagggttccaggagaaatactaagaatgaaattggagctgtcactgcagttatcgtaaggatattttaaaatcataagagagcatgatgaacaatttaataccaataaatttgaaaacaggtaagatggatgatttttagaaaaatgttaccaaaattgattcaagaaatagaaaatctaaacaagctcaagcgttaaaaaaattaaataggtaaaatatgtacatcaactgggcacagtggctcacgcctgtaatcccaacactttgggaggctgaagtggacagatcacttgaggtcaggaactagagaccagcctgaccaacacggtgaaaccctgtctttactaaaaatacaaaatgagccaggcatgatggggcatgcctgtgatcccagctacttgggaggctgaggcaggagaatcgcttgaacctgggaggtggaggttgcagtgagccgagactgtgccattgcactccagcctgggcaactagagcaaaactctgtcctaaaaaaaaaaaacaaaaaaaaaacaattatatatcaacaaaaaaaagaaaattttaaaaagtaacaatttgaaaaagtcaaataggcaatcaaaagtattcctttcaccagccactaaaaaggcacctgtacatgggaatggtagcaaaatgacagaagaggaaactctaacctctcatccaacacagaaaccgctaaaaccaggcagaagctgtctgcagagatgttgcaggtgctctaaaaggtgctctaaacaaccaccaaatgcatacagcaaccaggcaaatgcctgatagaggaaagccatcttcaagcccgcaggaaagttttstggcacatggtggcaacccagttcccagttcccagttcccttcctcaagctgcagggagcagaccagacatgatttgttctagtctagctgattcatacctgaaggattgatcctcatctccatctcacataacatgcaaggtgggcaagaaaaagaggtgggcacagctcatgaaagccacagagaggcaattaaggtaaaaatagataaattgcactatatacaaattaaagacttcagtgcatcaaaggatacagtcaacagagtgaaaagcaatctatggaataggagaaaatatttgcaaataacgggttaatcttcacaatatataaagaactcctgcaactcaacaacaaaaaaaaaccccagtttcaaactgagcaaagaacttgaataaacatttcttcaaaaaagatgatataaatgtccaataggcaaatgaaaagatgcttaacattactaatccttaggaagatgcaaatcaaaaccacaatgagatagcacctcagcacctcacacccattatgattgctactataaaaaaaaaaaaaaacccagaaaataacaagtgttagtaaggatgtggaaaattggaaccttgtgtctgcctcatgtaatgttgggaatgtaagatattgtagccacgatagaaaacagtgtggcagttcatcaaaaaatgaaaagtagaattactgtatgatccaacaattcctcttctgggtatatgccaaaaaaattgaaagcaggatctcaaaagaataattgtacatccacatttatagcagcattgttcacaatagccaaaaggcagaagcccaagtgttcatcagtggatgcataagaaacaaaatgtggtctatccatacagtggaatattattcacccttaaaaaggaaggagattctgatacatgtaacactgtggatgaactttgaaaacatcatgttaagtgaaataagccagaaaccaaaggacaaatatcatacgactacacttataagaggaacttagaatagacaaagtcacagagacaaactatagttgaattaccaagggtggagtaggcaggaagggagtggagaattattgtttaatggctacagagactcagttttggataatgagaacattctagaaattaatagtagtgatggctgcacagcattgcgaatgtacttcatgccactgaagtggacacttaaaaatagctaatatggtaaattttatgttatgtctatcaaacttttaaaggcaccctccacagatagttttagtagtaagttttaccaaacattataaagttttacaggaaaaaaaaagaaatctattcacctcattttacaaggctacattgatcttgacctaatactggtttaaaaaactcatttgtaaacaagtacataaaaatctgaggctgagcgcagtgactcatgcctgtaatcccaacactttggaaggccgaggggggcggatcacaaggtcaggagatcgagaccatcctggctaacacagtgaaaccccatctctactaaaaatacaaaaaattagccgggcgtggtggcatgtgcctgtagtcccagctactcgggaggctgaggcaggagaatcacttaaacctgggagaaagaggttgcagtgagccaagagtgcgccattgcactccagcctaggcaacagagtgagactctgtctaagaagaagaaaagaaaaaaaaactcagaaataagatatttcatcaagtcaaatttggtagtgtgtttttaaaacacacacacacataaccaagtgtggtttaacctaagaatgaaaggataaatgaatagcattaagtcttcttttttctaatccattaattttcttagtagtgttaaaaagcagtagggaagattcaatgccgagtaatgatttaaaaaaaaaaaaactcttcagaaaccaggaatagataactttcttaactatggaggttatctataaaaaacgtacaacaaatattgaatggtgaaaaccttagtttaaggcttaaatcaggtacaagacacacatgaatgctattactcttcaacagtgttctatgattcctagtcaagggaataaaataaaaaaaattacaagaattatacaggaagggacacattttgtttgcatgtcatacagttgtctacatagaaacatcaaagagagtcaataaactgttacaactcattcagcaaaattcctctttgtaagatccactcactgaaatctttagcatttgtatacccaatgataaacaattataaaatgtaacagaaaacatagtaaataatagtggattcaaggctagccatgtaatacagattgaacattcctaattttaatctgaaatgctccgatatcttaaactttttgagtgccaacctgtcaacacaagtggaaaattccacacctgacctcatgtgacagggcatagtcaaagcacaggtgcacgacacagttgatttagcgtccccaagggaaaaaaaagacccacccagcccccttcaactatagtataacttttccacgcacacccaaattcccccacacaagcacgcccacaatgtgtaataaaatggcacgtgtgcaggctggacgcacccaacgcagattccccacgatacctcacgtggggccgagaactccatgcattactcactgtggttttttgcttattctctgcagtgtcatgtaaaaatattactgaaaatgtcgaaaaggcctgcagatccccctatgtgtaacagtgatcagaaaaagaggaataatttatgtttatcaatagcacaaacagtcaacttgttggaggaactgaacagcagtataagtgtgaagcgtcttacagaagagtatggtgttgggatgaccaccatacatgacctgaagaaacagaaggatacgcttttgaagttctatgctgaatgtgatgagcagaagttaatgaaaaatagaaaaactctacgtaaagctaaaaatgaagatgtgaatagtgtattgaaaaactagatctgaaggcatcacactgaacccgtgccactcagtggtaggctgatcatgaaacaagcgaagatctatcctgatgaactgaaaattgaagggaactgtgaatattcaacaggctggttgcagaaatttaagaaatgacatggaattcaagttttaaagcatctgcagatcacaaggcagcgtcgaaactcattgacgagtttgccaagattatcgctaatgaaaatctgatgccagaacaagtctgtattgctgatgagacatgaccatttgggtgctactgccccagaaagatgctgactacagctgacgggacagcccctacaggaattaaggatgccaaggacagaatgactgcagtgctgtgcaaatgcagcaggcacgcataagtgtaaacctgctctcatgggcaaaagcttttgtccgtgctgttttcaaagagtaaatttcttaccagtccattattatgctaacaaaaaggcatagatcaccagggacatcttttctgatcggttttacaaacacttcgtacaggcctcttgtgctcgctgcagaaaagttggaccggatgatgacagcaagattttcttatgccttgactactgttctgctcatcctccagctgaaattctcatcaaagataatattgatgctgtgtactttcccccaaacgtgacttcattagttgagcctgtaaccagggtatctttagatcaatgraaagtaaatwtaaaaacactgtcttgaattgcacgctcgcagcagtgaacggaggtgtaggtgtagaagattttcaggagctgagcatgaaggatgccatacatgctgttgccaacacttgcaacacagtgactaaagacacagatgtgcgtgcctggcgtgacctctggcctacgactgtgttcagtgatgatgatgaaccaggtggtggtttagaagaattcagcttgtcaagtgagaagaaaaggatgtctgacctccaaaaaatataccttcagagttcatcagtcagcgggaagaagtacacattaatgtcatttttaacattgataatgaggctccggttgttcatttcattgactgttggggaaatagccagaatggttctgaatcaaggtgatcgtgatgatacgaccatgaagatgacgttaacactgcagaaaaagcacccgtggacagcgtggagctcaggtgtgatgggttaactgaggcccagagcagcgtgcattcacaacagaacaagcaatcatgtcagcttataaaatcaaagaaagaatcctaagacaaaaaagaaagaaaaaaaattagccgggcatggtgacacgtgactatagtcccagctgtgtgggaggctgaggtaagagtcttgcctgagcccaggagttagaggctgcagtgagccgtgatcatgccactgcacaccagcctgggaaacagcgaggccctgtctcaaaaaaacccaaaaaactaagtaaatattttgtacatgaaacaaactttgtgtacactgaaccaacagaaagcagctgtcggttctgagaccattgttagtggtgcagataccattaaaaagccccccagcagaatgcctcctcgtccccagaggacccacttcctgggcctgtaactgcttcttatgttccttctcacctaaaatgtaaaatgccgtgtcccgtaagctttgaatcaaagcacagcatggttgggagagcagaggcctgctgttgtttgttgttgctgctgttgttcagcagctgattgcggtctctgctgatgccactggctgcttagctcccctgagcacgtaagtcttcactgtgttaatggcatgtcttattttttactgtgaagtacttatgtgtgaataagtgtaaggaaatgactgcttggtagtagcatataaattcagagtcacgggcaggcacggtggctcacgcctgtaatcccagcactttgggaggccaaggcgggcagatcgcttgaggccaggagttcaacaccagcctggccaacatggcaaaaccccatctctactaaaaattacaaaaattagccgggcgtgatggcacatgcatgtagtcccagctacttgggaggctgaggcaggggaatcgcttgagcctgggaggtagagattgcagtgagccaagatttcaccactgcactccagcctgggtgacagagagactgtctcaaaaaaaaaaaaaaaaaagtcacagtcaggaatgagggtgatgccacacaaccactgattgtccacatgggggtgagggctgagatagtgatacctctgctttctgatggttccatgtacacagactttgtttcatgcacaaaatttgtttgtttattttttgaaacagagtttggctctgttgcccaggctggtgtacagtgctgcgatcatagctcattgcagcctttaactcctggcctcaagcgatcttcccacctcagcctccgttgtagctgggactacagtcatgctgtcgcacctggcaatcacaccagtctatgcacagaactatttaaaatactgtataaaattacctctaggctatgtgtataagatgcagatgaaacataaatgaattttggttttagactctggtcctatcttcaagatctctcattgtccattccaaaaatgccacccaccaccccccaaaaaaaatctggaattcaaaacatttctggtctccagcattttggataagggacacaccacctgtaatatccttttacacatttcctggatgggaaacagaagttggtgtggtaggagtcacacataaacggcagactttcttgtctgtgacacattcttaggatgtcctagagaagtatcagcgatgtgaatgtctccagtcaaatatcagagcagaaagaatatgttgagaactgctgtattattagactgggctactttcttcaaacaacacatggtatcaggtcattcattcatttacccagtagatatttcctacacacttgtcatatgccgagcatatcctaggcactgcaggtacagcaactgacaggaatatacagcctttgcccttgtgggacttaacatttaagagagaagacaggcagcaaacaatttctttaaaaatccttctggtggtaaatgcaatgaagaaaacagggtgagtatagagaggaggagtgaggtaggccccttgcacgtgagtggcatttgagctgaggcccagatgatgaagagaaggatggactcttgtaggtctattggactggcccttccaggaatggtaagggctgagaggtcaggagaagcggtaagtttagcgtggctgaaatgaagggagagaagacaaagcaataggaaatgaagctggagaagcaggcagcttcagacaggaccattccagaccactgacaccttaacagacaacagcaagaagtttgggttctgttctaaggataaatggaagtcacagaacgattttaagtgggaggattaggctgcggtatatgtttgtttactctgtttgtgtttatttttgttttaatggatacagagtctcccaatgttgcccaggctggttttgaactcctgggctcaacggatcctcctaactcggcctctcaaagtgctgacacatgtttttttaatggaagcagagaaagcagtctcggacctttgcagtggttcaggtgattagtgatgggggttaggaccagggacgtatcgatggaggtgttgtgaagttgtcatattttaaatatacatttcagagccaggtgcactcgctgatacattggatgtggcatattagagaaagaagactcgaaggtggcacctagtcttgtgttctgagcttccagaatgaggcatctagaagccaggacccgggagaagcacggaaggagcagtggtttattcagtctgcagaagcagtgcctacaggactgctgtgtgaaagaggacacatgtgatatgagcagatgaaaatcacacagcaggcagctctgggctcattatgagaaacgactctaggaatatttgtaacctgctgggctctactgctaagggctgccttaagccatgaagccgcagaggctgggtgaccaccgtcccacagtgagggagctgggcaattccttaccagagtggagatgtggctagatctcctagccctaacatgcttacttattttgataagcaaagatgaagctcacatgggtcccgtgtgctcttgaacttctgtacattgtaccattaaccacacttggatgctggcaatcgcagttttagttaaataaagtgacttgcccaccatactataaaaaattaattttggtagcatgttgattctgtatcctaaccataagaccacacagagccatggctagtaaactttagcttgtgcgtaaatgcctgccaagacctgctaaatactgttgcttacatttaaaaaaaaaaaaaaaattttttttttaatttaaatttcacggagctgctcaagggcagttcagcttcctattcatctctgtctccaccggccaggactggcattactctaacatctgtctacggccacattttatgggatgtttgaggattattcctatgaagtgacattggaatttggggatgtggctatgttcagatgccaaataaacttggatagaaatcatttttcctgtgtgtgtttacagttaggaacgtggggctgtgaggggctccctggacatgaccctggagctgtcggcccttgttcagtggtcagatgcgcttcagacctcccagagtgctgcccgcacactcagtcacagccccatgcgcacctcaacgccactgctcagaagtccagtgtaattcctcaggcagcatgtcctagagcaggccatgagaggtgtaaggtacagactttgttgtgaggttacatgtaggcttctgttccatcttgtctctgtttaaagatcgatacttctggcagcctttatccccaccacgataaatacgtggatggaaggatacatgcgtggaagggtggatgggtggatggttggatggatgggtagacgggtgcatgggtagatgggtagatgggtggatggagtgatatttgatttcatagtcaaagaactcaaacagtagacaagtacacagggtcctccagtcttacaacccttccttaactacaataaagatagaagtgtatcttctagatttcttttaaaaacatatttatgaatgtaaacatattatggtcaggtccagtgactcacatgtataatctaacactttgggaagccaaggtgagtggactgcttgatgccgggagtttgagaccagccttggcaacatagaaagaccgtgtccctacaaaaaaaattttaaaagtagcctggtgtcatggcacatgcctgtagtcctagctactcaggacgtcaaggtgggaggatcactcgaggacaggaattccaggctgcagtaagccatgatcataccactgcactccagtctgggcaatggatcaagatcctgtctctttaaaaaaaaaaaaaaacatatttacatagaaataaatgtatataaacacagatattgtttagggatttgtttttatatatattggagaatgacatgctttttcaggagcttttatttaaccctatgcctagaagatccttccagcttaacacatatacagctacttcattctttttaaccattgggaggtactgtaattaatttatgtgctttctgttattttcattgttttgctattgtatttacttatttattttagaaacaagatgtcactatgttgcccaggctggcctcaaactcttgggctcaagcagtcctcccaccttagcctcccaagtaggcgggactacaggcatgaaccctgcaatacggctggcttctgctattttaaactcgtgtgtgtgtgtgtgtgtgtgtgtgtgtgtgtgtgtgtgtgtgtgtgcgcgcgcgtgtgtgtgtgtgtgcgtgtgtgtgtgtgttttctaactgaacaatctgaattcaattttaagagattttcttgagctggaattattctagtccgagcccaggctcatgaagatttctgtaaaatacattccaagcagtgaaattactgtgccctaggatatgtgtacttaaattctgatacacaaggctgcagcaatttacactattactaacggtacataaagtcctatttcctatgtcctataaattcccatgtccagtactggacataacccatattttcaatattgggtgatccgattagttaaaaaaatagatctcattaatttctaattgcctgattactaaattatgaatgagtctgaatatcttagataggagatttatcattcgtgaattacctgtcctgatcccttaactgttttgaaattgggttatttatatttttcacatggttttacagcaatgtttacataatatggacattaaacttttgttgtgttataaaactctgtctctttagctgtgcttatggtgtcttaagtattaccaagtttttaatttttaactattattttttacaaaattaaacacctcttttcctccatggcacctacccttgtggttttgcttagaaaggccttcctcaccctctgagctttaaaaataatctcatattctcctatttatagttttaaaaaatatttagacctttaatgcatgtgcatttcacttactgtataatgtgaggggaccatgttgtttttaataactaatttattgacactgacctatattgccccctgtgagtcatctcttacattcccacatggtatgggtgtgtttctggttattctcgtccattgatctgtttgtctattctgtgctgacctctattttactgctataattgtacagactgttttgatatctggtatgtcaaattttttctcatcatttctctttttaaaaatcatcttcctatgcatttttttctttcctataaactttagaataaacatgtcgttttctttttgaaaagtttgaaatttttggattacattgaatttctagatgaatttggaaagagcatcattttttctgcatttttttatgatttttcaaaactgacacctagtcagaaaactaagtgtaaaaattgaatccatagagtttttacaacctggaagaaaatacaaatgtggctgaatgactttaaaccctgagtatcggaaaaggcttccacctacctatgactcaaaagccagatgcaataagacaaagtgttgatataatttgaatacataagaaattgaaacttatacatggcaaaagtttgcataagaaaagtcaagccaggtgtggtgggttatgtctataatcccagcattttggaagactgaggcacaggaagattgcttgagcccaggagttcgagatcagcctgggcaacaaagtgagacattggctctacaaaaaatcaaaacattaactgggtgtggtggtgcatacctgtagtcccggctacctgggaagctgagtctggaggatcacctgagtccaggagactgaggctgcagtgagtcatgtttgcaccaatgcagtctaacctgcgtgactgagcaagaccctatctcaaaaaaagaaaaaatatgtaaatcataataatacctgcttcactgttgtggagagaattaagtagtatgcctagtactaataatattgttataattatatacaatgtttttaactatatcatttcttatatatataagctatcacaaatgttagtgttcctcccttctgaaattcatctgagggtccctcactgacccaggcctcctgggtagaagcacatttgtattgagaagacaacagttaaattctgggacactatcttgagctataactaagataagtcatttttttcttccatttctaaaaatatttgtagattaaacccatttttttcttttttgtaccataccaccaggatagctttccaccttccatcactcatctgtgtgacttcttaagttccttcaaatgtaactctgtaattataattatatattcacacaatcattgtgattctttaattgcaattgatttaatctaccttatcatccaatcggtgctgacagtggatttcattcctttttttttctaacagtaggaatagaatgcagtgcgcttgccaggactgaggaaagagggaggggttgtttccgccagctgccaggatcacctgtgctgacccttcagcagcacctgcagcgctatcctgggccaggcgcaacttgtgattttcataaaatagtcgagtttcaaacggatgggactttagagcttctttaatttgagctatgaagaacagagttttagaaagtatgcttattcacttggaattccataaaaaatacctatgctgggtagataggatagcacggcctacctctcaccactggtgtcataattaaaactcatatatgtatttacttatactctgccttatgccaagagtactggaagtggtgagctaagattagaaattcttggctcctatgtcacagactggcaagcttcccaccctgcccactgagtgtcctgacacaacgggaacgtgccctgcatctaatgggacatgtggctaccaagcacttgaactggccagtgtgactgagaactgaatgtttcattgtattgaatttcgtttcacgttaatttaaaaaggtatgtgtgctctatggacgtgggggggcctatggacaacacagctcttggctatttgtttttaaatatagtttcatgtatatacaaacaggttatcactttcctatgtggctggctattatgaatgctaaactgcttttcgctctctctctagattccatcacccagcacaaggtctgtgccyctgaaaactacctattgtcacaatgacagtgacctcactggcctgtggtgactgcacacagctcgcaaaactgtctttggatgttcaaatgagaaacaaaactgtgaagagaaggaactggcgtatacaagatgacttctgatatcatgtttgccatgtgttgtggttcttaagaactcataggtgactttctgatgactgaatgtctgtttcagagacgcttcgggcctttttatttttattttattttttattttttgagacggagtcctgccctgtttcccaggctggagtgcaatggcacaatctcggctcactgcaacctccacctcccaggttcaagcgattctgctgcctcagcctcctgagtagctgggattacagatgtgtgccaccatgcctggctaatttttgtagttttagtagagacagggtttcgccatgttggccaggctggtctcaaacgcctgagctcaggtgatctgtcaggcctcttctatagaattccagtctttgtgtcttagtcatgatcataattgaaaggtcacagaacctttgtcattagagcacagtactgccaaataaagaatggaaattcaatgacattgttttattactgagaacaactagagaactctgcaagtttcttggcttagactcgatctttattaatacattatctattaggtaggaaagacatttgtcagctattaaggtgacttttatctagcggagattcctctcttaaagtaatgaaaggagataggtatggggggtgttatacaggataattggtgacatctgagtgtcttacttctgcaagcctgctttatggtgagcaaagcatcaccagcaagtgatcacaatgtccactggccgctttttgcctgccgtcctcgagatgaaattggcagttggggctgattcacagaaacaccgatttgtggctgagcacggtggctcacacctgtcatcccagccctttgggaggctgaggtggacagatcacttgaggtcaggagttcgagaccagcctgaccaacgcagcaaaacccatctctactaaaaatacaaaaatcagctgggtgtggtggcacacacctgtggtcccagctcctcaggagtctgaggcagaagaatcgcttgaacccaagaggcagaggttgcagtgagccaaggttgcagtgaatcaagattgctccactgcactccagcctgggcaacagagtaactctccttctcaaataaataaataaataaataagaaacactgatgtgtctgtcaccttctaaagaaatgaaatgctaggaagtcctagccagagtgatcaggcaagaataagccataaaaggcatccaaataggaaaagaagtcaaactgtctctcttcactgccgatatgattctatacctagaaaaccctaaagactctgccaaaaggctcctggaaccgataaatgacttaagtaaagtttcaggatagtaaatccatgtacaaaaatcagcatttccaaacacagtaacattcaagctgagcaccaaatcaagaacgcaatcccatttccaatagccacggaatgaaatacctaggaacacgtataaccaaggaggcaaaggatctctacaaggagaaccataaacgagatgctgagtcccagcgaggtcggaggtgccactgagccctcatcgtggtgccgttcccgctctgggttatttatctgttgctcatctcagctgttgttcctacctcaaatttcaagtccctcaacaaatataacagaaccacttctagaatgaacctttgagaagggaggtagcagtgcattgtataggaattggcattctatagaaaaccacagaaactggaaataatgaagggttgtctcttggttttaaaataatgtatacacctaaatcatccccttatgatactcatcctctaacagcaattgaacttcaatacaatgagtcattcctgagttcactcgcttcacattacatatgtttctctataaccacaagcatcctggcttggtagtgctcccacagcaccaaaaatccctgaggaggctgacaaacattgtgctgactcatgctggagacaagccacagagaacttccatcccccaccacatcagccacggagccagcccagcctctgcccacccaggcctcagtccccagtgttaagttctgatccctgatgctggcctgccagtggccagtcaagattctctttctgaaagctagtattttatgaggactgactgttgctagacattacactaagcacattatatgttgtacttcattttaccctttcaacaatcctattagtagcttactgtgggtctgcaaagccttactcaaaacatatagggctagaggttctcaggattctgaattttaaaaaaaatttgtaaaggcttatggctctcaccactgttattcaacgttgcattaaagtttctacccagagaaggcaataaaaggaaattaaagctatacagattggaagtgaagaaataaaagtctttattctcaagaatacaagacactatgtatagaaattgtaaggaatgcaaaaaaaaaaaaaaaaaaaagccctacaagaacttataacaagtttagcaagattgcaatatacaatcttgcaatcttcctaaagattatatacaaacctaacagaattgtatttatatatactgtcaataagcaattcaaaatgaaattaagaccacgattccatttaaaattgcatctaaaaataaacaaaataggaatagacttggcaacagttgtaacatctgtatactgaaacctgtaaaacattgctgaaagaagttaaagacttctttaaatagagacatatacaaagttcatagattagaagatgcaatattgttaagatgatagtcctcaaattgacgtatagattcaatgcaatccattaaaatctcagatggctttttatagaatttgaaaagctgatgctaaatcttttatgaaaatgcaaagaacctctagtagacaaaacaatttttttaagagcaaagttggaggatttatagaacctgattccaaaactgtcagtaaaactacaataattacaaagtatcagccaggtgccgtggctcacatctgtaataccagctctctgggaggctgaggcgggtggatcacttgaagtcgggagtttaagaccagcctggccaacttggtgaaaccttgtctctactagaaatacaaaaaattagccaggcatgatgg

SEQ ID NO: 2 - DNA, 3809 bp, Homo sapiens
  5′UTR 1..57
  CDS 58..2565
  3′UTR 2566..3809
  polyA_signal 3795..3800
  allele 285: 5-392-222  : polymorphic base G or T
  allele 968: 4-58-318  : polymorphic base G or T
  allele 997: 4-58-289  : polymorphic base G or C
  allele 2102: 5-398-203  : polymorphic base A or C
  allele 2283: 5-400-175  : polymorphic base C or T
  allele 2339: 5-400-231  : polymorphic base C or T
  allele 2475: 5-400-367  : polymorphic base A or C
  allele 2539: 5-402-144  : polymorphic base C or T
  variation 345: polymorphic base A or G
  variation 615: polymorphic base A or G
  variation 663: polymorphic base T or C
  variation 666: polymorphic base T or C
  variation 853: polymorphic base T or C
  variation 989: polymorphic base T or C
  variation 1309: polymorphic base T or C
  variation 1472: polymorphic base A or C
  variation 1839: polymorphic base A or G
  variation 1913: polymorphic base T or C
  variation 1998: polymorphic base A or G
  variation 2319: polymorphic base T or C
  variation 2359: polymorphic base A or G
  variation 2404: polymorphic base A or G
  variation 2423: polymorphic base T or C
  variation 2454: polymorphic base T or C
  variation 2497: polymorphic base A or G
  variation 2499: polymorphic base A or G
  variation 2533: polymorphic base T or C
  variation 2665: polymorphic base T or C
  variation 2768: insertion of T
  variation 2855: polymorphic base A or G
  variation 2858: polymorphic base A or G
  variation 2867: polymorphic base A or G
  variation 2870: polymorphic base T or A
  variation 2874: polymorphic base A or G
  variation 2881: polymorphic base A or G
  variation 2882: polymorphic base A or G
  variation 2898: polymorphic base A or G
  variation 2910: polymorphic base A or G
  variation 2933: polymorphic base A or G
  variation 2946: polymorphic base A or G
  variation 2957: polymorphic base T or C
  variation 2961: polymorphic base A or G
  variation 2981: polymorphic base A or G
  variation 3001: polymorphic base A or G
  variation 3006: polymorphic base T or C
  variation 3015: polymorphic base A or G
  variation 3027: polymorphic base A or G
gcgccgccaggctcgcaagcaccgcgtaggccagctggccggatcccgccgtctgtcatggcggcccccatcctgaaagatgtagtggcctatgttgaagtgtggtcatccaatggaacagaaaattattcaaagacatttacaacacagcttgtggatatgggggcaaaggtttcaaaaacttttaacaaacaagtaactcacgttatcttcaaagatggctaccagagcacttgggacaaagctcagaagagaggcgtaaagctcgtttcggtgctctgggtkgaaaaatgcaggacagctggagcacacattgatgaatcattgttccctgcagctaatatgaatgaacacttatcaagcctaattaaaaaaaaacgtaaatgtatgcagcccaaagattttaattttaaaacaccagaaaatgataagagatttcagaagaaatttgagaaaatggctaaagagctacaaaggcaaaaaacaaatctagatgatgatgtacctattctcttatttgaatctaatggttcattaatatatactcccacaattgaaattaatagtagtcaccacagcgcaatggagaagagattacaagagatgaaggagaaaagggaaaatctttcccccacctcttcccaaatgattcagcagtctcatgataatccaagtaactctctgtgtgaagcacctttgaacatttcacgtgatactttgtgttcagatgaatactttgctggtggcttacactcatcttttgatgatctttgtggaaactcaggatgtggaaatcaggaaaggaagttggaaggatccattaatgacattaaaagtgatgtgtgtatttcttcacttgtattgaaagcaaataatattcattcatcaccatctttcactcacctcgataaatcaagtcctcagaaatttctgagtaatctttcaaaggaagaaataaacttgcaaakaaatattgcaggtaaagtagtcacccctsaccaaaagcaggctgcaggtatgtctcaggagacgtttgaagagaagtatcgtttgtctcctaccttatcttcaacaaaaggccaccttttgatacattcaagacccaggagttcctcagtaaagagaaaaagagtatcacatggctcccattcacctccgaaggaaaaatgcaagagaaagaggagcaccaggagatctatcatgccgaggctgcagctgtgcaggtcggaaggcaggctgcagcacgtggcgggacctgccctggaggctcttagctgtggggagtcttcatatgatgactatttttcacctgataatcttaaggaaaggtattcagagaatcttcctcctgaatctcagctgccatcaagccctgctcagttgagctgcagaagtctttctaagaaggagagaacaagcatatttgaaatgtctgatttttcctgcgttggcaaaaaaaccagaacagttgacattaccaatttcacagcaaaaaccatctccagtcctcggaaaactggaaatggtgaaggccgtgcaacttcgagttgcgtgacttctgcccctgaagaagccctaaggtgttgtagacaggctgggaaagaagacgcatgcccagagggaaatggcttttcttacaccattgaggaccctgctcttccaaaaggacatgatgatgatttaactcctttggaaggaagccttgaagaaatgaaagaagcggttggtctgaaaagcacacagaacaaaggtaccacttccaaaatatcaaactcctctgaaggcgaagcccagagtgaacatgagccatgttttatagttgactgtaacatggagacgtctacagaagagaaggaaaacttacccggaggatacagtggaagtgttaaaaatagaccaacaaggcatgatgttttagatgactcatgtgacggctttaaggacctcatcaaacctcatgaggaattgaagaaaagtgggagaggcaaaaagccaacaagaacattagtcatgacaagcatgccatctgaaaagcagaatgtcgtcatccaggttgtggataaattgaaaggcttttcaattgcaccagacgtctgtgagamcacgactcacgtgctttccgggaagccacttcgcaccctgaatgtgctgctgggaattgcgcgtggctgctgggttctctcttatgattgggtgctatggtctttagaattgggtcactggatttctgaggagccgttcgaactgtctcaccacttccctgcagctcccctgtgccgaagygagtgccacttgtctgcagggccgtaccgcggaaccctctttgccgaccagccagygatgtttgtctcgcctgccagcagccccccagtggccaagctctgtgaactagtccacctgtgcggaggccgggtcagccaagtcccccgccaggccagcatcgtcatcgggccctacagcggaaagaagaaagcmacagtcaagtatctgtctgagaaatgggtcttagattccatcacccagcacaaggtctgtgccyctgaaaactacctattgtcacaatgacagtgacctcactggcctgtggtgactgcacacagctcgcaaaactgtctttggatgttcaaatgagaaacaaaactgtgaagagaaggaactggcgtatacaagatgacttctgatatcatgtttgccatgtgttgtggttcttaagaactcataggtgactttctgatgactgaatgtctgtttcagagacgcttcgggcctttttatttttattttattttttattttttgagacggagtcctgccctgtttcccaggctggagtgcaatggcacaatctcggctcactgcaacctccacctcccaggttcaagcgattctgctgcctcagcctcctgagtagctgggattacagatgtgtgccaccatgcctggctaatttttgtagttttagtagagacagggtttcgccatgttggccaggctggtctcaaacgcctgagctcaggtgatctgtcaggcctcttctatagaattccagtctttgtgtcttagtcatgatcataattgaaaggtcacagaacctttgtcattagagcacagtactgccaaataaagaatggaaattcaatgacattgttttattactgagaacaactagagaactctgcaagtttcttggcttagactcgatctttattaatacattatctattaggtaggaaagacatttgtcagctattaaggtgacttttatctagcggagattcctctcttaaagtaatgaaaggagataggtatggggggtgttatacaggataattggtgacatctgagtgtcttacttctgcaagcctgctttatggtgagcaaagcatcaccagcaagtgatcacaatgtccactggccgctttttgcctgccgtcctcgagatgaaattggcagttggggctgattcacagaaacaccgatttgtggctgagcacggtggctcacacctgtcatcccagccctttgggaggctgaggtggacagatcacttgaggtcaggagttcgagaccagcctgaccaacgcagcaaaacccatctctactaaaaatacaaaaatcagctgggtgtggtggcacacacctgtggtcccagctcctcaggagtctgaggcagaagaatcgcttgaacccaagaggcagaggttgcagtgagccaaggttgcagtgaatcaagattgctccactgcactccagcctgggcaacagagtaactctccttctcaaataaataaataaataaataagaaaca

SEQ ID NO: 3 - protein, 835 aa, Homo sapiens
  VARIANT 304: Xaa=Arg or Ile
  VARIANT 314: Xaa=His or Asp
  VARIANT 682: Xaa=Thr or Asn
  VARIANT 761: Xaa=Val or Ala
  VARIANT 828: Xaa=Pro or Ser
  VARIANT 91: Xaa=Met or Ile
  VARIANT 306: Xaa=Val or Ala
  VARIANT 413: Xaa=Pro or Ser
  VARIANT 528: Xaa=Asp or Gly
  VARIANT 614: Xaa=Val or Ala
  VARIANT 677: Xaa=Thr or Asn
  VARIANT 756: Xaa=Val or Ala
  VARIANT 758: Xaa=Val or Ala
  VARIANT 809: Xaa=Lys or Glu
  VARIANT 821: Xaa=Cys or Arg
MAAPILKDVVAYVEVWSSNGTENYSKTFTTQLVDMGAKVSKTFNKQVTHVIFKDGYQSTWDKAQKRGVKLVSVLWVEKCRTAGAHIDESLFPAANMNEHLSSLIKKKRKCMQPKDFNFKTPENDKRFQKKFEKMAKELQRQKTNLDDDVPILLFESNGSLIYTPTIEINSSHHSAMEKRLQEMKEKRENLSPTSSQMIQQSHDNPSNSLCEAPLNISRDTLCSDEYFAGGLHSSFDDLCGNSGCGNQERKLEGSINDIKSDVCISSLVLKANNIHSSPSFTHLDKSSPQKFLSNLSKEEINLQXNIAGKVVTPXQKQAAGMSQETFEEKYRLSPTLSSTKGHLLIHSRPRSSSVKRKRVSHGSHSPPKEKCKRKRSTRRSIMPRLQLCRSEGRLQHVAGPALEALSCGESSYDDYFSPDNLKERYSENLPPESQLPSSPAQLSCRSLSKKERTSIFEMSDFSCVGKKTRTVDITNFTAKTISSPRKTGNGEGRATSSCVTSAPEEALRCCRQAGKEDACPEGNGFSYTIEDPALPKGHDDDLTPLEGSLEEMKEAVGLKSTQNKGTTSKISNSSEGEAQSEHEPCFIVDCNMETSTEEKENLPGGYSGSVKNRPTRHDVLDDSCDGFKDLIKPHEELKKSGRGKKPTRTLVMTSMPSEKQNVVIQVVDKLKGFSIAPDVCEXTTHVLSGKPLRTLNVLLGIARGCWVLSYDWVLWSLELGHWISEEPFELSHHFPAAPLCRSECHLSAGPYRGTLFADQPXMFVSPASSPPVAKLCELVHLCGGRVSQVPRQASIVIGPYSGKKKATVKYLSEKWVLDSITQHKVCAXENYLLSQ

SEQ ID NO: 4 - DNA, 18 bp, Artificial Sequence
  sequencing oligonucleotide PrimerPU
tgtaaaacgacggccagt

SEQ ID NO: 5 - DNA, 18 bp, Artificial Sequence
  sequencing oligonucleotide PrimerRP
caggaaacagctatgacc

(19) United States
(12) Patent Application Publication
(10) Pub. No.: US 2003/0165826 A1
(43) Pub. Date: Sep. 4, 2003

(54) PG-3 and biallelic markers thereof

(76) Inventors: Caroline Barry, Les Ulis (FR); Ilya Chumakov, Vaux-le-Penil (FR)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/790,289

(22) Filed: Feb. 21, 2001

Related U.S. Application Data
(63) Continuation-in-part of application No. PCT/IB00/01098, filed on Jul. 28, 2000.
(60) Provisional application No. 60/149,941, filed on Aug. 19, 1999.

(51) Int. Cl. C12Q 1/68; A01K 67/00; C07H 21/04; C12N 9/02

(52) U.S. Cl. 435/6; 435/69.1; 435/189; 435/320.1; 435/325; 800/8; 536/23.2

(57) ABSTRACT

The invention concerns the genomic sequence and cDNA sequences of the PG-3 gene. The invention also concerns biallelic markers of the PG-3 gene. The invention also concerns polypeptides encoded by the PG-3 gene. The invention also deals with antibodies directed specifically against such polypeptides that are useful as diagnostic reagents.

### RELATED APPLICATIONS

The present application is a continuation-in-part of the PCT application N^{o} PCT/IB00/01098 filed on Jul. 28, 2000 which claims priority to U.S. Provisional Patent Application Serial No 60/149,941 filed on August, 19, 1999, the disclosures of which are incorporated herein by reference in their entireties.

### FIELD OF THE INVENTION

The present invention is directed to polynucleotides encoding a PG-3 polypeptide as well as the regulatory regions located at the 5′- and 3′-ends of said coding region. The invention also relates to polypeptides encoded by the PG-3 gene. The invention also relates to antibodies directed specifically against such polypeptides that are useful as diagnostic reagents. The invention further encompasses biallelic markers of the PG-3 gene useful in genetic analysis.

### BACKGROUND OF THE INVENTION

Cancer is one of the leading causes of death in industrialized countries. This makes cancer a serious burden in terms of public health, especially in view of the aging of the population. Indeed, over the next 25 years there will be a dramatic increase in the number of people developing cancer. Globally, 10 million new cancer patients are diagnosed each year and there will be 20 million new cancer diagnoses by the year 2020.

In spite of a large number of available therapeutic techniques including but not limited to surgery, chemotherapy, radiotherapy, bone marow transplantation, and in spite of encouraging results obtained with experimental protocols in immunotherapy or gene therapy, the overall survival rate of cancer patients does not reach 50% after 5 years . Therefore, there is a strong need for both a reliable diagnostic procedure which would enable early-stage cancer prognosis, and for preventive and curative treatments of the disease.

A cancer is a clonal proliferation of cells produced as a consequence of cumulative genetic damage that finally results in unrestrained cell growth, tissue invasion and metastasis (cell transformation). Regardless of the type of cancer, transformed cells carry damaged DNA as gross chromosomal translocations or, more subtly, as DNA amplification, rearrangement or even point mutations.

Cancer is caused by the dysregulation of the expression of certain genes. The development of a tumor requires an important succession of steps. Each of these comprises the dysregulation of a gene either involved in cell cycle activity or in genomic stability and the emergence of an abnormal mutated clone which overwhelms the other normal cell types because of a proliferative advantage. Cancer indeed happens because of a combination of two mechanisms.

Some mutations enhance cell proliferation, increasing the target population of cells for the next mutation. Other mutations affect the stability of the entire genome, increasing the overall mutation rate, as in the case of mismatch repair proteins (reviewed in Amheim N & Shibata D, 1997).

Recent studies have identified three groups of genes which are frequently mutated in cancer. The first two groups are involved in cell cycle activity, which is a mechanism that drives normal cell proliferation and ensures the normal development and homeostasis of the organism. Conversely, many of the properties of cancer cells—uncontrolled proliferation, increased mutation rate, abnormal translocations and gene amplifications—can be attributed directly to perturbations of the normal regulation or progression of the cycle.

The first group of genes, called oncogenes, are genes whose products activate cell proliferation. The normal non-mutant versions are called protooncogenes. The mutated forms are excessively or inappropriately active in promoting cell proliferation and act in the cell in a dominant way such that a single mutant allele is enough to affect the cell phenotype. Activated oncogenes are rarely transmitted as germline mutations since they are probably be lethal when expressed in all the cells in the organism. Therefore oncogenes can only be investigated in tumor tissues. Oncogenes and protooncogenes can be classified into several different categories according to their function. This classification includes genes that code for proteins involved in signal transduction such as: growth factors (i.e., sis, int-2); receptor and non-receptor protein-tyrosine kinases (i.e., erbB, src, bcr-abl, met, trk); membrane-associated G proteins (i.e., ras); cytoplasmic protein kinases (i.e., mitogen-activated protein kinase—MAPK—family, raf, mos, pak), or nuclear transcription factors (i.e., myc, myb, fos, jun, rel) (for review see Hunter T, 1991; Fanger G R et al., 1997; Weiss F U et al., 1997).

The second group of genes which are frequently mutated in cancer, called tumor suppressor genes, are genes whose products inhibit cell growth. Mutant versions in cancer cells have lost their normal function, and act in the cell in a recessive way such that both copies of the gene must be inactivated in order to change the cell phenotype. Most importantly, the tumor phenotype can be rescued by the wild type allele, as shown by cell fusion experiments first described by Harris and colleagues (Harris H et al., 1969). Germline mutations of tumor suppressor genes are transmitted and thus studied in both constitutional and tumor DNA from familial or sporadic cases. The current family of tumor suppressors includes DNA-binding transcription factors (i.e., p53, WT1), transcription regulators (i.e., RB, APC, and BRCA1), and protein kinase inhibitors (i.e., p16), among others (for review, see Haber D & Harlow E, 1997).

The third group of genes which are frequently mutated in cancer, called mutator genes, are responsible for maintaining genome integrity and/or low mutation rates. Loss of function of both alleles increases cell mutation rates, and as a consequence, proto-oncogenes and tumor suppressor genes are mutated. Mutator genes can also be classified as tumor suppressor genes, except for the fact that tumorigenesis caused by this class of genes cannot be suppressed simply by restoration of a wild-type allele, as described above. Genes whose inactivation may lead to a mutator phenotype include mismatch repair genes (i.e., MLH1, MSH2), DNA helicases (i.e., BLM, WRN) or other genes involved in DNA repair and genomic stability (i.e., p53, possibly BRCA1 and BRCA2) (For review see Haber D & Harlow E, 1997; Fishel & Wilson. 1997; Ellis, 1997).

The recent development of sophisticated techniques for genetic mapping has resulted in an ever expanding list of genes associated with particular types of human cancers. The human haploid genome contains an estimated 80,000 to 100,000 genes scattered on a 3×10⁹ base-long double-stranded DNA. Each human being is diploid, i.e., possesses two haploid genomes, one from paternal origin, the other from maternal origin. The sequence of a given genetic locus may vary between individuals in a population or between the two copies of the locus on the chromosomes of a single individual. Genetic mapping techniques often exploit these differences, which are called polymorphisms, to map the location of genes associated with human phenotypes.

One mapping technique, called the loss of heterozygosity (LOH) technique, is often employed to detect genes in which a loss of function results in a cancer, such as the tumor suppressor genes described above. Tumor suppressor genes often produce cancer via a two hit mechanism in which a first mutation, such as a point mutation (or a small deletion or insertion) inactivates one allele of the tumor suppressor gene. Often, this first mutation is inherited from generation to generation. A second mutation, often a spontaneous somatic mutation such as a deletion, which deletes all or part of the chromosome carrying the other copy of the tumor suppressor gene, results in a cell in which both copies of the tumor suppressor gene are inactive. As a consequence of the deletion in the tumor suppressor gene, one allele is lost for any genetic marker located close to the tumor suppressor gene. Thus, if the patient is heterozygous for a marker, the tumor tissue loses heterozygosity, becoming homozygous or hemizygous. This loss of heterozygosity generally provides strong evidence for the existence of a tumor suppressor gene in the lost region.

LOH has allowed the identification of several chromosomic regions associated with cancer. Indeed, substantial amounts of LOH data support the hypothesis that genes associated with distinct cancer types are located within 8p23 region of the human genome. Several regions of chromosome arm 8p were found to be frequently deleted in a variety of human malignacies including those of the prostate, head and neck, lung and colon. Emi et al. demonstrated the involvement of the 8p23.1-8p21.3 region in cases of hepatocellular carcinoma, colorectal cancer, and non-small cell lung cancer (Emi et al., 1992). Yaremko, et al., (1994) showed the existence of two major regions of LOH for chromosome 8 markers in a sample of 87 colorectal carcinomas. The most prominent loss was found for 8p23.1-pter, where 45% of informative cases demonstrated loss of alleles. Scholnick et al. (Scholnick et al, 1996 and Sunwoo et al., 1996) demonstrated the existence of three distinct regions of LOH for the markers of chromosome 8 in cases of squamous cell carcinoma of the supraglottic larynx. They showed that the allelic loss of 8p23 marker D8S264 serves as a statistically significant, independent predictor of poor prognosis for patients with supraglottic squamous cell carcinoma. The study of 51 squamous cell carcinomas of the head and neck and 29 oral squamous cell carcinoma cell lines showed a frequent allelic loss and homozygous deletion at 1 or more loci located in the 8p23 region (Ishwad C S et al., 1999). In addition, a high resolution deletion map of 150 squamous cell carcinomas of the larynx and oral cavity showed two distinct classes of deletion for the 8p23 region within the D8S264 to D8S1788 interval (Sunwoo et al., 1999).

In other studies, Nagai et al. (1997) demonstrated the highest loss of heterozygosity in the specific region of 8p23 by genome wide scanning of LOH in 120 cases of hepatocellular carcinoma (HCC). Further studies using high-density polymorphic marker analysis identified three minimal deleted areas on chromosome 8p, one of them being a 5 cM area in 8p23, probably indicative of the presence of a tumor suppressor loci for HCC (Pineau P, et al., 1999). Gronwald et al. (1997) also demonstrated 8p23-pter loss in renal clear cell carcinomas.

The same region is involved in specific cases of prostate cancer. Matsuyama et al. (1994) showed the specific deletion of the 8p23 band in prostate cancer cases, as monitored by FISH with D8S7 probe. They were able to document a substantial number of cases with deletions of 8p23 but retention of the 8p22 marker LPL. Moreover, Ichikawa et al. (1996) deduced the existence of a prostate cancer metastasis suppressor gene and localized it to 8p23-q12 by studies of metastasis suppression in highly metastatic rat prostate cells after transfer of human chromosomes. Recently Washburn et al. (1997) were able to find substantial numbers of tumors with the allelic loss specific to 8p23 by LOH studies of 31 cases of human prostate cancer. In these samples they were able to define the minimal overlapping region with deletions covering genetic interval D8S262-D8S277. In addition, using PCR analysis of polymorphic microsatellite repeat markers, 29% of 60 prostate tumors showed LOH, at the locus D8S262 of the 8p23 region (Perinchery et al., 1999).

Recent studies have also implicated the 8p23 region in other types of cancers such as fibrous histiocytomas, ovarian adenocarcinomas and gastric cancers. Indeed, comparative genomic hybridization data showed the involvment of the 8p23.1 region in fibrous histiocytomas and detected a minimal amplified region between D8S1819 and D8S550 containing a gene MASL1, the overexpression of which might be oncogenic (Sakabe et al., 1999). LOH was also observed for 27 ovarian adenocarcinomas on 8p. Detailed examination of nine tumours with partial deletions defined three regions of overlap including two in 8p23 (Wright et al., 1998). Comparative genomic hybridization of 58 primary gastric cancers detected gain of the 8p22-23 region in 24% of the tumors and even high-level amplification of the same region in 5% of the tumors. This amplified region was narrowed down to 8p23.1 by reverse-painting FISH to prophase chromosomes (Sakakura et al., 1999).

The present invention relates to PG-3 gene, a gene present in the 8p23 cancer candidate region, as well as diagnostic methods and reagents for detecting alleles of the PG-3 gene which may cause cancer, and therapies for treating cancer.

### SUMMARY OF THE INVENTION

The present invention pertains to nucleic acid molecules comprising the genomic sequence and the cDNA sequence of a novel human gene which encodes a PG-3 protein. The PG-3 gene is localized in the 8p23 candidate region shown to be involved in several types of cancer by LOH studies.

The PG-3 genomic sequence comprises regulatory sequences located upstream (5′-end) and downstream (3′-end) of the transcribed portion of said gene, these regulatory sequences being also part of the invention.

The invention also relates to the cDNA sequence encoding the PG-3 protein, as well as to the corresponding translation product.

Oligonucleotide probes or primers hybridizing specifically with a PG-3 genomic or cDNA sequence are also part of the present invention, as well as DNA amplification and detection methods using said primers and probes.

A further object of the invention relates to recombinant vectors comprising any of the nucleic acid sequences described herein, and in particular to recombinant vectors comprising a PG-3 regulatory sequence or a sequence encoding a PG-3 protein. The present invention also relates to host cells and transgenic non-human animals comprising said nucleic acid sequences or recombinant vectors.

The invention further encompasses biallelic markers of the PG-3 gene useful in genetic analysis.

Finally, the invention is directed to methods for the screening of substances or molecules that inhibit the expression of PG-3, as well as to methods for the screening of substances or molecules that interact with a PG-3 polypeptide or that modulate the activity of a PG-3 polypeptide.

### BRIEF DESCRIPTION OF THE DRAWINGS

FIG. 1 is a block diagram of an exemplary computer system.

FIG. 2 is a flow diagram illustrating one embodiment of a process 200 for comparing a new nucleotide or protein sequence with a database of sequences in order to determine the homology levels between the new sequence and the sequences in the database.

FIG. 3 is a flow diagram illustrating one embodiment of a process 250 in a computer for determining whether two sequences are homologous.

FIG. 4 is a flow diagram illustrating one embodiment of an identifier process 300 for detecting the presence of a feature in a sequence.

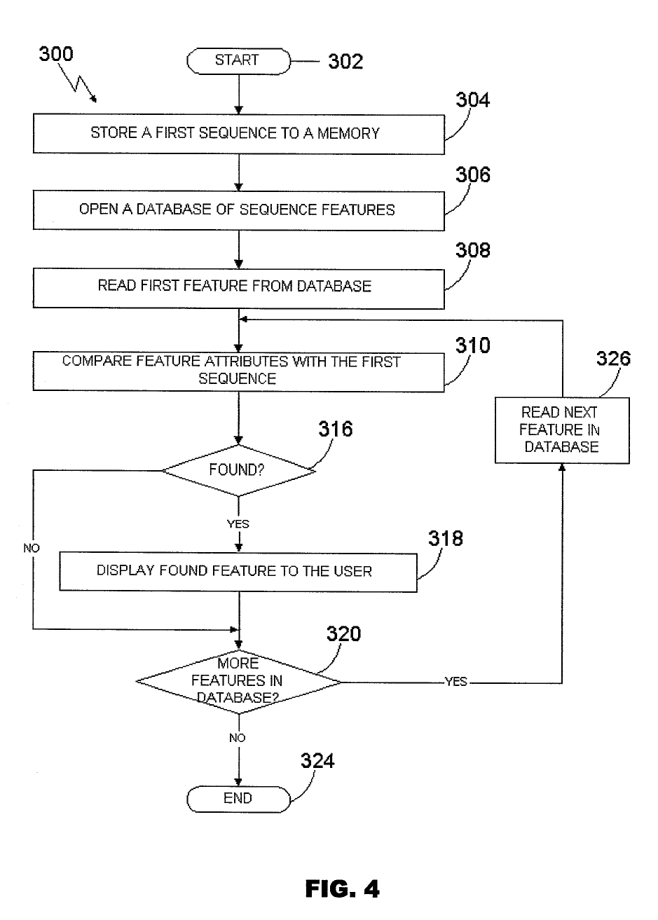

### BRIEF DESCRIPTION OF THE SEQUENCES PROVIDED IN THE SEQUENCE LISTING

SEQ ID No 1 is a genomic sequence of PG-3 comprising the 5′ regulatory region (upstream untranscribed region), the exons and introns, and the 3′ regulatory region (downstream untranscribed region).

SEQ ID No 2 is a cDNA sequence of PG-3.

SEQ ID No 3 is the amino acid sequence encoded by the cDNA of SEQ ID No 2.

SEQ ID No 4 is a primer containing the additional PU 5′ sequence further described in Example 2.

SEQ ID No 5 is a primer containing the additional RP 5′ sequence further described in Example 2.

In accordance with the regulations relating to Sequence Listings, the following codes have been used in the Sequence Listing to indicate the locations of biallelic markers within the sequences and to identify each of the alleles present at the polymorphic base. The code “r” in the sequences indicates that one allele of the polymorphic base is a guanine, while the other allele is an adenine. The code “y” in the sequences indicates that one allele of the polymorphic base is a thymine, while the other allele is a cytosine. The code “m” in the sequences indicates that one allele of the polymorphic base is an adenine, while the other allele is a cytosine. The code “k” in the sequences indicates that one allele of the polymorphic base is a guanine, while the other allele is a thymine. The code “s” in the sequences indicates that one allele of the polymorphic base is a guanine, while the other allele is a cytosine. The code “w” in the sequences indicates that one allele of the polymorphic base is an adenine, while the other allele is a thymine. The nucleotide code of the original allele for each biallelic marker is the following:

| Biallelic marker | Original allele |
|---|---|
| 5-390-177 | C |
| 5-391-43 | G |
| 5-392-222 | T |
| 5-392-280 | T |
| 4-59-27 | G |
| 4-58-289 | C |
| 4-54-199 | A |
| 4-54-180 | C |
| 4-51-312 | G |
| 99-86-266 | A |
| 4-88-107 | G |
| 5-397-141 | G |
| 5-398-203 | C |
| 99-12738-248 | A |
| 99-109-358 | C |
| 99-12749-175 | T |
| 4-21-154 | C |
| 4-21-317 | G |
| 4-23-326 | G |
| 99-12753-34 | A |
| 5-364-252 | G |
| 99-12755-280 | G |
| 99-12755-329 | C |
| 4-87-212 | A |
| 99-12757-318 | C |
| 99-12758-102 | G |
| 99-12758-136 | C |
| 4-105-98 | A |
| 4-105-86 | G |
| 4-45-49 | T |
| 4-44-277 | T |
| 4-86-60 | C |
| 4-84-334 | G |
| 99-78-321 | T |
| 99-12767-36 | G |
| 99-12767-143 | T |
| 99-12767-189 | T |
| 99-12767-380 | G |
| 4-80-328 | C |
| 4-36-384 | C |
| 4-36-264 | G |
| 4-36-261 | C |
| 4-35-333 | A |
| 4-35-240 | G |
| 4-35-173 | T |
| 4-35-133 | C |
| 99-12771-59 | T |
| 99-12774-334 | A |
| 99-12776-358 | G |
| 99-12781-113 | A |
| 4-104-298 | C |
| 4-104-254 | G |
| 4-104-250 | C |
| 4-104-214 | A |
| 99-12818-289 | T |
| 99-24807-271 | C |
| 99-24807-84 | G |
| 99-12831-157 | G |
| 99-12831-241 | C |
| 99-12832-387 | T |
| 99-12836-30 | G |
| 99-12844-262 | C |
| 4-24-74 | C |
| 4-24-246 | C |
| 4-24-314 | G |
| 4-27-190 | A |
| 5-400-145 | G |
| 5-400-149 | G |
| 5-400-175 | T |
| 5-400-231 | T |
| 5-400-367 | A |
| 99-12852-110 | T |
| 99-12852-325 | A |
| 4-37-326 | A |
| 4-37-107 | G |
| 5-270-92 | G |
| 99-12860-47 | G |
| 99-12860-57 | T |
| 5-402-144 | C |

In some instances, the polymorphic bases of the biallelic markers alter the identity of an amino acid in the encoded polypeptide. This is indicated in the accompanying Sequence Listing by use of the feature VARIANT, placement of an Xaa at the position of the polymorphic amino acid, and definition of Xaa as the two alternative amino acids. For example 1f one allele of a biallelic marker is the codon CAC, which encodes histidine, while the other allele of the biallelic marker is CAA, which encodes glutamine, the Sequence Listing for the encoded polypeptide will contain an Xaa at the location of the polymorphic amino acid. In this instance, Xaa would be defined as being histidine or glutamine.

### DETAILED DESCRIPTION

The present invention concerns polynucleotides and polypeptides related to the PG-3 gene. Oligonucleotide probes and primers hybridizing specifically with a genomic or a cDNA sequence of PG-3 are also part of the invention. A further object of the invention relates to recombinant vectors comprising any of the nucleic acid sequences described in the present invention, and in particular recombinant vectors comprising a regulatory region of PG-3 or a sequence encoding the PG-3 protein, as well as host cells comprising said nucleic acid sequences or recombinant vectors. The invention also encompasses methods of screening for molecules which regulates the expression of the PG-3 gene or which modulate the activity of the PG-3 protein. The invention also relates to antibodies directed specifically against such polypeptides that are useful as diagnostic reagents.

The invention also concerns PG-3-related biallelic markers which can be used in any method of genetic analysis including linkage studies in families, linkage disequilibrium studies in populations and association studies of case-control populations. An important aspect of the present invention is that biallelic markers allow association studies to be performed to identify genes involved in complex traits. These biallelic markers may lead to allelic variants of the PG-3 protein.

### Definitions

Before describing the invention in greater detail, the following definitions are set forth to illustrate and define the meaning and scope of the terms used to describe the invention herein.

The terms “PG-3 gene”, when used herein, encompasses genomic, mRNA and cDNA sequences encoding the PG-3 protein, including the untranscribed regulatory regions of the genomic DNA.

The term “PG-3 biological activity” is intended for polypeptides exhibiting an activity similar, but not necessarily identical, to an activity of the PG-3 polypeptide of the invention as described herein, especially in the section entitled “PG-3 polypeptide biological activities”. In contrast, the term “biological activity” refers to any activity that a polypeptide of the invention may have.

The term “heterologous protein”, when used herein, is intended to designate any protein or polypeptide other than the PG-3 protein. More particularly, the heterologous protein may be a compound which can be used as a marker in further experiments with a PG-3 regulatory region.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such a polynucleotide could be part of a vector and/or such a polynucleotide or polypeptide could be part of a composition, and still be isolated in that the vector or composition is not part of its natural environment.

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude. To illustrate, individual cDNA clones isolated from a cDNA library have been conventionally purified to electrophoretic homogeneity. The sequences obtained from these clones could not be obtained directly either from the library or from total human DNA. The cDNA clones are not naturally occurring as such, but rather are obtained via manipulation of a partially purified naturally occurring substance (messenger RNA). The conversion of mRNA into a cDNA library involves the creation of a synthetic substance (cDNA) and pure individual cDNA clones can be isolated from the synthetic library by clonal selection. Thus, creating a cDNA library from messenger RNA and subsequently isolating individual clones from that library results in an approximately 10⁴-10⁶ fold purification of the native message.

The term “purified” is further used herein to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. The term “purified” may also be used to specify the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polypeptide or polynucleotide typically comprises about 50%, preferably 60 to 90% weight/weight of a polypeptide or polynucleotide sample, respectively, more usually about 95%, and preferably is over about 99% pure. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity. Each number representing a percent purity, to the thousandth position, may be claimed as individual species of purity.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Creighton (1993); Seifter et al., (1990); Rattan et al., (1992).) Also included within the definition are polypeptides which contain one or more analogs of an amino acid (including, for example, non-naturally occurring amino acids, amino acids which only occur naturally in an unrelated biological system, modified amino acids from mammalian systems, etc. . . . ), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, this term means that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) # of the number of nucleic acid inserts in the population of recombinant backbone molecules.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (i.e. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar. For examples of analogous linking groups, purine, pyrimidines, and sugars see for example PCT publication No. WO 95/04064, which disclosure is hereby incorporated by reference in its entirety. Preferred modifications of the present invention include, but are not limited to, 5-fluorouracil, 5-bromouracil, 5-chlorouracil, 5-iodouracil, bypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v) ybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. The polynucleotide sequences of the invention may be prepared by any known method, including synthetic, recombinant, ex vivo generation, or a combination thereof, as well as utilizing any purification methods known in the art. Methylenemethylimino linked oligonucleosides as well as mixed backbone compounds having, may be prepared as described in U.S. Pat. Nos. 5,378,825; 5,386,023; 5,489,677; 5,602,240; and 5,610,289, which disclosures are hereby incorporated by reference in their entireties. Formacetal and thioformacetal linked oligonucleosides may be prepared as described in U.S. Pat. Nos. 5,264,562 and 5,264,564, which disclosures are hereby incorporated by reference in their entireties. Ethylene oxide linked oligonucleosides may be prepared as described in U.S. Pat. No. 5,223,618, which disclosure is hereby incorporated by reference in its entirety. Phosphinate oligonucleotides may be prepared as described in U.S. Pat. No. 5,508,270, which disclosure is hereby incorporated by reference in its entirety. Alkyl phosphonate oligonucleotides may be prepared as described in U.S. Pat. No. 4,469,863, which disclosure is hereby incorporated by reference in its entirety. 3′-Deoxy-3′-methylene phosphonate oligonucleotides may be prepared as described in U.S. Pat. Nos. 5,610,289 or 5,625,050 which disclosures are hereby incorporated by reference in their entireties. Phosphoramidite oligonucleotides may be prepared as described in U.S. Pat. No. 5,256,775 or U.S. Pat. No. 5,366,878 which disclosures are hereby incorporated by reference in their entireties. Alkylphosphonothioate oligonucleotides may be prepared as described in published PCT applications WO 94/17093 and WO 94/02499 which disclosures are hereby incorporated by reference in their entireties. 3′-Deoxy-3′-amino phosphoramidate oligonucleotides may be prepared as described in U.S. Pat. No. 5,476,925, which disclosure is hereby incorporated by reference in its entirety. Phosphotriester oligonucleotides may be prepared as described in U.S. Pat. No. 5,023,243, which disclosure is hereby incorporated by reference in its entirety. Borano phosphate oligonucleotides may be prepared as described in U.S. Pat. Nos. 5,130,302 and 5,177,198 which disclosures are hereby incorporated by reference in their entireties.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest. As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease, a beneficial response to or side effects related to a treatment or a vaccination. Said disease can be, without being limited to, cancer, developmental diseases, neurological diseases, disorders relating to abnormal cellular differentiation, proliferation, or degeneration, including but not limioted to hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease; said disease is preferably cancer or a disorder relating to abnormal cellular differentiation, proliferation, or degeneration, and even more preferably said disease is cancer of the prostate, head, neck, lung, liver, kidney, ovary, stomach or colon. Preferably, the term “trait” or “phenotype”, when used herein, encompasses, but is not limited to, diseases, early onsets of diseases, a beneficial response to or side effects related to treatment or a vaccination against diseases, a susceptibility to diseases, the level of aggressiveness of diseases, a modified or forthcoming expression of the PG-3 gene, a modified or forthcoming production of the PG-3 protein, or the production of a modified PG-3 protein.

The term “allele” is used herein to refer to variants of a nucleotide sequence. A biallelic polymorphism has two forms. Typically the first identified allele is designated as the original allele whereas other alleles are designated as alternative alleles. Diploid organisms may be bomozygous or heterozygous for an allelic form.

The term “heterozygosity rate” is used herein to refer to the incidence of individuals in a population which are heterozygous at a particular allele. In a biallelic system, the heterozygosity rate is on average equal to ²Pa(1-Pa), where Pa is the frequency of the least common allele. In order to be useful in genetic studies, a genetic marker should have an adequate level of heterozygosity to allow a reasonable probability that a randomly selected person will be heterozygous.

The term “genotype” as used herein refers the identity of the alleles present in an individual or a sample. In the context of the present invention, a genotype preferably refers to the description of the biallelic marker alleles present in an individual or a sample. The term

“genotyping” a sample or an individual for a biallelic marker consists of determining the specific allele or the specific nucleotide carried by an individual at a biallelic marker.

The term “mutation” as used herein refers to a difference in DNA sequence between or among different genomes or individuals which has a frequency below 1%.

The term “haplotype” refers to a combination of alleles present in an individual or a sample. In the context of the present invention, a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

The term “biallelic polymorphism” and “biallelic marker” are used interchangeably herein to refer to a single nucleotide polymorphism having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., 1995).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym of “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

The terms “comprising”, “consisting of” and “consisting essentially of” may be interchanged for one another throughout the instant application”. The term “having” has the same meaning as “comprising” and may be replaced with either the term “consisting of” or “consisting essentially of”.

Unless otherwise specified in the application, nucleotides and amino acids of polynucleotides and polypeptides respectively of the present invention are contiguous and not interrupted by heterologous sequences.

Identity Between Nucleic Acids or Polypeptides

The terms “percentage of sequence identity” and “Percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, CLUSTALW, FASTDB (Pearson and Lipman, 1988; Altschul et al., 1990; Thompson et al., 1994; Higgins et al., 1996; Altschul et al., 1993; Brutlag et al, 1990), the disclosures of which are incorporated by reference in their entireties.

In a particularly preferred embodiment, protein and nucleic acid sequence homologies are evaluated using the Basic Local Alignment Search Tool (“BLAST”) which is well known in the art (see, e.g., Karlin and Altschul, 1990; Altschul et al., 1990, 1993, 1997), the disclosures of which are incorporated by reference in their entireties. In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992; Henikoff and Henikoff, 1993), the disclosures of which are incorporated by reference in their entireties. Less preferably, the PAM or PAM250 matrices may also be used (see, e.g., Schwartz and Dayhoff, eds., 1978), the disclosure of which is incorporated by reference in its entirety. The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990), the disclosure of which is incorporated by reference in its entirety. The BLAST programs may be used with the default parameters or with modified parameters provided by the user.

Another preferred method for determining the best overall match between a query nucleotide sequence (a sequence of the present invention) and a subject sequence, also referred to as a global sequence alignment, can be determined using the FASTDB computer program based on the algorithm of Brutlag et al. (1990), the disclosure of which is incorporated by reference in its entirety. In a sequence alignment the query and subject sequences are both DNA sequences. An RNA sequence can be compared by first converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is 35 shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using 10, the specified parameters, to arrive at a final percent identity score. This corrected score is what is used for the purposes of the present invention. Only nucleotides outside the 5′ and 3′ nucleotides of the subject sequence, as displayed by the FASTDB alignment, which are not matched/aligned with the query sequence, are calculated for the purposes of manually adjusting the percent identity score. For example, a 90 nucleotide subject sequence is aligned to a 100 nucleotide query sequence to determine percent identity. The deletions occur at the 5′ end of the subject sequence and therefore, the FASTDB alignment does not show a matched/alignment of the first 10 nucleotides at 5′ end. The 10 unpaired nucleotides represent 10% of the sequence (number of nucleotides at the 5′ and 3′ ends not matched/total number of nucleotides in the query sequence) so 10% is subtracted from the percent identity score calculated by the FASTDB program. If the remaining 90 nucleotides were perfectly matched the final percent identity would be 90%. In another example, a 90 nucleotide subject sequence is compared with a 100 nucleotide query sequence. This time the deletions are internal deletions so that there are no nucleotides on the 5′ or 3′ of the subject sequence which are not matched/aligned with the query. In this case the percent identity calculated by FASTDB is not manually corrected. Once again, only nucleotides 5′ and 3′ of the subject sequence which are not matched/aligned with the query sequence are manually corrected. No other manual corrections are made for the purposes of the present invention.

Another preferred method for determining the best overall match between a query amino acid sequence (a sequence of the present invention) and a subject sequence, also referred to as a global sequence alignment, can be determined using the FASTDB computer program based on the algorithm of Brutlag et al. (1990). In a sequence alignment the query and subject sequences are both amino acid sequences. The result of said global sequence alignment is in percent identity. Preferred parameters used in a FASTDB amino acid alignment are: Matrix=PAM 0, k-tuple=2, Mismatch Penalty=1, Joining Penalty-20, Randomization Group25Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence due to N-or C-terminal deletions, not because of internal deletions, the results, in percent identity, must be manually corrected. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query amino acid residues outside the farthest N- and C-terminal residues of the subject sequence. For example, a 90 amino acid residue subject sequence is aligned with a 100-residue query sequence to determine percent identity. The deletion occurs at the N-terminus of the subject sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90-residue subject sequence is compared with a 100-residue query sequence. This time the deletions are internal so there are no residues at the N- or C-termini of the subject sequence, which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected. No other manual corrections are made for the purposes of the present invention.

The term “percentage of sequence similarity” refers to comparisons between polypeptide sequences and is determined by comparing two optimally aligned sequences over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which an identical or equivalent amino acid residue occurs in both sequences to yield the number of matched positions, dividing the number of matched positions by the total number of positions in the window of comparison and multiplying the result by 100 to yield the percentage of sequence similarity. Similarity is evaluated using any of the variety of sequence comparison algorithms and programs known in the art, including those described above in this section. Equivalent amino acid residues are defined herein.

Hybridization Conditions

Stringent Hybridization Conditions

“Stringent hybridization conditions” are defined as conditions in which only nucleic acids having a high level of identity to the probe are able to hybridize to said probe. These conditions may be calculated as follows:

For probes between 14 and 70 nucleotides in length the melting temperature (Tm) is calculated using the formula: Tm=81.5+16.6(log (Na⁺))+0.41 (fraction G+C)−(600/N) where N is the length of the probe.

If the hybridization is carried out in a solution containing formamide, the melting temperature may be calculated using the equation: Tₘ=81.5+16.6(log (Na⁺))+0.41(fraction G+C)−(0.63% formamide)−(600/N) where N is the length of the probe.

Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. The formulas for SSC and Denhardt's solutions are listed in Sambrook et al., 1986.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to nucleic acids containing sequences complementary thereto or homologous thereto. For probes over 200 nucleotides in length, the hybridization may be carried out at 15-25° C. below the Tm. For shorter probes, such as oligonucleotide probes, the hybridization may be conducted at 15-25° C. below the Tm. Preferably, for hybridizations in 6×SSC, the hybridization is conducted at approximately 68° C. Preferably, for hybridizations in 50% formamide containing solutions, the hybridization is conducted at approximately 42° C.

Following hybridization, the filter is washed in 2×SSC, 0.1% SDS at room temperature for 15 minutes. The filter is then washed with 0.1×SSC, 0.5% SDS at room temperature for 30 minutes to 1 hour. Thereafter, the solution is washed at the hybridization temperature in 0.1×SSC, 0.5% SDS. A final wash is conducted in 0.1×SSC at room temperature.

Nucleic acids which have hybridized to the probe are identified by autoradiography or other conventional techniques.

Other conditions of high stringency which may be used are well known in the art and are cited in Sambrook et al., 1989; and Ausubel et al., 1989. By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in prehybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in Hames and Higgins (1985) or in Sambrook et al.(1989).

Low and Moderate Conditions

Changes in the stringency of hybridization and signal detection are primarily accomplished through the manipulation of formamide concentration (lower percentages of formamide result in lowered stringency); salt conditions, or temperature. The above procedure may thus be modified to identify nucleic acids having decreasing levels of identity to the probe sequence. For example, the hybridization temperature may be decreased in increments of 5° C. from 65° C. to 42° C. in a hybridization buffer having a sodium concentration of approximately 1M. Following hybridization, the filter may be washed with 2×SSC, 0.5% SDS at the temperature of hybridization. These conditions are considered to be “moderate” conditions above 50° C. and “low” conditions below 50° C. Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of identity to the probe. Following hybridization, the filter may be washed with 6×SSC, 0.5% SDS at 50° C. These conditions are considered to be “moderate” conditions above 25% formamide and “low” conditions below 25% formamide. cDNAs or genomic DNAs which have hybridized to the probe are identified by autoradiography or other conventional techniques.

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### POLYNUCLEOTIDES OF THE INVENTION

1) Genomic Sequences of the PG-3 Gene

The present invention concerns the genomic sequence of PG-3. The present invention encompasses compositions containing the PG-3 gene, or PG-3 genomic sequences consisting of, consisting essentially of, or comprising the sequence of SEQ ID No 1, sequences complementary thereto, as well as fragments and variants thereof. These polynucleotides may be purified, isolated, or recombinant.

Particularly preferred nucleic acids of the invention include isolated, purified, or recombinant polynucleotides in compositions comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-97921, 98517-103471, 103603-108222, 108390-109221, 109324-114409, 114538-115723, 115957-122102, 122225-126876, 127033-157212, 157808-240825. Additional preferred nucleic acids of the invention include isolated, purified, or recombinant polynucleotides in compositions comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-10000, 10001-20000, 20001-30000, 30001-0000, 40001-50000, 50001-60000, 60001-70000, 70001-80000, 80001-90000, 90001-97921, 98517-103471, 103603-108222, 108390-109221, 109324-114409, 114538-115723, 115957-122102, 122225-126876, 127033-157212, 157808-159000, 159001-160000, 160001-170000, 170001-180000, 180001-190000, 190001-200000, 200001-210000, 210001-220000, 220001-230000, 230001-240825. It should be noted that nucleic acid fragments of any size and sequence may also be comprised by the polynucleotides described in this section.

The PG-3 genomic nucleic acid comprises 14 exons. The exon positions in SEQ ID No 1 are detailed below in Table A.

TABLE A

| | Position in SEQ ID No 1 | | | Position in SEQ ID No 1 | |
|---|---|---|---|---|---|
| Exon | Beginning | End | Intron | Beginning | End |
| A | 2001 | 2079 | A-B | 2080 | 4626 |
| B | 4627 | 4718 | B-C | 4719 | 10114 |
| C | 10115 | 10233 | C-D | 10234 | 26809 |
| D | 26810 | 26897 | D-E | 26898 | 31356 |
| E | 31357 | 31471 | E-F | 31472 | 34260 |
| F | 34261 | 34404 | F-S | 34405 | 37376 |
| S | 37377 | 37466 | S-T | 37467 | 39703 |
| T | 39704 | 40858 | T-G | 40859 | 50435 |
| G | 50436 | 50545 | G-H | 50546 | 72880 |
| H | 72881 | 72918 | H-I | 72919 | 75988 |
| I | 75989 | 76151 | I-J | 76152 | 95110 |
| J | 95111 | 95188 | J-K | 95189 | 216014 |
| K | 216015 | 216252 | K-L | 216253 | 237525 |
| L | 237526 | 238825 | | | |

Thus, the invention embodies compositions containing purified, isolated, or recombinant polynucleotides comprising a nucleotide sequence selected from the group consisting of the 14 exons of the PG-3 gene, or a sequence complementary thereto. The invention also relates to compositions containing purified, isolated, or recombinant nucleic acids comprising a combination of at least two exons of the PG-3 gene, wherein the polynucleotides are arranged within the nucleic acid, from the 5′-end to the 3′-end of said nucleic acid, in the same order as in SEQ ID No 1.

Intron A-B refers to the nucleotide sequence located between Exon A and Exon B, and so on. The position of the introns is detailed in Table A. The intron J-K is large. Indeed, it is 120 kb in length and comprises the whole angiopoietine gene.

Thus, the invention embodies compositions containing purified, isolated, or recombinant polynucleotides comprising a nucleotide sequence selected from the group consisting of the 13 introns of the PG-3 gene, or a sequence complementary thereto.

While this section is entitled “Genomic Sequences of PG-3,” it should be noted that nucleic acid fragments of any size and sequence may also be comprised by the polynucleotides described in this section, flanking the genomic sequences of PG-3 on either side or between two or more such genomic sequences.

2) PG-3 cDNA Sequences

The expression of the PG-3 gene has been shown to lead to the production of at least one mRNA species which nucleic acid sequence is set forth in SEQ ID No 2. Three cDNAs have been independently cloned. They all have the same size but exhibit strong polymorphism between each other and between each cDNA and the genomic seqeunce. These polymorphisms are indicated in the appended sequence listing by the use of the feature “variation” in SEQ ID No 2.

Another object of the invention is a composition comprising a purified, isolated, or recombinant nucleic acid comprising the nucleotide sequence of SEQ ID No 2, complementary sequences thereto, as well as allelic variants, and fragments thereof. Moreover, preferred polynucleotide compositions of the invention include purified, isolated, or recombinant PG-3 cDNAs consisting of, consisting essentially of, or comprising the sequence of SEQ ID No 2.

Preferred embodiments of the invention include compositions containing isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2: 1-500, 501-1000, 1001-1500, 1501-2000, 2001-2500, 2501-3000, 3001-3500, 3501-3809.

The cDNA of SEQ ID No 2 includes a 5′-UTR region starting from the nucleotide at position 1 and ending at the nucleotide in position 57 of SEQ ID No 2. The cDNA of SEQ ID No 2 includes a 3′-UTR region starting from the nucleotide at position 2566 and ending at the nucleotide at position 3809 of SEQ ID No 2. The polyadenylation signal starts from the nucleotide at position 3795 and ends at the nucleotide in position 3800 of SEQ ID No 2.

Consequently, the invention concerns a composition containing a purified, isolated, or recombinant nucleic acid comprising a nucleotide sequence of the 5′ UTR of the PG-3 cDNA, a sequence complementary thereto, or an allelic variant thereof. The invention also concerns a composition containing a purified, isolated, or recombinant nucleic acid comprising a nucleotide sequence of the 3UTR of the PG-3 cDNA, a sequence complementary thereto, or an allelic variant thereof.

While this section is entitled “PG-3 cDNA Sequences,” it should be noted that nucleic acid fragments of any size and sequence may also be comprised by the polynucleotides described in this section, flanking the PG-3 sequences on either side or between two or more such PG-3 sequences.

3) Coding Regions

The PG-3 open reading frame is contained in the corresponding mRNA of SEQ ID No 2. More precisely, the effective PG-3 coding sequence (CDS) includes the region between nucleotide position 58 (first nucleotide of the ATG codon) and nucleotide position 2565 (end nucleotide of the TGA codon) of SEQ ID No 2.

The present invention also embodies compositions containing isolated, purified, and recombinant polynucleotides which encode a polypeptide comprising a contiguous span of at least 6 amino acids, preferably at least 8 or 10 amino acids, more preferably at least 12, 15, 20, 25, 30, 40, 50, 100, 150, 200, 250, 300, 400, 500, 600, 700 or 800 amino acids of SEQ ID No 3. Preferably, the present invention also embodies compositions containing isolated, purified, and recombinant polynucleotides which encode a polypeptide comprising a contiguous span of at least 6 amino acids, preferably at least 8 or 10 amino acids, more preferably at least 12, 15, 20, 25, 30, 40, 50, or 100 amino acids of SEQ ID No 3, wherein wherein said contiguous span comprises at least 1, 2, 3, 5, or 10 of the following amino acid positions of SEQ ID No 3: 1-100, 101-200, 201-300, 301-400, 401-500, 501-600, 601-700, 701-835.

The above disclosed polynucleotide that contains the coding sequence of the PG-3 gene may be expressed in a desired host cell or a desired host organism, when this polynucleotide is placed under the control of suitable expression signals. The expression signals may be either the expression signals contained in the regulatory regions in the PG-3 gene of the invention or in contrast the signals may be exogenous regulatory nucleic sequences. Such a polynucleotide, when placed under the suitable expression signals, may also be inserted in a vector for its expression and/or amplification.

4) Regulatory Sequences Of PG-3

As mentioned, the genomic sequence of the PG-3 gene contains regulatory sequences both in the non-transcribed 5′-flanking region and in the non-transcribed 3′-flanking region that border the PG-3 coding region containing the 14 exons of this gene.

The 5′ regulatory region of the PG-3 gene is localized between the nucleotide in position 1 and the nucleotide in position 2000 of the nucleotide sequence of SEQ ID No 1. The 3′ regulatory region of the PG-3 gene is localized between nucleotide position 238826 and nucleotide position 240825 of SEQ ID No 1.

Polynucleotides derived from the 5′ and 3′ regulatory regions are useful in order to detect the presence of at least a copy of a nucleotide sequence of SEQ ID No 1 or a fragment thereof in a test sample.

The promoter activity of the 5′ regulatory regions contained in PG-3 can be assessed as described below.

In order to identify the relevant regulatory active polynucleotide fragments or variants of SEQ ID No 1, one of skill in the art will refer to the book of Sambrook et al.(1989) which describes the use of a recombinant vector carrying a marker gene (i.e. beta galactosidase, chloramphenicol acetyl transferase, etc.) the expression of which will be detected when placed under the control of a biologically active polynucleotide fragments or variants of SEQ ID No 1. Genomic sequences located upstream of the first exon of the PG-3 gene are cloned into a suitable promoter reporter vector, such as the pSEAP-Basic, pSEAP-Enhancer, pβgal-Basic, pβgal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, β galactosidase, or green fluorescent protein. The sequences upstream the PG-3 coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequences within the upstream genomic DNA may be further defined by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has reduced or obliterated promoter activity, such as described, for example, by Coles et al. (1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well-known to those skilled in the art and is described in WO 97/17359, U.S. Pat. No. 5,374,544; EP 582 796; U.S. Pat. No. 5,698,389; U.S. Pat. No. 5,643,746; U.S. Pat. No. 5,502,176; and U.S. Pat. No. 5,266,488.

The strength and the specificity of the promoter of the PG-3 gene can be assessed through the expression levels of a detectable polynucleotide operably linked to the PG-3 promoter in different types of cells and tissues. The detectable polynucleotide may be either a polynucleotide that specifically hybridizes with a predefined oligonucleotide probe, or a polynucleotide encoding a detectable protein, including a PG-3 polypeptide or a fragment or a variant thereof. This type of assay is well-known to those skilled in the art and is described in U.S. Pat. No. 5,502,176; and U.S. Pat. No. 5,266,488. Some of the methods are discussed in more detail below.

Polynucleotides carrying the regulatory elements located at the 5′ end and at the 3′ end of the PG-3 coding region may be advantageously used to control the transcriptional and translational activity of an heterologous polynucleotide of interest.

Thus, the present invention also concerns a purified or isolated nucleic acid comprising a polynucleotide which is selected from the group consisting of the 5′ and 3′ regulatory regions, or a sequence complementary thereto or a regulatory active fragment or variant thereof.

Preferred fragments of the 5′ regulatory region have a length of about 1500 or 1000 nucleotides, preferably of about 500 nucleotides, more preferably about 400 nucleotides, even more preferably 300 nucleotides and most preferably about 200 nucleotides.

Preferred fragments of the 3′ regulatory region are at least 50, 100, 150, 200, 300 or 400 bases in length.

“Regulatory active” polynucleotide derivatives of SEQ ID No 1 are polynucleotides comprising or alternatively consisting essentially of or consisting of a fragment of said polynucleotide which is functional as a regulatory region for expressing a recombinant polypeptide or a recombinant polynucleotide in a recombinant cell host. It could act either as an enhancer or as a repressor.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The regulatory polynucleotides of the invention may be prepared from the nucleotide sequence of SEQ ID No 1 by cleavage using suitable restriction enzymes, as described for example in the book of Sambrook et al. (1989). The regulatory polynucleotides may also be prepared by digestion of SEQ ID No 1 by an exonuclease enzyme, such as Bal31 (Wabiko et al., 1986). These regulatory polynucleotides can also be prepared by nucleic acid chemical synthesis, as described elsewhere in the specification.

The regulatory polynucleotides according to the invention may be part of a recombinant expression vector that may be used to express a coding sequence in a desired host cell or host organism. The recombinant expression vectors according to the invention are described elsewhere in the specification.

A preferred 5′-regulatory polynucleotide of the invention includes the 5′-untranslated region (5′-UTR) of the PG-3 cDNA, or a regulatory active fragment or variant thereof.

A preferred 3′-regulatory polynucleotide of the invention includes the 3′-untranslated region (3′-UTR) of the PG-3 cDNA, or a regulatory active fragment or variant thereof.

A further object of the invention relates to a purified or isolated nucleic acid comprising:

a) a nucleic acid comprising a regulatory nucleotide sequence selected from the group consisting of:

(i) a nucleotide sequence comprising a polynucleotide of the 5′ regulatory region or a complementary sequence thereto; or

(ii) a nucleotide sequence comprising a polynucleotide having at least 80, 85, 90, or 95% of nucleotide identity with the nucleotide sequence of the 5′ regulatory region or a complementary sequence thereto; or

(iii) a nucleotide sequence comprising a polynucleotide that hybridizes under stringent hybridization conditions with the nucleotide sequence of the 5′ regulatory region or a complementary sequence thereto; or

(iv) a regulatory active fragment or variant of the polynucleotides in (i), (ii) and (iii);

b) a polynucleotide encoding a desired polypeptide or a nucleic acid of interest, operably linked to the nucleic acid defined in (a) above;

c) optionally, a nucleic acid comprising a 3′-regulatory polynucleotide, preferably a 3′-regulatory polynucleotide of the PG-3 gene.

In a specific embodiment of the nucleic acid defined above, said nucleic acid includes the 5′-untranslated region (5′-UTR) of the PG-3 cDNA, or a regulatory active fragment or variant thereof.

In a second specific embodiment of the nucleic acid defined above, said nucleic acid includes the 3′-untranslated region (3′-UTR) of the PG-3 cDNA, or a regulatory active fragment or variant thereof.

The regulatory polynucleotide of the 5′ regulatory region, or its regulatory active fragments or variants, is operably linked at the 5′-end of the polynucleotide encoding the desired polypeptide or polynucleotide.

The regulatory polynucleotide of the 3′ regulatory region, or its regulatory active fragments or variants, is advantageously operably linked at the 3′-end of the polynucleotide encoding the desired polypeptide or polynucleotide.

The desired polypeptide encoded by the above-described nucleic acid may be of various nature or origin, encompassing proteins of prokaryotic or eukaryotic origin. Among the polypeptides which may be expressed under the control of a PG-3 regulatory region are bacterial, fungal or viral antigens. Also encompassed are eukaryotic proteins such as intracellular proteins, like “house keeping” proteins, membrane-bound proteins, like receptors, and secreted proteins like endogenous mediators such as cytokines. The desired polypeptide may be the PG-3 protein, especially the protein of the amino acid sequence of SEQ ID No 3, or a fragment or a variant thereof.

The desired nucleic acids encoded by the above-described polynucleotide, usually an RNA molecule, may be complementary to a desired coding polynucleotide, for example to the PG-3 coding sequence, and thus useful as an antisense polynucleotide.

Such a polynucleotide may be included in a recombinant expression vector in order to express the desired polypeptide or the desired nucleic acid in host cell or in a host organism. Suitable recombinant vectors that contain a polynucleotide such as described herein are disclosed elsewhere in the specification.

5) Polynucleotide Variants

The invention also relates to variants and fragments of the polynucleotides described herein, particularly of a PG-3 gene containing one or more biallelic markers according to the invention.

a) Allelic Variant

A variant of a polynucleotide may be a naturally occurring variant such as a naturally occurring allelic variant, or it may be a variant that is not known to occur naturally. By an “allelic variant” is intended one of several alternate forms of a gene occupying a given locus on a chromosome of an organism (see Lewin, 1990), the disclosure of which is incorporated by reference in its entirety. Diploid organisms may be homozygous or heterozygous for an allelic form. Non-naturally occurring variants of the polynucleotide may be made by art-known mutagenesis techniques, including those applied to polynucleotides, cells or organisms.

b) Degenerate Variant

In addition to the isolated polynucleotides of the present invention, and fragments thereof, the invention further includes polynucleotides which comprise a sequence substantially different from those described above but which, due to the degeneracy of the genetic code, still encode a PG-3 polypeptide of the present invention. These polynucleotide variants are referred to as “degenerate variants” throughout the instant application. That is, all possible polynucleotide sequences that encode the PG-3 polypeptides of the present invention are completed. This includes the genetic code and species-specific codon preferences known in the art. Thus, it would be routine for one skilled in the art to generate the degenerate variants described above, for instance, to optimize codon expression for a particular host (e.g., change codons in the human mRNA to those preferred by other mammalian or bacterial host cells).

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions. In the context of the present invention, preferred embodiments are those in which the polynucleotide variants encode polypeptides which retain substantially the same biological properties or activities as the PG-3 protein. More preferred polynucleotide variants are those containing conservative substitutions.

c) Similar Polynucleotides

Other embodiments of the present invention is a purified, isolated or recombinant polynucleotide which is at least 90%, 95%, 96%, 97%, 98% or 99% identical to a polynucleotide selected from the group consisting of sequences of SEQ ID Nos: 1 and 2, or a sequence complementary thereto, or a fragment thereof. The nucleotide differences with regard to the nucleotide sequence of SEQ ID No 1 may be generally randomly distributed throughout the entire nucleic acid. Nevertheless, preferred nucleic acids are those wherein the nucleotide differences are predominantly located outside the coding sequences contained in the exons of SEQ ID No: 1. The above polynucleotides are included regardless of whether they encode a polypeptide having a biological activity. This is because even where a particular nucleic acid molecule does not encode a polypeptide having activity, one of skill in the art would still know how to use the nucleic acid molecule, for instance, as a hybridization probe or primer. Uses of the nucleic acid molecules of the present invention that do not encode a polypeptide having a biological activity include, inter alia, isolating a PG-3 gene or allelic variants thereof from a DNA library, and detecting a copy of a PG-3 gene or PG-3 mRNA expression in biological samples, suspected of containing PG-3 mRNA or DNA by Northern Blot or PCR analysis.

The invention also pertains to a purified, isolated or recombinant nucleic acid molecules comprising a polynucleotide having at least 80, 85, 90, or 95% nucleotide identity with a polynucleotide selected from the group consisting of the 5′ and 3′ PG-3 regulatory regions, advantageously 99% nucleotide identity, preferably 99.5% nucleotide identity and most preferably 99.8% nucleotide identity with a polynucleotide selected from the group consisting of the 5′ and 3′ PG-3 regulatory regions, or a sequence complementary thereto or a variant thereof or a regulatory active fragment thereof.

The present invention is further directed to polynucleotides having sequences at least 50%. 60%, 70%, 80%, 90%, 95%, 96%, 97%, 98% or 99% identity to a polynucleotide selected from the group consisting of sequences of SEQ ID Nos: 1 and 2, where said polynucleotide do, in fact, encode a polypeptide having a PG-3 biological activity. Of course, due to the degeneracy of the genetic code, one of ordinary skill in the art will immediately recognize that a large number of the polynucleotides at least 50%. 60%, 70%, 80%, 90%, 95%, 96%, 97%, 98%, or 99% identical to a polynucleotide selected from the group consisting of sequences of SEQ ID Nos: 1 and 2 will encode a polypeptide having PG-3 biological activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having a PG-3 biological activity. This is because the skilled artisan is fully aware of amino acid substitutions that are either less likely or not likely to significantly effect protein function (e.g., replacing one aliphatic amino acid with a second aliphatic amino acid), as further described below. By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the PG-3 polypeptide. In other words, to obtain a polynucleotide having a nucleotide sequence at least 95% identical to a reference nucleotide sequence, up to 5% of the nucleotides in the reference sequence may be deleted, inserted, or substituted with another nucleotide. The query sequence may be an entire sequence selected from the group consisting of sequences of SEQ ID Nos: 1 and 2, or the ORF (open reading frame) of a polynucleotide sequence selected from said group, or any fragment specified as described herein.

d) Hybridizing Polynucleotides

In another aspect, the invention provides an isolated or purified nucleic acid molecule comprising a polynucleotide which hybridizes under stringent hybridization conditions to any polynucleotide of the present invention using any methods known to those skilled in the art including those disclosed herein.

An object of the invention relates to purified, isolated or recombinant nucleic acid molecules comprising a polynucleotide that hybridizes, under the stringent hybridization conditions defined herein, with a polynucleotide selected from the group consisting of SEQ ID Nos: 1 and 2, or a sequence complementary thereto or a variant thereof or a fragment thereof. Another object of the invention relates to purified, isolated or recombinant nucleic acids comprising a polynucleotide that hybridizes, under the stringent hybridization conditions defined herein, with a polynucleotide selected from the group consisting of the nucleotide sequences of the 5′- and 3′ regulatory regions, or a sequence complementary thereto or a variant thereof or a regulatory active fragment thereof.

Also contemplated are nucleic acid molecules that hybridize to the polynucleotides of the present invention at lower stringency hybridization conditions, preferably at moderate or low stringency conditions as defined herein. Such hybridizing polynucleotides may be of at least 15, 18, 20, 23, 25, 28, 30, 35, 40, 50, 75, 100, 200, 300, 500 or 1000 nucleotides in length.

Of course, a polynucleotide which hybridizes only to polyA+ sequences (such as any 3′ terminal polyA+ tract of a cDNA shown in the sequence listing), or to a 5′ complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone generated using oligo dT as a primer).

Of particular interest, are the polynucleotides hybridizing to any polynucleotide of the invention encoding PG-3 polypeptides, particularly PG-3 polypeptides exhibiting a PG-3 biological activity.

6) Polynucleotides Fragments

The present invention is further directed to polynucleotides encoding portions or fragments of the nucleotide sequences described herein. A polynucleotide fragment is a polynucleotide having a sequence that is entirely the same as part but not all of a given nucleotide sequence, preferably the nucleotide sequence of a PG-3 gene, and variants thereof. The fragment can be a portion of an intron or an exon of a PG-3 gene. It can be the open reading frame of a PG-3 gene. It can also be a portion of the regulatory regions of PG-3.

Preferably, such fragments comprise at least one of the PG-3-related biallelic markers, wherein said said PG-3-related biallelic marker is selected from the group consisting of A1 to A80 or the complements thereto or a biallelic marker in linkage disequilibrium with one or more of the biallelic markers A1 to A80; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith. A set of preferred fragments contain at least one of the biallelic markers A1 to A80 of the PG-3 gene which are described herein or the complements thereto.

Uses for the polynucleotide fragments of the present invention include probes, primers, molecular weight markers and for expressing the polypeptide fragments of the present invention. Fragments include portions of polynucleotides selected from the group consisting of a) the sequences of SEQ ID Nos:1 and 2, b) the polynucleotides encoding a polypeptide of SEQ ID No: 3, c) and variants of polynucleotides described in a) or b). Particularly included in the present invention is a purified or isolated polynucleotide comprising at least 8 consecutive bases of a polynucleotide of the present invention. In one aspect of this embodiment, the polynucleotide comprises at least 10, 12, 15, 18, 20, 25, 28, 30, 35, 40, 50, 75, 100, 150, 200, 300, 400, 500, 800, 1000, 1500, or 2000 consecutive nucleotides of a polynucleotide of the present invention.

In addition to the above preferred polynucleotide sizes, further preferred sub-genuses of polynucleotides comprise at least 8 nucleotides, wherein “at least 8” is defined as any integer between 8 and the integer representing the 3′ most nucleotide position as set forth in the sequence listing or elsewhere herein. Further included as preferred polynucleotides of the present invention are polynucleotide fragments at least 8 nucleotides in length, as described above, that are further specified in terms of their 5′ and 3′ position. The 5′ and 3′ positions are represented by the position numbers set forth in the appended sequence listing. For allelic, degenerate and other variants, position 1 is defined as the 5′ most nucleotide of the ORF, i.e., the nucleotide “A” of the start codon with the remaining nucleotides numbered consecutively. Therefore, every combination of a 5′ and 3′ nucleotide position that a polynucleotide fragment of the present invention, at least 8 contiguous nucleotides in length, could occupy on a polynucleotide of the invention is included in the invention as an individual species. The polynucleotide fragments specified by 5′ and 3′ positions can be immediately envisaged and are therefore not individually listed solely for the purpose of not unnecessarily lengthening the specifications.

It is noted that the above species of polynucleotide fragments of the present invention may alternatively be described by the formula “a to b”; where “a” equals the 5′ most nucleotide position and “b” equals the 3′ most nucleotide position of the polynucleotide; and further where “a” equals an integer between I and the number of nucleotides of the polynucleotide sequence of the present invention minus 8, and where “b” equals an integer between 9 and the number of nucleotides of the polynucleotide sequence of the present invention; and where “a” is an integer smaller then “b” by at least 8.

The present invention also provides for the exclusion of any species of polynucleotide fragments of the present invention specified by 5′ and 3′ positions or sub-genuses of polynucleotides specified by size in nucleotides as described above. Any number of fragments specified by 5′ and 3′ positions or by size in nucleotides, as described above, may be excluded.

Preferred fragments of the invention are polynucleotides comprising polynucleotides encoding domains of polypeptides. Such fragments may be used to obtain other polynucleotides encoding polypeptides having similar domains using hybridization or RT-PCR techniques. Alternatively, these fragments may be used to express a polypeptide domain which may present a specific biological property. Preferred domains for the PG-3 polypeptides of the invention, herein named “described PG-3 domains”, are those that comprise amino acids from positions 3 to 87, from position 642 to 730, and from position 753 to 833 of SEQ ID No:3. Thus, another object of the invention is an isolated, purified or recombinant polynucleotide encoding a polypeptide consisting of, consisting essentially of, or comprising a contiguous span of at least 5, 6, 8, 10, 12, 15, 20, 25, 30, 35, 40, 50, 60, 75, 100, 150 or 200 consecutive amino acids of SEQ ID Nos: 3, where said contiguous span comprises at least 1, 2, 3, 5, or 10 of the amino acid positions of a PG-3 described domain. The present invention also encompasses isolated, purified or recombinant polynucleotides encoding a polypeptide comprising a contiguous span of at least 5, 6, 8, 10, 12, 15, 20, 25, 30, 35, 40, 50, 60, 75, 100, 150 or 200 consecutive amino acids of SEQ ID No:3, where said contiguous span is a PG-3 described domain. The present invention also encompasses isolated, purified or recombinant polynucleotides encoding a polypeptide comprising a PG-3 described domain of SEQ ID Nos: 3.

The present invention further encompasses any combination of the polynucleotide fragments listed in this section.

Such fragments may be “free-standing”, i.e. not part of or fused to other polynucleotides, or they may be comprised within a single larger polynucleotide of which they form a part or region. Indeed, several of these fragments may be present within a single larger polynucleotide.

7) Polynucleotide Constructs

The terms “polynucleotide construct” and “recombinant polynucleotide” are used interchangeably herein to refer to linear or circular, purified or isolated polynucleotides that have been artificially designed and which comprise at least two nucleotide sequences that are not found as contiguous nucleotide sequences in their initial natural environment. DNA Construct That Enables Temporal And Spatial PG-3 Gene Expression In Recombinant Cell Hosts And In Transgenic Animals.

In order to study the physiological and phenotypic consequences of a lack of synthesis of the PG-3 protein, both at the cell level and at the multi cellular organism level, the invention also encompasses DNA constructs and recombinant vectors enabling a conditional expression of a specific allele of the PG-3 genomic sequence or cDNA and also of a copy of this genomic sequence or cDNA harboring substitutions, deletions, or additions of one or more bases as regards to the PG-3 nucleotide sequence of SEQ ID Nos 1 and 2, or a fragment thereof, these base substitutions, deletions or additions being located either in an exon, an intron or a regulatory sequence, but preferably in the 5′-regulatory sequence or in an exon of the PG-3 genomic sequence or within the PG-3 cDNA of SEQ ID No 2. In a preferred embodiment, the PG-3 sequence comprises a biallelic marker of the present invention. In a preferred embodiment, the PG-3 sequence comprises at least one of the biallelic markers A1 to A80.

The present invention embodies recombinant vectors comprising any one of the polynucleotides described in the present invention. More particularly, the polynucleotide constructs according to the present invention can comprise any of the polynucleotides described in the “Genomic Sequences Of The PG3 Gene” section, the “PG-3 cDNA Sequences” section, the “Coding Regions” section, and the “Oligonucleotide Probes And Primers” section.

A first preferred DNA construct is based on the tetracycline resistance operon tet from E. coli transposon Tn10 for controlling the PG-3 gene expression, such as described by Gossen et al.(1992, 1995) and Furth et al.(1994). Such a DNA construct contains seven tet operator sequences from Tn10 (tetop) that are fused to either a minimal promoter or a 5′-regulatory sequence of the PG-3 gene, said minimal promoter or said PG-3 regulatory sequence being operably linked to a polynucleotide of interest that codes either for a sense or an antisense oligonucleotide or for a polypeptide, including a PG-3 polypeptide or a peptide fragment thereof. This DNA construct is functional as a conditional expression system for the nucleotide sequence of interest when the same cell also comprises a nucleotide sequence coding for either the wild type (tTA) or the mutant (rTA) repressor fused to the activating domain of viral protein VP 16 of herpes simplex virus, placed under the control of a promoter, such as the HCMVIE1 enhancer/promoter or the MMTV-LTR. Indeed, a preferred DNA construct of the invention comprises both the polynucleotide containing the tet operator sequences and the polynucleotide containing a sequence coding for the tTA or the rTA repressor.

In a specific embodiment, the conditional expression DNA construct contains the sequence encoding the mutant tetracycline repressor rTA, the expression of the polynucleotide of interest is silent in the absence of tetracycline and induced in its presence.

DNA Constructs Allowing Homologous Recombination: Replacement Vectors

A second preferred DNA construct comprises, from 5′-end to 3′-end: (a) a first nucleotide sequence that is included within the PG-3 genomic sequence; (b) a nucleotide sequence comprising a positive selection marker, such as the marker for neomycine resistance (neo); and (c) a second nucleotide sequence that is included within the PG-3 genomic sequence, and is located on the genome downstream the first PG-3 nucleotide sequence (a).

In a preferred embodiment, this DNA construct also comprises a negative selection marker located upstream of the nucleotide sequence (a) or downstream from the nucleotide sequence (c). Preferably, the negative selection marker comprises of the thymidine kinase (tk) gene (Thomas et al., 1986), the hygromycine beta gene (Te Riele et al., 1990), the hprt gene (Van der Lugt et al., 1991; Reid et al., 1990) or the Diphteria toxin A fragment (Dt-A) gene (Nada et al., 1993; Yagi et al. 1990). Preferably, the positive selection marker is located within a PG-3 exon sequence so as to interrupt the sequence encoding a PG-3 protein. These replacement vectors are described, for example, by Thomas et al.(1986; 1987), Mansour et al.(1988) and Koller et al.(1992).

The first and second nucleotide sequences (a) and (c) may be indifferently located within a PG-3 regulatory sequence, an intronic sequence, an exon sequence or a sequence containing both regulatory and/or intronic and/or exon sequences. The size of the nucleotide sequences (a) and (c) ranges from 1 to 50 kb, preferably from 1 to 10 kb, more preferably from 2 to 6 kb and most preferably from 2 to 4 kb. DNA Constructs Allowing Homologous Recombination: Cre-LoxP System

These new DNA constructs make use of the site specific recombination system of the PI phage. The PI phage possesses a recombinase called Cre which interacts specifically with a 34 base pairs loxP site. The loxP site is composed of two palindromic sequences of 13 bp separated by a 8 bp conserved sequence (Hoess et al., 1986). The recombination by the Cre enzyme between two loxP sites having an identical orientation leads to the deletion of the DNA fragment.

The Cre-loxP system used in combination with a homologous recombination technique has been first described by Gu et al. (1993, 1994). Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host. The recombinase enzyme may be provided at the desired time either by (a) incubating the recombinant cell hosts in a culture medium containing this enzyme, by injecting the Cre enzyme directly into the desired cell, such as described by Araki et al (1995), or by lipofection of the enzyme into the cells, such as described by Baubonis et al. (1993); (b) transfecting the cell host with a vector comprising the Cre coding sequence operably linked to a promoter functional in the recombinant host cell, said promoter being optionally inducible, said vector being introduced in the recombinant cell host, such as described by Gu et al. (1993) and Sauer et al. (1988); (c) introducing in the genome of the cell host a polynucleotide comprising the Cre coding sequence operably linked to a promoter functional in the recombinant cell host, which promoter is optionally inducible, and said polynucleotide being inserted in the genome of the cell host either by a random insertion event or an homologous recombination event, such as described by Gu et al. (1994).

In a specific embodiment, the vector containing the sequence to be inserted in the PG-3 gene by homologous recombination is constructed in such a way that selectable markers are flanked by loxP sites of the same orientation, it is possible, by treatment by the Cre enzyme, to eliminate the selectable markers while leaving the PG-3 sequences of interest that have been inserted by an homologous recombination event. Again, two selectable markers are needed: a positive selection marker to select for the recombination event and a negative selection marker to select for the homologous recombination event. Vectors and methods using the Cre-loxP system are described by Zou et al. (1994).

Thus, a third preferred DNA construct of the invention comprises, from 5′-end to 3′-end: (a) a first nucleotide sequence that is included in the PG-3 genomic sequence; (b) a nucleotide sequence comprising a polynucleotide encoding a positive selection marker, said nucleotide sequence comprising additionally two sequences defining a site recognized by a recombinase, such as a loxP site, the two sites being placed in the same orientation; and (c) a second nucleotide sequence that is included in the PG-3 genomic sequence, and is located on the genome downstream of the first PG-3 nucleotide sequence (a).

The sequences defining a site recognized by a recombinase, such as a loxP site, are preferably located within the nucleotide sequence (b) at suitable locations bordering the nucleotide sequence for which the conditional excision is sought. In one specific embodiment, two loxP sites are located at each side of the positive selection marker sequence, in order to allow its excision at a desired time after the occurrence of the homologous recombination event.

In a preferred embodiment of a method using the third DNA construct described above, the excision of the polynucleotide fragment bordered by the two sites recognized by a recombinase, preferably two loxp sites, is performed at a desired time, due to the presence within the genome of the recombinant host cell of a sequence encoding the Cre enzyme operably linked to a promoter sequence, preferably an inducible promoter, more preferably a tissue-specific promoter sequence and most preferably a promoter sequence which is both inducible and tissue-specific, such as described by Gu et al (1994).

The presence of the Cre enzyme within the genome of the recombinant cell host may result from the breeding of two transgenic animals, the first transgenic animal bearing the PG-3-derived sequence of interest containing the loxP sites as described above and the second transgenic animal bearing the Cre coding sequence operably linked to a suitable promoter sequence, such as described by Gu et al. (1994).

Spatio-temporal control of the Cre enzyme expression may also be achieved with an adenovirus based vector that contains the Cre gene thus allowing infection of cells, or in vivo infection of organs, for delivery of the Cre enzyme, such as described by Anton et al. (1995) and Kanegae et al. (1995).

The DNA constructs described above may be used to introduce a desired nucleotide sequence of the invention, preferably a PG-3 genomic sequence or a PG-3 cDNA sequence, and most preferably an altered copy of a PG-3 genomic or cDNA sequence, within a predetermined location of the targeted genome, leading either to the generation of an altered copy of a targeted gene (knock-out homologous recombination) or to the replacement of a copy of the targeted gene by another copy sufficiently homologous to allow an homologous recombination event to occur (knock-in homologous recombination). In a specific embodiment, the DNA constructs described above may be used to introduce a PG-3 genomic sequence or a PG-3 cDNA sequence comprising at least one biallelic marker of the present invention, preferably at least one biallelic marker selected from the group consisting of A1 to A80.

Nuclear Antisense DNA Constructs

Other compositions comprise a vector of the invention comprising an oligonucleotide fragment of the nucleic acid sequence of SEQ ID No 2, preferably a fragment including the start codon of the PG-3 gene, as an antisense tool that inhibits the expression of the corresponding PG-3 gene. Preferred methods using antisense polynucleotide according to the present invention are described in the section entitled “Antisense Approach”.

8) Oligonucleotide Probes And Primers

Polynucleotides derived from the PG-3 gene are useful in order to detect the presence of at least a copy of a nucleotide sequence of SEQ ID No 1, or a fragment, complement, or variant thereof in a test sample.

a) Structural Definitions

Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-97921, 98517-103471, 103603-108222, 108390-109221, 109324-114409, 114538-115723, 115957-122102, 122225-126876, 127033-157212, 157808-240825. Additional preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 1 or the complements thereof, wherein said contiguous span comprises at least 1, 2, 3, 5, or 10 of the following nucleotide positions of SEQ ID No 1: 1-10000, 10001-20000, 20001-30000, 30001-40000, 40001-50000, 50001-60000, 60001-70000, 70001-80000, 80001-90000, 90001-97921, 98517-103471, 103603-108222, 108390-109221, 109324-114409, 114538-115723, 115957-122102, 122225-126876, 127033-157212, 157808-159000, 159001-160000, 160001-170000, 170001-180000, 180001-190000, 190001-200000, 200001-210000, 210001-220000, 220001-230000, 230001-240825.

Another object of the invention is a purified, isolated, or recombinant nucleic acid comprising the nucleotide sequence of SEQ ID No 2, complementary sequences thereto, as well as allelic variants, and fragments thereof. Moreover, preferred probes and primers of the invention include purified, isolated, or recombinant PG-3 cDNAs consisting of, consisting essentially of, or comprising the sequence of SEQ ID No 2. Particularly preferred probes and primers of the invention include isolated, purified, or recombinant polynucleotides comprising a contiguous span of at least 12, 15, 18, 20, 25, 30, 35, 40, 50, 60, 70, 80, 90, 100, 150, 200, 500, or 1000 nucleotides of SEQ ID No 2 or the complements thereof. Additional preferred embodiments of the invention include probes and primers comprising a contiguous span of at least 12, 15, 18, 20, 25, 30, 35, 40, 50, 60, 70, 80, 90, 100, 150, 200, 500, or 1000 nucleotides of SEQ ID No 2 or the complements thereof, wherein said contiguous span comprises at least 1, 2, 3, 5, or 10 of the following nucleotide positions of SEQ ID No 2: 1-500, 501-1000, 1001-1500, 1501-2000, 2001-2500, 2501-3000, 3001-3500, 3501-3809.

Thus, the invention also relates to nucleic acid probes characterized in that they hybridize specifically, under the stringent hybridization conditions defined above, with a nucleic acid selected from the group consisting of the nucleotide sequences 1-97921, 98517-103471, 103603-108222, 108390-109221, 109324-114409, 114538-115723, 115957-122102, 122225-126876, 127033-157212, 157808-240825 of SEQ ID No 1 or a variant thereof or a sequence complementary thereto. The invention relates to nucleic acid probes characterized in that they hybridize specifically, under the stringent hybridization conditions defined above, with a nucleic acid of SEQ ID No 2 or a variant or a fragment thereof or a sequence complementary thereto.

In one embodiment the invention encompasses isolated, purified, and recombinant polynucleotides consisting of, or consisting essentially of a contiguous span of at least 8, 10, 12, 15, 18, 20, 25, 30, 35, 40, or 50 nucleotides in length of any one of SEQ ID Nos 1 and 2 and the complement thereof, wherein said span includes a PG-3-related biallelic marker in said sequence; optionally, said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, said contiguous span is 18 to 35 nucleotides in length and said biallelic marker is within 4 nucleotides of the center of said polynucleotide; optionally, said polynucleotide comprises, consists essentially of, or consists of said contiguous span and said contiguous span is 25 nucleotides in length and said biallelic marker is at the center of said polynucleotide; optionally, the 3′ end of said contiguous span is present at the 3′ end of said polynucleotide; and optionally, the 3′ end of said contiguous span is located at the 3′ end of said polynucleotide and said biallelic marker is present at the 3′ end of said polynucleotide. In a preferred embodiment, said probes comprises, consists of, or consists essentially of a sequence selected from the following sequences: P1 to P4 and P6 to P80 and the complementary sequences thereto.

In another embodiment the invention encompasses isolated, purified or recombinant polynucleotides comprising, consisting of, or consisting essentially of a contiguous span of at least 8, 10, 12, 15, 18, 20, 25, 30, 35, 40, or 50 nucleotides in length of SEQ ID Nos 1 and 2, or the complements thereof, wherein the 3′ end of said contiguous span is located at the 3′ end of said polynucleotide, and wherein the 3′ end of said polynucleotide is located within 20 nucleotides upstream of a PG-3-related biallelic marker in said sequence; optionally, wherein said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, wherein the 3′ end of said polynucleotide is located 1 nucleotide upstream of said PG-3-related biallelic marker in said sequence; and optionally, wherein said polynucleotide consists essentially of a sequence selected from the following sequences: D1 to D4, D6 to D80, E1 to E4 and E6 to E80.

In a further embodiment, the invention encompasses isolated, purified, or recombinant polynucleotides comprising, consisting of, or consisting essentially of a sequence selected from the following sequences: B1 to B52 and C1 to C52.

In an additional embodiment, the invention encompasses polynucleotides for use in hybridization assays, sequencing assays, and enzyme-based mismatch detection assays for determining the identity of the nucleotide at a PG-3-related biallelic marker in SEQ ID Nos 1 and 2, as well as polynucleotides for use in amplifying segments of nucleotides comprising a PG-3-related biallelic marker in SEQ ID Nos 1 and 2; optionally, wherein said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith.

The invention concerns the use of the polynucleotides according to the invention for determining the identity of the nucleotide at a PG-3-related biallelic marker, preferably in hybridization assay, sequencing assay, microsequencing assay, or an enzyme-based mismatch detection assay and in amplifying segments of nucleotides comprising a PG-3-related biallelic marker.

b) Design of Primers and Probes

A probe or a primer according to the invention has between 8 and 1000 nucleotides in length, or is specified to be at least 12, 15, 18, 20, 25, 35, 40, 50, 60, 70, 80, 100, 250, 500 or 1000 nucleotides in length. More particularly, the length of these probes and primers can range from 8, 10, 15, 20, or 30 to 100 nucleotides, preferably from 10 to 50, more preferably from 15 to 30 nucleotides. Shorter probes and primers tend to lack specificity for a target nucleic acid sequence and generally require cooler temperatures to form sufficiently stable hybrid complexes with the template. Longer probes and primers are expensive to produce and can sometimes self-hybridize to form hairpin structures. The appropriate length for primers and probes under a particular set of assay conditions may be empirically determined by one of skill in the art. The formation of stable hybrids depends on the melting temperature (Tm) of the DNA. The Tm depends on the length of the primer or probe, the ionic strength of the solution and the G+C content. The higher the G+C content of the primer or probe, the higher is the melting temperature because G:C pairs are held by three H bonds whereas A:T pairs have only two. The GC content in the probes of the invention usually ranges between 10 and 75%, preferably between 35 and 60%, and more preferably between 40 and 55%.

For amplification purposes, pairs of primers with approximately the same Tm are preferable. Primers may be designed using the OSP software (Hillier and Green, 1991), the disclosure of which is incorporated by reference in its entirety, based on GC content and melting temperatures of oligonucleotides, or using PC-Rare (http://bioinformatics.weizmann.ac.il/software/PC-Rare/doc/manuel.html) based on the octamer frequency disparity method (Griffais et al., 1991), the disclosure of which is incorporated by reference in its entirety. DNA amplification techniques are well known to those skilled in the art. Amplification techniques that can be used in the context of the present invention include, but are not limited to, the ligase chain reaction (LCR) described in EP-A-320 308, WO 9320227 and EP-A439 182, the polymerase chain reaction (PCR, RT-PCR) and techniques such as the nucleic acid sequence based amplification (NASBA) described in Guatelli et al. (1990) and in Compton (1991), Q-beta amplification as described in European Patent Application No 4544610, strand displacement amplification as described in Walker et al. (1996) and EP A 684 315 and, target mediated amplification as described in PCT Publication WO 9322461, the disclosures of which are incorporated by reference in their entireties.

A preferred probe or primer consists of a nucleic acid comprising a polynucleotide selected from the group of the nucleotide sequences of P1 to P4 and P6 to P80 and the complementary sequence thereto, B1 to B52, C1 to C52, D1 to D4, D6 to D80, E1 to E4 and E6 to E80, for which the respective locations in the sequence listing are provided in Tables 1, 2, and 3.

c) Preparation of Primers and Probes

The primers and probes can be prepared by any suitable method, including, for example, cloning and restriction of appropriate sequences and direct chemical synthesis by a method such as the phosphodiester method of Narang et al. (1979), the phosphodiester method of Brown et al. (1979), the diethylphosphoramidite method of Beaucage et al. (1981) and the solid support method described in EP 0 707 592, which disclosures are hereby incorporated by reference in their entireties.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047, which disclosures are hereby incorporated by reference in their entireties. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified, U.S. patent application Ser. No. 07/049,061 filed Apr. 19, 1993, which disclosure is hereby incorporated by reference in its entirety, describes modifications, which can be used to render a probe non-extendable.

d) Labeling of Probes

Any of the polynucleotides of the present invention can be labeled, if desired, by incorporating any label known in the art to be detectable by spectroscopic, photochemical, biochemical, immunochemical, or chemical means. For example, useful labels include radioactive substances (including, ³²P, ³⁵S, ³H, ¹²⁵I), fluorescent dyes (including, 5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988), which disclosures are hereby incorporated by reference in their entireties. In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron), which disclosures are hereby incorporated by reference in their entireties.

The detectable probe may be single stranded or double stranded and may be made using techniques known in the art, including in vitro transcription, nick translation, or kinase reactions. A nucleic acid sample containing a sequence capable of hybridizing to the labeled probe is contacted with the labeled probe. If the nucleic acid in the sample is double stranded, it may be denatured prior to contacting the probe. In some applications, the nucleic acid sample may be immobilized on a surface such as a nitrocellulose or nylon membrane. The nucleic acid sample may comprise nucleic acids obtained from a variety of sources, including genomic DNA, cDNA libraries, RNA, or tissue samples.

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe may be cloned into vectors such as expression vectors, sequencing vectors, or in vitro transcription vectors to facilitate the characterization and expression of the hybridizing nucleic acids in the sample. For example, such techniques may be used to isolate and clone sequences in a genomic library or cDNA library which are capable of hybridizing to the detectable probe as described herein.

e) Immobilization of Probes

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in the PG-3 gene or mRNA using other techniques.

Any of the polynucleotides, primers and probes of the present invention can be conveniently immobilized on a solid support. The solid support is not critical and can be selected by one skilled in the art. Thus, latex particles, microparticles, magnetic beads, non-magnetic beads (including polystyrene beads), membranes (including nitrocellulose strips), plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes® and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also relates to a method for detecting the presence of a nucleic acid molecule comprising a nucleotide sequence selected from the group consisting of SEQ ID Nos 1 and 2, fragments thereof, variants thereof and complementary sequences thereto in a sample, said method comprising the following steps of:

a) bringing into contact a nucleic acid probe or a plurality of nucleic acid probes which can hybridize with said nucleotide sequence included in said nucleic acid molecule in said sample to be assayed; and

b) detecting the hybrid complex formed between said probe(s) and said nucleic acid molecule in said sample.

The invention further concerns a kit for detecting the presence of a nucleic acid molecule comprising a nucleotide sequence selected from the group consisting of SEQ ID Nos 1 and 2, fragments thereof, variants thereof and complementary sequences thereto in a sample, said kit comprising:

a) a nucleic acid probe or a plurality of nucleic acid probes which can hybridize with said nucleotide sequence included in said nucleic acid molecule in said sample to be assayed; and

b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of P1 to P4 and P6 to P80 and the complementary sequence thereto, B 1 to B52, C1 to C52, D1 to D4, D6 to D80, E1 to E4 and E6 to E80 or a biallelic marker selected from the group consisting of A1 to A80 and the complements thereto.

f) Oligonucleotide Arrays

A substrate comprising a plurality of oligonucleotide primers or probes of the invention may be used either for detecting or amplifying targeted sequences in the PG-3 gene and may also be used for detecting mutations in the coding or in the non-coding sequences of the PG-3 gene.

As used herein, the term “array” means a one dimensional, two dimensional, or multidimensional arrangement of nucleic acids of sufficient length to permit specific detection of gene expression. For example, the array may contain a plurality of nucleic acids derived from genes whose expression levels are to be assessed. The array may include a PG-3 genomic DNA, a PG-3 cDNA, sequences complementary thereto or fragments thereof. Preferably, the fragments are at least 12, 15, 18, 20, 25, 30, 35, 40 or 50 nucleotides in length. More preferably, the fragments are at least 100 nucleotides in length. Even more preferably, the fragments are more than 100 nucleotides in length. In some embodiments the fragments may be more than 500 nucleotides in length.

Any polynucleotide provided herein may be attached in overlapping areas or at random locations on the solid support. Alternatively, the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotide makes these “addressable” arrays particularly useful in hybridization assays. Any addressable array technology known in the art can be employed with the polynucleotides of the invention. One particular embodiment of these polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods which incorporate a combination of photolithographic methods and solid phase oligonucleotide synthesis (Fodor et al., 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

In another embodiment of the oligonucleotide arrays of the invention, an oligonucleotide probe matrix may advantageously be used to detect mutations occurring in the PG-3 gene and preferably in its regulatory region. For this particular purpose, probes are specifically designed to have a nucleotide sequence allowing their hybridization to the genes that carry known mutations (either by deletion, insertion or substitution of one or several nucleotides). By known mutations, it is meant, mutations on the PG-3 gene that have been identified according, for example to the technique used by Huang et al. (1996) or Samson et al. (1996).

Another technique that may be used to detect mutations in the PG-3 gene is the use of a high-density DNA array. Each oligonucleotide probe constituting a unit element of the high density DNA array is designed to match a specific subsequence of the PG-3 genomic DNA or cDNA. Thus, an array consisting of oligonucleotides complementary to subsequences of the target gene sequence is used to determine the identity of the target sequence within a sample, measure its amount, and detect differences between the target sequence and the sequence of the PG-3 gene in the sample. In one such design, termed 4L tiled array, a set of four probes (A, C, G, T), preferably 15-nucleotide oligomers, is used. In each set of four probes, the perfect complement will hybridize more strongly than mismatched probes. Consequently, a nucleic acid target of length L is scanned for mutations with a tiled array containing 4L probes, the whole probe set containing all the possible mutations in the known sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996.

Consequently, the invention concerns an array of nucleic acid molecules comprising at least one polynucleotide of the invention, particularly a probe or primer as described herein. Preferably, the invention concerns an array of nucleic acid comprising at least two polynucleotides of the invention, particularly probes or primers as described herein. Preferably, the invention concerns an array of nucleic acid comprising at least five polynucleotides of the invention, particularly probes or primers as described herein.

A preferred embodiment of the present invention is an array of polynucleotides of at least 12, 15, 18, 20, 25, 30, 35, 40, 50, 100 or 500 nucleotides in length which includes at least 1, 2, 5, 10, 15, 20, 35, 50 or 100 sequences selected from the group consisting of the polynucleotides of SEQ ID Nos: 1 and 2, the polynucleotides encoding the polypeptide of SEQ ID No 3, sequences fully complementary thereto, and fragments thereof.

A further object of the invention consists of an array of nucleic acid sequences comprising either at least one of the sequences selected from the group consisting of P1 to P4 and P6 to P80, B1 to B52, C1 to C52, D1 to D4, D6 to D80, E1 to E4 and E6 to E80, the sequences complementary thereto, a fragment thereof of at least 8, 10, 12, 15, 18, or 20 consecutive nucleotides thereof, or at least one sequence comprising a biallelic marker selected from the group consisting of A1 to A80 and the complements thereto.

The invention also pertains to an array of nucleic acid sequences comprising either at least two of the sequences selected from the group consisting of P1 to P4, P6 to P80, B1 to B52, 5 C1 to C52, D1 to D4, D6 to D80, E1 to E4 and E6 to E80, the sequences complementary thereto, a fragment thereof of at least 8 consecutive nucleotides thereof, or at least two sequences comprising a biallelic marker selected from the group consisting of A1 to A80 and the complements thereof.

### PG-3 Proteins and Polypeptide Fragments

The term “PG-3 polypeptides” is used herein to embrace all of the proteins and polypeptides of the present invention. Also forming part of the invention are polypeptides encoded by the polynucleotides of the invention, as well as fusion polypeptides comprising such polypeptides. The invention embodies PG-3 proteins from humans, including isolated or purified PG-3 proteins consisting, consisting essentially, or comprising the sequence of SEQ ID No 3. More particularly, the present invention concerns allelic variants of the PG-3 protein comprising at least one amino acid selected from the group consisting of an arginine or an isoleucine residue at the amino acid position 304 of the SEQ ID No 3, a histidine or an aspartic acid residue at the amino acid position 314 of the SEQ ID No 3, a threonine or an asparagine residue at the amino acid position 682 of the SEQ ID No 3, an alanine or a valine residue at the amino acid position 761 of the SEQ ID No 3, and a proline or a serine residue at the amino acid position 828 of the SEQ ID No 3. In addition, the invention also encompasses polypeptide variants of PG-3 comprising at least one amino acid selected from the group consisting of a methionine or an isoleucine residue at the position 91 of SEQ ID No 3, a valine or an alanine residue at the position 306 of SEQ ID No 3, a proline or a serine residue at the position 413 of SEQ ID No 3, a glycine or an aspartate residue at the position 528 of SEQ ID No 3, a valine or an alanine residue at the position 614 of SEQ ID No 3, a threonine or an asparagine residue at the position 677 of SEQ ID No 3, a valine or an alanine residue at the position 756 of SEQ ID No 3, a valine or an alanine residue at the position 758 of SEQ ID No 3, a lysine or a glutamate residue at the position 809 of SEQ ID No 3, and a cysteine or an arginine residue at the position 821 of SEQ ID No 3.

Variant Polypeptides

The present invention further provides for PG-3 polypeptides encoded by allelic and splice variants, orthologs, species homologues, and derivatives of the polypeptides described herein, including mutated PG-3 proteins. Procedures known in the art can be used to obtain, allelic variants, splice variants, orthologs, and/or species homologues of polynucleotides encoding polypeptide of SEQ ID No:3, using information from the sequences disclosed herein.

The invention also encompasses purified, isolated, or recombinant polypeptides comprising a sequence at least 50% identical, more preferably at least 60% identical, and still more preferably 70%, 75%, 80%, 85%, 90%, 95%, 96%, 97%, 98% or 99% identical to the polypeptide of SEQ ID No:3 or a fragment thereof.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, (indels) or substituted with another amino acid.

Further polypeptides of the present invention include polypeptides which have at least 90% similarity, more preferably at least 95% similarity, and still more preferably at least 96%, 97%, 98% or 99% similarity to those described above. By a polypeptide having an amino acid sequence at least, for example, 95% “similar” to a query amino acid sequence of the present invention, it is intended that the amino acid sequence of the subject polypeptide is similar (i.e. contain identical or equivalent amino acid residues) to the query sequence except that the subject polypeptide sequence may include up to five amino acid alterations per each 100 amino acids of the query amino acid sequence. In other words, to obtain a polypeptide having an amino acid sequence at least 95% similar to a query amino acid sequence, up to 5% (5 of 100) of the amino acid residues in the subject sequence may be inserted, deleted, (indels) or substituted with another non-equivalent amino acid.

These alterations of the reference sequence may occur at the amino or carboxy terminal positions of the reference amino acid sequence or anywhere between those terminal positions, interspersed either individually among residues in the reference sequence or in one or more contiguous groups within the reference sequence. The query sequence may be an entire amino acid sequence of SEQ ID No:3 or any fragment specified as described herein.

The variant polypeptides described herein are included in the present invention regardless of whether they have their normal biological activity. This is because even where a particular polypeptide molecule does not have a biological activity, one of skill in the art would still know how to use the polypeptide, for instance, as a vaccine or to generate antibodies. Other uses of the polypeptides of the present invention that do not have a biological activity include, inter alia, as epitope tags, in epitope mapping, and as molecular weight markers on SDS-PAGE gels or on molecular sieve gel filtration columns using methods known to those of skill in the art. As described below, the polypeptides of the present invention can also be used to raise polyclonal and monoclonal antibodies, which are useful in assays for detecting PG-3 protein expression or as agonists and antagonists capable of enhancing or inhibiting PG-3 protein function. Further, such polypeptides can be used in the yeast two-hybrid system to “capture” PG-3 protein binding proteins, which are also candidate agonists and antagonists according to the present invention (See, e.g., Fields et al. 1989), which disclosure is hereby incorporated by reference in its entirety.

Preparation of the Polypeptides of the Invention

The polypeptides of the present invention can be prepared in any suitable manner. Such polypeptides include isolated naturally occurring polypeptides, recombinantly produced polypeptides, synthetically produced polypeptides, or polypeptides produced by a combination of these methods. The polypeptides of the present invention are preferably provided in an isolated form, and may be partially or preferably substantially purified.

Consequently, the present invention also comprises methods of making the polypeptides of the invention, particularly polypeptides encoded by the sequences of SEQ ID Nos: 1 and 2, or fragments thereof and methods of making the polypeptide of SEQ ID No: 3 or fragments thereof. The methods comprise sequentially linking together amino acids to produce the nucleic polypeptides having the preceding sequences. In some embodiments, the polypeptides made by these methods are 150 amino acids or less in length. In other embodiments, the polypeptides made by these methods are 120 amino acids or less in length.

Isolation

From Natural Sources

The PG-3 proteins of the invention may be isolated from natural sources, including bodily fluids, tissues and cells, whether directly isolated or cultured cells, of humans or non-human animals. Methods for extracting and purifying natural proteins are known in the art, and include the use of detergents or chaotropic agents to disrupt particles followed by differential extraction and separation of the polypeptides by ion exchange chromatography, affinity chromatography, sedimentation according to density, and gel electrophoresis. See, for example, “Methods in Enzymology”, Abbondanzo, et al., Academic Press, 1993, for a variety of methods for purifying proteins, which disclosure is hereby incorporated by reference in its entirety. Polypeptides of the invention also can be purified from natural sources using antibodies directed against the polypeptides of the invention, such as those described herein, in methods which are well known in the art of protein purification.

From Recombinant Sources

Preferably, the PG-3 polypeptides of the invention are recombinantly produced using routine expression methods known in the art. The polynucleotide encoding the desired polypeptide is operably linked to a promoter into an expression vector suitable for any convenient host. Both eukaryotic and prokaryotic host systems are used in forming recombinant polypeptides. The polypeptide is then isolated from lysed cells or from the culture medium and purified to the extent needed for its intended use.

Any PG-3 polynucleotide, including the cDNA described in SEQ ID No 2, and allelic variants thereof may be used to express PG-3 polypeptides. The nucleic acid encoding the PG-3 polypeptide to be expressed is operably linked to a promoter in an expression vector using conventional cloning technology. The PG-3 insert in the expression vector may comprise the full coding sequence for the PG-3 protein or a portion thereof. For example, the PG-3 derived insert may encode a polypeptide comprising at least 6, 8, 10, 12, 15, 20, 25, 30, 35, 40, 50, 60, 75, 100, 150 200, 250, 300, 400, 500, 600, 700 or 800 consecutive amino acids of the PG-3 protein of SEQ ID No 3.

Consequently, a further embodiment of the present invention is a method of making comprising a PG-3 polypeptide, preferably a protein of SEQ ID No 3, said method comprising the steps of

a) obtaining a nucleic acid molecule encoding said PG-3 polypeptide, preferably said nucleic acid molecule is selected from the group consisting of the sequence of SEQ ID No:2 and sequences encoding the polypeptide of SEQ ID No 3;

b) inserting said nucleic acid molecule in an expression vector such said nucleic acid molecule is operably linked to a promoter; and

c) introducing said expression vector into a host cell whereby said host cell produces said PG-3 polypeptide.

In one aspect of this embodiment, the method further comprises the step of isolating the polypeptide. Another embodiment of the present invention is a polypeptide obtainable by the method described in the preceding paragraph.

The expression vector is any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence is optimized for the particular expression organism in which the expression vector is introduced, as explained in U.S. Pat. No. 5,082,767, which disclosure is hereby incorporated by reference in its entirety.

In one embodiment, the entire coding sequence of a PG-3 cDNA and the 3′UTR through the poly A signal of the cDNA is operably linked to a promoter in the expression vector. Alternatively, if the nucleic acid encoding a portion of the PG-3 protein lacks a methionine to serve as the initiation site, an initiating methionine can be introduced next to the first codon of the nucleic acid using conventional techniques. Similarly, if the insert from the PG-3 cDNA lacks a poly A signal, this sequence can be added to the construct by, for example, splicing out the Poly A signal from pSG5 (Stratagene) using BglI and SalI restriction endonuclease enzymes and incorporating it into the mammalian expression vector pXT1 (Stratagene). pXT1 contains the LTRs and a portion of the gag gene from Moloney Murine Leukemia Virus. The position of the LTRs in the construct allows efficient stable transfection. The vector includes the Herpes Simplex Thymidine Kinase promoter and the selectable neomycin gene. The nucleic acid encoding the PG-3 protein or a portion thereof is obtained by PCR from a vector containing the PG-3 cDNA of SEQ ID No: 2 using oligonucleotide primers complementary to the PG-3 cDNA or portion thereof and containing restriction endonuclease sequences for Pst I incorporated into the 5′ primer and BglII at the 5′ end of the corresponding cDNA 3′ primer, taking care to ensure that the sequence encoding the PG-3 protein or a portion thereof is positioned properly with respect to the poly A signal. The purified fragment obtained from the resulting PCR reaction is digested with PstI, blunt ended with an exonuclease, digested with Bgl II, purified and ligated to pXT1, now containing a poly A signal and digested with BglII.

In another embodiment, it is often advantageous to add to the recombinant polynucleotide additional nucleotide sequence which codes for secretory or leader sequences, pro-sequences, sequences which aid in purification, such as multiple histidine residues, or an additional sequence for stability during recombinant production.

As a control, the expression vector lacking a cDNA insert is introduced into host cells or organisms.

Transfection of a PG-3 expressing vector into mouse NTH 3T3 cells is but one embodiment of introducing polynucleotides into host cells. Introduction of a polynucleotide encoding a polypeptide into a host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al. (1986), which disclosure is hereby incorporated by reference in its entirety. For example, the expression vector is transfected into mouse NIH 3T3 cells using Lipofectin (Life Technologies, Inc., Grand Island, N.Y.) under conditions outlined in the product specification. Positive transfectants are selected after growing the transfected cells in 600 ug/ml G418 Sigma, St. Louis, Mo.). It is specifically contemplated that the polypeptides of the present invention may in fact be expressed by a host cell lacking a recombinant vector.

Recombinant cell extracts, or proteins from the culture medium if the expressed polypeptide is secreted, are then prepared and proteins separated by gel electrophoresis. If desired, the proteins may be ammonium sulfate precipitated or separated based on size or charge prior to electrophoresis. The proteins present are detected using techniques such as Coomassie or silver staining or using antibodies against the PG-3 protein of interest. Coomassie and silver staining techniques are familiar to those skilled in the art.

To confirm expression of the PG-3 protein or a portion thereof, the proteins expressed from the host cells or organisms containing an expression vector comprising an insert which encodes the PG-3 polypeptide or a portion thereof are compared to the proteins expressed from the control cells or organisms containing the expression vector without an insert. The presence of a band from the cells containing the expression vector which is absent in control cells indicates that the PG-3 cDNA is expressed. Generally, the band corresponding to the protein encoded by the PG-3 cDNA will have a mobility near that expected based on the number of amino acids in the open reading frame of the cDNA. However, the band may have a mobility different than that expected as a result of modifications such as glycosylation, ubiquitination, or enzymatic cleavage.

Alternatively, the PG-3 polypeptide to be expressed may also be a product of transgenic animals, i.e., as a component of the milk of transgenic cows, goats, pigs or sheeps which are characterized by somatic or germ cells containing a nucleotide sequence encoding the protein of interest.

A polypeptide of this invention can be recovered and purified from recombinant cell cultures by well-known methods including differential extraction, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. See, for example, “Methods in Enzymology”, supra for a variety of methods for purifying proteins. Most preferably, high performance liquid chromatography (“HPLC”) is employed for purification. A recombinantly produced version of a PG-3 polypeptide can be substantially purified using techniques described herein or otherwise known in the art, such as, for example, by the one-step method described in Smith and Johnson (1988), which disclosure is hereby incorporated by reference in its entirety. Polypeptides of the invention also can be purified from recombinant sources using antibodies directed against the polypeptides of the invention, such as those described herein, in methods which are well known in the art of protein purification.

Preferably, the recombinantly expressed PG-3 polypeptide is purified using standard immunochromatography techniques. In such procedures, a solution containing the protein of interest, such as the culture medium or a cell extract, is applied to a column having antibodies against the protein attached to the chromatography matrix. The recombinant protein is allowed to bind the immunochromatography column. Thereafter, the column is washed to remove non-specifically bound proteins. The specifically bound secreted protein is then released from the column and recovered using standard techniques.

If antibody production is not possible, the PG-3 cDNA sequence or fragment thereof may be incorporated into expression vectors designed for use in purification schemes employing chimeric polypeptides. In such strategies the coding sequence of the PG-3 cDNA or fragment thereof is inserted in frame with the gene encoding the other half of the chimera. The other half of the chimera may be beta-globin or a nickel binding polypeptide encoding sequence. A chromatography matrix having antibody to beta-globin or nickel attached thereto is then used to purify the chimeric protein. Protease cleavage sites may be engineered between the beta-globin gene or the nickel binding polypeptide and the PG-3 cDNA or fragment thereof. Thus, the two polypeptides of the chimera may be separated from one another by protease digestion. Antibodies capable of specifically recognizing the expressed PG-3 protein or a portion thereof are described below.

One useful expression vector for generating beta-globin chimerics is pSG5 (Stratagene), which encodes rabbit beta-globin. Intron II of the rabbit beta-globin gene facilitates splicing of the expressed transcript, and the polyadenylation signal incorporated into the construct increases the level of expression. These techniques as described are well known to those skilled in the art of molecular biology. Standard methods are published in methods texts such as Davis et al., (1986) and many of the methods are available from Stratagene, Life Technologies, Inc., or Promega. Polypeptide may additionally be produced from the construct using in vitro translation systems such as the In vitro Express™ Translation Kit (Stratagene).

Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

The above procedures may also be used to express a mutant PG-3 protein responsible for a detectable phenotype or a portion thereof.

From Chemical Synthesis

In addition, polypeptides of the invention, especially short protein fragments, can be chemically synthesized using techniques known in the art (See, e.g., Creighton, 1983; and Hunkapiller et al., 1984), which disclosures are hereby incorporated by reference in their entireties. For example, a polypeptide corresponding to a fragment of a polypeptide sequence of the invention can be synthesized by use of a peptide synthesizer. A variety of methods of making polypeptides are known to those skilled in the art, including methods in which the carboxyl terminal amino acid is bound to polyvinyl benzene or another suitable resin. The amino acid to be added possesses blocking groups on its amino moiety and any side chain reactive groups so that only its carboxyl moiety can react. The carboxyl group is activated with carbodiimide or another activating agent and allowed to couple to the immobilized amino acid. After removal of the blocking group, the cycle is repeated to generate a polypeptide having the desired sequence. Alternatively, the methods described in U.S. Pat. No. 5,049,656, which disclosure is hereby incorporated by reference in its entirety, may be used.

Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Modifications

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity. See U.S. Pat. No. 4,179,337. The chemical moieties for derivatization may be selected. See, U.S. Pat. No. 4,179,337, which disclosure is hereby incorporated by reference in its entirety. The chemical moieties for derivatization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on a biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, (coupling PEG to G-CSF), and Malik et al. (1992) (reporting pegylation of GM-CSF using tresyl chloride), which disclosures are hereby incorporated by reference in their entireties. For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Multimerization

The polypeptides of the invention may be in monomers or multimers (i.e., dimers, trimers, tetramers and higher multimers). Accordingly, the present invention relates to monomers and multimers of the polypeptides of the invention, their preparation, and compositions containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term “homomer”, refers to a multimer containing only polypeptides corresponding to the amino acid sequences of SEQ ID No 3 (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomenc multimer of the invention is at least a homodimer, at least a homotrimer, or at least a homotetramer.

As used herein, the term “heteromer” refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing, or contained in the polypeptide encoded by a deposited clone). In one instance, the covalent associations are cross-linking between cysteine residues located within the polypeptide sequences, which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925, which disclosure is hereby incorporated by reference in its entirety). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, oseteoprotegerin (see, e.g., International Publication No: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins, and have since been found in a variety of different proteins (Landschulz et al., 1988). Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (1994) and in U.S. patent application Ser. No. 08/446,922, which disclosure is hereby incorporated by reference in its entirety. Other peptides derived from naturally occurring trimeric proteins may be employed in preparing trimeric polypeptides of the invention. In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, 30 techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

Mutated Polypeptides

To improve or alter the characteristics of PG-3 polypeptides of the present invention, protein engineering may be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they may be purified in higher yields and show better solubility than the corresponding natural polypeptide, at least under certain purification and storage conditions. Further, the polypeptides of the present invention may be produced as multimers including dimers, trimers and tetramers. Multimerization may be facilitated by linkers or recombinantly though heterologous polypeptides such as Fc regions.

N- and C-Terminal Deletions

It is known in the art that one or more amino acids may be deleted from the N-terminus or C-terminus without substantial loss of biological function. For instance, Ron et al. (1993) reported modified KGF proteins that had heparin binding activity even if 3, 8, or 27 N-terminal amino acid residues were missing. Accordingly, the present invention provides polypeptides having one or more residues deleted from the amino terminus of the polypeptide of SEQ ID No:3. Similarly, many examples of biologically functional C-terminal deletion mutants are known. For instance, Interferon gamma shows up to ten times higher activities by deleting 810 amino acid residues from the C-terminus of the protein (See, e.g., Dobeli, et al. 1988), which disclosure is hereby incorporated by reference in its entirety. Accordingly, the present invention provides polypeptides having one or more residues deleted from the carboxy terminus of the polypeptide of SEQ ID No 3. The invention also provides polypeptides having one or more amino acids deleted from both the amino and the carboxyl termini as described below.

Other Mutations

Other mutants in addition to N- and C-terminal deletion forms of the protein discussed above are included in the present invention. It also will be recognized by one of ordinary skill in the art that some amino acid sequences of the PG-3 polypeptides of the present invention can be varied without significant effect of the structure or function of the protein. If such differences in sequence are contemplated, it should be remembered that there will be critical areas on the protein which determine activity. Thus, the invention further includes variations of the PG-3 polypeptides which show substantial PG-3 polypeptide activity. Such mutants include deletions, insertions, inversions, repeats, and substitutions selected according to general rules known in the art so as to have little effect on activity. For example, guidance concerning how to make phenotypically silent amino acid substitutions is provided.

There are two main approaches for studying the tolerance of an amino acid sequence to change (See, Bowie et al. 1994), which disclosure is hereby incorporated by reference in its entirety. The first method relies on the process of evolution, in which mutations are either accepted or rejected by natural selection.

The second approach uses genetic engineering to introduce amino acid changes at specific positions of a cloned gene and selections or screens to identify sequences that maintain functionality. These studies have revealed that proteins are surprisingly tolerant of amino acid substitutions. The studies indicate which amino acid changes are likely to be permissive at a certain position of the protein. For example, most buried amino acid residues require nonpolar side chains, whereas few features of surface side chains are generally conserved. Other such phenotypically silent substitutions are described by Bowie et al. (supra) and the references cited therein.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the fragment, derivative, analog, or homologue of the polypeptide of the present invention may be, for example:

one in which one or more of the amino acid residues are substituted with a conserved or non-conserved amino acid residue (preferably a conserved amino acid residue) and such substituted amino acid residue may or may not be one encoded by the genetic code; or

one in which one or more of the amino acid residues includes a substituent group; or

one in which the PG-3 polypeptide is fused with another compound, such as a compound to increase the half-life of the polypeptide (for example, polyethylene glycol); or

one in which the additional amino acids are fused to the above form of the polypeptide, such as an IgG Fc fusion region peptide or leader or secretory sequence or a sequence which is employed for purification of the above form of the polypeptide or a pro-protein sequence.

Such fragments, derivatives and analogs are deemed to be within the scope of those skilled in the art from the teachings herein.

Thus, the PG-3 polypeptides of the present invention may include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids generally represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

A specific embodiment of a modified PG-3 peptide molecule of interest according to the present invention, includes, but is not limited to, a peptide molecule which is resistant to proteolysis, is a peptide in which the —CONH— peptide bond is modified and replaced by a (CH2NH) reduced bond, a (NHCO) retro inverso bond, a (CH2—O) methylene-oxy bond, a (CH2—S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond. The invention also encompasses a human PG-3 polypeptide or a fragment or a variant thereof in which at least one peptide bond has been modified as described above.

Amino acids in the PG-3 proteins of the present invention that are essential for function can be identified by methods known in the art, such as site-directed mutagenesis or alanine-scanning mutagenesis (See, e.g., Cunningham et al., 1989), which disclosure is hereby incorporated by reference in its entirety. The latter procedure introduces single alanine mutations at every residue in the molecule. The resulting mutant molecules are then tested for a biological activity, preferably a PG-3 biological activity, using assays appropriate for measuring the function of the particular protein. Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic, (See, e.g., Pinckard et al., 1967; Robbins, et al., 1987; and Cleland, et al., 1993).

A further embodiment of the invention relates to a polypeptide which comprises the amino acid sequence of a PG-3 polypeptide having an amino acid sequence which contains at least one conservative amino acid substitution, but not more than 50 conservative amino acid substitutions, not more than 40 conservative amino acid substitutions, not more than 30 conservative amino acid substitutions, and not more than 20 conservative amino acid substitutions. Also provided are polypeptides which comprise the amino acid sequence of a PG-3 polypeptide, having at least one, but not more than 10, 9, 8, 7, 6, 5, 4, 3, 2 or 1 conservative amino acid substitutions.

Polypeptide Fragments

a) Structural Definition

The present invention is further directed to fragments of the amino acid sequences described herein such as the polypeptide of SEQ ID No 3. More specifically, the present invention embodies purified, isolated, and recombinant polypeptides comprising at least 5, 6, preferably at least 8 to 10, more preferably 12, 15, 20, 25, 30, 35, 40, 50, 60, 75, 100, 125, 150, 175, 200, 250, 300, 400, 500, 600, 700 or 800 consecutive amino acids of SEQ ID No 3, and other polypeptides of the present invention. The present invention also embodies isolated, purified, and recombinant polypeptides comprising a contiguous span of at least 6 amino acids, preferably at least 8 to 10 amino acids, more preferably at least 12, 15, 20, 25, 30, 40, 50, or 100 amino acids of SEQ ID No 3, wherein said contiguous span includes at least 1, 2, 3, 5 or 10 of the following amino acid positions of SEQ ID No 3: 1-100, 101-200, 201-300, 301-400, 401-500, 501-600, 601-700, 701-835. Ii other preferred embodiments the contiguous stretch of amino acids comprises the site of a mutation or functional mutation, including a deletion, addition, swap or truncation of the amino acids.

In addition to the above polypeptide fragments, further preferred sub-genuses of polypeptides comprise at least 6 amino acids, wherein “at least 6” is defined as any integer between 6 and the integer representing the C-terminal amino acid of the polypeptide of the present invention including the polypeptide sequences of the sequence listing below. Further included are species of polypeptide fragments at least 6 amino acids in length, as described above, that are further specified in terms of their N-terminal and C-terminal positions. However, included in the present invention as individual species are all polypeptide fragments, at least 6 amino acids in length, as described above, and may be particularly specified by a N-terminal and C-terminal position. That is, every combination of a N-terminal and C-terminal position that a fragment at least 6 contiguous amino acid residues in length could occupy, on any given amino acid sequence of the sequence listing or of the present invention is included in the present invention

The present invention also provides for the exclusion of any fragment species specified by N-terminal and C-terminal positions or of any fragment sub-genus specified by size in amino acid residues as described above. Any number of fragments specified by N-terminal and C-terminal positions or by size in amino acid residues as described above may be excluded as individual species.

The above polypeptide fragments of the present invention can be immediately envisaged using the above description and are therefore not individually listed solely for the purpose of not unnecessarily lengthening the specification. Moreover, the above fragments need not have a biological activity, although polypeptides having these activities are preferred embodiments of the invention, since they would be useful, for example, in immunoassays, in epitope mapping, epitope tagging, as vaccines, and as molecular weight markers. The above fragments may also be used to generate antibodies to a particular portion of the polypeptide. These antibodies can then be used in immunoassays well known in the art to distinguish between human and non-human cells and tissues or to determine whether cells or tissues in a biological sample are or are not of the same type which express the polypeptides of the present invention.

It is noted that the above species of polypeptide fragments of the present invention may alternatively be described by the formula “a to b”; where “a” equals the N-terminal most amino acid position and “b” equals the C-terminal most amino acid position of the polynucleotide; and further where “a” equals an integer between 1 and the number of amino acids of the polypeptide sequence of the present invention minus 6, and where “b” equals an integer between 7 and the number of amino acids of the polypeptide sequence of the present invention; and where “a” is an integer smaller then “b” by at least 6.

b) Domains

Preferred polynucleotide fragments of the invention are domains of polypeptides of the invention. Such domains may eventually comprise linear or structural motifs and signatures including, but not limited to, leucine zippers, helix-turn-helix motifs, post-translational modification sites such as glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites. Such domains may present a particular biological activity such as DNA or RNA-binding, secretion of proteins, transcription regulation, enzymatic activity, substrate binding activity, etc.

A domain has a size generally comprised between 3 and 1000 amino acids. In preferred embodiment, domains comprise a number of amino acids that is any integer between 6 and 200. Domains may be synthesized using any methods known to those skilled in the art, including those disclosed herein, particularly in the section entitled “Preparation of the polypeptides of the invention”. Methods for determining the amino acids which make up a domain with a particular biological activity include mutagenesis studies and assays to determine the biological activity to be tested.

Alternatively, the polypeptides of the invention may be scanned for motifs, domains and/or signatures in databases using any computer method known to those skilled in the art. Searchable databases include Prosite (Hofmann et al., 1999; Bucher and Bairoch, 1994), Pfam (Sonnhammer et al., 1997; Henikoff et al., 2000; Bateman et al., 2000), Blocks (Henikoff et al., 2000), Print (Attwood et al., 1996), Prodom (Sonnhammer and Kahn, 1994; Corpet et al. 2000), Sbase (Pongor et al., 1993; Murvai et al., 2000), Smart (Schultz et al., 1998), Dali/FSSP (Holm and Sander, 1996, 1997 and 1999), HSSP (Sander and Schneider, 1991), CATH (Orengo et al., 1997; Pearl et al., 2000), SCOP (Murzin et al., 1995; Lo Conte et al., 2000), COG (Tatusov et al., 1997 and 2000), specific family databases and derivatives thereof (Nevill-Manning et al., 1998; Yona et al., 1999; Attwood et al., 2000), each of which disclosures are hereby incorporated by reference in their entireties. For a review on available databases, see issue 1 of volume 28 of Nucleic Acid Research (2000), which disclosure is hereby incorporated by reference in its entirety.

Consequently, preferred polynucleotide fragments of the invention are domains of the polypeptide of SEQ ID No 3. Preferred domains for the PG-3 polypeptides of the invention, herein named “described PG-3 domains”, are those that comprise amino acids from positions 3 to 87, from position 642 to 730, and from position 753 to 833 of SEQ ID No 3.

Therefore, the present invention encompasses isolated, purified, or recombinant polypeptides which consist of, consist essentially of, or comprise a contiguous span of at least 6, preferably at least 8 to 10, more preferably 12, 15, 20, 25, 30, 35, 40, 50, 60, 75, 100, 125, 150, 175, 200, 225, 250, 275, or 300 amino acids of the polypeptide of SEQ ID No 3, where said contiguous span comprises at least 1, 2, 3, 5, or 10 amino acids positions of a PG-3 described domain. The present invention also encompasses isolated, purified, or recombinant polypeptides comprising, consisting essentially of, or consisting of a contiguous span of at least 6, preferably at least 8 to 10, more preferably 12, 15, 20, 25, 30, 35, 40, 45, 50, 60, 70, 80 or 90 amino acids of the polypeptide of SEQ ID No 3, where said contiguous span is a PG-3 described domain. The present invention also encompasses isolated, purified, or recombinant polypeptides which comprise, consist of or consist essentially PG-3 described domain of the polypeptide of SEQ ID No 3.

Polypeptides of the present invention that are not specifically described in this table are not considered as not belonging to a domain. This is because they may still be not recognized as such by the particular algorithms used or not be included in the particular database searched. In fact, all fragments of the polypeptides of the present invention, at least 6 amino acids residues in length, are included in the present invention as being a domain. The domains of the present invention preferably comprise 6 to 200 amino acids (i.e. any integer between 6 and 200, inclusive) of a polypeptide of the present invention. Also, included in the present invention are domain fragments between the integers of 6 and the full length PG-3 sequence of the sequence listing. All combinations of sequences between the integers of 6 and the full-length sequence of a PG-3 polypeptide are included. The domain fragments may be specified by either the number of contiguous amino acid residues (as a sub-genus) or by specific N-terminal and C-terminal positions (as species) as described above for the polypeptide fragments of the present invention. Any number of domain fragments of the present invention may also be excluded in the same manner.

c) Epitopes and Antibody Fusions:

A preferred embodiment of the present invention is directed to epitope-bearing polypeptides and epitope-bearing polypeptide fragments. These epitopes may be “antigenic epitopes” or both an “antigenic epitope” and an “immunogenic epitope”. An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (See, e.g., Geysen, et al., 1984), which disclosure is hereby incorporated by reference in its entirety. It is particularly noted that although a particular epitope may not be immunogenic, it is nonetheless useful since antibodies can be made to both immunogenic and antigenic epitopes.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means (See, e.g., Houghten, 1985), also further described in U.S. Pat. No. 4,631,21, which disclosures are hereby incorporated by reference in their entireties. Methods for determining the amino acids which make up an epitope include x-ray crystallography, 2-dimensional nuclear magnetic resonance, and epitope mapping, e.g., the Pepscan method described by Geysen et al. (1984); PCT Publication No. WO 84/03564; and PCT Publication No. WO 84/03506, which disclosures are hereby incorporated by reference in their entireties. Another example is the algorithm of Jameson and Wolf, (1988) (said reference incorporated by reference in its entirety). The Jameson-Wolf antigenic analysis, for example, may be performed using the computer program PROTEAN, using default parameters (Version 4.0 Windows, DNASTAR, Inc., 1228 South Park Street Madison, Wis.

Antigenic epitopes predicted by the Jameson-Wolf algorithm for the PG-3 polypeptide of SEQ ID No 3 are the fragments comprising the amino acids from position 17 to 29, 52 to 68, 104 to 127, 138 to 148, 188 to 195, 198 to 210, 238 to 254, 280 to 292, 336 to 341, 346 to 383, 386 to 395, 406 to 420, 419 to 438, 465 to 470, 480 to 497, 511 to 526, 532 to 544, 559 to 570, 568 to 580, 599 to 609, 610 to 618, 619 to 628, 636 to 647, 655 to 661, 747 to 754, or 799 to 808. As used herein, the term “epitope described for PG-3” refers to all preferred polynucleotide fragments described in the above list. It is pointed out that the immunogenic epitopes listed above describe only amino acid residues comprising epitopes predicted to have the highest degree of immunogenicity by a particular algorithm. Polypeptides of the present invention that are not specifically described as immunogenic are not considered non-antigenic. This is because they may still be antigenic in vivo but merely not recognized as such by the particular algorithm used. Alternatively, the polypeptides are most likely antigenic in vitro using methods such a phage display. Thus, listed above are the amino acid residues comprising only preferred epitopes, not a complete list. In fact, all fragments of the PG-3 polypeptides of the present invention, at least 6 amino acids residues in length, are included in the present invention as being useful as antigenic epitope. Amino acid residues comprising other immunogenic epitopes may be determined by algorithms similar to the Jameson-Wolf analysis or by in vivo testing for an antigenic response using the methods described herein or those known in the art.

Therefore, the present invention encompasses isolated, purified, or recombinant polypeptides which consist of, consist essentially of, or comprise a contiguous span of at least 6, preferably at least 8 to 10, more preferably 12, 15, 20, 25, 30, 35, 40, 50, 60, 75, 100, 125, 150, 175, 200, 225, 250, 275, or 300 amino acids of SEQ ID No 3, where said contiguous span comprises at least 1, 2, 3, 5, or 10 amino acids positions of an epitope described for PG-3. The present invention also encompasses isolated, purified, or recombinant polypeptides comprising, consisting essentially of, or consisting of a contiguous span of at least 6, preferably at least 7, or 8, more preferably 10, 12, 15, 18 or 20 amino acids of SEQ ID No 3, where said contiguous span is an epitope described for PG-3. The present invention also encompasses isolated, purified, or recombinant polypeptides which comprise, consist of or consist essentially of an epitope described for PG-3 of the sequence of SEQ ID No 3.

The epitope-bearing fragments of the present invention preferably comprises 6 to 50 amino acids (i.e. any integer between 6 and 50, inclusive) of a polypeptide of the present invention. Also, included in the present invention are antigenic fragments between the integers of 6and the full length PG-3 sequence of the sequence listing. All combinations of sequences between the integers of 6 and the full-length sequence of a PG-3 polypeptide are included. The epitope-bearing fragments may be specified by either the number of contiguous amino acid residues (as a sub-genus) or by specific N-terminal and C-terminal positions (as species) as described above for the polypeptide fragments of the present invention. Any number of epitope-bearing fragments of the present invention may also be excluded in the same manner.

Antigenic epitopes are useful, for example, to raise antibodies, including monoclonal antibodies that specifically bind the epitope (See, Wilson et al., 1984; and Sutcliffe, et al., 1983), which disclosures are hereby incorporated by reference in their entireties. The antibodies are then used in various techniques such as diagnostic and tissue/cell identification techniques, as described herein, and in purification methods such as immunoaffinity chromatography.

Similarly, immunogenic epitopes can be used to induce antibodies according to methods well known in the art (See, Sutcliffe et al., supra; Wilson et al., supra; Chow et al.;(1985) and Bittle, et al., (1985), which disclosures are hereby incorporated by reference in their entireties). A preferred immunogenic epitope includes the natural PG-3 protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.).

Epitope-bearing polypeptides of the present invention are used to induce antibodies according to methods well known in the art including, but not limited to, in vivo immunization, in vitro immunization, and phage display methods (See, e.g., Sutcliffe, et al., supra; Wilson, et al., supra, and Bittle, et al., supra). If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine residues may be coupled to a carrier using a linker such as -maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody, which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the PG-3 polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins (See, e.g., EPA 0,394,827; and Traunecker et al., 1988), which disclosures are hereby incorporated by reference in their entireties. Fusion proteins that have a disulfide-linked dimeric structure due to the IgG portion can also be more efficient in binding and neutralizing other molecules than monomeric polypeptides or fragments thereof alone (See, e.g., Fountoulakis et al., 1995), which disclosure is hereby incorporated by reference in its entirety. Nucleic acids encoding the above epitopes can also be recombined with a gene of interest as an epitope tag to aid in detection and purification of the expressed polypeptide.

Additional fusion proteins of the invention may be generated through the techniques of gene-shuffling, motif-shuffling, exon-shuffling, or codon-shuffling (collectively referred to as “DNA shuffling”). DNA shuffling may be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; and Patten, et al., (1997); Harayama, (1998); Hansson, et al (1999); and Lorenzo and Blasco, (1998). (Each of these documents are hereby incorporated by reference). In one embodiment, one or more components, motifs, sections, parts, domains, fragments, etc., of coding polynucleotides of the invention, or the polypeptides encoded thereby may be recombined with one or more components, motifs, sections, parts, domains, fragments, etc. of one or more heterologous molecules.

The present invention further encompasses any combination of the polypeptide fragments listed in this section.

PG-3 Polypeptide Biological Activities

It is believed that the PG3 polypeptide of the invention is involved in DNA repair, recombination and cell cycle control. Preferred polypeptides of the invention are those that comprise amino acids from positions 3 to 87, from position 642 to 730, and from position 753 to 833 of SEQ ID No:3. Other preferred polypeptides of the invention are any fragment of SEQ ID No 3 having any of the biological activities described herein.

Multimerization

The invention relates to compositions and methods using the PG-3 protein of the invention or fragments thereof, preferably PG-3 multimerizationd domains, more preferably PG-3 fragments that comprise amino acids from positions 3 to 87, from position 642 to 730, or from position 753 to 833 of SEQ ID No 3, to mediate multimerization of proteins of interest

Multimerization domains have been shown to be useful tools in several areas of biotechnology, especially in protein engineering, where their ability to mediate homo-dimerization or hetero-dimerization has found several applications. For example, Bosslet et al. have described the use of a pair of leucine zipper for in vitro diagnosis, in particular for the immunochemical detection and determination of an analyte in a biological liquid (U.S. Pat. No. 5,643,731)/Tso et al. have used leucine zippers for producing bispecific antibody heterodimers (U.S. Pat. No. 5,932,448)/Methods of preparing soluble oligomeric proteins using leucine zippers have been described by Conrad et al (U.S. Pat. No. 5,965,712), Ciardelli et al. (U.S. Pat. No. 5,837,816), Spriggs et al. (WO9410308)/Leucine zipper forming sequences have been used by Pelletier et al in protein fragment complementation assays to detect biomolecular interactions (WO9834120). Because of their usefulness in biotechnology, it is thus highly interesting to isolate new multimerization domains.

The multimerization activity of PG-3 or any proteins containing a PG-3 fragment, preferably PG-3 fragments from positions 3 to 87, from position 642 to 730, or from position 753 to 833 of SEQ ID No 3 may be assayed using any of the assays known to those skilled in the art including those disclosed in the references cited herein.

In a preferred embodiment, the invention relates to compositions and methods of using PG-3 or part thereof, preferably PG-3 fragments from positions 3 to 87, from position 642 to 730, or from position 753 to 833 of SEQ ID No 3, for preparing soluble multimeric proteins, which consist in multimers of fusion proteins containing PG-3 or part thereof fused to a protein of interest, using any technique known to those skilled in the art including those teached in international patent WO9410308, which disclosure is hereby incorporated by reference in its entirety. In another preferred embodiment, PG-3 or part thereof, preferably PG-3 fragments from positions 3 to 87, from position 642 to 730, or from position 753 to 833 of SEQ ID No 3, is used to produce bispecific antibody heterodimers using the teaching of U.S. Pat. No. 5,932,448, which disclosure is hereby incorporated by reference in its entirety. Briefly, PG-3 or part thereof is linked to an epitope binding component whereas a second multimerization domain is linked to a second epitope binding component with a different specificity. The second multimerization domain can either be the same or another PG-3 fragment, or an heterologous multimerization domain. Bispecific antibodies are formed by pairwise association of the multimerization domains, forming an heterodimer which links two distinct epitope binding components. In still another preferred embodiment, PG-3 or part thereof, preferably PG-3 fragments from positions 3 to 87, from position 642 to 730, or from position 753 to 833 of SEQ ID No 3, is used for detection and determination of an analyte in a biological liquid as described in U.S. Pat. No. 5,643,731, which disclosure is hereby incorporated by reference in its entirety. Briefly, a first PG-3 multimerization domain is immobilized on a solid support and the second multimerization domain is coupled to a specific binding partner for an analyte in a biological fluid. The two peptides are then brought into contact thereby immobilizing the binding partner on the solid phase. The biological sample is then contacted with the immobilized binding partner and the amount of analyte in the sample bound to the binding partner determined. The second multimerization domain can either be the same or another PG-3 fragment, or an heterologous multimerization domain. In still another preferred embodiment, PG-3 or part thereof, preferably PG-3 fragments from positions 3 to 87, from position 642 to 730, or from position 753 to 833 of SEQ ID No 3, may be used to synthesize novel nucleic acid binding proteins which are able to multimerize with proteins of interest, for example to inhibit and/or control cellular growth using any genetic engineering technique known to those skilled in the art including the ones described in the U.S. Pat. No. 5,942,433, which disclosure is hereby incorporated by reference in its entirety.

In another embodiment, the invention relates to compositions and methods using PG-3 or part thereof, preferably PG-3 fragments from positions 3 to 87, from position 642 to 730, or from position 753 to 833 of SEQ ID No 3, in protein fragment complementation assays to detect biomolecular interactions in vivo and in vitro as described in international patent WO9834120, which disclosures is hereby incorporated by reference in its entirety. Such assays may be used to study the equilibrium and kinetic aspects of molecular interactions including protein-protein, protein-nucleic acid, protein-carbohydrate and protein-small molecule interactions, for screening cDNA libraries for binding to a target protein with unknown proteins or libraries of small organic molecules for biological activity.

Still, another object of the present invention relates to the use of PG-3 or part thereof, preferably PG-3 fragments from positions 3 to 87, from position 642 to 730, or from position 753 to 833 of SEQ ID No 3 for identifying new multimerization domains using any techniques for detecting protein-protein interaction known to those skilled in the art. Among the traditional methods which may be employed are co-immunoprecipitation, crosslinking and co-purification through gradients or chromatographic columns of cell lysates. Once isolated as a protein interacting with PG-3, or part thereof, such an intracellular protein can be identified (e.g. its amino acid sequence determined) and can, in turn, be used, in conjunction with standard techniques, to identify other proteins with which it interacts. The amino acid sequence thus obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel et al., eds., Current Protocols in Molecular Biology, J. Wiley and Sons (New York, N.Y. 1993) and PR Protocols: A Guide to Methods and Applications, 1990, Innis, M. et al, eds. Academic Press, Inc., New York).

Alternatively, PG-3 or fragments therof, preferably PG-3 fragments from positions 3 to 87, from position 642 to 730, or from position 753 to 833 of SEQ ID No 3, could be used by those skilled in art as a “bait protein” in a well established yeast double hybridization system to identify its interacting protein partners in vivo from cDNA library derived from different tissues or cell types of a given organism. Alternatively, PG-3 or fragments therof, preferably PG-3 fragments from positions 3 to 87, from position 642 to 730, or from position 753 to 833 of SEQ ID No 3, could be used by those skilled in art in mammalian cell transfection experiments. When fused to a suitable peptide tag such as [His]₆ tag in a protein expression vector and introduced into culture cells, this expressed fusion protein can be immunoprecipitated with its potential interacting proteins by using anti-tag peptide antibody. This method could be chosen either to identify the associated partner or to confirm the results obtained by other methods such as those just mentioned.

Alternatively, methods may be employed which result in the simultaneous identification of genes which encode the intracellular proteins that can dimerize with the PG-3 or fragments therof, using any technique known to those skilled in the art. These methods include, for example, probing cDNA expression libraries, in a manner similar to the well known technique of antibody probing of lambda.gt11 libraries, using as a probe a labeled version of PG-3 protein or part thereof, or fusion protein, e.g., PG-3 or part thereof fused to a marker (e.g., an enzyme, fluor, luminescent protein, or dye), or an Ig-Fc domain (for technical details on screening of cDNA expression libraries, see Ausubel et al, supra). Alternatively, another method for the detection of protein interaction in vivo, the two-hybrid system, may be used.

Regulation of Ranscription

The invention relates to compositions and methods using PG3 polypeptides or part thereof, preferably fragments comprising a transcription regulation domain, more preferably PG-3 fragments that comprise amino acids from positions 3 to 87, from position 642 to 730, or from position 753 to 833 of SEQ ID No 3, to regulate gene transcription.

The transcription regulation activity of PG-3 or any proteins containing a PG-3 fragment, preferably PG-3 fragments from positions 3 to 87, from position 642 to 730, or from position 753 to 833 of SEQ ID No 3 may be assayed using any of the assays known to those skilled in the art including those disclosed in the references cited herein. Such assays include the yeast transcription assay described in Hayes et al., Cancer Res. 60:2411-2418 (2000) and in Miyake et al., J. Biol. Chem. 275:40169-40173 (2000).

One of the remarkable features of such domains of transcriptional factors in general is that “fusing” them to heterologous protein domains seldom affects their ability to regulate transcription when recruited to a wide variety of promoters. The high degree of functional independence exhibited by these regulation domains makes them valuable tools in various biological assays for analyzing gene expression and protein-protein or protein-RNA or protein-small molecule drug interactions. Several strategies to improve the potency of such transcription regulation domains and thereby the expression of genes under their control have been reported. These approaches generally involve increasing the number of copies of regulation domains fused to the DNA binding domain or generating transcriptional regulators containing synergizing combinations of regulation domains.

Therefore, in an additional embodiment, this invention provides compositions and methods containing new transcription factors comprising PG3 or part thereof, preferably fragments comprising a transcription regulation domain, more preferably PG-3 fragments from positions 3 to 87, from position 642 to 730, or from position 753 to 833 of SEQ ID No 3. Such transcription factors may be designed to regulate the expression of target genes of interest. Aspects of the invention are applicable to systems involving either covalent or non-covalent linking of the transcription regulation domain to a DNA binding domain. In practice, cells can be engineered by the introduction of recombinant nucleic acids encoding the fusion proteins containing at least two mutually heterologous domains, one of them being the regulation domain of the invention, and in some cases additional nucleic acid constructs, to render them capable of ligand-dependent regulation of transcription of a target gene. Administration of the ligand to the cells then regulates positively or negatively target gene transcription (all laboratory methods related to this embodiment are completely described in U.S. Pat. Nos. 6,015,709, which disclosure is hereby incorporated by reference in its entirety). Illustrative (non-limiting) examples of heterologous domains which can be included along with the regulation domain of the invention in various fusion proteins of this invention include another transcription regulatory domain (i.e., transcription activation domains such as a p65, VP16 or AP domain; transcription potentiating or synergizing domains; or transcription repression domains such as an ssn-6/TUP-1 domain or Kruppel family suppressor domain); a DNA binding domain such as a GAL4, lex A or a composite DNA binding domain such as a composite zinc finger domain or a ZFHD1 domain; or a ligand-binding domain comprising or derived from (a) an immunophilin, cyclophilin or FRB domain; (b) an antibiotic binding domain such as tetR: or (c) a hormone receptor such as a progesterone receptor or ecdysone receptor. A wide variety of ligand binding domains may be used in this invention, although ligand binding domains which bind to a cell permeant ligand are preferred. It is also preferred that the ligand have a molecular weight under about 5 kD, more preferably below 2.5 kD and optimally below about 1500 D. Non-proteinaceous ligands are also preferred. Examples of ligand binding domain/ligand pairs that may be used in the practice of this invention include, but are not limited to: FKBP:FK1012, FKBP:synthetic divalent FKBP ligands (see WO 96/0609 and WO 97/31898), FRP:rapamycin/FKBP (see e.g., WO 96/41865 and Rivera et al., “A humanized system for pharmacologic control of gene expression”, Nature Medicine 2(9):1028-1032 (1997)), cyclophilin:cyclosporin (see e.g. WO 94/18317), DHFR:methotrexate (see e.g. Licitra et al., 1996, Proc. Natl. Acad. Sci. U.S.A. 93:12817-12821), TetR:tetracycline or doxycycline or other analogs or mimics thereof (Gossen and Bujard, 1992, Proc. Natl. Acad. Sci. U.S.A. 89:5547; Gossen et al., 1995, Science 268:1766-1769; Kistner et al., 1996, Proc. Natl. Acad. Sci. U.S.A. 93:10933-10938), a progesterone receptor:RU486 (Wang et al., 1994, Proc. Natl. Acad. Sci. U.S.A. 91:8180-8184), ecodysone receptor: ecdysone or muristerone A or other analogs or mimics thereof (No et al., 1996, Proc. Natl. Acad. Sci. U.S.A. 93:3346-3351) and DNA gyrase:coumermycin (see e.g. Farrar et al., 1996, Nature 383:178-181). In many applications it ispreferable touse aDNA binding domain which is heterologous to the cells to be engineered. In the case of composite DNA binding domains, component peptide portions which are endogenous to the cells or organism to be engineered are generally preferred.

In another aspect of this embodiment, polynucleotides encoding transcription regulation domains as well as any other functional fragments of PG3 may be introduced into polynucleotides encoding fusion proteins for a variety of regulated gene expression systems, including both allostery-based systems such as those regulated by tetracycline, RU486 or ecdysone, or analogs or mimics thereof, and dimerization-based systems such as those regulated by divalent compounds like FK1012, FKCsA, rapamycin, AP1510 or coumermycin, or analogs or mimics thereof, all as described below (See also, Clackson, “Controlling mammalian gene expression with small molecules”, Current Opinion in Chem. Biol. 1:210-218 (1997)). The fusion proteins may comprise any combination of relevant components, including bundling domains, DNA binding domains, transcription activation (or repression) domains and ligand binding domains. Other heterologous domains may also be included.

Another embodiment of this invention relates to expression systems, preferably vectors and vector-containing cells, using PG3 or part thereof, preferably fragments comprising a transcription regulation domain, more preferably PG-3 fragments from positions 3 to 87, from position 642 to 730, or from position 753 to 833 of SEQ ID No 3. In this regard, recombinant nucleic acids are provided which encode fusion proteins containing the transcription regulation domain of the invention and at least one additional domain that is heterologous thereto, where the peptide sequence of said activation domain is itself eventually modified relative to the naturally occurring sequence from which it was derived to increase or decrease its potency as a transcriptional regulator relative to the counterpart comprising the native peptide sequence. Each of the recombinant nucleic acids of this invention may further comprise an expression control sequence operably linked to the coding sequence and may be provided within a DNA vector, e.g., for use in transducing prokaryotic or eukaryotic cells. Some of the recombinant nucleic acids of a given composition as described above, including any optional recombinant nucleic acids, may be present within a single vector or may be apportioned between two or more vectors. The recombinant nucleic acids may be provided as inserts within one or more recombinant viruses which may be used, for example, to transduce cells in vitro or cells present within an organism, including a human or non-human mamalian subject. It should be appreciated that non-viral approaches (naked DNA, liposomes or other lipid compositions, etc.) may be used to deliver recombinant nucleic acids of this invention to cells in a recipient organism. The resultant engineered cells and their progeny containing one or more of these recombinant nucleic acids or nucleic acid compositions of this invention may be used in a variety of important applications, including human gene therapy, analogous veterinary applications, the creation of cellular or animal models (including transgenic applications) and assay applications. Such cells are useful, for example, in methods involving the addition of a ligand, preferably a cell permeant ligand, to the cells (or administration of the ligand to an organism containing the cells) to regulate expression of a target gene.

In another embodiment, the present invention relates to compositions and methods using PG3 or part thereof, preferably fragments comprising a transcription regulation domain, more preferably PG-3 fragments from positions 3 to 87, from position 642 to 730, or from position 753 to 833 of SEQ ID No 3, to alter the expression of genes of interest in a target cells. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In still another embodiment, PG3 or part thereof, preferably fragments comprising a transcription regulation domain, more preferably PG-3 fragments from positions 3 to 87, from position 642 to 730, or from position 753 to 833 of SEQ ID No 3, may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease.

DNA Repair Activity

The invention relates to compositions and methods using the PG-3 protein of the invention or fragments thereof, preferably preferably PG-3 fragments that comprise amino acids from positions 3 to 87, from position 642 to 730, or from position 753 to 833 of SEQ ID No 3 to repair DNA breaks.

In one embodiment, cell lines may be genetically engineered in order to overexpress PG-3 or part thereof, preferably PG-3 fragments that comprise amino acids from positions 3 to 87, from position 642 to 730, or from position 753 to 833 of SEQ ID No 3 using genetic engineering techniques well known to those skilled in the art. Optionally, such cell lines may be engineered to overexpress fusion proteins comprising PG-3 or part thereof fused to a protein able to repair DNA damage. Exemplary DNA repair proteins for use in the present invention include those from the base excision repair (BER) pathway, e.g., AP endonucleases such as human APE (hAPE, Genbank Accession No. M80261) and related bacterial or yeast proteins such as APN-1 (e.g., Genbank Accession No. U33625 and M33667), exonuclease III (ExoIII, xth gene, Genbank Accession No. M22592,) bacterial endonuclease III (EndoIII, nth gene, Genbank Accession No. J02857), huEndoIII (Genbank Accession No. U79718), and endonuclease IV (EndoIV nfo gene Genbank Accession No. M22591). Additional BER proteins suitable for use in the invention include, for example, DNA glycosylases such as, formamidopyrimidine-DNA glycosylase (FPG, Genbank Accession No. X06036), human 3-alkyladenine DNA glycosylase (HAAG, also known as human methylpurine-DNA glycosylase (HMPG, Genbank Accession No. M74905), NTG-1 (Genbank Accession No. P31378 or 171860), SCR-1 (YAL015C), SCR-2 (Genbank Accession No. YOL043C), DNA ligase I (Genbank Accession No. M36067), .beta.-polymerase (Genbank Accession No. M13140 (human)) and 8-oxoguanine DNA glycosylase (OGG1 Genbank Accession No. U44855 (yeast); Y13479 (mouse); Y11731 (human)). Proteins for use in the invention from the direct reversal pathway include human MGMT (Genbank Accession No. M2997 1) and other similar proteins.

Such cell lines will exhibit a high level of DNA repair activity and will be more resistant to carcinogens inducing single stranded or double stranded DNA breaks. Such cell lines would thus provide an interesting model for carcinogen and drug testing.

### Antibodies That Bind PG3 Polypeptides of the Invention

Definitions

The present invention further relates to antibodies and T-cell antigen receptors (TCR), which specifically bind the polypeptides, and more specifically, the epitopes of the polypeptides of the present invention. The antibodies of the present invention include IgG (including IgG1, IgG2, IgG3, and IgG4), IgA (including IgA1 and IgA2), IgD, IgE, or IgM, and IgY. The term “antibody” (Ab) refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen, which allows an immunological reaction with the antigen. As used herein, the term “antibody” is meant to include whole antibodies, including single-chain whole antibodies, and antigen binding fragments thereof. In a preferred embodiment the antibodies are human antigen binding antibody fragments of the present invention include, but are not limited to, Fab, Fab′ F(ab)₂ and F(ab′)₂, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies, which specifically bind the polypeptides of the present invention. The present invention further includes antibodies that are anti-idiotypic to the antibodies of the present invention.

The antibodies of the present invention may be monospecific, bispecific, and trispecific or have greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, et al. (1991); U.S. Pat. Nos. 5,573,920, 4,474,893, 5,601,819, 4,714,681, 4,925,648; Kostelny et al. (1992), which disclosures are hereby incorporated by reference in their entireties.

Antibodies of the present invention may be described or specified in terms of the epitope(s) or epitope-bearing portion(s) of a polypeptide of the present invention, which are recognized or specifically bound by the antibody. The antibodies may specifically bind a complete protein encoded by a nucleic acid of the present invention, or a fragment thereof. Therefore, the epitope(s) or epitope bearing polypeptide portion(s) may be specified as described herein, e.g., by N-terminal and C-terminal positions, by size in contiguous amino acid residues, or otherwise described herein (including the sequence listing). Antibodies which specifically bind any epitope or polypeptide of the present invention may also be excluded as individual species. Therefore, the present invention includes antibodies that specifically bind specified polypeptides of the present invention, and allows for the exclusion of the same.

Thus, another embodiment of the present invention is a purified or isolated antibody capable of specifically binding to a polypeptide comprising a sequence of SEQ ID No 3. In one aspect of this embodiment, the antibody is capable of binding to an epitope-containing polypeptide comprising at least 6 consecutive amino acids, preferably at least 8 to 10 consecutive amino acids, more preferably at least 12, 15, 20, 25, 30, 40, 50, 100, 150, 200, 250, 300, 400, 500, 600, 700 or 800 consecutive amino acids of SEQ ID No 3.

Antibodies of the present invention may also be described or specified in terms of their cross-reactivity. Antibodies that do not specifically bind any other analog, ortholog, or homologue of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein, e.g., using FASTDB and the parameters set forth herein) to a polypeptide of the present invention are also included in the present invention. Further included in the present invention are antibodies, which only bind polypeptides encoded by polynucleotides, which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Any PG-3 polypeptide or whole protein may be used to generate antibodies capable of specifically binding to an expressed PG-3 protein or fragments thereof as described.

One antibody composition of the invention is capable of specifically binding to the PG-3 protein of SEQ ID No 3. For an antibody composition to specifically bind to the PG-3 protein, it must demonstrate at least a 5%, 10%, 15%, 20%, 25%, 50%, or 100% greater binding affinity for PG-3 protein than for another protein in an ELISA, RIA, or other antibody-based binding assay.

The invention also concerns antibody compositions which are specific for variants of the PG-3 protein, more particuarly variants comprising at least one amino acid selected from the group consisting of a methionine or an isoleucine residue at the position 91 of SEQ ID No 3, a valine or an alanine residue at the position 306 of SEQ ID No 3, a proline or a serine residue at the position 413 of SEQ ID No 3, a glycine or an aspartate residue at the position 528 of SEQ ID No 3, a valine or an alanine residue at the position 614 of SEQ ID No 3, a threonine or an asparagine residue at the position 677 of SEQ ID No 3, a valine or an alanine residue at the position 756 of SEQ ID No 3, a valine or an alanine residue at the position 758 of SEQ ID No 3, a lysine or a glutamate residue at the position 809 of SEQ ID No 3, and a cysteine or an arginine residue at the position 821 of SEQ ID No 3. More preferably, the invention encompasses antibody compositions which are specific for an allelic variant of the PG-3 protein, more particuarly a variant comprising at least one amino acid selected from the group consisting of an arginine or an isoleucine residue at the amino acid position 304 of SEQ ID No 3, a histidine or an aspartic acid residue at the amino acid position 314 of SEQ ID No 3, a threonine or an asparagine residue at the amino acid position 682 of SEQ ID No 3, an alanine or a valine residue at the amino acid position 761 of SEQ ID No 3, and a proline or a serine residue at the amino acid position 828 of SEQ ID No 3.

In a preferred embodiment, the invention concerns antibody compositions, either polyclonal or monoclonal, capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, or 100 amino acids of SEQ ID No 3; preferably, said epitope comprises at least 1, 2, 3, 5 or 10 of the following amino acid positions of SEQ ID No 3:1-100, 101-200, 201-300, 301400, 401-500, 501-600, 601-700, 701-835.

The invention also concerns a purified or isolated antibody capable of specifically binding to a mutated PG-3 protein or to a fragment or variant thereof comprising an epitope of the mutated PG-3 protein. In another preferred embodiment, the present invention concerns an antibody capable of binding to a polypeptide comprising at least 10 consecutive amino acids of a PG-3 protein and including at least one of the amino acids which can be encoded by the trait causing mutations.

In a preferred embodiment, the invention concerns the use in the manufacture of antibodies of a polypeptide comprising a contiguous span of at least 6 amino acids, preferably at least 8 to 10 amino acids, more preferably at least 12, 15, 20, 25, 30, 40, 50, or 100 amino acids of SEQ ID No 3; preferably, said contiguous span comprises at least 1, 2, 3, 5 or 10 of the following amino acid positions of SEQ ID No 3: 1-100, 101-200, 201-300, 301-400, 401-500, 501-600, 601-700, 701-835.

The antibodies of the invention may be labeled using any one of the radioactive, fluorescent or enzymatic labels known in the art.

Consequently, the invention is also directed to a method for specifically detecting the presence of a PG-3 polypeptide according to the invention in a biological sample, said method comprising the following steps:

a) bringing said biological sample into contact with a polyclonal or monoclonal antibody that specifically binds to a PG-3 polypeptide comprising an amino acid sequence of SEQ ID No 3, or to a peptide fragment or to a variant thereof; and

b) detecting the antigen-antibody complex formed.

The invention also concerns a diagnostic kit for detecting the presence of a PG-3 polypeptide according to the present invention in a biological sample in vitro, wherein said kit comprises:

a) a polyclonal or monoclonal antibody that specifically binds to a PG-3 polypeptide comprising the amino acid sequence of SEQ ID No 3, or to a peptide fragment or to a variant thereof; optionally the antibody may be labeled; and

b) a reagent allowing the detection of the antigen-antibody complexes formed, said reagent optionally carrying a label, or being able to be recognized itself by a labeled reagent (particularly in the case when the above-mentioned monoclonal or polyclonal antibody itself is not labeled).

Preparation of Antibodies

The antibodies of the present invention may be prepared by any suitable method known in the art. Some of these methods are described in more detail in the example entitled “PREPARATION OF ANTIBODY COMPOSITIONS TO THE PG-3 PROTEIN”. For example, a polypeptide of the present invention or an antigenic fragment thereof can be administered to an animal in order to induce the production of sera containing “polyclonal antibodies”. As used herein, the term “monoclonal antibody” is not limited to antibodies produced through hybridoma technology but it rather refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (See, e.g., Harlow et al. 1988; Hammerling, et al, 1981). (Said references incorporated by reference in their entireties.) Fab and F(ab′)₂ fragments may be produced, for example, from hybridoma-produced antibodies by proteolytic cleavage, using enzymes such as papain (to produce Fab fragments) or pepsin (to produce F(ab′)₂ fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle, which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman et al. (1995); Ames, et al. (1995); Keffleborough, et al. (1994); Persic, et al. (1997); Burton et al. (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab° F.(ab)₂ and F(ab′)₂ fragments can also be employed using methods known in the art such as those disclosed in WO 92/22324; Mullinax et al. (1992); and Sawai et al. (1995); and Better et al. (1988) (said references incorporated by reference in their entireties).

Examples of techniques which can be used to produce single-chain Fvs and antibodies include those described in U.S. Pat. Nos. 4,946,778 and 5,258,498; Huston et al. (1991); Shu et al. (1993); and Skerra et al. (1988), which disclosures are hereby incorporated by reference in their entireties. For some uses, including in vivo use of antibodies in humans and in vitro detection assays, it may be preferable to use chimeric, humanized, or human antibodies. Methods for producing chimeric antibodies are known in the art. See e.g., Morrison (1985); Oi et al. (1986); Gillies et al. (1989); and U.S. Pat. No. 5,807,715, which disclosures are hereby incorporated by reference in their entireties. Antibodies can be humanized using a variety of techniques including CDR-grafting (EP 0 239 400; WO 91/09967; U.S. Pat. No. 5,530,101; and 5,585,089), veneering or resurfacing, (EP 0 592 106; EP 0 519 596; Padlan, 1991; Studnicka et al., 1994; Roguska et al., 1994), and chain shuffling (U.S. Pat. No. 5,565,332), which disclosures are hereby incorporated by reference in their entireties. Human antibodies can be made by a variety of methods known in the art including phage display methods described above. See also, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies of the present invention may be recombinantly fused or conjugated to molecules useful as labels in detection assays and effector molecules such as beterologous polypeptides, drugs, or toxins. See, e.g., WO 92/08495; WO 91/14438; WO 89/12624; U.S. Pat. No. 5,314,995; and EP 0 396 387, which disclosures are hereby incorporated by reference in their entireties. Fused antibodies may also be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in vitro immunoassays and purification methods using methods known in the art (See e.g., Harper et al. supra; WO 93/21232; EP 0 439 095; Naramura, M. et al. 1994; U.S. Pat. No. 5,474,981; Gillies et al., 1992; Fell et al., 1991) (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half-life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi et al. (1991); Zheng et al. (1995); and Vil et al. (1992) (said references incorporated by reference in their entireties).

Non-human animals or mammals, whether wild-type or transgenic, which express a different species of PG-3 than the one to which antibody binding is desired, and animals which do not express PG-3 (i.e. a PG-3 knock out animal as described herein) are particularly useful for preparing antibodies. PG-3 knock out animals will recognize all or most of the exposed regions of a PG-3 protein as foreign antigens, and therefore produce antibodies with a wider array of PG-3 epitopes. Moreover, smaller polypeptides with only 10 to 30 amino acids may be useful in obtaining specific binding to any one of the PG-3 proteins. In addition, the humoral immune system of animals which produce a species of PG-3 that resembles the antigenic sequence will preferentially recognize the differences between the animal's native PG-3 species and the antigen sequence, and produce antibodies to these unique sites in the antigen sequence. Such a technique will be particularly useful in obtaining antibodies that specifically bind to any one of the PG-3 proteins.

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

The antibodies of the invention may be labeled by any one of the radioactive, fluorescent or enzymatic labels known in the art.

### PG-3-Related Biallelic Markers

Advantages Of The Biallelic Markers Of The Present Invention

The PG-3-related biallelic markers of the present invention offer a number of important advantages over other genetic markers such as RFLP (Restriction fragment length polymorphism) and VNTR (Variable Number of Tandem Repeats) markers.

The first generation of markers were RFLPs, which are variations that modify the length of a restriction fragment. But methods used to identify and to type RFLPs are relatively wasteful of materials, effort, and time. The second generation of genetic markers were VNTRs, which can be categorized as either minisatellites or microsatellites. Minisatellites are tandemly repeated DNA sequences present in units of 5-50 repeats which are distributed along regions of the human chromosomes ranging from 0.1 to 20 kilobases in length. Since they present many possible alleles, their informative content is very high. Minisatellites are scored by performing Southern blots to identify the number of tandem repeats present in a nucleic acid sample from the individual being tested. However, there are only 10⁴ potential VNTRs that can be typed by Southern blotting. Moreover, both RFLP and VNTR markers are costly and time-consuming to develop and assay in large numbers.

Single nucleotide polymorphisms (SNPs) or biallelic markers can be used in the same manner as RFLPs and VNTRs but offer several advantages. SNPs are densely spaced in the human genome and represent the most frequent type of variation. An estimated number of more than 10⁷ sites are scattered along the 3×10⁹ base pairs of the human genome. Therefore, SNPs occur at a greater frequency and with greater uniformity than RFLP or VNTR markers which means that there is a greater probability that such a marker will be found in close proximity to a genetic locus of interest. SNPs are less variable than VNTR markers but are mutationally more stable.

Also, the different forms of a characterized single nucleotide polymorphism, such as the biallelic markers of the present invention, are often easier to distinguish and can therefore be typed easily on a routine basis. Biallelic markers have single nucleotide based alleles and they have only two common alleles, which allows highly parallel detection and automated scoring. The biallelic markers of the present invention offer the possibility of rapid, high throughput genotyping of a large number of individuals.

Biallelic markers are densely spaced in the genome, sufficiently informative and can be assayed in large numbers. The combined effects of these advantages make biallelic markers extremely valuable in genetic studies. Biallelic markers can be used in linkage studies in families, in allele sharing methods, in linkage disequilibrium studies in populations, in association studies of case-control populations or of trait positive and trait negative populations. An important aspect of the present invention is that biallelic markers allow association studies to be performed to identify genes involved in complex traits. Association studies examine the frequency of marker alleles in unrelated case- and control-populations and are generally employed in the detection of polygenic or sporadic traits. Association studies may be conducted within the general population and are not limited to studies performed on related individuals in affected families (linkage studies). Biallelic markers in different genes can be screened in parallel for direct association with disease or response to a treatment. This multiple gene approach is a powerful tool for a variety of human genetic studies as it provides the necessary statistical power to examine the synergistic effect of multiple genetic factors on a particular phenotype, drug response, sporadic trait, or disease state with a complex genetic etiology.

Candidate Gene Of The Present Invention

Different approaches can be employed to perform association studies: genome-wide association studies, candidate region association studies and candidate gene association studies. Genome-wide association studies rely on the screening of genetic markers evenly spaced and covering the entire genome. The candidate gene approach is based on the study of genetic markers specifically located in genes potentially involved in a biological pathway related to the trait of interest. In the present invention, PG-3 is a good candidate gene for cancer or a disorder relating to abnormal cellular differentiation. The candidate gene analysis clearly provides a short-cut approach to the identification of genes and gene polymorphisms related to a particular trait when some information concerning the biology of the trait is available. However, it should be noted that all of the biallelic markers disclosed in the instant application can be employed as part of genome-wide association studies or as part of candidate region association studies and such uses are specifically contemplated in the present invention and claims.

PG-3-Related Biallelic Markers and Polynucleotides Related Thereto

The invention also concerns PG-3-related biallelic markers. As used herein the term “PG-3-related biallelic marker” relates to a set of biallelic markers in linkage disequilibrium with the PG-3 gene. The term PG-3-related biallelic marker includes the biallelic markers designated A1 to A80.

A portion of the biallelic markers of the present invention are disclosed in Table 2. Their locations in the PG-3 gene are indicated in Table 2 and also as a single base polymorphism in the features of SEQ ID Nos 1 and 2 listed in the accompanying Sequence Listing. The pairs of primers allowing the amplification of a nucleic acid containing the polymorphic base of one PG-3 biallelic marker are listed in Table 1 of Example 2.

Eight PG-3-related biallelic markers A3, A6, A7, A14, A70, A71, A72 and A80, are located in the exonic regions of the genomic sequence of PG-3 at the following positions: 10228, 39944, 39973, 76060, 216026, 216082, 216218 and 237555 of the SEQ ID No 1. They are located in exons C, T, I, K and L of the PG-3 gene. Their respective positions in the cDNA and protein sequences are given in Table 2.

The invention also relates to a purified and/or isolated nucleotide sequence comprising a polymorphic base of a PG-3-related biallelic marker, preferably of a biallelic marker selected from the group consisting of A1 to A80, and the complements thereof. The sequence is between 8 and 1000 nucleotides in length, and preferably comprises at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 and 2 or a variant thereof or a complementary sequence thereto. These nucleotide sequences comprise the polymorphic base of either allele 1 or allele 2 of the considered biallelic marker. Optionally, said biallelic marker may be within 6, 5, 4, 3, 2, or 1 nucleotides of the center of said polynucleotide or at the center of said polynucleotide. Optionally, the 3′ end of said contiguous span may be present at the 3′ end of said polynucleotide. Optionally, biallelic marker may be present at the 3′ end of said polynucleotide. Optionally, said polynucleotide may further comprise a label. Optionally, said polynucleotide can be attached to solid support. In a further embodiment, the polynucleotides defined above can be used alone or in any combination.

The invention also relates to a purified and/or isolated nucleotide sequence comprising a sequence between 8 and 1000 nucleotides in length, and preferably at least 8, 10, 12, 15, 18, 20, 25, 35, 40, 50, 60, 70, 80, 100, 250, 500 or 1000 contiguous nucleotides of a nucleotide sequence selected from the group consisting of SEQ ID Nos 1 and 2 or a variant thereof or a complementary sequence thereto. Optionally, the 3′ end of said polynucleotide may be located within or at least 2, 4, 6, 8, 10, 12, 15, 18, 20, 25, 50, 100, 250, 500, or 1000 nucleotides upstream of a PG-3-related biallelic marker in said sequence. Optionally, said PG-3-related biallelic marker is selected from the group consisting of A1 to A80; optionally, the 3′ end of said polynucleotide may be located 1 nucleotide upstream of a PG-3-related biallelic marker in said sequence. Optionally, said polynucleotide may further comprise a label. Optionally, said polynucleotide can be attached to solid support. In a further embodiment, the polynucleotides defined above can be used alone or in any combination.

In a preferred embodiment, the sequences comprising a polymorphic base of one of the biallelic markers listed in Table 2 are selected from the group consisting of the nucleotide sequences comprising, consisting essentially of, or consisting of the amplicons listed in Table 1 or a variant thereof or a complementary sequence thereto.

The invention further concerns a nucleic acid encoding the PG-3 protein, wherein said nucleic acid comprises a polymorphic base of a biallelic marker selected from the group consisting of A1 to A80 and the complements thereof.

The invention also encompasses the use of any polynucleotide for, or any polynucleotide for use in, determining the identity of one or more nucleotides at a PG-3-related biallelic marker. In addition, the polynucleotides of the invention for use in determining the identity of one or more nucleotides at a PG-3-related biallelic marker encompass polynucleotides with any further limitation described in this disclosure, or those following, specified alone or in any combination. Optionally, said PG-3-related biallelic marker is selected from the group consisting of A1 to A80, and the complements thereof, or optionally the biallelic markers in linkage disequilibrium therewith; optionally, said PG-3-related biallelic marker is selected from the group consisting of A1 to A5 and A8 to A80, and the complements thereof, or optionally the biallelic markers in linkage disequilibrium therewith; optionally, said PG-3-related biallelic marker is selected from the group consisting A6 and A7, and the complements thereof, or optionally the biallelic markers in linkage disequilibrium therewith; optionally, said polynucleotide may comprise a sequence disclosed in the present specification; optionally, said polynucleotide may comprise, consist of, or consist essentially of any polynucleotide described in the present specification; optionally, said determining may involve a hybridization assay, sequencing assay, microsequencing assay, or an enzyme-based mismatch detection assay; optionally, said polynucleotide may be attached to a solid support, array, or addressable array; optionally, said polynucleotide may be labeled. A preferred polynucleotide may be used in a hybridization assay for determining the identity of the nucleotide at a PG-3-related biallelic marker. Another preferred polynucleotide may be used in a sequencing or microsequencing assay for determining the identity of the nucleotide at a PG-3-related biallelic marker. A third preferred polynucleotide may be used in an enzyme-based mismatch detection assay for determining the identity of the nucleotide at a PG-3-related biallelic marker. A fourth preferred polynucleotide may be used in amplifying a segment of polynucleotides comprising a PG-3-related biallelic marker. Optionally, any of the polynucleotides described above may be attached to a solid support, array, or addressable array; optionally, said polynucleotide may be labeled.

Additionally, the invention encompasses the use of any polynucleotide for, or any polynucleotide for use in amplifying a segment of nucleotides comprising a PG-3-related biallelic marker. In addition, the polynucleotides of the invention for use in amplifying a segment of nucleotides comprising a PG-3-related biallelic marker encompass polynucleotides with any further limitation described in this disclosure, or those following, specified alone or in any combination: Optionally, said PG-3-related biallelic marker is selected from the group consisting of A1 to A80, and the complements thereof, or optionally the biallelic markers in linkage disequilibrium therewith; optionally, said PG-3-related biallelic marker is selected from the group consisting of A1 to A5 and A8 to A80, and the complements thereof, or optionally the biallelic markers in linkage disequilibrium therewith; optionally, said PG-3-related biallelic marker is selected from the group consisting A6 and A7, and the complements thereof, or optionally the biallelic markers in linkage disequilibrium therewith; optionally, said polynucleotide may comprise a sequence disclosed in the present specification; optionally, said polynucleotide may comprise, consist of, or consist essentially of any polynucleotide described in the present specification; optionally, said amplifying may involve PCR or LCR. Optionally, said polynucleotide may be attached to a solid support, array, or addressable array. Optionally, said polynucleotide may be labeled.

The primers for amplification or sequencing reaction of a polynucleotide comprising a biallelic marker of the invention may be designed from the disclosed sequences for any method known in the art. A preferred set of primers are fashioned such that the 3′ end of the contiguous span of identity with a sequence selected from the group consisting of SEQ ID Nos 1 and 2 or a sequence complementary thereto or a variant thereof is present at the 3′ end of the primer. Such a configuration allows the 3′ end of the primer to hybridize to a selected nucleic acid sequence and dramatically increases the efficiency of the primer for amplification or sequencing reactions. Allele specific primers may be designed such that a polymorphic base of a biallelic marker is at the 3′ end of the contiguous span and the contiguous span is present at the 3′ end of the primer. Such allele specific primers tend to selectively prime an amplification or sequencing reaction so long as they are used with a nucleic acid sample that contains one of the two alleles present at a biallelic marker. The 3′ end of the primer of the invention may be located within or at least 2, 4, 6, 8, 10, 12, 15, 18, 20, 25, 50, 100, 250, 500, or 1000 nucleotides upstream of a PG-3-related biallelic marker in said sequence or at any other location which is appropriate for their intended use in sequencing, amplification or the location of novel sequences or markers. Thus, another set of preferred amplification primers comprise an isolated polynucleotide consisting essentially of a contiguous span of at least 8, 10, 12, 15, 18, 20, 25, 30, 35, 40, or 50 nucleotides in length of a sequence selected from the group consisting of SEQ ID Nos 1 and 2 or a sequence complementary thereto or a variant thereof, wherein the 3′ end of said contiguous span is located at the 3′ end of said polynucleotide, and wherein the 3′ end of said polynucleotide is located upstream of a PG-3-related biallelic marker in said sequence. Preferably, those amplification primers comprise a sequence selected from the group consisting of the sequences B1 to B52 and C1 to C52. Primers with their 3′ ends located 1 nucleotide upstream of a biallelic marker of PG-3 have a special utility as microsequencing assays. Preferred microsequencing primers are described in Table 4. Optionally, said PG-3-related biallelic marker is selected from the group consisting of A1 to A80, and the complements thereof, or optionally the biallelic markers in linkage disequilibrium therewith; optionally, said PG-3-related biallelic marker is selected from the group consisting of A1 to A5 and A8 to A80, and the complements thereof, or optionally the biallelic markers in linkage disequilibrium therewith; optionally, said PG-3-related biallelic marker is selected from the group consisting A6 and A7, and the complements thereof, or optionally the biallelic markers in linkage disequilibrium therewith; optionally, microsequencing primers are selected from the group consisting of the nucleotide sequences of D1 to D4, D6 to D80, E1 to E4 and E6 to E80. More preferred microsequencing primers are selected from the group consisting of the nucleotides sequences of D14, D46, D68, D70, D71, E3, E6, E7, E11, E13, E42, E44, E72 and E75.

The probes of the present invention may be designed from the disclosed sequences for use in any method known in the art, particularly methods for testing if a marker disclosed herein is present in a sample. A preferred set of probes may be designed for use in the hybridization assays of the invention in any manner known in the art such that they selectively bind to one allele of a biallelic marker, but not the other under any particular set of assay conditions. Preferred hybridization probes comprise the polymorphic base of either allele 1 or allele 2 of the relevant biallelic marker. Optionally, said biallelic marker may be within 6, 5, 4, 3, 2, or 1 nucleotides of the center of the hybridization probe or at the center of said probe. In a preferred embodiment, the probes are selected from the group consisting of the sequences P1 to P4 and P6 to P80 and the complementary sequence thereto.

It should be noted that the polynucleotides of the present invention are not limited to having the exact flanking sequences surrounding the polymorphic bases which are enumerated in Sequence Listing. Rather, it will be appreciated that the flanking sequences surrounding the biallelic markers may be lengthened or shortened to any extent compatible with their intended use and the present invention specifically contemplates such sequences. The flanking regions outside of the contiguous span need not be homologous to native flanking sequences which actually occur in human subjects. The addition of any nucleotide sequence which is compatible with the polynucleotide's intended use is specifically contemplated.

Primers and probes may be labeled or immobilized on a solid support as described in the section entitled “Oligonucleotide probes and primers”.

The polynucleotides of the invention which are attached to a solid support encompass polynucleotides with any further limitation described in this disclosure, or those following, alone or in any combination: optionally, said polynucleotides may be attached individually or in groups of at least 2, 5, 8, 10, 12, 15, 20, or 25 distinct polynucleotides of the invention to a single solid support. Optionally, polynucleotides other than those of the invention may attached to the same solid support as polynucleotides of the invention. Optionally, when multiple polynucleotides are attached to a solid support they may be attached at random locations, or in an ordered array. Optionally, said ordered array may be addressable.

The present invention also encompasses diagnostic kits comprising one or more polynucleotides of the invention with a portion or all of the necessary reagents and instructions for genotyping a test subject by determining the identity of a nucleotide at a PG-3-related biallelic marker. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or an enzyme-based mismatch detection assay method.

### Methods for De Novo Identification of Biallelic Markers

Any of a variety of methods can be used to screen a genomic fragment for single nucleotide polymorphisms, including methods such as differential hybridization with oligonucleotide probes, detection of changes in the mobility measured by gel electrophoresis or direct sequencing of the amplified nucleic acid. A preferred method for identifying biallelic markers involves comparative sequencing of genomic DNA fragments from an appropriate number of unrelated individuals.

In a first embodiment, DNA samples from unrelated individuals are pooled together, following which the genomic DNA of interest is amplified and sequenced. The nucleotide sequences thus obtained are then analyzed to identify significant polymorphisms. One of the major advantages of this method resides in the fact that the pooling of the DNA samples substantially educes the number of DNA amplification reactions and sequencing reactions, which must be carried out. Moreover, this method is sufficiently sensitive so that a biallelic marker obtained thereby usually demonstrates a sufficient frequency of its less common allele to be useful in conducting association studies.

In a second embodiment, the DNA samples are not pooled and are therefore amplified and sequenced individually. This method is usually preferred when biallelic markers need to be identified in order to perform association studies within candidate genes. Preferably, highly relevant gene regions such as promoter regions or exon regions may be screened for biallelic markers. A biallelic marker obtained using this method may show a lower degree of informativeness for conducting association studies, e.g. if the frequency of its less frequent allele is less than about 10%. Such a biallelic marker will, however, be sufficiently informative to conduct association studies and it will further be appreciated that including less informative biallelic markers in the genetic analysis studies of the present invention, may, in some cases, allow the direct identification of causal mutations, which may, depending on their penetrance, be rare mutations.

The following is a description of the various parameters of a preferred method used by the inventors for the identification of the biallelic markers of the present invention.

Genomic DNA Samples

The genomic DNA samples from which the biallelic markers of the present invention are generated are preferably obtained from unrelated individuals corresponding to a heterogeneous population of known ethnic background. The number of individuals from whom DNA samples are obtained can vary substantially, but is preferably from about 10 to about 1000, or preferably from about 50 to about 200 individuals. It is usually preferred to collect DNA samples from at least about 100 individuals in order to have sufficient polymorphic diversity in a given population to identify as many markers as possible and to generate statistically significant results.

As for the source of the genomic DNA to be subjected to analysis, any test sample can be foreseen without any particular limitation. These test samples include biological samples, which can be tested by the methods of the present invention described herein, and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like; biological fluids such as cell culture supernatants; fixed tissue specimens including tumor and non-tumor tissue and lymph node tissues; bone marrow aspirates and fixed cell specimens. The preferred source of genomic DNA used in the present invention is from peripheral venous blood of each donor. Techniques to prepare genomic DNA from biological samples are well known to the skilled technician. Details of a preferred embodiment are provided in Example 1. The person skilled in the art can choose to amplify pooled or unpooled DNA samples.

DNA Amplification

The identification of biallelic markers in a sample of genomic DNA may be facilitated through the use of DNA amplification methods. DNA samples can be pooled or unpooled for the amplification step. DNA amplification techniques are well known to those skilled in the art.

Amplification techniques that can be used in the context of the present invention include, but are not limited to, the ligase chain reaction (LCR) described in EP-A-320 308, WO 9320227 and EP-A439 182, the polymerase chain reaction (PCR, RT-PCR) and techniques such as the nucleic acid sequence based amplification (NASBA) described in Guatelli J. C., et al. (1990) and in Compton J. (1991), Q-beta amplification as described in European Patent Application No 4544610, strand displacement amplification as described in Walker et al. (1996) and EP A 684 315 and, target mediated amplification as described in PCT Publication WO 9322461.

LCR and Gap LCR are exponential amplification techniques, both of which utilize DNA ligase to join adjacent primers annealed to a DNA molecule. In Ligase Chain Reaction (LCR), probe pairs are used which include two primary (first and second) and two secondary (third and fourth) probes, all of which are employed in molar excess to target. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes, which can be ligated to form a complementary, secondary ligated product. It is important to realize that the ligated products are functionally equivalent to either the target or its complement. By repeated cycles of hybridization and ligation, amplification of the target sequence is achieved. A method for multiplex LCR has also been described (WO 9320227). Gap LCR (GLCR) is a version of LCR where the probes are not adjacent but are separated by 2 to 3 bases.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770 or, to use Asymmetric Gap LCR (RT-AGLCR) as described by Marshall et al. (1994). AGLCR is a modification of GLCR that allows the amplification of RNA.

The PCR technology is the preferred amplification technique used in the present invention. A variety of PCR techniques are familiar to those skilled in the art. For a review of PCR technology, see White (1992) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188.

The PCR technology is the preferred amplification technique used to identify new biallelic markers. A typical example of a PCR reaction suitable for the purposes of the present invention is provided in Example 2.

One of the aspects of the present invention is a method for the amplification of the human PG-3 gene, particularly of a fragment of the genomic sequence of SEQ ID No 1 or of the cDNA sequence of SEQ ID No 2, or a fragment or a variant thereof in a test sample, preferably using the PCR technology. This method comprises the steps of:

a) contacting a test sample with amplification reaction reagents comprising a pair of amplification primers as described above which are located on either side of the polynucleotide region to be amplified, and

b) optionally, detecting the amplification products.

The invention also concerns a kit for the amplification of a PG-3 gene sequence, particularly of a portion of the genomic sequence of SEQ ID No 1 or of the cDNA sequence of SEQ ID No 2, or a variant thereof in a test sample, wherein said kit comprises:

a) a pair of oligonucleotide primers located on either side of the PG-3 region to be amplified;

b) optionally, the reagents necessary for performing the amplification reaction.

In one embodiment of the above amplification method and kit, the amplification product is detected by hybridization with a labeled probe having a sequence which is complementary to the amplified region. In another embodiment of the above amplification method and kit, primers comprise a sequence which is selected from the group consisting of the nucleotide sequences of B1 to B52, C1 to C52, D1 to D4, D6 to D80, E1 to E4, and E6 to E80.

In a first embodiment of the present invention, biallelic markers are identified using genomic sequence information generated by the inventors. Sequenced genomic DNA fragments are used to design primers for the amplification of 500 bp fragments. These 500 bp fragments are amplified from genomic DNA and are scanned for biallelic markers. Primers may be designed using the OSP software (Hillier L. and Green P., 1991). All primers may contain, upstream of the specific target bases, a common oligonucleotide tail that serves as a sequencing primer. Those skilled in the art are familiar with primer extensions, which can be used for these purposes.

Preferred primers, useful for the amplification of genomic sequences encoding the candidate genes, focus on promoters, exons and splice sites of the genes. A biallelic marker presents a higher probability to be a causal mutation if it is located in these functional regions of the gene. Preferred amplification primers of the invention include the nucleotide sequences B 1 to B52 and C1 to C52, detailed further in Example 2, Table 1.

Sequencing of Amplified Genomic DNA and Identification of Single Nucleotide Polymorphisms

The amplification products generated as described above, are then sequenced using any method known and available to the skilled technician. Methods for sequencing DNA using either the dideoxy-mediated method (Sanger method) or the Maxam-Gilbert method are widely known to those of ordinary skill in the art. Such methods are disclosed in Sambrook et al. (1989) for example. Alternative approaches include hybridization to high-density DNA probe arrays as described in Chee et al. (1996).

Preferably, the amplified DNA is subjected to automated dideoxy terminator sequencing reactions using a dye-primer cycle sequencing protocol. The products of the sequencing reactions are run on sequencing gels and the sequences are determined using gel image analysis. The polymorphism search is based on the presence of superimposed peaks in the electrophoresis pattern resulting from different bases occurring at the same position. Because each dideoxy terminator is labeled with a different fluorescent molecule, the two peaks corresponding to a biallelic site present distinct colors corresponding to two different nucleotides at the same position on the sequence. However, the presence of two peaks can be an artifact due to background noise. To exclude such an artifact, the two DNA strands are sequenced and a comparison between the peaks is carried out. In order to confirm that a sequence is polymorphic, the polymorphism is be detected on both strands.

The above procedure permits those amplification products which contain biallelic markers to be identified. The detection limit for the frequency of biallelic polymorphisms detected by sequencing pools of 100 individuals is approximately 0.1 for the minor allele, as verified by sequencing pools of known allelic frequencies. However, more than 90% of the biallelic polymorphisms detected by the pooling method have a frequency for the minor allele higher than 0.25. Therefore, the biallelic markers selected by this method have a frequency of at least 0.1 for the minor allele and less than 0.9 for the major allele. Preferably, the biallelic markers selected by this method have a frequency of at least 0.2 for the minor allele and less than 0.8 for the major allele, more preferably at least 0.3 for the minor allele and less than 0.7 for the major allele. Thus, the biallelic markers preferably have a heterozygosity rate higher than 0. 18, more preferably higher than 0.32, still more preferably higher than 0.42.

In another embodiment, biallelic markers are detected by sequencing individual DNA samples. In some embodiments, the frequency of the minor allele of such a biallelic marker may be less than 0.1.

Validation of the Biallelic Markers of the Present Invention

The polymorphisms are evaluated for their usefulness as genetic markers by validating that both alleles are present in a population. Validation of the biallelic markers is accomplished by genotyping a group of individuals by a method of the invention and demonstrating that both alleles are present. Microsequencing is a preferred method of genotyping alleles. The validation by genotyping step may be performed on individual samples derived from each individual in the group or by genotyping a pooled sample derived from more than one individual. The group can be as small as one individual if that individual is heterozygous for the allele in question. Preferably the group contains at least three individuals, more preferably the group contains five or six individuals, so that a single validation test will be more likely to result in the validation of more of the biallelic markers that are being tested. It should be noted, however, that when the validation test is performed on a small group it may result in a false negative result if as a result of sampling error none of the individuals tested carries one of the two alleles. Thus, the validation process is less useful in demonstrating that a particular initial result is an artifact, than it is at demonstrating that there is a bona fide biallelic marker at a particular position in a sequence. All of the genotyping, haplotyping, association, and interaction study methods of the invention may optionally be performed solely with validated biallelic markers.

Evaluation of the Frequency of the Biallelic Markers of the Present Invention

The validated biallelic markers are further evaluated for their usefulness as genetic markers by determining the frequency of the least common allele at the biallelic marker site. The higher the frequency of the less common allele, the greater the usefulness of the biallelic marker in association and interaction studies. The identification of the least common allele is accomplished by genotyping a group of individuals by a method of the invention and demonstrating that both alleles are present. The determination of marker frequency by genotyping may be performed using individual samples derived from each individual in the group or by genotyping a pooled sample derived from more than one individual. The group must be large enough to be representative of the population as a whole. Preferably the group contains at least 20 individuals, more preferably the group contains at least 50 individuals, most preferably the group contains at least 100 individuals. Of course the larger the group the greater the accuracy of the frequency determination because of reduced sampling error. A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

### Methods for Genotyping an Individual for Biallelic Markers

Methods are provided to genotype a biological sample for one or more biallelic markers of the present invention, all of which may be performed in vitro. Such methods of genotyping comprise determining the identity of a nucleotide at a PG-3 biallelic marker site by any method known in the art. These methods find use in genotyping case-control populations in association studies as well as individuals in the context of detection of alleles of biallelic markers which are known to be associated with a given trait, in which case both copies of the biallelic marker present in individual's genome are determined so that an individual may be classified as homozygous or heterozygous for a particular allele.

These genotyping methods can be performed on nucleic acid samples derived from a single individual or pooled DNA samples.

Genotyping can be performed using methods similar to those described above for the identification of the biallelic markers, or using other genotyping methods such as those further described below. In preferred embodiments, the comparison of sequences of amplified genomic fragments from different individuals is used to identify new biallelic markers whereas microsequencing is used for genotyping known biallelic markers in diagnostic and association study applications.

In one embodiment, the invention encompasses methods of genotyping comprising determining the identity of a nucleotide at a PG-3-related biallelic marker or the complement thereof in a biological sample; optionally, the PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, the biological sample is derived from a single subject; optionally, the identity of the nucleotides at said biallelic marker is determined for both copies of said biallelic marker present in said individual's genome; optionally, said biological sample is derived from multiple subjects; optionally, the genotyping methods of the invention encompass methods with any further limitation described in this disclosure, or those following, alone or in any combination; optionally, said method is performed in vitro; optionally, the method further comprises amplifying a portion of said sequence comprising the biallelic marker prior to said determining step; optionally, the amplification is performed by PCR, LCR, or replication of a recombinant vector comprising an origin of replication and said fragment in a host cell; optionally, the determination involves a hybridization assay, a sequencing assay, a microsequencing assay, or an enzyme-based mismatch detection assay.

Source of Nucleic Acids for genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

Amplification of DNA Fragments Comprising Biallelic Markers

Methods and polynucleotides are provided to amplify a segment of nucleotides comprising one or more biallelic marker of the present invention. It will be appreciated that amplification of DNA fragments comprising biallelic markers may be used in various methods and for various purposes and is not restricted to genotyping. Nevertheless, many genotyping methods, although not all, require the previous amplification of the DNA region carrying the biallelic marker of interest. Such methods specifically increase the concentration or total number of sequences that span the biallelic marker or include that site and sequences located either distal or proximal to it. Diagnostic assays may also rely on amplification of DNA segments carrying a biallelic marker of the present invention. Amplification of DNA may be achieved by any method known in the art. Amplification techniques are described above in the section entitled, “DNA amplification.”

Some of these amplification methods are particularly suited for the detection of single nucleotide polymorphisms and allow the simultaneous amplification of a target sequence and the identification of the polymorphic nucleotide as further described below.

The identification of biallelic markers as described above allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention.

In some embodiments, the present invention provides primers for amplifying a DNA fragment containing one or more biallelic markers of the present invention. Preferred amplification primers are listed in Example 2. It will be appreciated that the primers listed are merely exemplary and that any other set of primers which produce amplification products containing one or more biallelic markers of the present invention are also of use.

The spacing of the primers determines the length of the segment to be amplified. In the context of the present invention, amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support as described in the section “Oligonucleotide probes and primers”.

Methods of Genotyping DNA samples for Biallelic Markers

Any method known in the art can be used to identify the nucleotide present at a biallelic marker site. Since the biallelic marker allele to be detected has been identified and specified in the present invention, detection will prove simple for one of ordinary skill in the art by employing any of a number of techniques. Many genotyping methods require the previous amplification of the DNA region carrying the biallelic marker of interest. While the amplification of target or signal is often preferred at present, ultrasensitive detection methods which do not require amplification are also encompassed by the present genotyping methods. Methods well-known to those skilled in the art that can be used to detect biallelic polymorphisms include methods such as, conventional dot blot analyzes, single strand conformational polymorphism analysis (SSCP) described by Orita et al. (1989), denaturing gradient gel electrophoresis (DGGE), heteroduplex analysis, mismatch cleavage detection, and other conventional techniques as described in Sheffield et al. (1991), White et al. (1992), Grompe et al. (1989 and 1993). Another method for determining the identity of the nucleotide present at a particular polymorphic site employs a specialized exonuclease-resistant nucleotide derivative as described in U.S. Pat. No. 4,656,127.

Preferred methods involve directly determining the identity of the nucleotide present at a biallelic marker site by sequencing assay, enzyme-based mismatch detection assay, or hybridization assay. The following is a description of some preferred methods. A highly preferred method is the microsequencing technique. The term “sequencing” is generally used herein to refer to polymerase extension of duplex primer/template complexes and includes both traditional sequencing and microsequencing.

1) Sequencing Assays

The nucleotide present at a polymorphic site can be determined by sequencing methods. In a preferred embodiment, DNA samples are subjected to PCR amplification before sequencing as described above. DNA sequencing methods are described in the section entitled “Sequencing Of Amplified Genomic DNA And Identification Of Single Nucleotide Polymorphisms”.

Preferably, the amplified DNA is subjected to automated dideoxy terminator sequencing reactions using a dye-primer cycle sequencing protocol. Sequence analysis allows the identification of the base present at the biallelic marker site.

2) Microsequencing Assays

In microsequencing methods, the nucleotide at a polymorphic site in a target DNA is detected by a single nucleotide primer extension reaction. This method involves appropriate microsequencing primers which hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883. Alternatively capillary electrophoresis can be used in order to process a higher number of assays simultaneously. An example of a typical microsequencing procedure that can be used in the context of the present invention is provided in Example 4.

Different approaches can be used for the labeling and detection of ddNTPs. A homogeneous phase detection method based on fluorescence resonance energy transfer has been described by Chen and Kwok (1997) and Chen et al. (1997). In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff and Smirnov, 1997).

Microsequencing may be achieved by the established microsequencing method or by developments or derivatives thereof. Alternative methods include several solid-phase microsequencing techniques. The basic microsequencing protocol is the same as described previously, except that the method is conducted as a heterogeneous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner, oligonucleotides or templates may be attached to a solid support in a high-density format. In such solid phase microsequencing reactions, incorporated ddNTPs can be radiolabeled (Syvänen, 1994) or linked to fluorescein (Livak and Hainer, 1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Harju et al., 1993) or biotinylated ddNTP and horseradish peroxidase-conjugated streptavidin with o-phenylenediamine as a substrate (WO 92/15712). As yet another alternative solid-phase microsequencing procedure, Nyren et al. (1993) described a method relying on the detection of DNA polymerase activity by an enzymatic luminometric inorganic pyrophosphate detection assay (ELIDA).

Pastinen et al. (1997) describe a method for multiplex detection of single nucleotide polymorphism in which the solid phase minisequencing principle is applied to an oligonucleotide array format. High-density arrays of DNA probes attached to a solid support (DNA chips) are further described below.

In one aspect the present invention provides polynucleotides and methods to genotype one or more biallelic markers of the present invention by performing a microsequencing assay. Preferred microsequencing primers include the nucleotide sequences D1 to D4 and D6 to D80 and E1 to E4 and E6 to E80. It will be appreciated that the microsequencing primers listed in Example 4 are merely exemplary and that any primer having a 3′ end immediately adjacent to the polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers listed in Example 4, or fragments comprising at least 8, 12, 15, 20, 25, 30, 40, or 50 consecutive nucleotides thereof, to the extent that such lengths are consistent with the primer described, and having a 3′ terminus immediately upstream of the corresponding biallelic marker, for determining the identity of a nucleotide at a biallelic marker site.

3) Mismatch Detection Assays Based on Polymerases and Ligases

In one aspect the present invention provides polynucleotides and methods to determine the allele of one or more biallelic markers of the present invention in a biological sample, by mismatch detection assays based on polymerases and/or ligases. These assays are based on the specificity of polymerases and ligases. Polymerization reactions place particularly stringent requirements on correct base pairing of the 3′ end of the amplification primer and the joining of two oligonucleotides hybridized to a target DNA sequence is quite sensitive to mismatches close to the ligation site, especially at the 3′ end. Methods, primers and various parameters to amplify DNA fragments comprising biallelic markers of the present invention are further described above in the section entitled “Amplification Of DNA Fragments Comprising Biallelic Markers”.

Allele Specific Amplification Primers

Discrimination between the two alleles of a biallelic marker can also be achieved by allele specific amplification, a selective strategy whereby one of the alleles is amplified without amplification of the other allele. For allele specific amplification, at least one member of the pair of primers is sufficiently complementary with a region of a PG-3 gene comprising the polymorphic base of a biallelic marker of the present invention to hybridize therewith and to initiate the amplification. Such primers are able to discriminate between the two alleles of a biallelic marker.

This is accomplished by placing the polymorphic base at the 3′ end of one of the amplification primers. Because the extension progresses from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well within the ordinary skill in the art.

Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al. (1990). In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in the section entitled “DNA Amplification”. LCR uses two pairs of probes to exponentially amplify a specific target. The sequences of each pair of oligonucleotides are selected to permit the pair to hybridize to abutting sequences of the same strand of the target. Such hybridization forms a substrate for a template-dependant ligase In accordance with the present invention, LCR can be performed with oligonucleotides having the proximal and distal sequences of the same strand of a biallelic marker site. In one embodiment, either oligonucleotide will be designed to include the biallelic marker site. In such an embodiment, the reaction conditions are selected such that the oligonucleotides can be ligated together only if the target molecule either contains or lacks the specific nucleotide that is complementary to the biallelic marker on the oligonucleotide. In an alternative embodiment, the oligonucleotides will not include the biallelic marker, such that when they hybridize to the target molecule, a “gap” is created as described in WO 90/01069. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

4) Hybridization Assay Methods

A preferred method of determining the identity of the nucleotide present at a biallelic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook et al., 1989).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a biallelic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridization can be performed in solution, it is preferred to employ a solid-phase hybridization assay. The target DNA comprising a biallelic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., 1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., 1995). In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., 1998).

The polynucleotides provided herein can be used to produce probes which can be used in hybridization assays for the detection of biallelic marker alleles in biological samples. These probes preferably comprise between 8 and 50 nucleotides and are sufficiently complementary to a sequence comprising a biallelic marker of the present invention to hybridize thereto and preferably sufficiently specific to be able to discriminate the targeted sequence for only one nucleotide variation. A particularly preferred probe is 25 nucleotides in length. Preferably the biallelic marker is within 4 nucleotides of the center of the polynucleotide probe. In particularly preferred probes, the biallelic marker is at the center of said polynucleotide. Preferred probes comprise a nucleotide sequence selected from the group consisting of amplicons listed in Table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. Preferred probes comprise a nucleotide sequence selected from the group consisting of P1 to P4 and P6 to P80 and the sequences complementary thereto. In preferred embodiments the polymorphic base(s) are within 5, 4, 3, 2, 1, nucleotides of the center of the said polynucleotide, more preferably at the center of said polynucleotide.

Preferably the probes of the present invention are labeled or immobilized on a solid support. Labels and solid supports are further described in the section entitled “Oligonucleotide Probes and Primers”. The probes can be non-extendable as described in the section entitled “Oligonucleotide Probes and Primers”.

By assaying the hybridization to an allele specific probe, one can detect the presence or absence of a biallelic marker allele in a given sample. High-Throughput parallel hybridization in array format is specifically encompassed within “hybridization assays” and is described below.

5) Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant strains, and in the protease gene of HIV-1 virus (Hacia et al., 1996; Shoemaker et al., 1996; Kozal et al., 1996). Chips of various formats for use in detecting biallelic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280, describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of nucleotides. Tiling strategies are further described in PCT application No. WO 95/11995. In a particular aspect, arrays are tiled for a number of specific, identified biallelic marker sequences. In particular, the array is tiled to include a number of detection blocks, each detection block being specific for a specific biallelic marker or a set of biallelic markers. For example, a detection block may be tiled to include a number of probes, which span the sequence segment that includes a specific polymorphism. To obtain probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences about 15 nucleotides in length. In further embodiments, the chip may comprise an array including at least one of the sequences selected from the group consisting of amplicons listed in Table 1 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention. Solid supports and polynucleotides of the present invention attached to solid supports are further described in the section entitled “Oligonucleotide Probes And Primers”.

6) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts.

For genotyping biallelic markers, the microfluidic system may integrate nucleic acid amplification, microsequencing, capillary electrophoresis and a detection method such as laser-induced fluorescence detection.

### Methods of Genetic Analysis Using the Biallelic Markers of the Present Invention

Different methods are available for the genetic analysis of complex traits (see Lander and Schork, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury et al., 1993). In general, the biallelic markers of the present invention find use in any method known in the art to demonstrate a statistically significant correlation between a genotype and a phenotype. The biallelic markers may be used in parametric and non-parametric linkage analysis methods. Preferably, the biallelic markers of the present invention are used to identify genes associated with detectable traits using association studies, an approach which does not require the use of affected families and which permits the identification of genes associated with complex and sporadic traits.

The genetic analysis using the biallelic markers of the present invention may be conducted on any scale. The whole set of biallelic markers of the present invention or any subset of biallelic markers of the present invention corresponding to the candidate gene may be used. Further, any set of genetic markers including a biallelic marker of the present invention may be used. A set of biallelic polymorphisms that could be used as genetic markers in combination with the biallelic markers of the present invention has been described in WO 98/20165. As mentioned above, it should be noted that the biallelic markers of the present invention may be included in any complete or partial genetic map of the human genome. These different uses are specifically contemplated in the present invention and claims.

Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those traits (Weir, 1996). The classical method for linkage analysis is the logarithm of odds (lod) score method (see Morton, 1955; Ott, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (1996).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The biallelic markers of the present invention may be used in both parametric and non-parametric linkage analysis. Preferably biallelic markers may be used in non-parametric methods which allow the mapping of genes involved in complex traits. The biallelic markers of the present invention may be used in both IBD- and IBS-methods to map genes affecting a complex trait. In such studies, taking advantage of the high density of biallelic markers, several adjacent biallelic marker loci may be pooled to achieve the efficiency attained by multi-allelic markers (Zhao et al, 1998).

Population Association Studies

The present invention comprises methods for detecting an association between the PG-3 gene and a detectable trait using the biallelic markers of the present invention. In one embodiment the present invention comprises methods to detect an association between a biallelic marker allele or a biallelic marker haplotype and a trait. Further, the invention comprises methods to identify a trait causing allele in linkage disequilibrium with any biallelic marker allele of the present invention.

As described above, alternative approaches can be employed to perform association studies: genome-wide association studies, candidate region association studies and candidate gene association studies. In a preferred embodiment, the biallelic markers of the present invention are used to perform candidate gene association studies. The candidate gene analysis clearly provides a short-cut approach to the identification of genes and gene polymorphisms related to a particular trait when some information concerning the biology of the trait is available. Further, the biallelic markers of the present invention may be incorporated in any map of genetic markers of the human genome in order to perform genome-wide association studies. Methods to generate a high-density map of biallelic markers has been described in U.S. Provisional Patent application serial No. 60/082,614. The biallelic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the biallelic markers of the present invention can therefore be used to refine the location of a trait causing allele in a candidate region identified by Linkage Analysis methods. Moreover, once a chromosome segment of interest has been identified, the presence of a candidate gene such as a candidate gene of the present invention, in the region of interest can provide a shortcut to the identification of the trait causing allele. Biallelic markers of the present invention can be used to demonstrate that a candidate gene is associated with a trait. Such uses are specifically contemplated in the present invention.

Determining the Frequency of a Biallelic Marker Allele or of a Biallelic Marker Haplotype in a Population

Association studies explore the relationships among frequencies for sets of alleles between loci.

### Determining the Frequency of an Allele in a Population

Allelic frequencies of the biallelic markers in a populations can be determined using one of the methods described above under the heading “Methods for genotyping an individual for biallelic markers”, or any genotyping procedure suitable for this intended purpose. Genotyping pooled samples or individual samples can determine the frequency of a biallelic marker allele in a population. One way to reduce the number of genotypings required is to use pooled samples. A drawback in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a biallelic marker or of a genotype in a given population.

The invention also relates to methods of estimating the frequency of an allele in a population comprising: a) genotyping individuals from said population for said biallelic marker according to the method of the present invention; b) determining the proportional representation of said biallelic marker in said population. In addition, the methods of estimating the frequency of an allele in a population of the invention encompass methods with any further limitation described in this disclosure, or those following, specified alone or in any combination; optionally, the PG-3-related biallelic marker is selected from the group consisting of A1 to A80, and the complements thereof, or optionally the biallelic marker is one of the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, the determination of the frequency of a biallelic marker allele in a population may be accomplished by determining the identity of the nucleotides for both copies of said biallelic marker present in the genome of each individual in said population and calculating the proportional representation of said nucleotide at said PG-3-related biallelic marker for the population; optionally, the determination of the proportional representation may be accomplished by performing a genotyping method of the invention on a pooled biological sample derived from a representative number of individuals, or each individual, in said population, and calculating the proportional amount of said nucleotide compared with the total.

### Determining the Frequency of a Haplotype in a Population

The gametic phase of haplotypes is unknown when diploid individuals are heterozygous at more than one locus. Using genealogical information in families gametic phase can sometimes be inferred (Perlin et al, 1994). When no genealogical information is available different strategies may be used. One possibility is that the multiple-site heterozygous diploids can be eliminated from the analysis, keeping only the homozygotes and the single-site heterozygote individuals, but this approach might lead to a possible bias in the sample composition and the underestimation of low-frequency haplotypes. Another possibility is that single chromosomes can be studied independently, for example, by asymmetric PCR amplification (see Newton et al, 1989; Wu et al., 1989) or by isolation of single chromosome by limit dilution followed by PCR amplification (see Ruano et al., 1990). Further, a sample may be haplotyped for sufficiently close biallelic markers by double PCR amplification of specific alleles (Sarkar, G. and Sommer S., 1991). These approaches are not entirely satisfying either because of their technical complexity, the additional cost they entail, their lack of generalization at a large scale, or the possible biases they introduce. To overcome these difficulties, an algorithm to infer the phase of PCR-amplified DNA genotypes introduced by Clark, A. G. (1990) may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognized haplotypes. For each positive identification, the complementary haplotype is added to the list of recognized haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., 1977) leading to maximum-likelihood estimates of haplotype frequencies under the assumption of Hardy-Weinberg proportions (random mating) is used (see Excoffier L. and Slatkin M., 1995). The EM algorithm is a generalized iterative maximum-likelihood approach to estimation that is useful when data are ambiguous and/or incomplete. The EM algorithm is used to resolve heterozygotes into haplotypes. Haplotype estimations are further described below under the heading “Statistical Methods.” Any other method known in the art to determine or to estimate the frequency of a haplotype in a population may be used.

The invention also encompasses methods of estimating the frequency of a haplotype for a set of biallelic markers in a population, comprising the steps of: a) genotyping at least one PG-3-related biallelic marker according to a method of the invention for each individual in said population; b) genotyping a second biallelic marker by determining the identity of the nucleotides at said second biallelic marker for both copies of said second biallelic marker present in the genome of each individual in said population; and c) applying a haplotype determination method to the identities of the nucleotides determined in steps a) and b) to obtain an estimate of said frequency. In addition, the methods of estimating the frequency of a haplotype of the invention encompass methods with any further limitation described in this disclosure, or those following, alone or in any combination: optionally, said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, said haplotype determination method is performed by asymmetric PCR amplification, double PCR amplification of specific alleles, the Clark algorithm, or an expectation-maximization algorithm.

Linkage Disequilibrium Analysis

Linkage disequilibrium is the non-random association of alleles at two or more loci and represents a powerful tool for mapping genes involved in disease traits (see Ajioka R. S. et al., 1997). Biallelic markers, because they are densely spaced in the human genome and can be genotyped in greater numbers than other types of genetic markers (such as RFLP or VNTR markers), are particularly useful in genetic analysis based on linkage disequilibrium.

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods”.

Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a biallelic marker allele) in linkage disequilibrium with the trait-causing allele will suffice to suggest the presence of a trait-related gene in that particular region. Case-control populations can be genotyped for biallelic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically biallelic markers) to be analyzed as an alternative to screening all possible functional polymorphisms in order to find trait-causing alleles. Association studies compare the frequency of marker alleles in unrelated case-control populations, and represent powerful tools for the dissection of complex traits.

### Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected, trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. The terms “trait positive population”, “case population” and “affected population” are used interchangeably herein.

An important step in the dissection of complex traits using association studies is the choice of case-control populations (see Lander and Schork, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations consist of phenotypically homogeneous populations. Trait positive and trait negative populations consist of phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with biallelic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of control individuals are included in such studies.

Association Analysis

The invention also comprises methods of detecting an association between a genotype and a phenotype, comprising the steps of: a) determining the frequency of at least one PG-3-related biallelic marker in a trait positive population according to a genotyping method of the invention; b) determining the frequency of said PG-3-related biallelic marker in a control population according to a genotyping method of the invention; and c) determining whether a statistically significant association exists between said genotype and said phenotype. In addition, the methods of detecting an association between a genotype and a phenotype of the invention encompass methods with any further limitation described in this disclosure, or those following, specified alone or in any combination: optionally, wherein said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, said control population may be a trait negative population, or a random population; optionally, each of said genotyping steps a) and b) may be performed on a pooled biological sample derived from each of said populations; optionally, each of said genotyping of steps a) and b) is performed separately on biological samples derived from each individual in said population or a subsample thereof; optionally, said trait is susceptibility to cancer or a disorder relating to abnormal cellular differentiation.

The general strategy to perform association studies using biallelic markers derived from a region carrying a candidate gene is to scan two groups of individuals (case-control populations) in order to measure and statistically compare the allele frequencies of the biallelic markers of the present invention in both groups.

If a statistically significant association with a trait is identified for at least one or more of the analyzed biallelic markers, one can assume that: either the associated allele is directly responsible for causing the trait (i.e. the associated allele is the trait causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker, and performing further association studies with the polymorphisms that are revealed in an iterative manner.

Association studies are usually run in two successive steps. In a first phase, the frequencies of a reduced number of biallelic markers from the candidate gene are determined in the trait positive and control populations. In a second phase of the analysis, the position of the genetic loci responsible for the given trait is further refined using a higher density of markers from the relevant region. However, if the candidate gene under study is relatively small in length, as is the case for PG-3, a single phase may be sufficient to establish significant associations.

Haplotype Analysis

As described above, when a chromosome carrying a disease allele first appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified biallelic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of said haplotype in a control population, according to a method of the invention for estimating the frequency of a haplotype; and c) determining whether a statistically significant association exists between said haplotype and said phenotype. In addition, the methods of detecting an association between a haplotype and a phenotype of the invention encompass methods with any further limitation described in this disclosure, or those following: optionally, said PG-3-related biallelic marker is selected from the group consisting of A1 to A80, and the complements thereof, or optionally the biallelic markers in linkage disequilibrium therewith; optionally, wherein said PG-3-related biallelic marker is selected from the group consisting of A1 to A5 and A8 to A80, and the complements thereof, or optionally the biallelic markers in linkage disequilibrium therewith; optionally, wherein said PG-3-related biallelic marker is selected from the group consisting of A6 and A7, and the complements thereof, or optionally the biallelic markers in linkage disequilibrium therewith; optionally, said control population is a trait negative population, or a random population. Optionally, said method comprises the additional steps of determining the phenotype in said trait positive and said control populations prior to step c) optionally, said trait is susceptibility to cancer or a disorder relating to abnormal cellular differentiation.

Interaction Analysis

The biallelic markers of the present invention may also be used to identify patterns of biallelic markers associated with detectable traits resulting from polygenic interactions. The 35 analysis of genetic interaction between alleles at unlinked loci requires individual genotyping using the techniques described herein. The analysis of allelic interaction among a selected set of biallelic markers with an appropriate level of statistical significance can be considered as a haplotype analysis. Interaction analysis consists in stratifying the case-control populations with respect to a given haplotype for the first loci and performing a haplotype analysis with the second loci with each subpopulation.

Statistical methods used in association studies are further described below.

Testing for Linkage in the Presence of Association

The biallelic markers of the present invention may further be used in TDT (transmission/disequilibrium test). TDT tests for both linkage and association and is not affected by population stratification. TDT requires data for affected individuals and their parents or data from 10 unaffected sibs instead of from parents (see Spielmann S. et al., 1993; Schaid D. J. et al., 1996, Spielmann S. and Ewens W. J., 1998). Such combined tests generally reduce the false-positive errors produced by separate analyses.

### Statistical Methods

In general, any method known in the art to test whether a trait and a genotype show a statistically significant correlation may be used.

1) Methods In Linkage Analysis

Statistical methods and computer programs useful for linkage analysis are well-known to those skilled in the art (see Terwilliger J. D. and Ott J., 1994; Ott J., 1991).

2) Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K.; 1997; Weir, B. S., 1996) Preferably, maximum-likelihood haplotype frequencies are computed using an Expectation-Maximization (EM) algorithm (see Dempster et al, 1977; Excoffier L. and Slatkin M., 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E., et al., 1994) or the Arlequin program (Schneider et al., 1997). The EM algorithm is a generalized iterative maximum likelihood approach to estimation and is briefly described below.

Please note that in the present section, “Methods To Estimate Haplotype Frequencies In A Population,”, phenotypes will refer to multi-locus genotypes with unknown haplotypic phase. Genotypes will refer to mutli-locus genotypes with known haplotypic phase.

Suppose one has a sample of N unrelated individuals typed for K markers. The data observed are the unknown-phase K-locus phenotypes that can be categorized with F different phenotypes. Further, suppose that we have H possible haplotypes (in the case of K biallelic markers, we have for the maximum number of possible haplotypes H=2^{K}).

For phenotype j with cj possible genotypes, we have:

$${P}_{j} = {\sum }_{i = 1}^{{c}_{j}} P ( genotype ( i ) ) = {\sum }_{i = 1}^{{c}_{j}} P ( {h}_{k} , {h}_{l} ) . \qquad Equation 1$$

Here, Pⱼ is the probability of the j^{th} phenotype, and P(hₖ,hₗ) is the probability of the i^{th} genotype composed of haplotypes hₖ and hₗ. Under random mating (i.e. Hardy-Weinberg Equilibrium), P(hₖhₗ) is expressed as:

P(hₖ,hₗ)=P(hₖ)² for hₖ=hₗ, and

P(hₖ,hₗ)=2P(hₖ)P(hₗ) for hₖ≠hₗ. Equation 2

The E-M algorithm is composed of the following steps: first, the genotype frequencies are estimated from a set of initial values of haplotype frequencies. These haplotype frequencies are denoted P₁⁽⁰⁾, P₂⁽⁰⁾, P₃⁽⁰⁾, . . . , P_{H}⁽⁰⁾. The initial values for the haplotype frequencies may be obtained from a random number generator or in some other way well known in the art. This step is referred to the Expectation step. The next step in the method, called the Maximization step, consists of using the estimates for the genotype frequencies to re-calculate the haplotype frequencies. The first iteration haplotype frequency estimates are denoted by P₁⁽¹⁾, P₂⁽¹⁾, P₃⁽¹⁾, . . . , P_{H}⁽¹⁾. In general, the Expectation step at the s^{th} iteration consists of calculating the probability of placing each phenotype into the different possible genotypes based on the haplotype frequencies of the previous iteration:

$${P ( {h}_{k} , {h}_{l} )}^{( s )} = \frac{{n}_{j}}{N} [ \frac{{{P}_{j} ( {h}_{k} , {h}_{l} )}^{( s )}}{{P}_{j}} ] , \qquad Equation 3$$

where nⱼ is the number of individuals with the j^{th} phenotype and Pⱼ(hₖ,hₗ)^{(s)} is the probability of genotype h_{g},hₗ in phenotype j. In the Maximization step, which is equivalent to the gene-counting method (Smith, 1957), the haplotype frequencies are re-estimated based on the genotype estimates:

$${P}_{t}^{( s + 1 )} = \frac{1}{2} {\sum }_{j = 1}^{F} {\sum }_{i = 1}^{{c}_{j}} {δ}_{it} {{P}_{j} ( {h}_{k} , {h}_{l} )}^{( s )} . \qquad Equation 4$$

Here, δᵢₜ is an indicator variable which counts the number of occurrences that haplotype t is present in i^{th} genotype; it takes on values 0, 1, and 2.

The E-M iterations cease when the following criterion has been reached. Using Maximum Likelihood Estimation (MLE) theory, one assumes that the phenotypes j are distributed multinomially. At each iteration s, one can compute the likelihood function L. Convergence is achieved when the difference of the log-likehood between two consecutive iterations is less than some small number, preferably 10⁻⁷.

3) Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of biallelic markers comprising at least one of the biallelic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ; aᵢ,bⱼ; bᵢ,aⱼ and bᵢ,bⱼ), according to the Piazza formula:

Δₐᵢₐⱼ=√θ4−√(θ4+θ3)(θ4+θ2), where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2=+−=frequency of genotypes having allele aᵢ at Mᵢ and not having allele aⱼ at Mⱼ

Linkage disequilibrium (LD) between pairs of biallelic markers (Mᵢ, Mⱼ) can also be calculated for every allele combination (ai,aj; ai,bj; bᵢ,aⱼ and bᵢ,bⱼ), according to the maximum-likelihood estimate (MLE) for delta (the composite genotypic disequilibrium coefficient), as described by Weir (Weir B. S., 1996). The MLE for the composite linkage disequilibrium is:

Dₐᵢₐⱼ=(2n₁+n₂+n₃+n₄/2)/N−2(pr(aᵢ).pr(aⱼ))

Where nᵢ=Σ phenotype (aᵢ/aᵢ, aⱼ/aⱼ), n₂=Σ phenotype (aᵢ/aᵢ, aⱼ/bⱼ), n₃=Σ phenotype (aᵢ/bi, aⱼ/aⱼ), n4=Σ phenotyped (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of biallelic markers, Mᵢ(aᵢ/bᵢ) and Mⱼ(aⱼ/bⱼ), fitting the Hardy-Weinberg equilibrium, one can estimate the four possible haplotype frequencies in a given population according to the approach described above.

The estimation of gametic disequilibrium between ai and aj is simply:

Dₐᵢₐⱼ=pr(haplotype(aᵢ,aⱼ))−pr(aᵢ).pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of biallelic marker only one measure of disequilibrium is necessary to describe the association between Mᵢ and Mⱼ.

Then a normalized value of the above is calculated as follows:

D′ₐᵢₐⱼ=Dₐᵢₐⱼmax(−pr(aᵢ).pr(aⱼ), −pr(bᵢ).pr(bⱼ)) with Dₐᵢₐⱼ<0

D′ₐᵢₐⱼ=Dₐᵢₐⱼ/max(pr(bᵢ).pr(aⱼ),pr(aᵢ).pr(bⱼ)) with Dₐᵢₐⱼ>0

The skilled person will readily appreciate that other linkage disequilibrium calculation methods can be used.

Linkage disequilibrium among a set of biallelic markers having an adequate heterozygosity rate can be determined by genotyping between 50 and 1000 unrelated individuals, preferably between 75 and 200, more preferably around 100.

4) Testing For Association

Methods for determining the statistical significance of a correlation between a phenotype and a genotype, in this case an allele at a biallelic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a biallelic marker allele in case and control populations and comparing these frequencies with a statistical test to determine if their is a statistically significant difference in frequency which would indicate a correlation between the trait and the biallelic marker allele under study. Similarly, a haplotype analysis is performed by estimating the frequencies of all possible haplotypes for a given set of biallelic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a biallelic marker association is preferably about 1×10⁻² or less, more preferably about 1×10⁻⁴ or less, for a single biallelic marker analysis and about 1×10⁻³ or less, still more preferably 1×10⁻⁶ or less and most preferably of about 1×10⁻⁸ or less, for a haplotype analysis involving two or more markers. These values are believed to be applicable to any association studies involving single or multiple marker combinations.

The skilled person can use the range of values set forth above as a starting point in order to carry out association studies with biallelic markers of the present invention. In doing so, significant associations between the biallelic markers of the present invention and a trait can be revealed and used for diagnosis and drug screening purposes.

Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient. This experiment is reiterated preferably at least between 100 and 10000 times. The repeated iterations allow the determination of the probability to obtain the tested haplotype by chance.

Assessment of Statistical Association

To address the problem of false positives similar analysis may be performed with the same case-control populations in random genomic regions. Results in random regions and the candidate region are compared as described in a co-pending US Provisional Patent Application entitled “Methods, Software And Apparati For Identifying Genomic Regions Harboring A Gene Associated With A Detectable Trait,” U.S. Serial No. 60/107,986, filed Nov. 10, 1998, and a second U.S. Provisional Patent Application also entitled “Methods, Software And Apparati For Identifying Genomic Regions Harboring A Gene Associated With A Detectable Trait,” U.S. Serial No. 60/140,785, filed Jun. 23, 1999.

5) Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R⁻) is the probability for individuals without the risk factor, then the relative risk is simply the ratio of the two probabilities, that is:

RR=P(R⁺)/^{P}(R⁻)

In case-control studies, direct measures of the relative risk cannot be obtained because of the sampling design. However, the odds ratio allows a good approximation of the relative risk for low-incidence diseases and can be calculated:

$$\begin{matrix}OR = [ \frac{{F}^{+}}{1 - {F}^{+}} ] / [ \frac{{F}^{-}}{( 1 - {F}^{-} )} ] \\ OR = ( {F}^{+} \text{/} ( 1 - {F}^{+} ) ) \text{/} ( {F}^{-} \text{/} ( 1 - {F}^{-} ) )\end{matrix}$$

F⁺ is the frequency of the exposure to the risk factor in cases and F⁻ is the frequency of the exposure to the risk factor in controls. F⁺ and F⁻ are calculated using the allelic or haplotype frequencies of the study and further depend on the underlying genetic model (dominant, recessive, additive . . . ).

One can further estimate the attributable risk (AR) which describes the proportion of individuals in a population exhibiting a trait due to a given risk factor. This measure is important in quantifying the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−1)/(P_{E}(RR−1)+1)

AR is the risk attributable to a biallelic marker allele or a biallelic marker haplotype. P_{E} is the frequency of exposure to an allele or a haplotype within the population at large; and RR is the relative risk which, is approximated with the odds ratio when the trait under study has a relatively low incidence in the general population

### Identification of Biallelic Markers in Linkage Disequilibrium With the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before, any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers which are in linkage disequilibrium with the biallelic markers A1 to A80 and which are expected to present similar characteristics in terms of their respective association with a given trait.

Identification of Functional Mutations

Mutations in the PG-3 gene which are responsible for a detectable phenotype or trait may be identified by comparing the sequences of the PG-3 gene from trait positive and control individuals. Once a positive association is confirmed with a biallelic marker of the present invention, the identified locus can be scanned for mutations. In a preferred embodiment, functional regions such as exons and splice sites, promoters and other regulatory regions of the PG-3 gene are scanned for mutations. In a preferred embodiment the sequence of the PG-3 gene is compared in trait positive and control individuals. Preferably, trait positive individuals carry the haplotype shown to be associated with the trait and trait negative individuals do not carry the haplotype or allele associated with the trait. The detectable trait or phenotype may comprise a variety of manifestations of altered PG-3 function.

The mutation detection procedure is essentially similar to that used for biallelic marker identification. The method used to detect such mutations generally comprises the following steps:

amplification of a region of the PG-3 gene comprising a biallelic marker or a group of biallelic markers associated with the trait from DNA samples of trait positive patients and trait-negative controls using any of the methods disclosed herein;

sequencing of the amplified region;

comparison of DNA sequences from trait positive and control individuals;

determination of mutations specific to trait-positive patients.

In one embodiment, said biallelic marker is selected from the group consisting of A1 to A80, and the complements thereof. It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results. Polymorphisms are considered as candidate “trait-causing” mutations when they exhibit a statistically significant correlation with the detectable phenotype.

### Biallelic Markers of the Invention in Methods of Genetic Diagnostics

The biallelic markers of the present invention can also be used to develop diagnostics tests capable of identifying individuals who express a detectable trait as the result of a specific genotype or individuals whose genotype places them at risk of developing a detectable trait at a subsequent time. The trait analyzed using the present diagnostics may be any detectable trait, including diseases such as cancer or a disorder relating to abnormal cellular differentiation. Such a diagnosis can be useful in the staging, monitoring, prognosis and/or prophylactic or curative therapy of diseases.

The diagnostic techniques of the present invention may employ a variety of methodologies to determine whether a test subject has a biallelic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids.

The present invention provides diagnostic methods to determine whether an individual is at risk of developing a disease or suffers from a disease resulting from a mutation or a polymorphism in the PG-3 gene. The present invention also provides methods to determine whether an individual has a susceptibility to diseases such as cancer or a disorder relating to abnormal cellular differentiation.

These methods involve obtaining a nucleic acid sample from the individual and, determining, whether the nucleic acid sample contains at least one allele or at least one biallelic marker haplotype, indicative of a risk of developing the trait or indicative that the individual expresses the trait as a result of possessing a particular PG-3 polymorphism or mutation (trait-causing allele).

Preferably, in such diagnostic methods, a nucleic acid sample is obtained from the individual and this sample is genotyped using methods described above in Methods Of Genotyping DNA Samples For Biallelic markers. The diagnostics may be based on a single biallelic marker or a on group of biallelic markers.

In each of these methods, a nucleic acid sample is obtained from the test subject and the biallelic marker pattern of one or more of the biallelic markers A1 to A80 is determined.

In one embodiment, a PCR amplification is conducted on the nucleic acid sample to amplify regions in which polymorphisms associated with a detectable phenotype have been identified. The amplification products are sequenced to determine whether the individual possesses one or more PG-3 polymorphisms associated with a detectable phenotype. The primers used to generate amplification products may comprise the primers listed in Table 1. Alternatively, the nucleic acid sample is subjected to microsequencing reactions as described above to determine whether the individual possesses one or more PG-3 polymorphisms associated with a detectable phenotype resulting from a mutation or a polymorphism in the PG-3 gene. The primers used in the microsequencing reactions may include the primers listed in Table 4. In another embodiment, the nucleic acid sample is contacted with one or more allele specific oligonucleotide probes which, specifically hybridize to one or more PG-3 alleles associated with a detectable phenotype. The probes used in the hybridization assay may include the probes listed in Table 3. In another embodiment, the nucleic acid sample is contacted with a second PG-3 oligonucleotide capable of producing an amplification product when used with the allele specific oligonucleotide in an amplification reaction. The presence of an amplification product in the amplification reaction indicates that the individual possesses one or more PG-3 alleles associated with a detectable phenotype.

In a preferred embodiment the identity of the nucleotide present at, at least one, biallelic marker selected from the group consisting of Al to An and the complements thereof, is determined and the detectable trait is diseases such as cancer or a disorder relating to abnormal cellular differentiation. Diagnostic kits comprise any of the polynucleotides of the present invention.

These diagnostic methods are extremely valuable as they can, in certain circumstances, be used to initiate preventive treatments or to allow an individual carrying a significant haplotype to foresee warning signs such as minor symptoms.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the markers of the present invention. One or more markers indicative of either response to an agent acting against a disease, preferably cancer or a disorder relating to abnormal cellular differentiation, or to side effects to an agent acting against a disease, preferably cancer or a disorder relating to abnormal cellular differentiation, may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

### Recombinant Vectors

The term “vector” is used herein to designate either a circular or a linear DNA or RNA molecule, which is either double-stranded or single-stranded, and which comprise at least one polynucleotide of interest that is sought to be transferred in a cell host or in a unicellular or multicellular host organism.

The present invention encompasses a family of recombinant vectors that comprise a regulatory polynucleotide derived from the PG-3 genomic sequence, and/or a coding polynucleotide from either the PG-3 genomic sequence or the cDNA sequence.

Generally, a recombinant vector of the invention may comprise any of the polynucleotides described herein, including regulatory sequences, coding sequences and polynucleotide constructs, as well as any PG-3 primer or probe as defined above. More particularly, the recombinant vectors of the present invention can comprise any of the polynucleotides described in the “Genomic Sequences Of The PG3 Gene” section, the “PG-3 cDNA Sequences” section, the “Coding Regions” section, the “Polynucleotide constructs” section, and the “Oligonucleotide Probes And Primers” section.

In a first preferred embodiment, a recombinant vector of the invention is used to amplify the inserted polynucleotide derived from a PG-3 genomic sequence of SEQ ID No 1 or a PG-3 cDNA, for example the cDNA of SEQ ID No 2 in a suitable cell host, this polynucleotide being amplified at every time that the recombinant vector replicates.

A second preferred embodiment of the recombinant vectors according to the invention comprises expression vectors comprising either a regulatory polynucleotide or a coding nucleic acid of the invention, or both. Within certain embodiments, expression vectors are employed to express the PG-3 polypeptide, which can then be purified and, for example be used in ligand screening assays or as an immunogen in order to raise specific antibodies directed against the PG-3 protein. In other embodiments, the expression vectors are used for constructing transgenic animals and also for gene therapy. Expression requires that appropriate signals are provided in the vectors, said signals including various regulatory elements, such as enhancers/promoters from both viral and mammalian sources that drive expression of the genes of interest in host cells. Dominant drug selection markers for establishing permanent, stable cell clones expressing the products are generally included in the expression vectors of the invention, as they are elements that link expression of the drug selection markers to expression of the polypeptide.

More particularly, the present invention relates to expression vectors which include nucleic acids encoding a PG-3 protein, preferably the PG-3 protein of the amino acid sequence of SEQ ID No 3 or variants or fragments thereof.

The invention also pertains to a recombinant expression vector useful for the expression of the PG-3 coding sequence, wherein said vector comprises a nucleic acid of SEQ ID No 2.

Recombinant vectors comprising a nucleic acid containing a PG-3-related biallelic marker are also part of the invention. In a preferred embodiment, said biallelic marker is selected from the group consisting of A1 to A80, and the complements thereof.

Some of the elements which can be found in the vectors of the present invention are described in further detail in the following sections.

The present invention also encompasses primary, secondary, and immortalized homologously recombinant host cells of vertebrate origin, preferably mammalian origin and particularly human origin, that have been engineered to: a) insert exogenous (heterologous) polynucleotides into the endogenous chromosomal DNA of a targeted gene, b) delete endogenous chromosomal DNA, and/or c) replace endogenous chromosomal DNA with exogenous polynucleotides. Insertions, deletions, and/or replacements of polynucleotide sequences may be to the coding sequences of the targeted gene and/or to regulatory regions, such as promoter and enhancer sequences, operably associated with the targeted gene.

The present invention further relates to a method of making a homologously recombinant host cell in vitro or in vivo, wherein the expression of a targeted gene not normally expressed in the cell is altered. Preferably the alteration causes expression of the targeted gene under normal growth conditions or under conditions suitable for producing the polypeptide encoded by the targeted gene. The method comprises the steps of: (a) transfecting the cell in vitro or in vivo with a polynucleotide construct, the polynucleotide construct comprising: (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination.

The present invention further relates to a method of altering the expression of a targeted gene in a cell in vitro or in vivo wherein the gene is not normally expressed in the cell, comprising the steps of: (a) transfecting the cell in vitro or in vivo with a a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; and (b) maintaining the transfected cell in vitro or in vivo under conditions appropriate for homologous recombination, thereby producing a homologously recombinant cell; and (c) maintaining the homologously recombinant cell in vitro or in vivo under conditions appropriate for expression of the gene.

The present invention further relates to a method of making a polypeptide of the present invention by altering the expression of a targeted endogenous gene in a cell in vitro or in vivo wherein the gene is not normally expressed in the cell, comprising the steps of: a) transfecting the cell in vitro with a a polynucleotide construct, the a polynucleotide construct comprising: (i) a targeting sequence; (ii) a regulatory sequence and/or a coding sequence; and (iii) an unpaired splice donor site, if necessary, thereby producing a transfected cell; (b) maintaining the transfected cell in vitro or in vivo under conditions appropriate for homologous recombination, thereby producing a homologously recombinant cell; and c) maintaining the homologously recombinant cell in vitro or in vivo under conditions appropriate for expression of the gene thereby making the polypeptide.

The present invention further relates to a polynucleotide construct which alters the expression of a targeted gene in a cell type in which the gene is not normally expressed. This occurs when a polynucleotide construct is inserted into the chromosomal DNA of the target cell, wherein a polynucleotide construct comprises: a) a targeting sequence; b) a regulatory sequence and/or coding sequence; and c) an unpaired splice-donor site, if necessary. Further included are a polynucleotide constructs, as described above, wherein the construct further comprises a polynucleotide which encodes a polypeptide and is in-frame with the targeted endogenous gene after homologous recombination with chromosomal DNA.

The compositions may be produced, and methods performed, by techniques known in the art, such as those described in U.S. Pat. Nos. 6,054,288; 6,048,729; 6,048,724; 6,048,524; 5,994,127; 5,968,502; 5,965,125; 5,869,239; 5,817,789; 5,783,385; 5,733,761; 5,641,670; 5,580,734; International Publication Nos.: WO96/29411, WO 94/12650; and scientific articles including Koller et al., 1989.

1. General Features of the Expression Vectors of the Invention

A recombinant vector according to the invention comprises, but is not limited to, a YAC (Yeast Artificial Chromosome), a BAC (Bacterial Artificial Chromosome), a phage, a phagemid, a cosmid, a plasmid or even a linear DNA molecule which may consist of a chromosomal, non-chromosomal, semi-synthetic and synthetic DNA. Such a recombinant vector can comprise a transcriptional unit comprising an assembly of:

(1) a genetic element or elements having a regulatory role in gene expression, for example promoters or enhancers. Enhancers are cis-acting elements of DNA, usually from about 10 to 300 bp in length that act on the promoter to increase the transcription;

(2) a structural or coding sequence which is transcribed into mRNA and eventually translated into a polypeptide, said structural or coding sequence being operably linked to the regulatory elements described in (1); and

(3) appropriate transcription initiation and termination sequences.

Structural units intended for use in yeast or eukaryotic expression systems preferably include a leader sequence enabling extracellular secretion of translated protein by a host cell. Alternatively, when a recombinant protein is expressed without a leader or transport sequence, it may include a N-terminal residue. This residue may or may not be subsequently cleaved from the expressed recombinant protein to provide a final product.

Generally, recombinant expression vectors will include origins of replication, selectable markers permitting transformation of the host cell, and a promoter derived from a highly expressed gene to direct transcription of a downstream structural sequence. The heterologous structural sequence is assembled in appropriate phase with translation initiation and termination sequences, and preferably a leader sequence capable of directing secretion of the translated protein into the periplasmic space or the extracellular medium. In a specific embodiment wherein the vector is adapted for transfecting and expressing desired sequences in mammalian host cells, preferred vectors will comprise an origin of replication in the desired host, a suitable promoter and enhancer, and also any necessary ribosome binding sites, polyadenylation signal, splice donor and acceptor sites, transcriptional termination sequences, and 5′-flanking non-transcribed sequences. DNA sequences derived from the SV40 viral genome, for example SV40 origin, early promoter, enhancer, splice and polyadenylation signals may be used to provide the required non-transcribed genetic elements.

The in vivo expression of a PG-3 polypeptide of SEQ ID No 3 or fragments or variants thereof may be useful in order to correct a genetic defect related to the expression of the native gene in a host organism or to the production of a biologically inactive PG-3 protein.

Consequently, the present invention also deals with recombinant expression vectors mainly designed for the in vivo production of the PG-3 polypeptide of SEQ ID No 3 or fragments or variants thereof by the introduction of the appropriate genetic material in the organism of the patient to be treated. This genetic material may be introduced in vitro in a cell that has been previously extracted from the organism, the modified cell being subsequently reintroduced in the said organism, directly in vivo into the appropriate tissue.

2. Regulatory Elements

Promoters

The suitable promoter regions used in the expression vectors according to the present invention are chosen taking into account the cell host in which the heterologous gene has to be expressed. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell, such as, for example, a human or a viral promoter.

A suitable promoter may be heterologous with respect to the nucleic acid for which it controls the expression or alternatively can be endogenous to the native polynucleotide containing the coding sequence to be expressed. Additionally, the promoter is generally heterologous with respect to the recombinant vector sequences within which the construct promoter/coding sequence has been inserted.

Promoter regions can be selected from any desired gene using, for example, CAT (chloramphenicol transferase) vectors and more preferably pKK232-8 and pCM7 vectors.

Preferred bacterial promoters are the Lac, LacZ, the T3 or T7 bacteriophage RNA polymerase promoters, the gpt, lambda PR, PL and trp promoters (EP 0036776), the polyhedrin promoter, or the pl0 protein promoter from baculovirus (Kit Novagen) (Smith et al., 1983; O'Reilly et al., 1992), the lambda PR promoter or also the trc promoter.

Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

The choice of a promoter is well within the ability of a person skilled in the field of genetic egineering. For example, one may refer to the book of Sambrook et al. (1989) or also to the procedures described by Fuller et al (1996).

Other Regulatory Elements

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

3. Selectable Markers

Such markers would confer an identifiable change to the cell permitting easy identification of cells containing the expression construct. The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. coli, or levan saccharase for mycobacteria, this latter marker being a negative selection marker.

4. Preferred Vectors.

Bacterial Vectors

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), and GEM1 (Promega Biotec, Madison, Wis., USA).

Large numbers of other suitable vectors are known to those of skill in the art, and commercially available, such as the following bacterial vectors: pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIAexpress).

Bacteriophage Vectors

The P1 bacteriophage vector may contain large inserts ranging from about 80 to about 100 kb.

The construction of P1 bacteriophage vectors such as pl58 or pl58/neo8 are notably described by Sternberg (1992, 1994). Recombinant P1 clones comprising PG-3 nucleotide sequences may be designed for inserting large polynucleotides of more than 40 kb (Linton et al., 1993). To generate P1 DNA for transgenic experiments, a preferred protocol is the protocol described by McCormick et al. (1994). Briefly, E. coli (preferably strain NS3529) harboring the PI plasmid are grown overnight in a suitable broth medium containing 25 μg/ml of kanamycin. The P1 DNA is prepared from the E. Coli by alkaline lysis using the Qiagen Plasmid Maxi kit (Qiagen, Chatsworth, Calif., USA), according to the manufacturer's instructions. The P1 DNA is purified from the bacterial lysate on two Qiagen-tip 500 columns, using the washing and elution buffers contained in the kit. A phenol/chloroform extraction is then performed before precipitating the DNA with 70% ethanol. After solubilizing the DNA in TE (10 mM Tris-HCl, pH 7.4, 1 mM EDTA), the concentration of the DNA is assessed by spectrophotometry.

When the goal is to express a P1 clone comprising PG-3 nucleotide sequences in a transgenic animal, typically in transgenic mice, it is desirable to remove vector sequences from the P1 DNA fragment, for example by cleaving the P1 DNA at rare-cutting sites within the P1 polylinker (SfI, NotI or SalI). The PI insert is then purified from vector sequences on a pulsed-field agarose gel, using methods similar using methods similar to those originally reported for the isolation of DNA from YACs (Schedl et al., 1993a; Peterson et al., 1993). At this stage, the resulting purified insert DNA can be concentrated, if necessary, on a Millipore Ultrafree-MC Filter Unit (Millipore, Bedford, Mass., USA—30,000 molecular weight limit) and then dialyzed against microinjection buffer (10 mM Tris-HCl, pH 7.4; 250 μM EDTA) containing 100 mM NaCl, 30 μM spermine, 70 μM spermidine on a microdyalisis membrane (type VS, 0.025 μM from Millipore). The intactness of the purified P1 DNA insert is assessed by electrophoresis on 1% agarose (Sea Kem GTG; FMC Bio-products) pulse-field gel and staining with ethidium bromide.

Baculovirus Vectors

A suitable vector for the expression of the PG-3 polypeptide of SEQ ID No 3 or fragments or variants thereof is a baculovirus vector that can be propagated in insect cells and in insect cell lines. A specific suitable host vector system is the pVL1392/1393 baculovirus transfer vector (Pharmingen) that is used to transfect the SF9 cell line (ATCC N^{o} CRL 1711) which is derived from Spodoptera frugiperda.

Other suitable vectors for the expression of the PG-3 polypeptide of SEQ ID No 3 or fragments or variants thereof in a baculovirus expression system include those described by Chai et al.(1993), Vlasak et al.(1983) and Lenhard et al.(1996).

Viral Vectors

In one specific embodiment, the vector is derived from an adenovirus. Preferred adenovirus vectors according to the invention are those described by Feldman and Steg (1996) or Ohno et al. (1994). Another preferred recombinant adenovirus according to this specific embodiment of the present invention is the human adenovirus type 2 or 5 (Ad 2 or Ad 5) or an adenovirus of animal origin (French patent application N°FR-93.05954).

Retrovirus vectors and adeno-associated virus vectors are generally understood to be the recombinant gene delivery systems of choice for the transfer of exogenous polynucleotides in vivo, particularly to mammals, including humans. These vectors provide efficient delivery of genes into cells, and the transferred nucleic acids are stably integrated into the chromosomal DNA of the host.

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al. (1996), PCT Application No WO 93/25234, PCT Application No WO 94/06920, Roux et al., 1989, Julan et al., 1992 and Neda et al., 1991.

Yet another viral vector system that is contemplated by the invention consists in the adeno-associated virus (AAV). The adeno-associated virus is a naturally occurring defective virus that requires another virus, such as an adenovirus or a herpes virus, as a helper virus for efficient replication and a productive life cycle (Muzyczka et al., 1992). It is also one of the few viruses that may integrate its DNA into non-dividing cells, and exhibits a high frequency of stable integration (Flotte et al., 1992; Samulski et al., 1989; McLaughlin et al., 1989). One advantageous feature of AAV derives from its reduced efficacy for transducing primary cells relative to transformed cells.

BAC Vectors

The bacterial artificial chromosome (BAC) cloning system (Shizuya et al, 1992) has been developed to stably maintain large fragments of genomic DNA (100-300 kb) in E. coli. A preferred BAC vector consists of pBeloBAC11 vector that has been described by Kim et al (1996). BAC libraries are prepared with this vector using size-selected genomic DNA that has been partially digested using enzymes that permit ligation into either the Bam HI or HindIII sites in the vector. Flanking these cloning sites are T7 and SP6 RNA polymerase transcription initiation sites that can be used to generate end probes by either RNA transcription or PCR methods. After the construction of a BAC library in E. coli, BAC DNA is purified from the host cell as a supercoiled circle. Converting these circular molecules into a linear form precedes both size determination and introduction of the BACs into recipient cells. The cloning site is flanked by two Not I sites, permitting cloned segments to be excised from the vector by Not I digestion. Alternatively, the DNA insert contained in the pBeloBAC 11 vector may be linearized by treatment of the BAC vector with the commercially available enzyme lambda terminase that leads to the cleavage at the unique cosN site, but this cleavage method results in a full length BAC clone containing both the insert DNA and the BAC sequences.

5. Delivery of the Recombinant Vectors

In order to effect expression of the polynucleotides and polynucleotide constructs of the invention, these constructs must be delivered into a cell. This delivery may be accomplished in vitro, as in laboratory procedures for transforming cell lines, or in vivo or ex vivo, as in the treatment of certain diseases states.

One mechanism is viral infection where the expression construct is encapsulated in an infectious viral particle.

Several non-viral methods for the transfer of polynucleotides into cultured mammalian cells are also contemplated by the present invention, and include, without being limited to, calcium phosphate precipitation (Graham et al., 1973; Chen et al., 1987;), DEAE-dextran (Gopal, 1985), electroporation (Tur-Kaspa et al., 1986; Potter et al., 1984), direct microinjection (Harland et al., 1985), DNA-loaded liposomes (Nicolau et al., 1982; Fraley et al., 1979), and receptor-mediated transfection (Wu and Wu, 1987; 1988). Some of these techniques may be successfully adapted for in vivo or ex vivo use.

Once the expression polynucleotide has been delivered into the cell, it may be stably integrated into the genome of the recipient cell. This integration may be in the cognate location and orientation via homologous recombination (gene replacement) or it may be integrated in a random, non specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle.

One specific embodiment for a method for delivering a protein or peptide to the interior of a cell of a vertebrate in vivo comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect. This is particularly applicable for transfer in vitro but it may be applied to in vivo as well.

Compositions for use in vitro and in vivo comprising a “naked” polynucleotide are described in PCT application N^{o} WO 90/11092 (Vical Inc.), and also in PCT application No. WO 95/11307 (Institut Pasteur, INSERM, Universite d'Ottawa); as well as in the articles of Tacson et al. (1996), and of Huygen et al. (1996).

In still another embodiment of the invention, the transfer of a naked polynucleotide of the invention, including a polynucleotide construct of the invention, into cells may be proceeded with a particle bombardment (biolistic), said particles being DNA-coated microprojectiles accelerated to a high velocity allowing them to pierce cell membranes and enter cells without killing them, such as described by Klein et al (1987).

In a further embodiment, the polynucleotide of the invention may be entrapped in a liposome (Ghosh and Bacchawat, 1991; Wong et al., 1980; Nicolau et al., 1987)

In a specific embodiment, the invention provides a composition for the in vivo production of the PG-3 protein or polypeptide described herein. It comprises a naked polynucleotide operatively coding for this polypeptide, in solution in a physiologically acceptable carrier, and suitable for introduction into a tissue to cause cells of the tissue to express the said protein or polypeptide.

The amount of vector to be injected to the desired host organism varies according to the site of injection. As an indicative dose, it will be injected between 0, 1 and 100 μg of the vector in an animal body, preferably a mammal body, for example a mouse body.

In another embodiment of the vector according to the invention, it may be introduced in vitro in a host cell, preferably in a host cell previously harvested from the animal to be treated and more preferably a somatic cell such as a muscle cell. In a subsequent step, the cell that has been transformed with the vector coding for the desired PG-3 polypeptide or the desired fragment thereof is reintroduced into the animal body in order to deliver the recombinant protein within the body either locally or systemically.

### Cell Hosts

Another object of the invention consists of a host cell that has been transformed or transfected with one of the polynucleotides described herein, and in particular a polynucleotide either comprising a PG-3 regulatory polynucleotide or the coding sequence for the PG-3 polypeptide in a polynucleotide selected from the group consisting of SEQ ID Nos 1 and 2 or a fragment or a variant thereof. Also included are host cells that are transformed (prokaryotic cells) or that are transfected (eukaryotic cells) with a recombinant vector such as one of those described above. More particularly, the cell hosts of the present invention can comprise any of the polynucleotides described in the “Genomic Sequences Of The PG3 Gene” section, the “PG-3 cDNA Sequences” section, the “Coding Regions” section, the “Polynucleotide constructs” section, and the “Oligonucleotide Probes And Primers” section.

A further recombinant cell host according to the invention comprises a polynucleotide containing a biallelic marker selected from the group consisting of A1 to A80, and the complements thereof.

An additional recombinant cell host according to the invention comprises any of the vectors described herein, more particularly any of the vectors described in the “Recombinant Vectors” section.

Preferred host cells used as recipients for the expression vectors of the invention are the following:

a) Prokaryotic host cells: Escherichia coli strains (I.E.DH5-α strain), Bacillus subtilis, Salmonella typhimurium, and strains from species like Pseudomonas, Streptomyces and Staphylococcus.

b) Eukaryotic host cells: HeLa cells (ATCC N^{o} CCL2; N^{o} CCL2.1; N^{o} CCL2.2), Cv 1 cells (ATCC N^{o} CCL70), COS cells (ATCC N^{o} CRL1650; N^{o} CRL1651), Sf-9 cells (ATCC N^{o} CRL1711), C127 cells (ATCC N^{o} CRL-1804), 3T3 (ATCC N^{o} CRL-6361), CHO (ATCC N^{o} CCL-61), human kidney 293 (ATCC N^{o} 45504; N^{o} CRL-1573) and BHK (ECACCN^{o} 84100501; N^{o} 84111301).

c) Other mammalian host cells.

The PG-3 gene expression in mammalian, and typically human, cells may be rendered defective, or alternatively expression may be provided by the insertion of a PG-3 genomic or cDNA sequence with the replacement of the PG-3 gene counterpart in the genome of an animal cell by a PG-3 polynucleotide according to the invention. These genetic alterations may be generated by homologous recombination events using specific DNA constructs that have been previously described.

One kind of cell hosts that may be used are mammalian zygotes, such as murine zygotes. For example, murine zygotes may undergo microinjection with a purified DNA molecule of interest, for example a purified DNA molecule that has previously been adjusted to a concentration range from 1 ng/ml—for BAC inserts—3 ng/μl—for P1 bacteriophage inserts—in 10 mM Tris-HCl, pH 7.4, 250 μM EDTA containing 100 mM NaCl, 30 μM spermine, and 70 μM spermidine. When the DNA to be microinjected has a large size, polyamines and high salt concentrations can be used in order to avoid mechanical breakage of this DNA, as described by Schedl et al (1993b).

Anyone of the polynucleotides of the invention, including the DNA constructs described herein, may be introduced in an embryonic stem (ES) cell line, preferably a mouse ES cell line. ES cell lines are derived from pluripotent, uncommitted cells of the inner cell mass of pre-implantation blastocysts. Preferred ES cell lines are the following: ES-E14TG2a (ATCC n^{o} CRL-1821), ES-D3 (ATCC n^{o} CRL1934 and n^{o} CRL-11632), YS001 (ATCC n^{o} CRL-11776), 36.5 (ATCC n^{o} CRL-11116). To maintain ES cells in an uncommitted state, they are cultured in the presence of growth inhibited feeder cells which provide the appropriate signals to preserve this embryonic phenotype and serve as a matrix for ES cell adherence. Preferred feeder cells consist of primary embryonic fibroblasts that are established from tissue of day 13-day 14 embryos of virtually any mouse strain, that are maintained in culture, such as described by Abbondanzo et al. (1993) and are inhibited in growth by irradiation, such as described by Robertson (1987), or by the presence of an inhibitory concentration of LF, such as described by Pease and Williams (1990).

The constructs in the host cells can be used in a conventional manner to produce the gene product encoded by the recombinant sequence.

Following transformation of a suitable host and growth of the host to an appropriate cell density, the selected promoter is induced by appropriate means, such as temperature shift or chemical induction, and cells are cultivated for an additional period.

Cells are typically harvested by centrifugation, disrupted by physical or chemical means, and the resulting crude extract retained for further purification.

Microbial cells employed in the expression of proteins can be disrupted by any convenient method, including freeze-thaw cycling, sonication, mechanical disruption, or use of cell lysing agents. Such methods are well known by the skill artisan.

### Transgenic Animals

The terms “transgenic animals” or “host animals” are used herein designate animals that have their genome genetically and artificially manipulated so as to include one of the nucleic acids according to the invention. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention. In one embodiment, the invention encompasses non-human host mammals and animals comprising a recombinant vector of the invention or a PG-3 gene disrupted by homologous recombination with a knock out vector.

The transgenic animals of the invention all include within a plurality of their cells a cloned recombinant or synthetic DNA sequence, more specifically one of the purified or isolated nucleic acids comprising a PG-3 coding sequence, a PG-3 regulatory polynucleotide, a polynucleotide construct, or a DNA sequence encoding an antisense polynucleotide such as described in the present specification.

Generally, a transgenic animal according the present invention comprises any one of the polynucleotides, the recombinant vectors and the cell hosts described in the present invention. More particularly, the transgenic animals of the present invention can comprise any of the polynucleotides described in the “Genomic Sequences Of The PG3 Gene” section, the “PG-3 cDNA Sequences” section, the “Coding Regions” section, the “Polynucleotide constructs” section, the “Oligonucleotide Probes And Primers” section, the “Recombinant Vectors” section and the “Cell Hosts” section.

A further transgenic animals according to the invention contains in their somatic cells and/or in their germ line cells a polynucleotide comprising a biallelic marker selected from the group consisting of A1 to A80, and the complements thereof.

In a first preferred embodiment, these transgenic animals may be good experimental models in order to study the diverse pathologies related to cell differentiation, in particular concerning the transgenic animals within the genome of which has been inserted one or several copies of a polynucleotide encoding a native PG-3 protein, or alternatively a mutant PG-3 protein.

In a second preferred embodiment, these transgenic animals may express a desired polypeptide of interest under the control of the regulatory polynucleotides of the PG-3 gene, leading to good yields in the synthesis of this protein of interest, and eventually a tissue specific expression of this protein of interest.

The design of the transgenic animals of the invention may be made according to the conventional techniques well known from the one skilled in the art. For more details regarding the production of transgenic animals, and specifically transgenic mice, it may be referred to U.S. Pat. Nos. 4,873,191, issued Oct. 10, 1989; U.S. Pat. No. 5,464,764, issued Nov. 7, 1995; and U.S. Pat. No. 5,789,215, issued Aug. 4, 1998; these documents disclosing methods producing transgenic mice.

Transgenic animals of the present invention are produced by the application of procedures which result in an animal with a genome that has incorporated exogenous genetic material. The procedure involves obtaining the genetic material, or a portion thereof, which encodes either a PG-3 coding sequence, a PG-3 regulatory polynucleotide or a DNA sequence encoding a PG-3 antisense polynucleotide such as described in the present specification.

A recombinant polynucleotide of the invention is inserted into an embryonic or ES stem cell line. The insertion is preferably made using electroporation, such as described by Thomas et al. (1987). The cells subjected to electroporation are screened (e.g. by selection via selectable markers, by PCR or by Southern blot analysis) to find positive cells which have integrated the exogenous recombinant polynucleotide into their genome, preferably via an homologous recombination event. An illustrative positive-negative selection procedure that may be used according to the invention is described by Mansour et al. (1988).

Then, the positive cells are isolated, cloned and injected into 3.5 days old blastocysts from mice, such as described by Bradley (1987). The blastocysts are then inserted into a female host animal and allowed to grow to term.

Alternatively, the positive ES cells are brought into contact with embryos at the 2.5 days old 8-16 cell stage (morulae) such as described by Wood et al. (1993) or by Nagy et al. (1993), the ES cells being internalized to colonize extensively the blastocyst including the cells which will give rise to the germ line.

The offspring of the female host are tested to determine which animals are transgenic e.g. include the inserted exogenous DNA sequence and which are wild-type.

Thus, the present invention also concerns a transgenic animal containing a nucleic acid, a recombinant expression vector or a recombinant host cell according to the invention.

Recombinant Cell Lines Derived from the Transgenic Animals of the Invention.

A further object of the invention consists of recombinant host cells obtained from a transgenic animal described herein. In one embodiment the invention encompasses cells derived from non-human host mammals and animals comprising a recombinant vector of the invention or a PG-3 gene disrupted by homologous recombination with a knock out vector.

Recombinant cell lines may be established in vitro from cells obtained from any tissue of a transgenic animal according to the invention, for example by transfection of primary cell cultures with vectors expressing onc-genes such as SV40 large T antigen, as described by Chou (1989) and Shay et al. (1991).

### Methods for Screening Substances Interacting with a PG-3 Polypeptide

For the purpose of the present invention, a ligand means a molecule, such as a protein, a peptide, an antibody or any synthetic chemical compound capable of binding to the PG-3 protein or one of its fragments or variants or to modulate the expression of the polynucleotide coding for PG-3 or a fragment or variant thereof. These molecules may be used in therapeutic compositions, preferably therapeutic compositions acting against cancer or a disorder relating to abnormal cellular differentiation.

In the ligand screening method according to the present invention, a biological sample or a defined molecule to be tested as a putative ligand of the PG-3 protein is brought into contact with the corresponding purified PG-3 protein, for example the corresponding purified recombinant PG-3 protein produced by a recombinant cell host as described hereinbefore, in order to form a complex between this protein and the putative ligand molecule to be tested.

As an illustrative example, to study the interaction of the PG-3 protein, or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3, with drugs or small molecules, such as molecules generated through combinatorial chemistry approaches, the microdialysis coupled to HPLC method described by Wang et al. (1997) or the affinity capillary electrophoresis method described by Bush et al. (1997).

In further methods, peptides, drugs, fatty acids, lipoproteins, or small molecules which interact with the PG-3 protein, or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3 may be identified using assays such as the following. The molecule to be tested for binding is labeled with a detectable label, such as a fluorescent, radioactive, or enzymatic tag and placed in contact with immobilized PG-3 protein, or a fragment thereof under conditions which permit specific binding to occur. After removal of non-specifically bound molecules, bound molecules are detected using appropriate means.

Another object of the present invention consists of methods and kits for the screening of candidate substances that interact with PG-3 polypeptide.

The present invention pertains to methods for screening substances of interest that interact with a PG-3 protein or one fragment or variant thereof. By their capacity to bind covalently or non-covalently to a PG-3 protein or to a fragment or variant thereof, these substances or molecules may be advantageously used both in vitro and in vivo.

In vitro, said interacting molecules may be used as detection means in order to identify the presence of a PG-3 protein in a sample, preferably a biological sample.

A method for the screening of a candidate substance comprises the following steps:

a) providing a polypeptide consisting of a PG-3 protein or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3.

b) obtaining a candidate substance;

c) bringing into contact said polypeptide with said candidate substance;

d) detecting the complexes formed between said polypeptide and said candidate substance.

The invention further concerns a kit for the screening of a candidate substance interacting with the PG-3 polypeptide, wherein said kit comprises:

a) a PG-3 protein having an amino acid sequence selected from the group consisting of the amino acid sequences of SEQ ID No 3 or a peptide fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3;

b) optionally means useful to detect the complex formed between the PG-3 protein or a peptide fragment or a variant thereof and the candidate substance.

In a preferred embodiment of the kit described above, the detection means consist in monoclonal or polyclonal antibodies directed against the PG-3 protein or a peptide fragment or a variant thereof.

Various candidate substances or molecules can be assayed for interaction with a PG-3 polypeptide. These substances or molecules include, without being limited to, natural or synthetic organic compounds or molecules of biological origin such as polypeptides. When the candidate substance or molecule consists of a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

The invention also pertains to kits useful for performing the hereinbefore described screening method. Preferably, such kits comprise a PG-3 polypeptide or a fragment or a variant thereof, and optionally means useful to detect the complex formed between the PG-3 polypeptide or its fragment or variant and the candidate substance. In a preferred embodiment the detection means consist in monoclonal or polyclonal antibodies directed against the corresponding PG-3 polypeptide or a fragment or a variant thereof.

A. Candidate Ligands Obtained from Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode for peptides of 8 to 20 amino acids in length (Oldenburg K. R. et al., 1992; Valadon P., et al., 1996; Lucas A. H., 1994; Westerink M. A. J., 1995; Felici F. et al., 1991). According to this particular embodiment, the recombinant phages expressing a protein that binds to the immobilized PG-3 protein is retained and the complex formed between the PG-3 protein and the recombinant phage may be subsequently immunoprecipitated by a polyclonal or a monoclonal antibody directed against the PG-3 protein.

Once the ligand library in recombinant phages has been constructed, the phage population is brought into contact with the immobilized PG-3 protein. Then the preparation of complexes is washed in order to remove the non-specifically bound recombinant phages. The phages that bind specifically to the PG-3 protein are then eluted by a buffer (acid pH) or immunoprecipitated by the monoclonal antibody produced by the hybridoma anti-PG-3, and this phage population is subsequently amplified by an over-infection of bacteria (for example E. coli). The selection step may be repeated several times, preferably 2-4 times, in order to select the more specific recombinant phage clones. The last step consists in characterizing the peptide produced by the selected recombinant phage clones either by expression in infected bacteria and isolation, expressing the phage insert in another host-vector system, or sequencing the insert contained in the selected recombinant phages.

B. Candidate Ligands Obtained by Competition Experiments.

Alternatively, peptides, drugs or small molecules which bind to the PG-3 protein, or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3, may be identified in competition experiments. In such assays, the PG-3 protein, or a fragment thereof, is immobilized to a surface, such as a plastic plate. Increasing amounts of the peptides, drugs or small molecules are placed in contact with the immobilized PG-3 protein, or a fragment thereof, in the presence of a detectable labeled known PG-3 protein ligand. For example, the PG-3 ligand may be detectably labeled with a fluorescent, radioactive, or enzymatic tag. The ability of the test molecule to bind the PG-3 protein, or a fragment thereof, is determined by measuring the amount of detectably labeled known ligand bound in the presence of the test molecule. A decrease in the amount of known ligand bound to the PG-3 protein, or a fragment thereof, when the test molecule is present indicated that the test molecule is able to bind to the PG-3 protein, or a fragment thereof.

C. Candidate Ligands Obtained by Affinity Chromatography.

Proteins or other molecules interacting with the PG-3 protein, or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3, can also be found using affinity columns which contain the PG-3 protein, or a fragment thereof. The PG-3 protein, or a fragment thereof, may be attached to the column using conventional techniques including chemical coupling to a suitable column matrix such as agarose, Affi Gel®, or other matrices familiar to those of skill in art. In some embodiments of this method, the affinity column contains chimeric proteins in which the PG-3 protein, or a fragment thereof, is fused to glutathion S transferase (GST). A mixture of cellular proteins or pool of expressed proteins as described above is applied to the affinity column. Proteins or other molecules interacting with the PG-3 protein, or a fragment thereof, attached to the column can then be isolated and analyzed on 2-D electrophoresis gel as described in Ramunsen et al. (1997). Alternatively, the proteins retained on the affinity column can be purified by electrophoresis based methods and sequenced. The same method can be used to isolate antibodies, to screen phage display products, or to screen phage display human antibodies.

D. Candidate Ligands Obtained by Optical Biosensor Methods

Proteins interacting with the PG-3 protein, or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3, can also be screened by using an Optical Biosensor as described in Edwards and Leatherbarrow (1997) and also in Szabo et al. (1995). This technique permits the detection of interactions between molecules in real time, without the need of labeled molecules. This technique is based on the surface plasmon resonance (SPR) phenomenon. Briefly, the candidate ligand molecule to be tested is attached to a surface (such as a carboxymethyl dextran matrix). A light beam is directed towards the side of the surface that does not contain the sample to be tested and is reflected by said surface. The SPR phenomenon causes a decrease in the intensity of the reflected light with a specific association of angle and wavelength. The binding of candidate ligand molecules cause a change in the refraction index on the surface, which change is detected as a change in the SPR signal. For screening of candidate ligand molecules or substances that are able to interact with the PG-3 protein, or a fragment thereof, the PG-3 protein, or a fragment thereof, is immobilized onto a surface. This surface consists of one side of a cell through which flows the candidate molecule to be assayed. The binding of the candidate molecule on the PG-3 protein, or a fragment thereof, is detected as a change of the SPR signal. The candidate molecules tested may be proteins, peptides, carbohydrates, lipids, or small molecules generated by combinatorial chemistry. This technique may also be performed by immobilizing eukaryotic or prokaryotic cells or lipid vesicles exhibiting an endogenous or a recombinantly expressed PG-3 protein at their surface.

The main advantage of the method is that it allows the determination of the association rate between the PG-3 protein and molecules interacting with the PG-3 protein. It is thus possible to select specifically ligand molecules interacting with the PG-3 protein, or a fragment thereof, through strong or conversely weak association constants.

E. Candidate Ligands Obtained Through a Two-Hybrid Screening Assay.

The yeast two-hybrid system is designed to study protein-protein interactions in vivo (Fields and Song, 1989), and relies upon the fusion of a bait protein to the DNA binding domain of the yeast Ga14 protein. This technique is also described in the U.S. Pat. No. 5,667,973, and the U.S. Pat. No. 5,283,173.

The general procedure of library screening by the two-hybrid assay may be performed as described by Harper et al. (1993) or as described by Cho et al. (1998) or also Fromont-Racine et al. (1997).

The bait protein or polypeptide consists of a PG-3 polypeptide or a fragment comprising a contiguous span of at least 6 amino acids, preferably at least 8 to 10 amino acids, more preferably at least 12, 15, 20, 25, 30, 40, 50, 100, 150, 200, 250, 300, 400, 500, 600, 700 or 800 amino acids of SEQ ID No 3.

More precisely, the nucleotide sequence encoding the PG-3 polypeptide or a fragment or variant thereof is fused to a polynucleotide encoding the DNA binding domain of the GAL4 protein, the fused nucleotide sequence being inserted in a suitable expression vector, for example pAS2 or pM3.

Then, a human cDNA library is constructed in a specially designed vector, such that the human EDNA insert is fused to a nucleotide sequence in the vector that encodes the transcriptional domain of the GAL4 protein. Preferably, the vector used is the pACT vector. The polypeptides encoded by the nucleotide inserts of the human cDNA library are termed “pray” poypeptides.

A third vector contains a detectable marker gene, such as beta galactosidase gene or CAT gene that is placed under the control of a regulation sequence that is responsive to the binding of a complete Gal4 protein containing both the transcriptional activation domain and the DNA binding domain. For example, the vector pG5EC may be used.

Two different yeast strains are also used. As an illustrative but non-limiting example the two different yeast strains may be the followings:

Y190, the phenotype of which is (MATa, Leu2-3, 112 ura3-12, trpl-901, his3-D200, ade2-101, gal4Dgal180D URA3 GAL-LacZ, LYS GAL-HIS3, cyh^{r});

Y187, the phenotype of which is (MA Ta gal4 gal80his3 trp1-901 ade2-101 ura3-52 leu2-3, -112 URA3 GAL-lacZmet⁻), which is the opposite mating type of Y190.

Briefly, 20 μg of pAS2/PG-3 and 20 pg of pACT-cDNA library are co-transformed into yeast strain Y190. The transformants are selected for growth on minimal media lacking histidine, leucine and tryptophan, but containing the histidine synthesis inhibitor 3-AT (50 mM). Positive colonies are screened for beta galactosidase by filter lift assay. The double positive colonies (His⁺, beta-gal⁺) are then grown on plates lacking histidine, leucine, but containing tryptophan and cycloheximide (10 mg/ml) to select for loss of pAS2/PG-3 plasmids bu retention of pACT-cDNA library plasmids. The resulting Y190 strains are mated with Y187 strains expressing PG-3 or non-related control proteins; such as cyclophilin B, lamin, or SNF1, as Gal4 fusions as described by Harper et al. (1993) and by Bram et al. (1993), and screened for beta galactosidase by filter lift assay. Yeast clones that are beta gal-after mating with the control Gal4 fusions are considered false positives.

In another embodiment of the two-hybrid method according to the invention, interaction between the PG-3 or a fragment or variant thereof with cellular proteins may be assessed using the Matchmaker Two Hybrid System 2 (Catalog No. K1604-1, Clontech). As described in the manual accompanying the Matchmaker Two Hybrid System 2 (Catalog No. K1604-1, Clontech), nucleic acids encoding the PG-3 protein or a portion thereof, are inserted into an expression vector such that they are in frame with DNA encoding the DNA binding domain of the yeast transcriptional activator GAL4. A desired cDNA, preferably human cDNA, is inserted into a second expression vector such that they are in frame with DNA encoding the activation domain of GAL4. The two expression plasmids are transformed into yeast and the yeast are plated on selection medium which selects for expression of selectable markers on each of the expression vectors as well as GAL4 dependent expression of the HIS3 gene. Transformants capable of growing on medium lacking histidine are screened for GAL4 dependent lacZ expression. Those cells which are positive in both the histidine selection and the lacZ assay contain interaction between PG-3 and the protein or peptide encoded by the initially selected cDNA insert.

### Method for Screening Substances Interacting with the Regulatory Sequences of the PG-3 Gene

The present invention also concerns a method for screening substances or molecules that are able to interact with the regulatory sequences of the PG-3 gene, such as for example promoter or enhancer sequences.

Nucleic acids encoding proteins which are able to interact with the regulatory sequences of the PG-3 gene, more particularly a nucleotide sequence selected from the group consisting of the polynucleotides of the 5′ and 3′ regulatory region or a fragment or variant thereof, and preferably a variant comprising one of the biallelic markers of the invention, may be identified by using a one-hybrid system, such as that described in the booklet enclosed in the Matchmaker One-Hybrid System kit from Clontech (Catalog Ref. n^{o} K1603-1). Briefly, the target nucleotide sequence is cloned upstream of a selectable reporter sequence and the resulting DNA construct is integrated in the yeast genome (Saccharomyces cerevisiae). The yeast cells containing the reporter sequence in their genome are then transformed with a library consisting of fusion molecules between cDNAs encoding candidate proteins for binding onto the regulatory sequences of the PG-3 gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the PG-3 gene. Then, the cDNAs encoding the fusion proteins are sequenced and may be cloned into expression or transcription vectors in vitro. The binding of the encoded polypeptides to the target regulatory sequences of the PG-3 gene may be confirmed by techniques familiar to the one skilled in the art, such as gel retardation assays or DNAse protection assays.

Gel retardation assays may also be performed independently in order to screen candidate molecules that are able to interact with the regulatory sequences of the PG-3 gene, such as described by Fried and Crothers (1981), Garner and Revzin (1981) and Dent and Latchman (1993). These techniques are based on the principle according to which a DNA fragment, which is bound to a protein, migrates slower than the same unbound DNA fragment. Briefly, the target nucleotide sequence is labeled. Then the labeled target nucleotide sequence is brought into contact with either a total nuclear extract from cells containing transcription factors, or with different candidate molecules to be tested. The interaction between the target regulatory sequence of the PG-3 gene and the candidate molecule or the transcription factor is detected after gel or capillary electrophoresis through a retardation in the migration.

### Method for Screening Ligands That Modulate the Expression of the PG-3 Gene

Another subject of the present invention is a method for screening molecules that modulate the expression of the PG-3 protein. Such a screening method comprises the steps of:

a) cultivating a prokaryotic or an eukaryotic cell that has been transfected with a nucleotide sequence encoding the PG-3 protein or a variant or a fragment thereof, placed under the control of its own promoter;

b) bringing into contact the cultivated cell with a molecule to be tested;

c) quantifying the expression of the PG-3 protein or a variant or a fragment thereof.

In an embodiment, the nucleotide sequence encoding the PG-3 protein or a variant or a fragment thereof comprises an allele of at least one of the biallelic markers A1 to A80, and the complements thereof.

Using DNA recombination techniques well known by the one skill in the art, the PG-3 protein encoding DNA sequence is inserted into an expression vector, downstream from its promoter sequence. As an illustrative example, the promoter sequence of the PG-3 gene is contained in the nucleic acid of the 5′ regulatory region.

The quantification of the expression of the PG-3 protein may be realized either at the mRNA level or at the protein level. In the latter case, polyclonal or monoclonal antibodies may be used to quantify the amounts of the PG-3 protein that have been produced, for example in an ELISA or a RIA assay.

In a preferred embodiment, the quantification of the PG-3 mRNA is realized by a quantitative PCR amplification of the cDNA obtained by a reverse transcription of the total mRNA of the cultivated PG-3-transfected host cell, using a pair of primers specific for PG-3.

The present invention also concerns a method for screening substances or molecules that are able to increase, or in contrast to decrease, the level of expression of the PG-3 gene. Such a method may allow the one skilled in the art to select substances exerting a regulating effect on the expression level of the PG-3 gene and which may be useful as active ingredients included in pharmaceutical compositions for treating patients suffering from cancer or a disorder relating to abnormal cellular differentiation.

Thus, another aspect of the present invention is a method for screening a candidate substance or molecule for the ability to modulate the expression of the PG-3 gene, comprising the following steps:

a) providing a recombinant cell host containing a nucleic acid, wherein said nucleic acid comprises a nucleotide sequence of the 5′ regulatory region or a regulatory active fragment or variant thereof located upstream of a polynucleotide encoding a detectable protein;

b) obtaining a candidate substance; and

c) determining the ability of the candidate substance to modulate the expression levels of the polynucleotide encoding the detectable protein.

In a further embodiment, the nucleic acid comprising the nucleotide sequence of the 5′ regulatory region or a regulatory active fragment or variant thereof also includes a 5UTR region of the PG-3 cDNA of SEQ ID No 2, or one of its regulatory active fragments or variants thereof.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a regulatory active fragment or variant thereof located upstream and operably linked to a polynucleotide encoding a detectable protein or the PG-3 protein or a fragment or a variant thereof.

In another embodiment of a method for the screening of a candidate substance or molecule for the ability to modulate the expression of the PG-3 gene, the method comprises the following steps:

a) providing a recombinant host cell containing a nucleic acid, wherein said nucleic acid comprises a 5′UTR sequence of the PG-3 cDNA of SEQ ID No 2, or one of its regulatory active fragments or variants, the 5′UTR sequence or its regulatory active fragment or variant being operably linked to a polynucleotide encoding a detectable protein;

b) obtaining a candidate substance; and

c) determining the ability of the candidate substance to modulate the expression levels of the polynucleotide encoding the detectable protein.

In a specific embodiment of the above screening method, the nucleic acid that comprises a nucleotide sequence selected from the group consisting of the 5°UTR sequence of the PG-3 cDNA of SEQ ID No 2 or one of its regulatory active fragments or variants, includes a promoter sequence which is endogenous with respect to the PG-35′UTR sequence.

In another specific embodiment of the above screening method, the nucleic acid that comprises a nucleotide sequence selected from the group consisting of the 5′UTR sequence of the PG-3 cDNA of SEQ ID No 2 or one of its regulatory active fragments or variants, includes a promoter sequence which is exogenous with respect to the PG-3 5′UTR sequence defined therein.

In a further preferred embodiment, the nucleic acid comprising the 5′-UTR sequence of the PG-3 cDNA or SEQ ID No 2 or the regulatory active fragments thereof includes a biallelic marker selected from the group consisting of A1 to A80 or the complements thereof.

The invention further encompasses a kit for the screening of a candidate substance for the ability to modulate the expression of the PG-3 gene, wherein said kit comprises a recombinant vector that comprises a nucleic acid including a 5′UTR sequence of the PG-3 cDNA of SEQ ID No 2, or one of their regulatory active fragments or variants, the 5′UTR sequence or its regulatory active fragment or variant being operably linked to a polynucleotide encoding a detectable protein.

For the design of suitable recombinant vectors useful for performing the screening methods described above, the section of the present specification wherein the preferred recombinant vectors of the invention are detailed is pertinent.

Expression levels and patterns of PG-3 may be analyzed by solution hybridization with long probes as described in International Patent Application No. WO 97/05277. Briefly, the PG-3 cDNA or the PG-3 genomic DNA described above, or fragments thereof, is inserted at a cloning site immediately downstream of a bacteriophage (T3, T7 or SP6) RNA polymerase promoter to produce antisense RNA. Preferably, the PG-3 insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridization is performed under standard stringent conditions (40-50° C. for 16 hours in an 80% formamide, 0. 4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of PG-3 gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the PG-3 genomic DNA, the PG-3 cDNA sequences or the sequences complementary thereto or fragments thereof, particularly those comprising at least one of the biallelic markers according the present invention, preferably at least one of the biallelic markers A1 to A80. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of PG-3 gene expression may be performed with a complementary DNA microarray as described by Schena et al.(1995 and 1996). Full-length PG-3 cDNAs or fragments thereof are amplified by PCR and arrayed from a 96-well microtiter plate onto silylated microscope slides using high-speed robotics. Printed arrays are incubated in a humid chamber to allow rehydration of the array elements and rinsed, once in 0. 2% SDS for 1 min, twice in water for 1 min and once for 5 min in sodium borohydride solution. The arrays are submerged in water for 2 min at 95° C., transferred into 0.2% SDS for 1 min, rinsed twice with water, air-dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of PG-3 gene expression may also be performed with full length PG-3 cDNAs or fragments thereof in complementary DNA arrays as described by Pietu et al.(1996). The full length PG-3 cDNA or fragments thereof is PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the PG-3 genomic DNA, the PG-3 cDNA, or fragments thereof can be done through high density nucleotide arrays as described by Lockhart et al. (1996) and Sosnowski et al. (1997). Oligonucleotides of 15-50 nucleotides from the sequences of the PG-3 genomic DNA, the PG-3 cDNA sequences particularly those comprising at least one of biallelic markers according the present invention, preferably at least one biallelic marker selected from the group consisting of A1 to A80, or the sequences complementary thereto, are synthesized directly on the chip (Lockhart et al., supra) or synthesized and then addressed to the chip (Sosnowski et al., supra). Preferably, the oligonucleotides are about 20 nucleotides in length.

PG-3 cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowski et al., 1997), the dyes or labeling compounds are detected and quantified. Duplicate hybridizations are performed. Comparative analysis of the intensity of the signal originating from cDNA probes on the same target oligonucleotide in different cDNA samples indicates a differential expression of PG-3 mRNA.

### Methods for Inhibiting the Expression of a PG-3 Gene

Other therapeutic compositions according to the present invention comprise advantageously an oligonucleotide fragment of the nucleic sequence of PG-3 as an antisense tool or a triple helix tool that inhibits the expression of the corresponding PG-3 gene. A preferred fragment of the nucleic sequence of PG-3 comprises an allele of at least one of the biallelic markers A1 to A80.

Antisense Approach

In antisense approaches, nucleic acid sequences complementary to an mRNA are hybridized to the mRNA intracellularly, thereby blocking the expression of the protein encoded by the mRNA. The antisense nucleic acid molecules to be used in gene therapy may be either DNA or RNA sequences. Preferred methods using antisense polynucleotide according to the present invention are the procedures described by Sczakiel et al. (1995), which disclosure is hereby incorporated by reference in its entirety.

Preferably, the antisense tools are chosen among the polynucleotides (15-200 bp long) that are complementary to PG-3 mRNA, more preferably to the 5′end of the PG-3 mRNA. In another embodiment, a combination of different antisense polynucleotides complementary to different parts of the desired targeted gene are used.

Other preferred antisense polynucleotides according to the present invention are sequences complementary to either a sequence of PG-3 mRNAs comprising the translation initiation codon ATG or a sequence of PG-3 genomic DNA containing a splicing donor or acceptor site.

Preferably, the antisense polynucleotides of the invention have a 3′ polyadenylation signal that has been replaced with a self-cleaving ribozyme sequence, such that RNA polymerase II transcripts are produced without poly(A) at their 3′ ends, these antisense polynucleotides being incapable of export from the nucleus, such as described by Liu et al.(1994), which disclosure is hereby incorporated by reference in its entirety. In a preferred embodiment, these PG-3 antisense polynucleotides also comprise, within the ribozyme cassette, a histone stem-loop structure to stabilize cleaved transcripts against 3′-5′ exonucleolytic degradation, such as the structure described by Eckner et al. (1991), which disclosure is hereby incorporated by reference in its entirety.

The antisense nucleic acids should have a length and melting temperature sufficient to permit formation of an intracellular duplex having sufficient stability to inhibit the expression of the PG-3 mRNA in the duplex. Strategies for designing antisense nucleic acids suitable for use in gene therapy are disclosed in Green et al., (1986) and Izant and Weintraub, (1984), the disclosures of which are incorporated herein by reference.

In some strategies, antisense molecules are obtained by reversing the orientation of the PG-3 coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of PG-3 antisense nucleic acids in vivo by operably linking DNA containing the antisense sequence to a promoter in a suitable expression vector.

Alternatively, oligonucleotides which are complementary to the strand normally transcribed in the cell may be synthesized in vitro. Thus, the antisense nucleic acids are complementary to the corresponding mRNA and are capable of hybridizing to the mRNA to create a duplex. In some embodiments, the antisense sequences may contain modified sugar phosphate backbones to increase stability and make them less sensitive to RNase activity. Examples of modifications suitable for use in antisense strategies include 2′ O-methyl RNA oligonucleotides and Protein-nucleic acid (PNA) oligonucleotides. Further examples are described by Rossi et al., (1991), which disclosure is hereby incorporated by reference in its entirety.

Various types of antisense oligonucleotides complementary to the sequence of the PG-3 cDNA or genomic DNA may be used. In one preferred embodiment, stable and semi-stable antisense oligonucleotides described in International Application No. PCT WO94/23026, hereby incorporated by reference, are used. In these molecules, the 3′ end or both the 3′ and 5′ ends are engaged in intramolecular hydrogen bonding between complementary base pairs. These molecules are better able to withstand exonuclease attacks and exhibit increased stability compared to conventional antisense oligonucleotides.

In another preferred embodiment, the antisense oligodeoxynucleotides against herpes simplex virus types 1 and 2 described in International Application No. WO 95/04141, hereby incorporated by reference, are used.

In yet another preferred embodiment, the covalently cross-linked antisense oligonucleotides, described in International Application No. WO 96/31523, hereby incorporated by reference, are used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522, incorporated by reference, may also be used. These molecules are stable to degradation and contain at least one transcription control recognition sequence which binds to control proteins and are effective as decoys therefor. These molecules may contain “hairpin” structures, “dumbbell” structures, “modified dumbbell” structures, “cross-linked” decoy structures and “loop” structures.

In another preferred embodiment, the cyclic double-stranded oligonucleotides described in European Patent Application No. 0 572 287 A2, hereby incorporated by reference are used. These ligated oligonucleotide “dumbbells” contain the binding site for a transcription factor and inhibit expression of the gene under control of the transcription factor by sequestering the factor.

Use of the closed antisense oligonucleotides disclosed in International Application No. WO 92/19732, hereby incorporated by reference, is also contemplated. Because these molecules have no free ends, they are more resistant to degradation by exonucleases than are conventional oligonucleotides. These oligonucleotides may be multifunctional, interacting with several regions which are not adjacent to the target mRNA.

The appropriate level of antisense nucleic acids required to inhibit gene expression may be determined using in vitro expression analysis. The antisense molecule may be introduced into the cells by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral protein, or as an oligonucleotide operably linked to a promoter contained in an expression vector. The expression vector may be any of a variety of expression vectors known in the art, including retroviral or viral vectors, vectors capable of extrachromosomal replication, or integrating vectors. The vectors may be DNA or RNA.

The antisense molecules are introduced onto cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher may be possible after testing the toxicity of the oligonucleotide in laboratory animals. It is additionally contemplated that cells from the vertebrate are removed, treated with the antisense oligonucleotide, and reintroduced into the vertebrate.

In a preferred application of this invention, the polypeptide encoded by the gene is first identified, so that the effectiveness of antisense inhibition on translation can be monitored using techniques that include but are not limited to antibody-mediated tests such as RIAs and ELISA, functional assays, or radiolabeling.

An alternative to the antisense technology that is used according to the present invention comprises using ribozymes that will bind to a target sequence via their complementary polynucleotide tail and that will cleave the corresponding RNA by hydrolyzing its target site (namely “hammerhead ribozymes”). Briefly, the simplified cycle of a hammerhead ribozyme comprises (1) sequence specific binding to the target RNA via complementary antisense sequences; (2) site-specific hydrolysis of the cleavable motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Rossi et al, (1991) and Sczakiel et al. (1995), the specific preparation procedures being referred to in said articles being herein incorporated by reference.

Triple Helix Approach

The PG-3 genomic DNA may also be used to inhibit the expression of the PG-3 gene based on intracellular triple helix formation.

Triple helix oligonucleotides are used to inhibit transcription from a genome. They are particularly useful for studying alterations in cell activity when it is associated with a particular gene.

Similarly, a portion of the PG-3 genomic DNA can be used to study the effect of inhibiting PG-3 transcription within a cell. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences from the PG-3 genomic DNA are contemplated within the scope of this invention.

To carry out gene therapy strategies using the triple helix approach, the sequences of the PG-3 genomic DNA are first scanned to identify 10-mer to 20-mer homopyrimidine or homopurine stretches which could be used in triple-helix based strategies for inhibiting PG-3 expression. Following identification of candidate homopyrimidine or homopurine stretches, their efficiency in inhibiting PG-3 expression is assessed by introducing varying amounts of oligonucleotides containing the candidate sequences into tissue culture cells which express the PG-3 gene.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for altered cell function or reduced PG-3 expression using techniques such as Northern blotting, RNase protection assays, or PCR based strategies to monitor the transcription levels of the PG-3 gene in cells which have been treated with the oligonucleotide.

The oligonucleotides which are effective in inhibiting gene expression in tissue culture cells may then be introduced in vivo using the techniques described above in the antisense approach at a dosage calculated based on the in vitro results, as described in antisense approach.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al.(1989), which is hereby incorporated by this reference.

### Computer-Related Embodiments

As used herein the term “nucleic acid codes of the invention” encompass the nucleotide sequences comprising, consisting essentially of, or consisting of any one of the following: a) a contiguous span of at least 12, 15, 18, 20, 25, 30, 35, 40, 50, 60, 70, 80, 90, 100, 150, 200, 500, or 1000 nucleotides of SEQ ID No 1, wherein said contiguous span comprises at least 1, 2, 3, 5, or 10 of the following nucleotide positions of SEQ ID No 1: 1-97921, 98517-103471, 103603-108222, 108390-109221, 109324-114409, 114538-115723, 115957-122102, 122225-126876, 127033-157212, 157808-240825; b) a contiguous span of at least 12, 15, 18, 20, 25, 30, 35, 40, 50, 60, 70, 80, 90, 100, 150, 200, 500, or 1000 nucleotides of SEQ ID No 2 or the complements thereof; and, c) a nucleotide sequence complementary to any one of the preceding nucleotide sequences.

The “nucleic acid codes of the invention” further encompass nucleotide sequences homologous to:

a) a contiguous span of at least 12, 15, 18, 20, 25, 30, 35, 40, 50, 60, 70, 80, 90, 100, 150, 200, 500, or 1000 nucleotides of SEQ ID No 1, wherein said contiguous span comprises at least 1, 2, 3, 5, or 10 of the following nucleotide positions of SEQ ID No 1: 1-97921, 98517-103471, 103603-108222, 108390-109221, 109324-114409, 114538-115723, 115957-122102, 122225-126876, 127033-157212, 157808-240825;

b) a contiguous span of at least 12, 15, 18, 20, 25, 30, 35, 40, 50, 60, 70, 80, 90, 100, 150, 200, 500, or 1000 nucleotides of SEQ ID No 2 or the complements thereof; and,

c) c) sequences complementary to all of the preceding sequences.

Homologous sequences refer to a sequence having at least 99%, 98%, 97%, 96%, 95%, 90%, 85%, 80%, or 75% homology to these contiguous spans. Homology may be determined using any method described herein, including BLAST2N with the default parameters or with any modified parameters. Homologous sequences also may include RNA sequences in which uridines replace the thymines in the nucleic acid codes of the invention. It will be appreciated that the nucleic acid codes of the invention can be represented in the traditional single character format (See the inside back cover of Stryer, Lubert. 1995) or in any other format or code which records the identity of the nucleotides in a sequence.

As used herein the term “polypeptide codes of the invention” encompass the polypeptide sequences comprising a contiguous span of at least 6, 8, 10, 12, 15, 20, 25, 30, 40, 50, or 100 amino acids of SEQ ID No 3. It will be appreciated that the polypeptide codes of the invention can be represented in the traditional single character format or three-letter format (See the inside back cover of Stryer, Lubert.) or in any other format or code which records the identity of the polypeptides in a sequence.

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 2, 5, 10, 15, 20, 25, 30, or 50 nucleic acid codes of the invention. Another aspect of the present invention is a computer readable medium having recorded thereon at least 2, 5, 10, 15, 20, 25, 30, or 50 polypeptide codes of the invention.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

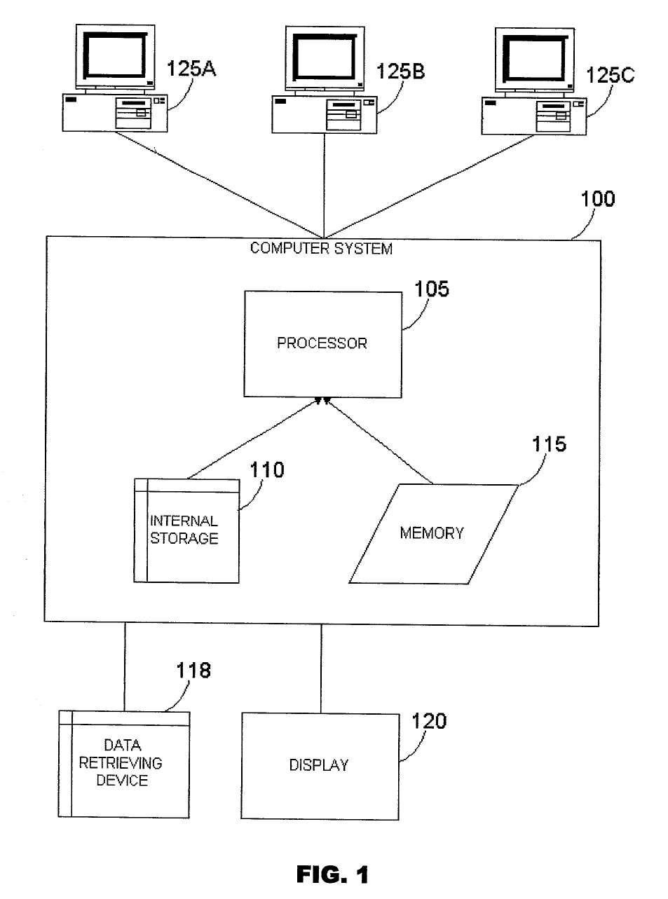

Embodiments of the present invention include systems, particularly computer systems which store and manipulate the sequence information described herein. One example of a computer system 100 is illustrated in block diagram form in FIG. 1. As used herein, “a computer system” refers to the hardware components, software components, and data storage components used to analyze the nucleotide sequences of the nucleic acid codes of the invention or the amino acid sequences of the polypeptide codes of the invention. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data-retrieving device 118 for reading the data stored on the internal data storage devices 110.

The data-retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data-retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the nucleic acid codes of the invention or the amino acid sequences of the polypeptide codes of the invention (such as search tools, compare tools, and modeling tools, etc.) may reside in main memory 115 during execution.

In some embodiments, the computer system 100 may further comprise a sequence comparer for comparing the above-described nucleic acid codes of the invention or the polypeptide codes of the invention stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including, but not limited to, peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

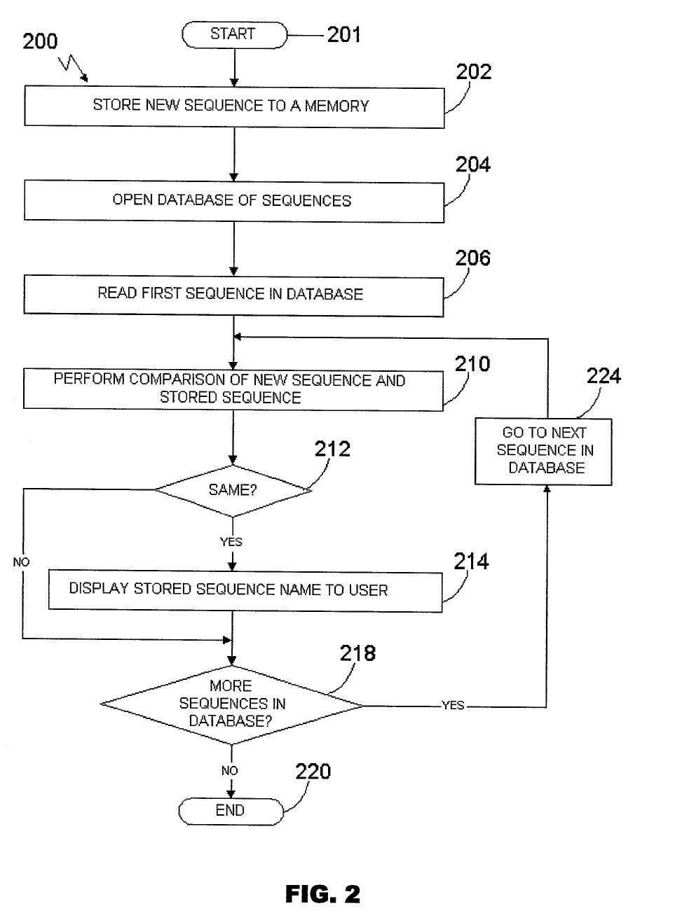

FIG. 2 is a flow diagram illustrating one embodiment of a process 200 for comparing a new nucleotide or protein sequence with a database of sequences in order to determine the homology levels between the new sequence and the sequences in the database. The database of sequences can be a private database stored within the computer system 100, or a public database such as GENBANK, PIR OR SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison. The sequence comparer may indicate a homology level between the sequences compared or identify structural motifs in the nucleic acid code of the invention and polypeptide codes of the invention or it may identify structural motifs in sequences which are compared to these nucleic acid codes and polypeptide codes. In some embodiments, the data storage device may have stored thereon the sequences of at least 2, 5, 10, 15, 20, 25, 30, or 50 of the nucleic acid codes of the invention or polypeptide codes of the invention.

Another aspect of the present invention is a method for determining the level of homology between a nucleic acid code of the invention and a reference nucleotide sequence, comprising the steps of reading the nucleic acid code and the reference nucleotide sequence through the use of a computer program which determines homology levels and determining homology between the nucleic acid code and the reference nucleotide sequence with the computer program. The computer program may be any of a number of computer programs for determining homology levels, including those specifically enumerated herein, including BLAST2N with the default parameters or with any modified parameters. The method may be implemented using the computer systems described above. The method may also be performed by reading 2, 5, 10, 15, 20, 25, 30, or 50 of the above described nucleic acid codes of the invention through the use of the computer program and determining homology between the nucleic acid codes and reference nucleotide sequences.

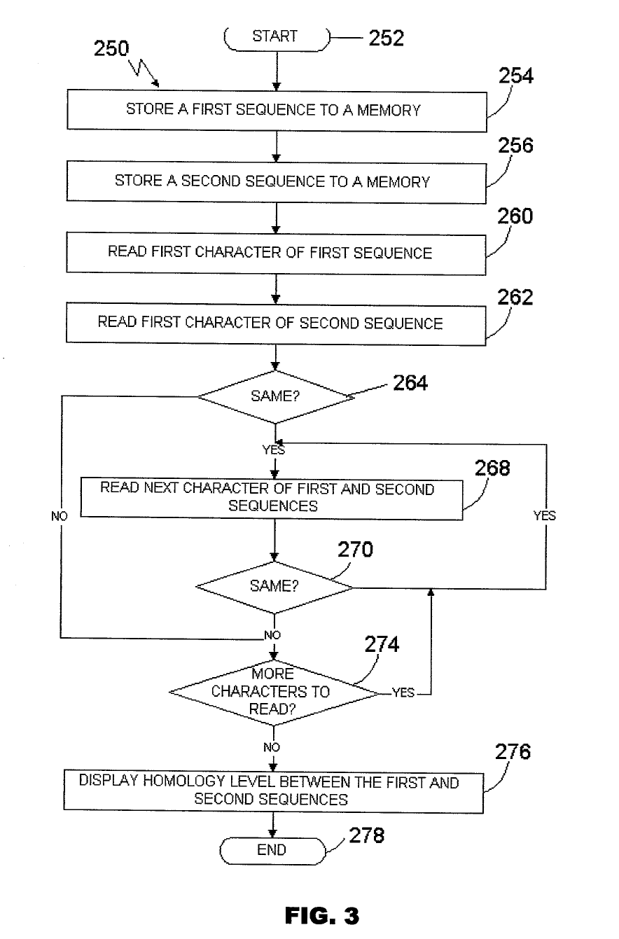

FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with a every character in a second sequence, the homology level would be 100%.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion.

Another aspect of the present invention is a method for determining the level of homology between a polypeptide code of the invention and a reference polypeptide sequence, comprising the steps of reading the polypeptide code of the invention and the reference polypeptide sequence through use of a computer program which determines homology levels and determining homology between the polypeptide code and the reference polypeptide sequence using the computer program.

Accordingly, another aspect of the present invention is a method for determining whether a nucleic acid code of the invention differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms. The method may be implemented by the computer systems described above, and the method illustrated in FIG. 3. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program.

In other embodiments the computer based system may further comprise an identifier for identifying features within the nucleotide sequences of the nucleic acid codes of the invention or the amino acid sequences of the polypeptide codes of the invention.

An “identifier” refers to one or more programs which identifies certain features within the above-described nucleotide sequences of the nucleic acid codes of the invention or the amino acid sequences of the polypeptide codes of the invention. In one embodiment, the identifier Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence homology.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a fall-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into interresidue distance restraints and fed into the distance geometry program DRAGON, together with auxiliary information obtained from secondary structure predictions. The program combines the restraints in an unbiased manner and rapidly generates a large number of low-resolution model confirmations. In a third step, these low resolution model confirmations are converted into full-atom models and subjected to energy minimization using the molecular modeling package QUANTA. (See e.g., Asódi, et al., (1997)).

The results of the molecular modeling analysis may then be used in rational drug design techniques to identify agents which modulate the activity of the polypeptide codes of the invention.

Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer programidentifies structural motifs in a polypeptide sequence. In another embodiment, the computer program comprises a molecular modeling program. The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program.

The nucleic acid codes of the invention or the polypeptide codes of the invention may be stored and manipulated in a variety of data processor programs in a variety of formats. For example, they may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the nucleic acid codes of the invention or the polypeptide codes of the invention. The following list is intended not to limit the invention but to provide guidance to programs and databases which are useful with the nucleic acid codes of the invention or the polypeptide codes of the invention. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, 1990), FASTA (Pearson and Lipman, 1988), FASTDB (Brutlag et al., 1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, the Genseqn database and the Genseqp databases. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Throughout this application, various publications, patents and published patent applications are cited. The disclosures of these publications, patents and published patent specification referenced in this application are hereby incorporated by reference into the present disclosure to more fully describe the sate of the art to which this invention pertains.

### EXAMPLES

### Example 1

### Identification of Biallelic Markers—DNA Extraction

Donors were unrelated and healthy. They presented a sufficient diversity for being representative of a French heterogeneous population. The DNA from 100 individuals was extracted and tested for the detection of the biallelic markers.

30 ml of peripheral venous blood were taken from each donor in the presence of EDTA. Cells (pellet) were collected after centrifugation for 10 minutes at 2000 rpm. Red cells were lysed by a lysis solution (50 ml final volume: 10 mM Tris pH 7.6; 5 MM MgCl₂; 10 mM NaCl). The solution was centrifuged (10 minutes, 2000 rpm) as many times as necessary to eliminate the residual red cells present in the supernatant, after resuspension of the pellet in the lysis solution.

The pellet of white cells was lysed overnight at 42° C. with 3.7 ml of lysis solution composed of:

3 ml TE 10-2 (Tris-HCl 10 mM, EDTA 2 mM)/NaCl 0 4 M

200 μl SDS 10%

500 μl K-proteinase (2 mg K-proteinase in TE 10-2/NaCl 0.4 M).

For the extraction of proteins, 1 ml saturated NaCl (6M) (1/3.5 v/v) was added. After vigorous agitation, the solution was centrifuged for 20 minutes at 10000 rpm.

For the precipitation of DNA, 2 to 3 volumes of 100% ethanol were added to the previous supernatant, and the solution was centrifuged for 30 minutes at 2000 rpm. The DNA solution was rinsed three times with 70% ethanol to eliminate salts, and centrifuged for 20 minutes at 2000 rpm. The pellet was dried at 37° C., and resuspended in 1 ml TE 10-1 or 1 ml water. The DNA concentration was evaluated by measuring the OD at 260 nm (1 unit OD=50 μg/ml DNA).

To determine the presence of proteins in the DNA solution, the OD 260/OD 280 ratio was determined. Only DNA preparations having a OD 260/OD 280 ratio between 1.8 and 2 were used in the subsequent examples described below.

The pool was constituted by mixing equivalent quantities of DNA from each individual.

### Example 2

### Identification of Biallelic Markers: Amplification of Genomic DNA by PCR

The amplification of specific genomic sequences of the DNA samples of example 1 was carried out on the pool of DNA obtained previously. In addition, 50 individual samples were similarly amplified.

PCR assays were performed using the following protocol:

| | |
|---|---|
| Final volume | 25 μl |
| DNA | 2 ng/μl |
| MgCl₂ | 2 mM |
| dNTP (each) | 200 μM |
| primer (each) | 2.9 ng/μl |
| Ampli Taq Gold DNA polymerase | 0.05 unit/μl |
| PCR buffer (10x = 0.1 M TrisHCl pH8.3 0.5 M KCl) | 1x |

Each pair of first primers was designed using the sequence information of the PG-3 gene disclosed herein and the OSP software (Hillier & Green, 1991). This first pair of primers was about 20 nucleotides in length and had the sequences disclosed in Table 1 in the columns labeled PU and RP.

TABLE 1

| | | | | | | | Complementary | |
|---|---|---|---|---|---|---|---|---|
| | | | | Position range | | | position range of | |
| | Position range | | PU | of amplification | | RP | amplification | |
| | of the amplicon | | primer | primer in SEQ | | primer | primer in SEQ | |
| Amplicon | in SEQ ID No: 1 | | name | ID No: 1 | | name | ID No: 1 | |
| 5-390 | 1823 | 2125 | B1 | 1823 | 1840 | C1 | 2108 | 2125 |
| 5-391 | 4559 | 4908 | B2 | 4559 | 4577 | C2 | 4891 | 4908 |
| 5-392 | 10007 | 10430 | B3 | 10007 | 10025 | C3 | 10411 | 10430 |
| 4-59 | 39556 | 39970 | B4 | 39556 | 39574 | C4 | 39953 | 39970 |
| 4-58 | 39877 | 40259 | B5 | 39877 | 39896 | C5 | 40242 | 40259 |
| 4-54 | 41137 | 41581 | B6 | 41137 | 41154 | C6 | 41564 | 41581 |
| 4-51 | 42122 | 42543 | B7 | 42122 | 42141 | C7 | 42526 | 42543 |
| 99-86 | 67289 | 67741 | B8 | 67289 | 67309 | C8 | 67724 | 67741 |
| 4-88 | 69182 | 69626 | B9 | 69182 | 69200 | C9 | 69609 | 69626 |
| 5-397 | 72698 | 73117 | B10 | 72698 | 72715 | C10 | 73099 | 73117 |
| 5-398 | 75858 | 76306 | B11 | 75858 | 75877 | C11 | 76289 | 76306 |
| 99-12738 | 81006 | 81485 | B12 | 81006 | 81025 | C12 | 81466 | 81485 |
| 99-109 | 83564 | 84007 | B13 | 83564 | 83582 | C13 | 83990 | 84007 |
| 99-12749 | 91743 | 92142 | B14 | 91743 | 91763 | C14 | 92123 | 92142 |
| 4-21 | 95196 | 95619 | B15 | 95196 | 95214 | C15 | 95600 | 95619 |
| 4-23 | 95865 | 96229 | B16 | 95865 | 95882 | C16 | 96210 | 96229 |
| 99-12753 | 97261 | 97747 | B17 | 97261 | 97278 | C17 | 97728 | 97747 |
| 5-364 | 97831 | 98275 | B18 | 97831 | 97849 | C18 | 98256 | 98275 |
| 99-12755 | 98638 | 99131 | B19 | 98638 | 98656 | C19 | 99111 | 99131 |
| 4-87 | 103376 | 103818 | B20 | 103376 | 103395 | C20 | 103801 | 103818 |
| 99-12757 | 104081 | 104636 | B21 | 104081 | 104100 | C21 | 104619 | 104636 |
| 99-12758 | 106272 | 106799 | B22 | 106272 | 106291 | C22 | 106780 | 106799 |
| 4-105 | 108200 | 108412 | B23 | 108200 | 108218 | C23 | 108390 | 108412 |
| 4-45 | 108223 | 108520 | B24 | 108223 | 108246 | C24 | 108499 | 108520 |
| 4-44 | 109123 | 109471 | B25 | 109123 | 109142 | C25 | 109454 | 109471 |
| 4-86 | 114217 | 114663 | B26 | 114217 | 114234 | C26 | 114646 | 114663 |
| 4-84 | 115630 | 116049 | B27 | 115630 | 115647 | C27 | 116031 | 116049 |
| 99-78 | 121991 | 122401 | B28 | 121991 | 122011 | C28 | 122384 | 122401 |
| 99-12767 | 123089 | 123583 | B29 | 123089 | 123106 | C29 | 123565 | 123583 |
| 4-80 | 126711 | 127065 | B30 | 126711 | 126729 | C30 | 127048 | 127065 |
| 4-36 | 128162 | 128590 | B31 | 128162 | 128179 | C31 | 128573 | 128590 |
| 4-35 | 128480 | 128926 | B32 | 128480 | 128497 | C32 | 128909 | 128926 |
| 99-12771 | 130747 | 131273 | B33 | 130747 | 130764 | C33 | 131254 | 131273 |
| 99-12774 | 132873 | 133325 | B34 | 132873 | 132892 | C34 | 133305 | 133325 |
| 99-12776 | 135029 | 135478 | B35 | 135029 | 135048 | C35 | 135458 | 135478 |
| 99-12781 | 139277 | 139742 | B36 | 139277 | 139296 | C36 | 139724 | 139742 |
| 4-104 | 157181 | 157832 | B37 | 157181 | 157199 | C37 | 157814 | 157832 |
| 99-12818 | 172692 | 173091 | B38 | 172692 | 172709 | C38 | 173072 | 173091 |
| 99-24807 | 180248 | 180892 | B39 | 180248 | 180268 | C39 | 180874 | 180892 |
| 99-12827 | 184662 | 185156 | B40 | 184662 | 184680 | C40 | 185138 | 185156 |
| 99-12831 | 190178 | 190663 | B41 | 190178 | 190196 | C41 | 190643 | 190663 |
| 99-12832 | 191011 | 191460 | B42 | 191011 | 191030 | C42 | 191441 | 191460 |
| 99-12836 | 195099 | 195587 | B43 | 195099 | 195116 | C43 | 195568 | 195587 |
| 99-12844 | 203585 | 204115 | B44 | 203585 | 203602 | C44 | 204095 | 204115 |
| 4-24 | 210079 | 210495 | B45 | 210079 | 210096 | C45 | 210476 | 210495 |
| 4-27 | 210979 | 211401 | B46 | 210979 | 210996 | C46 | 211382 | 211401 |
| 5-400 | 215852 | 216271 | B47 | 215852 | 215870 | C47 | 216253 | 216271 |
| 99-12852 | 216213 | 216728 | B48 | 216213 | 216231 | C48 | 216708 | 216728 |
| 4-37 | 221530 | 221973 | B49 | 221530 | 221549 | C49 | 221956 | 221973 |
| 5-270 | 225554 | 225845 | B50 | 225554 | 225572 | C50 | 225827 | 225845 |
| 99-12860 | 229341 | 229790 | B51 | 229341 | 229359 | C51 | 229770 | 229790 |
| 5-402 | 237412 | 237766 | B52 | 237412 | 237429 | C52 | 237747 | 237766 |

Preferably, the primers contained a common oligonucleotide tail upstream of the specific bases targeted for amplification which was useful for sequencing.

Primers PU contain the following additional PU 5′ sequence: 5 TGTAAAACGACGGCCAGT; primers RP contain the following RP 5′ sequence: CAGGAAACAGCTATGACC. The primer containing the additional PU 5′ sequence is listed in SEQ ID No 4. The primer containing the additional RP 5′ sequence is listed in SEQ ID No 5.

The synthesis of these primers was performed following the phosphoramidite method, on a GENSET UFPS 24.1 synthesizer.

DNA amplification was performed on a Genius II thermocycler. After heating at 95° C. for 10 min, 40 cycles were performed. Each cycle comprised: 30 sec at 95° C., 54° C. for 1 min, and 30 sec at 72° C. For final elongation, 10 min at 72° C. ended the amplification. The quantities of the amplification products obtained were determined on 96-well microtiter plates, using a fluorometer and Picogreen as intercalant agent (Molecular Probes).

### Example 3

### Identification of Biallelic Markers—Sequencing of Amplified Genomic DNA and Identification of Polymorphisms

The sequencing of the amplified DNA obtained in example 2 was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version)).

The sequence data were further evaluated to detect the presence of biallelic markers within the amplified fragments. The polymorphism search was based on the presence of superimposed peaks in the electrophoresis pattern resulting from different bases occurring at the same position as described previously.

In the 52 fragments of amplification, 80 biallelic markers were detected. The localization of these biallelic markers are as shown in Table 2.

TABLE 2

| | | | | | | BM position in | | Position of |
|---|---|---|---|---|---|---|---|---|
| | | | Localization | Polymorphism | | SEQ ID | | amino acid in |
| Amplicon | BM | Marker name | in PG-3 gene | all1 | all2 | No: 1 | No: 2 | SEQ ID No: 3 |
| 5-390 | A1 | 5-390-177 | 5′ regulatory | G | C | 1999 | | |
| 5-391 | A2 | 5-391-43 | Intron A-B | A | G | 4601 | | |
| 5-392 | A3 | 5-392-222 | Exon C | G | T | 10228 | 285 | 76 = V |
| 5-392 | A4 | 5-392-280 | Intron C-D | G | T | 10286 | | |
| 5-392 | A5 | 5-392-364 | Intron C-D | G | — | 10370 | | |
| 4-59 | A6 | 4-58-318 | Exon T | G | T | 39944 | 968 | 304 = R or I |
| 4-58 | A7 | 4-58-289 | Exon T | G | C | 39973 | 997 | 314 = H or D |
| 4-54 | A8 | 4-54-199 | Intron T-G | A | C | 41385 | | |
| 4-54 | A9 | 4-54-180 | Intron T-G | A | C | 41404 | | |
| 4-51 | A10 | 4-51-312 | Intron T-G | G | C | 42232 | | |
| 99-86 | A11 | 99-86-266 | Intron G-H | A | G | 67475 | | |
| 4-88 | A12 | 4-88-107 | Intron G-H | A | G | 69521 | | |
| 5-397 | A13 | 5-397-141 | Intron G-H | G | T | 72838 | | |
| 5-398 | A14 | 5-398-203 | Exon I | A | C | 76060 | 2102 | 682 = T or N |
| 99-12738 | A15 | 99-12738-248 | Intron I-J | A | C | 81253 | | |
| 99-109 | A16 | 99-109-358 | Intron I-J | A | C | 83921 | | |
| 99-12749 | A17 | 99-12749-175 | Intron I-J | C | T | 91917 | | |
| 4-21 | A18 | 4-21-154 | Intron J-K | C | T | 95349 | | |
| 4-21 | A19 | 4-21-317 | Intron J-K | G | T | 95511 | | |
| 4-23 | A20 | 4-23-326 | Intron J-K | A | G | 96190 | | |
| 99-12753 | A21 | 99-12753-34 | Intron J-K | A | T | 97294 | | |
| 5-364 | A22 | 5-364-252 | Intron J-K | G | T | 98024 | | |
| 99-12755 | A23 | 99-12755-280 | Intron J-K | A | G | 98914 | | |
| 99-12755 | A24 | 99-12755-329 | Intron J-K | A | C | 98963 | | |
| 4-87 | A25 | 4-87-212 | Intron J-K | A | G | 103593 | | |
| 99-12757 | A26 | 99-12757-318 | Intron J-K | C | T | 104398 | | |
| 99-12758 | A27 | 99-12758-102 | Intron J-K | A | G | 106373 | | |
| 99-12758 | A28 | 99-12758-136 | Intron J-K | C | T | 106407 | | |
| 4-105 | A29 | 4-105-98 | Intron J-K | A | G | 108315 | | |
| 4-105 | A30 | 4-105-86 | Intron J-K | A | G | 108327 | | |
| 4-45 | A31 | 4-45-49 | Intron J-K | C | T | 108472 | | |
| 4-44 | A32 | 4-44-277 | Intron J-K | C | T | 109196 | | |
| 4-86 | A33 | 4-86-60 | Intron J-K | G | C | 114604 | | |
| 4-84 | A34 | 4-84-334 | Intron J-K | A | G | 115716 | | |
| 99-78 | A35 | 99-78-321 | Intron J-K | A | T | 122083 | | |
| 99-12767 | A36 | 99-12767-36 | Intron J-K | G | C | 123124 | | |
| 99-12767 | A37 | 99-12767-143 | Intron J-K | C | T | 123231 | | |
| 99-12767 | A38 | 99-12767-189 | Intron J-K | C | T | 123277 | | |
| 99-12767 | A39 | 99-12767-380 | Intron J-K | A | G | 123468 | | |
| 4-80 | A40 | 4-80-328 | Intron J-K | C | T | 126738 | | |
| 4-36 | A41 | 4-36-384 | Intron J-K | G | C | 128210 | | |
| 4-36 | A42 | 4-36-264 | Intron J-K | A | G | 128330 | | |
| 4-36 | A43 | 4-36-261 | Intron J-K | A | C | 128333 | | |
| 4-35 | A44 | 4-35-333 | Intron J-K | A | C | 128594 | | |
| 4-35 | A45 | 4-35-240 | Intron J-K | G | C | 128687 | | |
| 4-35 | A46 | 4-35-173 | Intron J-K | A | T | 128754 | | |
| 4-35 | A47 | 4-35-133 | Intron J-K | C | T | 128794 | | |
| 99-12771 | A48 | 99-12771-59 | Intron J-K | G | T | 130805 | | |
| 99-12774 | A49 | 99-12774-334 | Intron J-K | A | C | 133206 | | |
| 99-12776 | A50 | 99-12776-358 | Intron J-K | A | G | 135386 | | |
| 99-12781 | A51 | 99-12781-113 | Intron J-K | A | G | 139389 | | |
| 4-104 | A52 | 4-104-298 | Intron J-K | G | C | 157535 | | |
| 4-104 | A53 | 4-104-254 | Intron J-K | A | G | 157579 | | |
| 4-104 | A54 | 4-104-250 | Intron J-K | C | T | 157583 | | |
| 4-104 | A55 | 4-104-214 | Intron J-K | A | G | 157619 | | |
| 99-12818 | A56 | 99-12818-289 | Intron J-K | C | T | 172980 | | |
| 99-24807 | A57 | 99-24807-271 | Intron J-K | C | T | 180622 | | |
| 99-24807 | A58 | 99-24807-84 | Intron J-K | A | G | 180809 | | |
| 99-12831 | A59 | 99-12831-157 | Intron J-K | A | G | 190334 | | |
| 99-12831 | A60 | 99-12831-241 | Intron J-K | C | T | 190418 | | |
| 99-12832 | A61 | 99-12832-387 | Intron J-K | C | T | 191397 | | |
| 99-12836 | A62 | 99-12836-30 | Intron J-K | G | C | 195128 | | |
| 99-12844 | A63 | 99-12844-262 | Intron J-K | G | C | 203846 | | |
| 4-24 | A64 | 4-24-74 | Intron J-K | C | T | 210151 | | |
| 4-24 | A65 | 4-24-246 | Intron J-K | C | T | 210321 | | |
| 4-24 | A66 | 4-24-314 | Intron J-K | G | C | 210389 | | |
| 4-27 | A67 | 4-27-190 | Intron J-K | A | G | 211168 | | |
| 5-400 | A68 | 5-400-145 | Intron J-K | A | G | 215996 | | |
| 5-400 | A69 | 5-400-149 | Intron J-K | G | C | 216000 | | |
| 5-400 | A70 | 5-400-175 | Exon K | C | T | 216026 | 2283 | 742 = S |
| 5-400 | A71 | 5-400-231 | Exon K | C | T | 216082 | 2339 | 761 = A or V |
| 5-400 | A72 | 5-400-367 | Exon K | A | C | 216218 | 2475 | 806 = A |
| 99-12852 | A73 | 99-12852-110 | Intron K-L | G | T | 216322 | | |
| 99-12852 | A74 | 99-12852-325 | Intron K-L | A | G | 216537 | | |
| 4-37 | A75 | 4-37-326 | Intron K-L | A | C | 221649 | | |
| 4-37 | A76 | 4-37-107 | Intron K-L | A | G | 221867 | | |
| 5-270 | A77 | 5-270-92 | Intron K-L | G | C | 225645 | | |
| 99-12860 | A78 | 99-12860-47 | Intron K-L | A | G | 229387 | | |
| 99-12860 | A79 | 99-12860-57 | Intron K-L | A | T | 229397 | | |
| 5-402 | A80 | 5-402-144 | Exon L | C | T | 237555 | 2539 | 828 = P or S |

BM refers to “biallelic marker”.
All1 and all2 refer respectively to allele 1 and allele 2 of the biallelic marker.

TABLE 3

| | | Position range of probes | | |
|---|---|---|---|---|
| BM | Marker name | in SEQ ID No 1 | | Probes |
| A1 | 5-390-177 | 1987 | 2011 | P1 |
| A2 | 5-391-43 | 4589 | 4613 | P2 |
| A3 | 5-392-222 | 10216 | 10240 | P3 |
| A4 | 5-392-280 | 10274 | 10298 | P4 |
| A6 | 4-58-318 | 39932 | 39956 | P6 |
| A7 | 4-58-289 | 39961 | 39985 | P7 |
| A8 | 4-54-199 | 41373 | 41397 | P8 |
| A9 | 4-54-180 | 41392 | 41416 | P9 |
| A10 | 4-51-312 | 42220 | 42244 | P10 |
| A11 | 99-86-266 | 67463 | 67487 | P11 |
| A12 | 4-88-107 | 69509 | 69533 | P12 |
| A13 | 5-397-141 | 72826 | 72850 | P13 |
| A14 | 5-398-203 | 76048 | 76072 | P14 |
| A15 | 99-12738-248 | 81241 | 81265 | P15 |
| A16 | 99-109-358 | 83909 | 83933 | P16 |
| A17 | 99-12749-175 | 91905 | 91929 | P17 |
| A18 | 4-21-154 | 95337 | 95361 | P18 |
| A19 | 4-21-317 | 95499 | 95523 | P19 |
| A20 | 4-23-326 | 96178 | 96202 | P20 |
| A21 | 99-12753-34 | 97282 | 97306 | P21 |
| A22 | 5-364-252 | 98012 | 98036 | P22 |
| A23 | 99-12755-280 | 98902 | 98926 | P23 |
| A24 | 99-12755-329 | 98951 | 98975 | P24 |
| A25 | 4-87-212 | 103581 | 103605 | P25 |
| A26 | 99-12757-318 | 104386 | 104410 | P26 |
| A27 | 99-12758-102 | 106361 | 106385 | P27 |
| A28 | 99-12758-136 | 106395 | 106419 | P28 |
| A29 | 4-105-98 | 108303 | 108327 | P29 |
| A30 | 4-105-86 | 108315 | 108339 | P30 |
| A31 | 4-45-49 | 108460 | 108484 | P31 |
| A32 | 4-44-277 | 109184 | 109208 | P32 |
| A33 | 4-86-60 | 114592 | 114616 | P33 |
| A34 | 4-84-334 | 115704 | 115728 | P34 |
| A35 | 99-78-321 | 122071 | 122095 | P35 |
| A36 | 99-12767-36 | 123112 | 123136 | P36 |
| A37 | 99-12767-143 | 123219 | 123243 | P37 |
| A38 | 99-12767-189 | 123265 | 123289 | P38 |
| A39 | 99-12767-380 | 123456 | 123480 | P39 |
| A40 | 4-80-328 | 126726 | 126750 | P40 |
| A41 | 4-36-384 | 128198 | 128222 | P41 |
| A42 | 4-36-264 | 128318 | 128342 | P42 |
| A43 | 4-36-261 | 128321 | 128345 | P43 |
| A44 | 4-35-333 | 128582 | 128606 | P44 |
| A45 | 4-35-240 | 128675 | 128699 | P45 |
| A46 | 4-35-173 | 128742 | 128766 | P46 |
| A47 | 4-35-133 | 128782 | 128806 | P47 |
| A48 | 99-12771-59 | 130793 | 130817 | P48 |
| A49 | 99-12774-334 | 133194 | 133218 | P49 |
| A50 | 99-12776-358 | 135374 | 135398 | P50 |
| A51 | 99-12781-113 | 139377 | 139401 | P51 |
| A52 | 4-104-298 | 157523 | 157547 | P52 |
| A53 | 4-104-254 | 157567 | 157591 | P53 |
| A54 | 4-104-250 | 157571 | 157595 | P54 |
| A55 | 4-104-214 | 157607 | 157631 | P55 |
| A56 | 99-12818-289 | 172968 | 172992 | P56 |
| A57 | 99-24807-271 | 180610 | 180634 | P57 |
| A58 | 99-24807-84 | 180797 | 180821 | P58 |
| A59 | 99-12831-157 | 190322 | 190346 | P59 |
| A60 | 99-12831-241 | 190406 | 190430 | P60 |
| A61 | 99-12832-387 | 191385 | 191409 | P61 |
| A62 | 99-12836-30 | 195116 | 195140 | P62 |
| A63 | 99-12844-262 | 203834 | 203858 | P63 |
| A64 | 4-24-74 | 210139 | 210163 | P64 |
| A65 | 4-24-246 | 210309 | 210333 | P65 |
| A66 | 4-24-314 | 210377 | 210401 | P66 |
| A67 | 4-27-190 | 211156 | 211180 | P67 |
| A68 | 5-400-145 | 215984 | 216008 | P68 |
| A69 | 5-400-149 | 215988 | 216012 | P69 |
| A70 | 5-400-175 | 216014 | 216038 | P70 |
| A71 | 5-400-231 | 216070 | 216094 | P71 |
| A72 | 5-400-367 | 216206 | 216230 | P72 |
| A73 | 99-12852-110 | 216310 | 216334 | P73 |
| A74 | 99-12852-325 | 216525 | 216549 | P74 |
| A75 | 4-37-326 | 221637 | 221661 | P75 |
| A76 | 4-37-107 | 221855 | 221879 | P76 |
| A77 | 5-270-92 | 225633 | 225657 | P77 |
| A78 | 99-12860-47 | 229375 | 229399 | P78 |
| A79 | 99-12860-57 | 229385 | 229409 | P79 |
| A80 | 5-402-144 | 237543 | 237567 | P80 |

### Example 4

### Validation of the Polymorphisms Through Microsequencing

The biallelic markers identified in example 3 were further confirmed and their respective frequencies were determined through microsequencing. Microsequecing was carried out for each individual DNA sample described in Example 1.

Amplification from genomic DNA of individuals was performed by PCR as described above for the detection of the biallelic markers with the same set of PCR primers (Table

The preferred primers used in microsequencing were about 19 nucleotides in length and hybridized just upstream of the considered polymorphic base. According to the invention, the primers used in microsequencing are detailed in Table 4.

TABLE 4

| | | | | | | Complementary | |
|---|---|---|---|---|---|---|---|
| | | | Position range of | | | position | |
| | | | microsequencing | | | range of microsequencing | |
| | | | primer mis 1 in | | | primer mis. 2 in SEQ ID | |
| Marker name | BM | Mis 1 | SEQ ID No 1 | | Mis 2 | No 1 | |
| 5-390-177 | A1 | D1 | 1980 | 1998 | E1 | 2000 | 2018 |
| 5-391-43 | A2 | D2 | 4582 | 4600 | E2 | 4602 | 4620 |
| 5-392-222 | A3 | D3 | 10209 | 10227 | E3 | 10229 | 10247 |
| 5-392-280 | A4 | D4 | 10267 | 10285 | E4 | 10287 | 10305 |
| 4-58-318 | A6 | D6 | 39925 | 39943 | E6 | 39945 | 39963 |
| 4-58-289 | A7 | D7 | 39954 | 39972 | E7 | 39974 | 39992 |
| 4-54-199 | A8 | D8 | 41366 | 41384 | E8 | 41386 | 41404 |
| 4-54-180 | A9 | D9 | 41385 | 41403 | E9 | 41405 | 41423 |
| 4-51-312 | A10 | D10 | 42213 | 42231 | E10 | 42233 | 42251 |
| 99-86-266 | A11 | D11 | 67456 | 67474 | E11 | 67476 | 67494 |
| 4-88-107 | A12 | D12 | 69502 | 69520 | E12 | 69522 | 69540 |
| 5-397-141 | A13 | D13 | 72819 | 72837 | E13 | 72839 | 72857 |
| 5-398-203 | A14 | D14 | 76041 | 76059 | E14 | 76061 | 76079 |
| 99-12738-248 | A15 | D15 | 81234 | 81252 | E15 | 81254 | 81272 |
| 99-109-358 | A16 | D16 | 83902 | 83920 | E16 | 83922 | 83940 |
| 99-12749-175 | A17 | D17 | 91898 | 91916 | E17 | 91918 | 91936 |
| 4-21-154 | A18 | D18 | 95330 | 95348 | E18 | 95350 | 95368 |
| 4-21-317 | A19 | D19 | 95492 | 95510 | E19 | 95512 | 95530 |
| 4-23-326 | A20 | D20 | 96171 | 96189 | E20 | 96191 | 96209 |
| 99-12753-34 | A21 | D21 | 97275 | 97293 | E21 | 97295 | 97313 |
| 5-364-252 | A22 | D22 | 98005 | 98023 | E22 | 98025 | 98043 |
| 99-12755-280 | A23 | D23 | 98895 | 98913 | E23 | 98915 | 98933 |
| 99-12755-329 | A24 | D24 | 98944 | 98962 | E24 | 98964 | 98982 |
| 4-87-212 | A25 | D25 | 103574 | 103592 | E25 | 103594 | 103612 |
| 99-12757-318 | A26 | D26 | 104379 | 104397 | E26 | 104399 | 104417 |
| 99-12758-102 | A27 | D27 | 106354 | 106372 | E27 | 106374 | 106392 |
| 99-12758-136 | A28 | D28 | 106388 | 106406 | E28 | 106408 | 106426 |
| 4-105-98 | A29 | D29 | 108296 | 108314 | E29 | 108316 | 108334 |
| 4-105-86 | A30 | D30 | 108308 | 108326 | E30 | 108328 | 108346 |
| 4-45-49 | A31 | D31 | 108453 | 108471 | E31 | 108473 | 108491 |
| 4-44-277 | A32 | D32 | 109177 | 109195 | E32 | 109197 | 109215 |
| 4-86-60 | A33 | D33 | 114585 | 114603 | E33 | 114605 | 114623 |
| 4-84-334 | A34 | D34 | 115697 | 115715 | E34 | 115717 | 115735 |
| 99-78-321 | A35 | D35 | 122064 | 122082 | E35 | 122084 | 122102 |
| 99-12767-36 | A36 | D36 | 123105 | 123123 | E36 | 123125 | 123143 |
| 99-12767-143 | A37 | D37 | 123212 | 123230 | E37 | 123232 | 123250 |
| 99-12767-189 | A38 | D38 | 123258 | 123276 | E38 | 123278 | 123296 |
| 99-12767-380 | A39 | D39 | 123449 | 123467 | E39 | 123469 | 123487 |
| 4-80-328 | A40 | D40 | 126719 | 126737 | E40 | 126739 | 126757 |
| 4-36-384 | A41 | D41 | 128191 | 128209 | E41 | 128211 | 128229 |
| 4-36-264 | A42 | D42 | 128311 | 128329 | E42 | 128331 | 128349 |
| 4-36-261 | A43 | D43 | 128314 | 128332 | E43 | 128334 | 128352 |
| 4-35-333 | A44 | D44 | 128575 | 128593 | E44 | 128595 | 128613 |
| 4-35-240 | A45 | D45 | 128668 | 128686 | E45 | 128688 | 128706 |
| 4-35-173 | A46 | D46 | 128735 | 128753 | E46 | 128755 | 128773 |
| 4-35-133 | A47 | D47 | 128775 | 128793 | E47 | 128795 | 128813 |
| 99-12771-59 | A48 | D48 | 130786 | 130804 | E48 | 130806 | 130824 |
| 99-12774-334 | A49 | D49 | 133187 | 133205 | E49 | 133207 | 133225 |
| 99-12776-358 | A50 | D50 | 135367 | 135385 | E50 | 135387 | 135405 |
| 99-12781-113 | A51 | D51 | 139370 | 139388 | E51 | 139390 | 139408 |
| 4-104-298 | A52 | D52 | 157516 | 157534 | E52 | 157536 | 157554 |
| 4-104-254 | A53 | D53 | 157560 | 157578 | E53 | 157580 | 157598 |
| 4-104-250 | A54 | D54 | 157564 | 157582 | E54 | 157584 | 157602 |
| 4-104-214 | A55 | D55 | 157600 | 157618 | E55 | 157620 | 157638 |
| 99-12818-289 | A56 | D56 | 172961 | 172979 | E56 | 172981 | 172999 |
| 99-24807-271 | A57 | D57 | 180603 | 180621 | E57 | 180623 | 180641 |
| 99-24807-84 | A58 | D58 | 180790 | 180808 | E58 | 180810 | 180828 |
| 99-12831-157 | A59 | D59 | 190315 | 190333 | E59 | 190335 | 190353 |
| 99-12831-241 | A60 | D60 | 190399 | 190417 | E60 | 190419 | 190437 |
| 99-12832-387 | A61 | D61 | 191378 | 191396 | E61 | 191398 | 191416 |
| 99-12836-30 | A62 | D62 | 195109 | 195127 | E62 | 195129 | 195147 |
| 99-12844-262 | A63 | D63 | 203827 | 203845 | E63 | 203847 | 203865 |
| 4-24-74 | A64 | D64 | 210132 | 210150 | E64 | 210152 | 210170 |
| 4-24-246 | A65 | D65 | 210302 | 210320 | E65 | 210322 | 210340 |
| 4-24-314 | A66 | D66 | 210370 | 210388 | E66 | 210390 | 210408 |
| 4-27-190 | A67 | D67 | 211149 | 211167 | E67 | 211169 | 211187 |
| 5-400-145 | A68 | D68 | 215977 | 215995 | E68 | 215997 | 216015 |
| 5-400-149 | A69 | D69 | 215981 | 215999 | E69 | 216001 | 216019 |
| 5-400-175 | A70 | D70 | 216007 | 216025 | E70 | 216027 | 216045 |
| 5-400-231 | A71 | D71 | 216063 | 216081 | E71 | 216083 | 216101 |
| 5-400-367 | A72 | D72 | 216199 | 216217 | E72 | 216219 | 216237 |
| 99-12852-110 | A73 | D73 | 216303 | 216321 | E73 | 216323 | 216341 |
| 99-12852-325 | A74 | D74 | 216518 | 216536 | E74 | 216538 | 216556 |
| 4-37-326 | A75 | D75 | 221630 | 221648 | E75 | 221650 | 221668 |
| 4-37-107 | A76 | D76 | 221848 | 221866 | E76 | 221868 | 221886 |
| 5-270-92 | A77 | D77 | 225626 | 225644 | E77 | 225646 | 225664 |
| 99-12860-47 | A78 | D78 | 229368 | 229386 | E78 | 229388 | 229406 |
| 99-12860-57 | A79 | D79 | 229378 | 229396 | E79 | 229398 | 229416 |
| 5-402-144 | A80 | D80 | 237536 | 237554 | E80 | 237556 | 237574 |

Mis 1 and Mis 2 respectively refer to microsequencing primers which hybridized with the non-coding strand of the PG-3 gene or with the coding strand of the PG-3 gene.

The microsequencing reaction was performed as follows:

After purification of the amplification products, the microsequencing reaction mixture was prepared by adding, in a 20 μl final volume: 10 pmol microsequencing oligonucleotide, 1 U Thermosequenase (Amersham E79000G), 1.25 μl Thermosequenase buffer (260 mM Tris HCl pH 9.5, 65 mM MgCl₂), and the two appropriate fluorescent ddNTPs (Perkin Elmer, Dye Terminator Set 401095) complementary to the nucleotides at the polymorphic site of each biallelic marker tested, following the manufacturer's recommendations. After 4 minutes at 94° C., 20 PCR cycles of 15 sec at 55° C., 5 sec at 72° C., and 10 sec at 94° C. were carried out in a Tetrad PTC-225 thermocycler (MJ Research). The unincorporated dye terminators were then removed by ethanol precipitation. Samples were finally resuspended in formamide-EDTA loading buffer and heated for 2 min at 95° C. before being loaded on a polyacrylamide sequencing gel. The data were collected by an ABI PRISM 377 DNA sequencer and processed using the GENESCAN software (Perkin Elmer).

Following gel analysis, data were automatically processed with software that allows the determination of the alleles of biallelic markers present in each amplified fragment.

The software evaluates such factors as whether the intensities of the signals resulting from the above microsequencing procedures are weak, normal, or saturated, or whether the signals are ambiguous. In addition, the software identifies significant peaks (according to shape and height criteria). Among the significant peaks, peaks corresponding to the targeted site are identified based on their position. When two significant peaks are detected for the same position, each sample is categorized classification as homozygous or heterozygous type based on the height ratio.

### Example 5

### Preparation of Antibody Compositions to the PG-3 Protein

Substantially pure protein or polypeptide is isolated from transfected or transformed cells containing an expression vector encoding the PG-3 protein or a portion thereof. The concentration of protein in the final preparation is adjusted, for example, by concentration on an Amicon filter device, to the level of a few micrograms/ml. Monoclonal or polyclonal antibody to the protein can then be prepared as follows:

A. Monoclonal Antibody Production by Hybridoma Fusion

Monoclonal antibody to epitopes in the PG-3 protein or a portion thereof can be prepared from murine hybridomas according to the classical method of Kohler, G. and Milstein, C., (1975) or derivative methods thereof. Also see Harlow, E., and D. Lane. 1988.

Briefly, a mouse is repetitively inoculated with a few micrograms of the PG-3 protein or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, (1980), and derivative methods thereof. Selected positive clones can be expanded and their monoclonal antibody product harvested for use. Detailed procedures for monoclonal antibody production are described in Davis, L., et al. (1986).

B. Polyclonal Antibody Production by Immunization

Polyclonal antiserum containing antibodies to heterogeneous epitopes in the PG-3 protein or a portion thereof can be prepared by immunizing suitable non-human animal with the PG-3 protein or a portion thereof, which can be unmodified or modified to enhance immunogenicity. A suitable non-human animal is preferably a non-human mammal is selected, usually a mouse, rat, rabbit, goat, or horse. Alternatively, a crude preparation, which has been enriched for PG-3 concentration, can be used to generate antibodies. Such proteins, fragments or preparations are introduced into the non-human mammal in the presence of an appropriate adjuvant (e.g. aluminum hydroxide, RIBI, etc.) which is known in the art. In addition the protein, fragment or preparation can be pretreated with an agent which will increase antigenicity, such agents are known in the art and include, for example, methylated bovine serum albumin (mBSA), bovine serum albumin (BSA), Hepatitis B surface antigen, and keyhole limpet hemocyanin (KLH). Serum from the immunized animal is collected, treated and tested according to known procedures. If the serum contains polyclonal antibodies to undesired epitopes, the polyclonal antibodies can be purified by immunoaffinity chromatography.

Effective polyclonal antibody production is affected by many factors related both to the antigen and the host species. Also, host animals vary in response to site of inoculations and dose, with both inadequate or excessive doses of antigen resulting in low titer antisera. Small doses (ng level) of antigen administered at multiple intradermal sites appears to be most reliable. Techniques for producing and processing polyclonal antisera are known in the art, see for example, Mayer and Walker (1987). An effective immunization protocol for rabbits can be found in Vaitukaitis, J., et al. (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O., et al., (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., (1980).

Antibody preparations prepared according to either the monoclonal or the polyclonal protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

While the preferred embodiment of the invention has been illustrated and described, it will be appreciated that various changes can be made therein by the one skilled in the art without departing from the spirit and scope of the invention.

### REFERENCES

Abbondanzo S J et al., 1993, Methods in Enzymology, Academic Press, New York, pp 803-823

Ajioka R. S. et al., Am. J. Hum. Genet., 60:1439-1447, 1997

Altschul et al., 1990, J. Mol. Biol. 215(3):403410

Altschul et al, 1993, Nature Genetics 3:266-272

Altschul et al., 1997, Nuc. Acids Res. 25:3389-3402

Ames et al., (1995), J. Immunol. Meth. 184:177-186.

Anton M. et al., 1995, J. Virol., 69: 4600-4606

ArakiKetal. (1995)Proc. Natl. Acad. Sci. USA. 92(1):160-4.

Arnheim N & Shibata D, Curr. Op. Genetics & Development, 1997, 7:364-370

Ashkenazi et al., (1991), Proc. Natl. Acad. Sci. USA 88:10535-10539.

Aszódi et al., Proteins:Structure, Function, and Genetics, Supplement 1:38-42 (1997)

Attwood et al., (1996) Nucleic Acids Res. 24(1):182-8.

Attwood et al., (2000) Nucleic Acids Res. 28(1):225-7

Ausubel et al. (1989)Current Protocols in Molecular Biology, Green Publishing Associates and Wiley Interscience, N.Y.

Bateman et al., (2000) Nucleic Acids Res. 28(1):263-6

Baubonis W. (1993) Nucleic Acids Res. 21(9):2025-9.

Beaucage et al., Tetrahedron Lett 1981, 22: 1859-1862

Better et al., (1988), Science. 240:1041-1043.

Bittle et al., (1985), Virol. 66:2347-2354.

Bochar et al., (2000) Cell 102:257-265

Bowie et al., (1994), Science. 247:1306-1310.

Bradley A., 1987, Production and analysis of chimaeric mice. In: E. J. Robertson (Ed.), Teratocarcinomas and embryonic stem cells: A practical approach. IRL Press, Oxford, pp.113.

Bram R J et al., 1993, Mol. Cell Biol., 13: 4760-4769

Brinkman et al., (1995) J Immunol Methods. 182:41-50.

Brown E L, Belagaje R, Ryan M J, Khorana H G, Methods Enzymol 1979;68:109-151

Brutlag et al. Comp. App. Biosci. 6:237-245, 1990

Bucher and Bairoch (1994) Proceedings 2nd International Conference on Intelligent Systems for Molecular Biology. Altman et al, Eds., ppS3-61, AAAlPress, Menlo Park.

Burton et al. (1994), Adv. Immunol. 57:191-280

Bush et al., 1997, J. Chromatogr., 777: 311-328.

Chai H. et al. (1993) Biotechnol. Appl. Biochem. 18:259-273.

Chee et al. (1996) Science. 274:610-614.

Chen and Kwok Nucleic Acids Research 25:347-353 1997

Chen et al. (1987) Mol. Cell. Biol. 7:2745-2752.

Chen et al. Proc. Natl. Acad. Sci. USA 94/20 10756-10761,1997

Cho R J et al., 1998, Proc. Natl. Acad. Sci. USA, 95(7): 3752-3757.

Chou J. Y., 1989, Mol. Endocrinol., 3: 1511-1514.

Chow et al., (1985), Proc. Natl. Acad. Sci. USA. 82:910-914.

Clark A. G. (1990) Mol. Biol. Evol. 7:111-122.

Cleland et al., (1993), Crit. Rev. Therapeutic Drug Carrier Systems. 10:307-377.

Coles R, Caswell R, Rubinsztein D C, Hum Mol Genet 1998;7:791-800

Compton J. (1991) Nature. 350(6313):91-92.

Corpet et al. (2000) Nucleic Acids Res. 28(1):267-9

Creighton (1983), Proteins: Structures and Molecular Principles, W. H. Freeman & Co. 2nd Ed., T. E., New York

Creighton, (1993), Posttranslational Covalent Modification of Proteins, W. H. Freeman and Company, New York B. C. Johnson, Ed., Academic Press, New York 1-12

Cunningham et al. (1989), Science 244:1081-1085.

Davis L. G., M. D. Dibner, and J. F. Battey, Basic Methods in Molecular Biology, ed., Elsevier Press, NY, 1986

Dempster et al., (1977) J. R. Stat. Soc., 39B:1-38.

Dent D S & Latchman D S (1993) The DNA mobility shift assay. In: Transcription Factors: A Practical Approach (Latchman DS, ed.) pp 1-26. Oxford: IRL Press

Eckner R. et al. (1991) EMBO J. 10:3513-3522.

Edwards et Leatherbarrow, Analytical Biochemistry, 246, 1-6 (1997)

Ellis N A, 1997, Curr.Op.Genet.Dev.7: 354-363

Emi M, et al., Cancer Res. Oct. 1, 1992; 52(19): 5368-5372

Engvall, E., Meth. Enzymol. 70:419 (1980)

Excoffier L. and Slatkin M. (1995) Mol. Biol. Evol., 12(5): 921-927.

Fanger G R et al., 1997 Curr.Op.Genet.Dev.7:67-74

Feldman and Steg, 1996, Medecine/Sciences, synthese, 12:47-55

Felici F., 1991, J. Mol. Biol., Vol. 222:301-310

Fell et al., (1991), J. Immunol. 146:2446-2452.

Fields and Song, 1989, Nature, 340: 245-246

Fishel R & Wilson T. 1997, Curr.Op.Genet.Dev.7: 105-113;

Fisher, D., Chap. 42 in: Manual of Clinical hmnunology, 2d Ed. Rose and Friedman, Eds.) Amer. Soc. For Microbiol., Washington, D.C. (1980)

Flotte et al. (1992) Am. J. Respir. Cell Mol. Biol. 7:349-356.

Fodor et al. (1991) Science 251:767-777.

Fountoulakis et al., (1995) Biochem. 270:3958-3964.

Fraley et al. (1979) Proc. Natl. Acad. Sci. USA. 76:3348-3352.

Fried M, Crothers D M, Nucleic Acids Res 1981;9:6505-6525

Fromont-Racine M. et al., 1997, Nature Genetics, 16(3): 277-282.

Fuller S. A. et al. (1996) Immunology in Current Protocols in Molecular Biology, Ausubel et al. Eds, John Wiley & Sons, Inc., USA.

Furth P. A. et al. (1994) Proc. Natl. Acad. Sci USA. 91:9302-9306.

Garner M M, Revzin A, Nucleic Acids Res 1981;9:3047-3060

GeysenH. Mario et al. 1984. Proc. Natl. Acad. Sci. U.S.A. 81:3998-4002

Ghosh and Bacchawat, 1991, Targeting of liposomes to hepatocytes, IN: Liver Diseases, Targeted diagnosis and therapy using specific rceptors and ligands. Wu et al. Eds., Marcel Dekeker, New York, pp. 87-104.

Gillies et al., (1989), J. Immunol Methods. 125:191-202.

Gillies et al., (1992), Proc Natl Acad Sci USA 89:1428-1432.

Gonnet et al., 1992, Science 256:1443-1445

Gopal (1985) Mol Cell. Biol., 5:1188-1190.

Gossen M. et al. (1992) Proc. Natl. Acad. Sci. USA. 89:5547-5551.

Gossen M. et al. (1995) Science. 268:1766-1769.

Graham et al. (1973) Virology 52:456-457.

Green et al., Ann. Rev. Biochem. 55:569-597 (1986)

Griffais et al., (1991) Nucleic Acids Res. 19: 3887-3891

Griffin et al. Science 245:967-971 (1989)

Grompe, M. (1993) Nature Genetics. 5:111-117.

Grompe, M. et al. (1989) Proc. Natl. Acad. Sci. U.S.A. 86:5855-5892.

Gronwald J, et al., Cancer Res. Feb. 1, 1997; 57(3): 481-487

Gu H. et al. (1993) Cell 73:1155-1164.

Gu H. et al. (1994) Science 265:103-106.

Guatelli J C et al. (1990) Proc. Natl. Acad. Sci. USA. 35:273-286.

Haber D & Harlow E, 1997, Nature Genet. 16:320-322

Hacia J G, Brody L C, Chee M S, Fodor S P, Collins F S, Nat Genet 1996;14(4):441-447

Haff L. A. and SmimovI. P. (1997) Genome Research, 7:378-388.

Hames B. D. and Higgins S. J. (1985) Nucleic AcidHybridization: A Practical Approach. Hames and Higgins Ed., IRL Press, Oxford.

Hammerling (1981), Monoclonal Antibodies and T-Cell Hybridomas, Elsevier, N.Y. 563-681.

Hansson et al., (1999), J. Mol. Biol. 287:265-276.

Haravama (1998), Trends Biotechnol. 16(2): 76-82.

Harju L, Weber T, Alexandrova L, Lukin M, Ranki M, Jalanko A, Clin Chem 1993;39(11Pt 1):2282-2287

Harland et al. (1985) J. Cell. Biol. 101:1094-1095.

Harlow, E., and D. Lane. 1988. Antibodies A Laboratory Manual. Cold Spring Harbor Laboratory. pp. 53-242

Harper J W et al., 1993, Cell, 75: 805-816

Harris H et al.,1969,Nature 223:363-368

Hawley M E. et al. (1994) Am. J. Phys. Anthropol. 18:104.

Henikoff and Henikoff, 1993, Proteins 17:49-61

Henikoff et al., (2000) Electrophoresis 21(9): 1700-6

Henikoff et al., (2000) Nucleic Acids Res. 28(1):228-30

Higgins et al., 1996, Methods Enzymol. 266:383-402

Hillier L. and Green P. Methods Appl., 1991, 1: 124-8.

Hoess et al. (1986) Nucleic Acids Res. 14:2287-2300.

Hofmann et al., (1999) Nucl. Acids Res. 27:215-219;

Holm and Sander (1996) Nucleic Acids Res. 24(1):206-9

Holm and Sander (1997) Nucleic Acids Res. 25(1):231-4

Holm and Sander (1999) Nucleic Acids Res. 27(1):244-7

Hoppe et al., (1994), FEBS Letters. 344:191.

Houghten (1985), Proc. Natl. Acad. Sci. USA 82:5131-5135.

Huang L. et al. (1996) Cancer Res 56(5):1137-1141.

Hunkapiller et al., (1984) Nature. 310(5973): 105-11.

Hunter T, 1991 Cell 64:249

Huston et al., (1991), Meth. Enymol. 203:46_{—}88.

Huygen et al. (1996) Nature Medicine. 2(8):893-898.

Ichikawa T, et al., Prostate Suppl. 1996; 6: 31-35

Ishwad C S, et al., Int. J. Cancer. Jan. 5, 1999; 80(1): 25-31

Izant J G, Weintraub H, Cell 1984 April;36(4):1007-15

Jameson and Wolf, (1988), Comp. Appl. Biosci. 4:181-186

Julan et al. (1992) J. Gen. Virol. 73:3251-3255.

Kanegae Y. et al, Nucl. Acids Res. 23:3816-3821(1995).

Karlin and Altschul, 1990,Proc. Natl. Acad. Sci. USA 87:2267-2268

Kettleborough et al., (1994), Eur. L Immunol. 24:952-958.

Khoury J. et al., Fundamentals of Genetic Epidemiology, Oxford University Press, NY, 1993

Kim U-J. et al. (1996) Genomics 34:213-218.

Klein et al. (1987) Nature. 327:70-73.

Kohler, G. and Milstein, C., Nature 256:495 (1975)

Koller et al. Proc. Natl. Acad. Sci. USA 86:8932-8935 (1989)

Koller et al. (1992) Annu. Rev. Immunol. 10:705-730.

Kostelny et al., (1992), J. Immunol. 148:1547-1553.

Kozal M J, Shah N, Shen N, Yang R, Fucini R, Merigan T C, Richman D D, Morris D, Hubbell E, Chee M, Gingeras T R, Nat Med 1996; 2(7):753-759

Landegren U. et al. (1998) Genome Research, 8:769-776.

Lander and Schork, Science, 265, 2037-2048, 1994

Landschulz et al., (1988), Science. 240:1759.

Lange K. (1997) Mathematical and Statistical Methods for Genetic Analysis. Springer, New York.

Lenhard T. et al. (1996) Gene. 169:187-190.

Lewin, (1989), Proc. Natl. Acad. Sci. USA 86:9832-8935.

Linton M. F. et al. (1993) J. Clin. Invest. 92:3029-3037.

Liu Z. et al. (1994) Proc. Natl. Acad. Sci. USA. 91: 4528-4262.

Livak et al., Nature Genetics, 9:341-342, 1995

Livak K J, Hainer J W, Hum Mutat 1994;3(4):379-385

Lockhart et al. Nature Biotechnology 14: 1675-1680, 1996

Lo Conte et al., (2000) Nucleic Acids Res. 28(1):257-9.

Lorenzo and Blasco (1998) Biotechniques. 24(2):308-313.

Lucas A. H., 1994, In: Development and Clinical Uses of Haempophilus b Conjugate;

Maliketal., (1992), Exp. Hematol. 20:1028-1035.

Mansour S. L. et al. (1988) Nature. 336:348-352.

Marshall R. L. et al. (1994) PCR Methods and Applications. 4:80-84.

Matsuyama H, et al., Oncogene 1994 October; 9(10): 3071-3076

McCormick et al. (1994) Genet. Anal. Tech. Appl. 11:158-164.

McLaughlin B. A. et al (1996) Am. J. Hum. Genet. 59:561-569.

Morton N. E., Am.J. Hum.Genet., 7:277-318, 1955

Mullinax et al., (1992), BioTechniques. 12(6):864-869.

Murvai et al., (2000) Nucleic Acids Res. 28(1):260-2

Murzin et al., (1995) J. Mol. Biol. 247(4):536-40

Muzyczka et al (1992) Curr. Topics in Micro. and Immunol. 158:97-129.

Nada S. et al. (1993) Cell 73:1125-1135.

Nagai H, et al., Oncogene Jun. 19, 1997; 14(24): 2927-2933

Nagy A. et al., 1993, Proc. Natl. Acad. Sci. USA, 90: 8424-8428.

Narang S A, Hsiung H M, Brousseau R, Methods Enzymol 1979;68:90-98

Naramura et al., (1994), Immunol. Lett. 39:91-99.

Neda et al. (1991) J. Biol. Chem. 266:14143-14146.

Nevill-Manning et al., (1998) Proc. Natl. Acad. Sci. USA. 95, 5865-5871

Newton et al. (1989) Nucleic Acids Res. 17:2503-2516.

Nickerson D. A. et al. (1990) Proc. Natl. Acad. Sci. U.S.A. 87:8923-8927.

Nicolau C. et al., 1987, Methods Enzymol., 149:157-76.

Nicolau et al. (1982) Biochim. Biophys. Acta. 721:185-190.

Nyren P, Pettersson B, Uhlen M, Anal Biochem 1993;208(1):171-175

O'Reilly et al. (1992) Baculovirus Expression Vectors: A Laboratory Manual. W. H. Freeman and Co., New York.

Ohno et al. (1994) Science. 265:781-784.

Oi et al., (1986), BioTechniques 4:214.

Oldenburg K. R. et al., 1992, Proc. Natl. Acad. Sci., 89:5393-5397.

Orengo et al., (1997) Structure. 5(8):1093-108

Orita et al. (1989) Proc. Natl. Acad. Sci. U.S.A.86: 2776-2770.

Ott J., Analysis of Human Genetic Linkage, John Hopkins University Press, Baltimore, 1991

Ouchterlony, O. et al, Chap. 19 in: Handbook of Experimental Immunology D. Wier (ed) Blackwell (1973)

Padlan, (1991), Molec. Immunol. 28(4/5):489-498.

Parnley and Smith, Gene, 1988, 73:305-318

Pastinen et al., Genome Research 1997; 7:606-614

Patten, et al. (1997), Curr Opinion Biotechnol. 8:724-733.

Pearl et al., (2000) Biochem Soc Trans. 28(2):269-75

Pearson and Lipman, 1988, Proc. Natl. Acad. Sci. USA 85(8):2444-2448

Pease S. ans William R.S., 1990, Exp. Cell. Res., 190:209-211.

Perinchery G, et al., Int. J. Oncol. 1999 March; 14(3): 495-500

Perlin et al. (1994) Am. J. Hum. Genet. 55:777-787.

Persic et al., (1997), Gene. 1879-81

Peterson et al., 1993, Proc. Natl. Acad. Sci. USA, 90: 7593-7597.

Pietu et al. Genome Research 6:492-503, 1996

Pinckard et al., (1967), Clin. Exp. Immunol 2:331-340.

Pineau P, et al., Oncogene 1999 May 20, 18(20): 3127-3134

Pongor et al. (1993) Protein Eng. 6(4):391-5

Potter et al. (1984) Proc. Natl. Acad. Sci. U.S.A. 81(22):7161-7165.

Ramunsen et al., 1997, Electrophoresis, 18: 588-598.

Reid L. H. et al. (1990) Proc. Natl. Acad. Sci. U.S.A. 87:4299-4303.

Risch, N. and Merikangas, K. (Science, 273:1516-1517, 1996

Robbins et al., (1987), Diabetes. 36:838-845.

Robertson E., 1987, Embryo-derived stem cell lines. In: E. J. Robertson Ed. Teratocarcinomas and embrionic stem cells: a practical approach. IRL Press, Oxford, pp. 71.

Roguska et al., (1994), Proc. Natl. Acad. Sci. U.S.A. 91:969-973.

Ron et al., (1993), Biol. Chem., 268 2984-2988.

Rossi et al., Pharmacol. Ther. 50:245-254, (1991)

Roth J. A. et al. (1996) Nature Medicine. 2(9):985-991.

Rouxetal. (1989) Proc. Natl. Acad. Sci. U.S.A. 86:9079-9083.

Ruano et al. (1990) Proc. Natl. Acad. Sci. U.S.A. 87:6296-6300.

Sakabe T, et al., Cancer Res. Feb. 1, 1999; 59(3): 511-515

Sakakura C, et al., Genes Chromosomes Cancer 1999 April; 24(4): 299-305

Sambrook, J., Fritsch, E. F., and T. Maniatis. (1989) Molecular Cloning: A Laboratory Manual. 2ed. Cold Spring Harbor Laboratory, Cold Spring Harbor, N.Y.

Samson M, et al. (1996) Nature, 382(6593):722-725.

Samulskietal. (1989) J. Virol. 63:3822-3828.

Sanchez-Pescador R. (1988) J. Clin. Microbiol. 26(10):1934-1938.

Sander and Schneider (1991) Proteins. 9(1):56-68.)

Sarkar, G. and Sommer S. S. (1991) Biotechniques.

Sauer B.et al. (1988) Proc. Natl. Acad. Sci. U.S.A. 85:5166-5170.

Sawai et al., (1995), AJRI 34:26-34.

Schaid D. J. et al., Genet. Epidemiol.,13:423-450, 1996

Schedl A. et al., 1993a, Nature, 362: 258-261.

Schedl et al., 1993b, Nucleic Acids Res., 21: 4783-4787.

Schena et al. Science 270:467-470, 1995

Schena et al, 1996, Proc Natl Acad Sci USA, 93(20):10614-10619.

Schneider et al.(1997) Arlequin: A Software For Population Genetics Data Analysis. University of Geneva.

Scholnick S B, et al., J. Natl. Cancer Inst. Nov. 20, 1996; 88(22): 1676-1682

Schultz et al., (1998) Proc Natl Acad Sci USA 95, 5857-5864

Schwartz and Dayhoff, eds., 1978, Matrices for Detecting Distance Relationships: Atlas of Protein Sequence and Structure, Washington: National Biomedical Research Foundation

Sczakiel G. et al. (1995) Trends Microbiol. 3(6):213-217.

Shay J. W. et al., 1991, Biochem. Biophys. Acta, 1072:1-7.

Sheffield, V. C. et al. (1991) Proc. Natl. Acad. Sci. U.S.A. 49:699-706.

Shizuya et al. (1992) Proc. Natl. Acad. Sci. U.S.A. 89:8794-8797.

Shoemaker D D, et al., Nat Genet 1996;14(4):450-456

Shu et al., (1993), Proc. Natl. Acad. Sci. U.S.A. 90:7995-7999.

Skerra et al., (1988), Science 240:1038-1040.

Smith (1957) Ann. Hum. Genet. 21:254-276.

Smith et al. (1983) Mol. Cell. Biol. 3:2156-2165.

Sonnhammer and Kahn D (1994) Protein Sci. 3(3):482-92

Sonnhammer et al., (1997) Proteins. 28(3):405-20

Sosnowski R G, et al., Proc Natl Acad Sci USA 1997;94:1119-1123

Sowdhamini et al., Protein Engineering 10:207, 215 (1997)

Spielmann S. and Ewens W. J., Am. J. Hum. Genet., 62:450-458, 1998

Spielmann S. et al., Am. J. Hum. Genet., 52:506-516, 1993

Stemberg N. L. (1994) Mamm. Genome. 5:397-404.

Stemberg N. L. (1992) Trends Genet. 8:1-16.

Studnicka et al., (1994), Protein Engineering. 7(6):805-814.

Stryer, L., Biochemistry, 4th edition, 1995, W. H Freeman & Co., New York.

Sunwoo J B, et al., Genes Chromosomes Cancer 1996 July; 16(3):164-169

Sunwoo J B, et al., Oncogene Apr. 22, 1999; 18(16): 2651-2655

Sutcliffe et al., (1983), Science. 219:660-666.

Syvanen A C, Clin Chim Acta 1994;226(2):225-236

Szabo A. et al. Curr Opin Struct Biol 5, 699-705 (1995)

Tacson et al. (1996) Nature Medicine. 2(8):888-892.

Tatusov et al., (1997) Science, 278, 631:637

Tatusov et al., (2000) Nucleic Acids Res. 28(1):33-6.)

Te Riele et al. (1990) Nature. 348:649-651.

Terwilliger J. D. and Ott J., Handbook ofHuman Genetic Linkage, John Hopkins University Press, London, 1994

Thomas K. R. et al. (1986) Cell. 44:419-428.

Thomas K. R. et al. (1987) Cell. 51:503-512.

Thompson et al., 1994, Nucleic Acids Res. 22(2):4673-4680

Traunecker et al., (1988), Nature. 331:84-86.

Tur-Kaspa et al. (1986) Mol. Cell. Biol. 6:716-718.

Tutt et al., (1991), J. Immunol. 147:60-69.

Tyagi et al. (1998) Nature Biotechnology. 16:49-53.

Urdea M. S. (1988) Nucleic Acids Research. 11:4937-4957.

Urdea M. S. et al.(1991) Nucleic Acids Symp. Ser. 24:197-200.

Vaitukaitis,J.etal. J. Clin. Endocrinol. Metab. 33:988-991(1971)

Valadon P., et al., 1996, J. Mol. Biol., 261:11-22.

Van der Lugt et al. (1991) Gene. 105:263-267.

Vil et al., (1992) Proc Natl Acad Sci U S 89:11337-11341.

Vlasak R.et al. (1983) Eur. J. Biochem. 135:123-126.

Wabiko et al. (1986) DNA.5(4):305-314.

Walker et al. (1996) Clin. Chem. 42:9-13.

Wang et al., 1997, Chromatographia, 44: 205-208.

Washburn J, Woino K, and Macoska J, Proceedings of American Association for Cancer Research, March 1997; 38

Weir, B. S. (1996) Genetic data Analysis II: Methods for Discrete population genetic Data, Sinauer Assoc., Inc., Sunderland, Mass., U.S.A.

Weiss F U et al., 1997 Curr.Op.Genet.Dev.7:80-86

Westerink M. A. J., 1995, Proc. Natl. Acad. Sci., 92:4021-4025

White, M. B. et al. (1992) Genomics. 12:301-306.

Wilson et al., (1984) Cell. 37(3):767-78.

Wong et al. (1980) Gene. 10:87-94.

Wood S. A. et al., 1993, Proc. Natl. Acad. Sci. USA, 90: 4582-4585.

Wright K, et al., Oncogene Sep. 3, 1998; 17(9): 1185-1188

Wu and Wu (1987) J. Biol. Chem. 262:4429-4432.

Wu and Wu (1988) Biochemistry. 27:887-892.

Wu et al. (1989) Proc. Natl. Acad. Sci. U.S.A. 86:2757.

Yagi T. et al. (1990) Proc. Natl. Acad. Sci. U.S.A. 87:9918-9922.

Yaremko M L, et al., Genes Chromosomes Cancer 1994 May;10(1):1-6

Yona et al., (1999) Proteins. 37(3):360-78

Zhao et al., Am. J. Hum. Genet., 63:225-240, 1998

Zheng, X. X. et al. (1995), J. Immunol. 154:5590-5600.

Zou Y. R. et al. (1994) Curr. Biol. 4:1099-1103.

What is claimed is:

1. A composition comprising an isolated, purified or recombinant nucleic acid molecule comprising a polynucleotide sequence selected from the group consisting of:
a) a contiguous span of at least 200 nucleotides of SEQ ID No 1 or the complement thereof, wherein said contiguous span comprises at least one of the following nucleotide positions of SEQ ID No 1: 1-97921, 98517-103471, 103603-108222, 108390-109221, 109324-114409, 114538-115723, 115957-122102, 122225-126876, 127033-157212, 157808-240825;
b) a contiguous span of at least 15 nucleotides of SEQ ID No 2 or the complement thereof;
c) a contiguous span of at least 15 nucleotides of anyone of SEQ ID Nos 1 and 2 or the complements thereof, wherein said span includes a PG-3-related biallelic marker selected from the group consisting of A1 to A5 and A8 to A80, and the complements thereof;
d) a polynucleotide consisting essentially of a sequence selected from the following sequences: P1 to P4 and P6 to P80, and the complementary sequences thereto;
e) a polynucleotide consisting essentially of a sequence selected from the following sequences: D1 to D4, D6 to D80, E1 to E4, and E6 to E80;
f) a polynucleotide consisting essentially of a sequence selected from the following sequences: B1 to B52 and C1 to C52; and
g) a polynucleotide which encodes a polypeptide comprising a contiguous span of at least 6 amino acids of SEQ ID No 3.

2. A composition comprising an isolated recombinant vector, wherein said vector comprises a polynucleotide according to claim 1.

3. A composition comprising an isolated host cell, wherein said host cell contains either the recombinant vector of claim 2 or a PG-3 gene operably linked to a heterologous regulatory element.

4. A non-human host animal comprising either the recombinant vector of claim 2 or a PG-3 gene disrupted by homologous recombination with a knock out vector, comprising a polynucleotide according to claim 1.

5. A composition comprising an isolated, purified, or recombinant polypeptide comprising a 2 contiguous span of at least 6 amino acids of SEQ ID No 3.

6. A composition comprising an isolated or purified antibody capable of selectively binding to an epitope-containing fragment of the polypeptide of claim 5.

7. A method of genotyping comprising determining the identity of a nucleotide at a PG-3-related biallelic marker or the complement thereof in a biological sample.

8. A method of genotyping according to claim 7, wherein said biological sample is from a single individual.

9. A method of genotyping according to claim 7, further comprising amplifying a portion of said sequence comprising said biallelic marker prior to said determining step.

10. A method of estimating the frequency of an allele of a PG-3-related biallelic marker in a population comprising:
a) genotyping individuals from said population for said biallelic marker according to the method of claim 7; and
b) determining the proportional representation of said biallelic marker in said population.

11. A method of detecting an association between a genotype and a trait, comprising the steps of:
a) determining the frequency of at least one PG-3-related biallelic marker in a trait positive population according to the method of claim 10;
b) determining the frequency of at least one PG-3-related biallelic marker in a control population according to the method of claim 10; and
c) determining whether a statistically significant association exists between said genotype and said trait.

12. A method of estimating the frequency of a haplotype for a set of biallelic markers in a population, comprising:
a) genotyping at least one PG-3-related biallelic marker according to claim 8 for each individual in said population;
b) genotyping a second biallelic marker by determining the identity of the nucleotides at said second biallelic marker for both copies of said second biallelic marker present in the genome of each individual in said population; and
c) applying a haplotype determination method to the identities of the nucleotides determined in steps a) and b) to obtain an estimate of said frequency.

13. A method of detecting an association between a haplotype and a trait, comprising the steps of:
a) estimating the frequency of at least one haplotype in a trait positive population according to the method of claim 12;
b) estimating the frequency of said haplotype in a control population according to the method of claim 12; and
c) determining whether a statistically significant association exists between said haplotype and said trait.
